(19) United States
(12) Patent Application Publication
(10) Pub. No.: US 2014/0155376 A1
(43) Pub. Date: Jun. 5, 2014

(54) PYRROLOPYRAZINE KINASE INHIBITORS

(71) Applicant: Hoffmann-La Roche Inc., Nutley, NJ (US)

(72) Inventors: Robert Than Hendricks, San Carlos, CA (US); Johannes Hermann, Morris Plains, NJ (US); Saul Jaime-Figueroa, Morris Plains, NJ (US); Rama Kondru, Morris Plains, NJ (US); Yan Lou, Clifton, NJ (US); Stephen Lynch, Westfield, NJ (US); Timothy D. Owens, Jersey City, NJ (US); Michael Soth, Glen Rock, NJ (US); Calvin Yee, Nutley, NJ (US)

(73) Assignee: Hoffmann-La Roche Inc., Nutley, NJ (US)

(21) Appl. No.: 14/172,144

(22) Filed: Feb. 4, 2014

Related U.S. Application Data
(63) Continuation of application No. 13/110,062, filed on May 18, 2011.
(60) Provisional application No. 61/475,281, filed on Apr. 14, 2011.
(60) Provisional application No. 61/346,503, filed on May 20, 2010.

(51) Int. Cl. C07D 487/04; C07D 519/00

(52) U.S. Cl. CPC C07D 487/04; C07D 519/00
USPC 514/210.18; 544/350; 544/58.2; 514/249; 514/210.21; 514/228.5

(57) ABSTRACT

The present invention relates to the use of novel pyrrolopyrazine derivatives of Formula I,

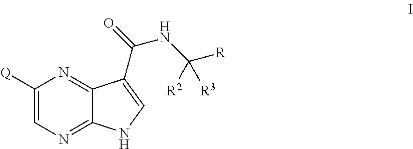

wherein the variables Q and R, R², and R³ are defined as described herein, which inhibit JAK and SYK and are useful for the treatment of auto-immune and inflammatory diseases.

### CROSS-REFERENCE TO RELATED APPLICATIONS

This application is entitled to the benefit of U.S. provisional patent application Ser. No. 61/475,281 filed on Apr. 14, 2011, and U.S. provisional patent application Ser. No. 61/346,503 filed on May 20, 2010.

This application is related to U.S. application Ser. No. 12/378,837, filed on Feb. 20, 2009, Ser. No. 12/378,869, filed on Feb. 20, 2009, 12/378,971, filed on Feb. 20, 2009, Ser. No. 12/378,977, filed on Feb. 20, 2009, and Ser. No. 12/378,978, filed on Feb. 20, 2009, each of which is expressly incorporated herein by reference.

### FIELD OF THE INVENTION

The present invention relates to the use of novel pyrrolopyrazine derivatives which are JAK and SYK inhibitors and selectively inhibit JAK3 and are useful for the treatment of auto-immune and inflammatory diseases.

### BACKGROUND OF THE INVENTION

Protein kinases constitute one of the largest families of human enzymes and regulate many different signaling processes by adding phosphate groups to proteins; particularly tyrosine kinases phosphorylate proteins on the alcohol moiety of tyrosine residues. The tyrosine kinase family includes members that control cell growth, migration, and differentiation. Abnormal kinase activity has been implicated in a variety of human diseases including cancers, autoimmune and inflammatory diseases. Since protein kinases are among the key regulators of cell signaling they provide a means to modulate cellular function with small molecule inhibitors of kinase activity and thus make good drug design targets. In addition to treatment of kinase-mediated disease processes, selective and efficacious inhibitors of kinase activity are also useful for investigation of cell signaling processes and identification of other cellular targets of therapeutic interest.

The JAKs (JAnus Kinases) are a family of cytoplasmic protein tyrosine kinases including JAK1, JAK2, JAK3 and TYK2. Each of the JAKs is preferentially associated with the intracytoplasmic portion of discrete cytokine receptors (Annu. Rev. Immunol. 16 (1998), pp. 293-322). The JAKs are activated following ligand binding and initiate signaling by phosphorylating cytokine receptors that, per se, are devoid of intrinsic kinase activity. This phosphorylation creates docking sites on the receptors for other molecules known as STAT proteins (signal transducers and activators of transcription) and the phosphorylated JAKs bind various STAT proteins. STAT proteins, or STATs, are DNA binding proteins activated by phosphorylation of tyrosine residues, and function both as signaling molecules and transcription factors and ultimately bind to specific DNA sequences present in the promoters of cytokine-responsive genes (Leonard et al., (2000), J. Allergy Clin. Immunol. 105:877-888).

JAK/STAT signaling has been implicated in the mediation of many abnormal immune responses such as allergies, asthma, autoimmune diseases such as transplant (allograft) rejection, rheumatoid arthritis, amyotrophic lateral sclerosis and multiple sclerosis, as well as in solid and hematologic malignancies such as leukemia and lymphomas.

Thus, the JAKs and STATs are components of multiple potertially intertwined signal-transduction pathways (Oncogene 19 (2000), pp. 5662-5679), which indicates the difficulty of specifically targeting one element of the JAK-STAT pathway without interfering with other signal transduction pathways.

The JAK kinases, including JAK3, are abundantly expressed in primary leukemic cells from children with acute lymphoblastic leukemia, the most common form of childhood cancer, and studies have correlated STAT activation in certain cells with signals regulating apoptosis (Demoulin et al., (1996), Mol. Cell. Biol. 16:4710-6; Jurlander et al., (1997), Blood. 89:4146-52; Kaneko et al., (1997), Clin. Exp. Immun. 109:185-193; and Nakamura et al., (1996), J. Biol. Chem. 271: 19483-8). They are also known to be important to lymphocyte differentiation, function and survival. JAK3 in particular plays an essential role in the function of lymphocytes, macrophages, and mast cells. Given the importance of this JAK kinase, compounds which modulate the JAK pathway, including those selective for JAK3, can be useful for treating diseases or conditions where the function of lymphocytes, macrophages, or mast cells is involved (Kudlacz et al., (2004) Am. J. Transplant 4:51-57; Changelian (2003) Science 302:875-878). Conditions in which targeting of the JAK pathway or modulation of the JAK kinases, particularly JAK3, are contemplated to be therapeutically useful include, leukemia, lymphoma, transplant rejection (e.g., pancreas islet transplant rejection, bone marrow transplant applications (e.g., graft-versus-host disease), autoimmune diseases (e.g., diabetes), and inflammation (e.g., asthma, allergic reactions). Conditions which can benefit for inhibition of JAK3 are discussed in greater detail below.

However, in contrast to the relatively ubiquitous expression of JAK1, JAK2 and Tyk2, JAK3 has a more restricted and regulated expression. Whereas some JAKs (JAK1, JAK2, Tyk2) are used by a variety of cytokine receptors, JAK3 is used only by cytokines that contain a γc in their receptor. JAK3, therefore, plays a role in cytokine signaling for cytokines which receptor was shown to date to use the common gamma chain; IL-2, IL-4, IL-7, IL-9, IL-15 and IL-21. JAK1 interacts with, among others, the receptors for cytokines IL-2, IL-4, IL-7, IL-9 and IL-21, while JAK2 interacts with, among others, the receptors for IL-9 and TNF-alpha. Upon the binding of certain cytokines to their receptors (e.g., IL-2, IL-4, IL-7, IL-9, IL-15 and IL-21), receptor oligomerization occurs, resulting in the cytoplasmic tails of associated JAK kinases being brought into proximity and facilitating the trans-phosphorylation of tyrosine residues on the JAK kinase. This trans-phosphorylation results in the activation of the JAK kinase.

Animal studies have suggested that JAK3 not only plays a critical role in B and T lymphocyte maturation, but that JAK3 is constitutively required to maintain T cell function. Modulation of immune activity through this novel mechanism can prove useful in the treatment of T cell proliferative disorders such as transplant rejection and autoimmune diseases.

In particular, JAK3 has been implicated in a variety of biological processes. For example, the proliferation and survival of murine mast cells induced by IL-4 and IL-9 have been shown to be dependent on JAK3- and gamma chain-signaling (Suzuki et al., (2000), Blood 96:2172-2180). JAK3 also plays a crucial role in IgE receptor-mediated mast cell degranulation responses (Malaviya et al., (1999), Biochem. Biophys. Res. Commun. 257:807-813), and inhibition of JAK3 kinase has been shown to prevent type I hypersensitivity reactions, including anaphylaxis (Malaviya et al., (1999), J. Biol. Chem. 274:27028-27038). JAK3 inhibition has also been shown to result in immune suppression for allograft rejection (Kirken, (2001), Transpl. Proc. 33:3268-3270). JAK3 kinases have also been implicated in the mechanism involved in early and late stages of rheumatoid arthritis (Muller-Ladner et al., (2000), J. Immunal. 164:3894-3901); familial amyotrophic lateral sclerosis (Trieu et al., (2000), Biochem Biophys. Res. Commun. 267:22-25); leukemia (Sudbeck et al., (1999), Clin. Cancer Res. 5:1569-1582); mycosis fungoides, a form of T-cell lymphoma (Nielsen et al., (1997), Prac. Natl. Acad. Sci. USA 94:6764-6769); and abnormal cell growth (Yu et al., (1997), J. Immunol. 159:5206-5210; Catlett-Falcone et al., (1999), Immunity 10:105-115).

JAK3 inhibitors are useful therapy as immunosuppressive agents for organ transplants, xeno transplantation, lupus, multiple sclerosis, rheumatoid arthritis, psoriasis, Type I diabetes and complications from diabetes, cancer, asthma, atopic dermatitis, autoimmune thyroid disorders, ulcerative colitis, Crohn's disease, Alzheimer's disease, Leukemia and other indications where immunosuppression would be desirable.

Non-hematopoietic expression of JAK3 has also been reported, although the functional significance of this has yet to be clarified (J. Immunol. 168 (2002), pp. 2475-2482). Because bone marrow transplants for SCID are curative (Blood 103 (2004), pp. 2009-2018), it seems unlikely that JAK3 has essential non-redundant functions in other tissues or organs. Hence, in contrast with other targets of immunosuppressive drugs, the restricted distribution of JAK3 is appealing. Agents that act on molecular targets with expression limited to the immune system might lead to an optimal efficacy:toxicity ratio. Targeting JAK3 would, therefore, theoretically offer immune suppression where it is needed (i.e. on cells actively participating in immune responses) without resulting in any effects outside of these cell populations. Although defective immune responses have been described in various STAT^{-/-} strains (J. Investig. Med. 44 (1996), pp. 304-311; Curr. Opin. Cell Biol. 9 (1997), pp. 233-239), the ubiquitous distribution of STATs and the fact that those molecules lack enzymatic activity that could be targeted with small-molecule inhibitors has contributed to their non-selection as key targets for immunosuppression.

SYK (Spleen Tyrosine Kinase) is a non-receptor tyrosine kinase that is essential for B-cell activation through BCR signaling. SYK become activated upon binding to phosphoryated BCR and thus initiates the early signaling events following BCR activation. Mice deficient in SYK exhibit an early block in B-cell development (Cheng et al. Nature 378:303, 1995; Turner et al. Nature 378:298, 1995). Therefore inhibition of SYK enzymatic activity in cells is proposed as a treatment for autoimmune disease through its effects on autoantibody production.

In addition to the role of SYK in BCR signaling and B-cell activation, it also plays a key role in FcεRI mediated mast cell degranulation and eosinophil activation. Thus, SYK is implicated in allergic disorders including asthma (reviewed in Wong et al. Expert Opin Investig Drugs 13:743, 2004). SYK binds to the phosphorylated gamma chain of FcεRI via its SH2 domains and is essential for downstream signaling (Taylor et al. Mol. Cell. Biol. 15:4149, 1995). SYK deficient mast cells demonstrate defective degranulation, arachidonic acid and cytokine secretion (Costello et al. Oncogene 13:2595, 1996). This also has been shown for pharmacologic agents that inhibit SYK activity in mast cells (Yamamoto et al. J Pharmacol Exp Ther 306:1174, 2003). Treatment with SYK antisense oligonucleotides inhibits antigen-induced infiltration of eosinophils and neutrophils in an animal model of asthma (Sterton et al. J Immunol 169:1028, 2002). SYK deficient eosinophils also show impaired activation in response to Fc R stimulation (Lach-Trifilieffe et al. Blood 96:2506, 2000). Therefore, small molecule inhibitors of SYK will be useful for treatment of allergy-induced inflammatory diseases including asthma.

In view of the numerous conditions that are contemplated to benefit by treatment involving modulation of the JAK and/or SYK pathways it is immediately apparent that new compounds that modulate JAK and/or SYK pathways and methods of using these compounds should provide substantial therapeutic benefits to a wide variety of patients. Provided herein are novel pyrrolopyrazine derivatives for use in the treatment of conditions in which targeting of the JAK and/or SYK pathways or inhibition of JAK or SYK kinases, particularly JAK3, and are therapeutically useful for the treatment of auto-immune and inflammatory diseases.

### SUMMARY OF THE INVENTION

The novel pyrrolopyrazine derivatives provided herein selectively inhibit JAK3 and are useful for the treatment of auto-immune and inflammatory diseases. The compounds of the invention modulate the JAK and/or SYK pathways and are useful novel pyrrolopyrazine derivatives for the treatment of auto-immune and inflammatory diseases, wherein preferred compounds selectively inhibit JAK3. For example, the compounds of the invention may inhibit JAK3 and SYK, wherein preferred compounds are selective for JAK3 of the JAK kinases and are useful novel pyrrolopyrazine derivatives for the treatment of auto-immune and inflammatory diseases. The amide linker at the 7-position of the 5H-pyrrolo[2,3-b]pyrazines affords the compounds of formula I and I′ unexpected increased potency in inhibition of JAK and Syk kinases compared to 5H-pyrrolo[2,3-b]pyrazines with other moieties at that position. Furthermore, the compounds of the invention may inhibit JAK3 and JAK2, wherein preferred compounds are selective for JAK3 of the JAK kinases, and are useful novel pyrrolopyrazine derivatives for the treatment of auto-immune and inflammatory diseases. Similarly, the compounds of the invention may inhibit JAK3 and JAK1, wherein preferred compounds are selective for JAK3 of the JAK kinases, and are useful novel pyrrolopyrazine derivatives for the treatment of auto-immune and inflammatory diseases.

The application provides a compound of Formula I

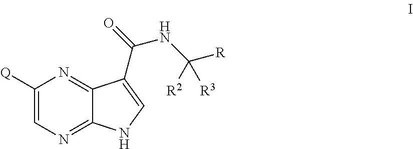

wherein:
R is H, cyano, lower alkyl, R′ or

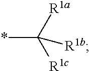

R′ is cycloalkyl, heterocycloalkyl, heteroaryl, or phenyl, wherein each is optionally substituted with one or more R″;
   each R″ is independently halo, hydroxy, cyano, lower alkyl, lower haloalkyl, lower alkoxy, lower hydroxyalkyl, cycloalkyl, C(═O)R′″, or S(═O)₂R′″;
      each R′″ is independently OH or lower alkyl;
R^{1a} and R^{1b} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R′″, S(═O)₂R′″ or CH₂S(═O)₂R′″;
R^{1c} is phenyl, cycloalkyl, heterocycloalkyl, or heteroaryl, optionally substituted with one or more R^{1d};
   each R^{1d} is independently hydroxy, halo, lower alkyl, lower hydroxyalkyl, lower halo alkyl, or lower alkoxy;
      R² is H, hydroxy lower alkyl, lower haloalkyl, or lower alkyl;
      R³ is H, hydroxy, cyano, cyano lower alkyl, or R^{3′};
each R^{3′} is independently lower alkyl, hydroxy lower alkyl, lower alkoxy, lower haloalkyl, lower haloalkoxy, phenyl lower alkyl, cycloalkyl or cycloalkyl lower alkyl, each optionally substituted with one or more R^{3″};
   each R^{3″} is independently lower alkyl, halo, hydroxy, lower alkoxy, lower haloalkyl, lower hydroxy alkyl, oxo, amino, cyano, cyano lower alkyl, S(═O)₂R^{3′″}, C(═O)R^{3′″}, cycloalkyl, heterocycloalkyl, heteroaryl, or heterocycloalkenyl;
   each R^{3′″} is independently H, hydroxy or lower alkyl;

### Q is Q², Q³, or Q⁴;

Q² is heterocycloalkyl, cycloalkyl, cycloalkenyl, heterocycloalkyl phenyl, heteroaryl, biaryl, or heterobiaryl, optionally substituted with one or more Q^{2a};
   Q^{2a} is Q^{2b} or Q^{2c};
      each Q^{2b} is independently halogen, oxo, hydroxy, —CN, —SCH₃, —S(O)₂CH₃, or —S(═O)CH₃;
      each Q^{2c} is independently Q_{2d} or Q^{2e};
      or two Q^{2a} come together to form a bicyclic ring system, optionally substituted with one or more Q^{2b} or Q^{2c};
         each Q^{2d} is independently —O(Q^{2e}), —S(═O)₂(Q^{2e}), C(═O)N(Q^{2e})₂, —S(O)₂(Q^{2e}), —C(═O)(Q^{2e}, —C(═O)O(Q^{2e}, —N(Q^{2e})C(═O)(Q^{2e}), —N(Q^{2e})C(═O)O(Q^{2e}, or —N(Q^{2e})C(═O)N(Q^{2e})₂;
         each Q^{2e} is independently H or Q^{2e′};
         each Q^{2e′} is independently lower alkyl, phenyl, benzyl, 5,6,7,8-Tetrahydro-naphthalene, lower haloalkyl, lower alkoxy, cycloalkyl, cycloalkenyl, heterocycloalkyl, spirocyclic heterocycloalkyl, or heteroaryl, optionally substituted with one or more Q^{2f};
         each Q^{2f} is independently Q^{2g} or Q^{2h};
         each Q^{2g} is independently halogen, hydroxy, cyano, oxo, —S(═O)₂(Q²ⁱ ), —S(═O)₂N(Q^{2i′})₂, —C(═O)OH, C(═O)N(Q^{2i′})₂, or —C(═O)(Q^{2i′});
         each Q^{2h} is independently lower alkyl, lower alkenyl, lower haloalkyl, lower alkoxy, amino, phenyl, benzyl, cycloalkyl, heterocycloalkyl, or heteroaryl, optionally substituted with one or more Q²ⁱ; and
         each Q²ⁱ is independently halogen, hydroxy, cyano, lower alkyl, lower haloalkyl, or lower alkoxy;
         each Q^{2i′} is independently H or lower alkyl;
Q³ is —O-Q^{3a}, —S-Q^{3a}, —C(═O)(Q^{3a}), —O(CH₂)ₘC(═O)(Q^{3a}), —S(═O)(Q^{3a}), —S(═O)₂(Q^{3a}), —N(Q^{3a})₂, —N(Q^{3a})S(═O)₂(Q^{3a}), —N(Q^{3a})C(═O)(Q^{3a}), —C(═O)N(Q^{3a})₂, N(Q^{3a})C(═O)N(Q^{3a})₂ or —N(Q^{3a})(CH₂)ₘC(═O)N(Q^{3a})₂;
   each Q^{3a} is independently Q^{3b} or Q^{3c};
   each m is independently 0, 1, or 2;
      each Q^{3b} is independently H;
      each Q^{3c} is independently lower alkyl, lower haloalkyl, phenyl, 5,6,7,8-Tetrahydro-naphthalene, naphthalene, 2,2-Dimethyl-2,3-dihydro-benzofuranyl, indanyl, indenyl, indolyl, cycloalkyl, heterocycloalkyl, or heteroaryl, optionally substituted with one or more Q^{3d}; and
         each Q^{3d} is independently Q^{3e} or Q^{3f};
         each Q^{3e} is independently halogen, oxo, cyano, hydroxy, —NHS(═O)₂(Q^{3f}), —NHC(═O)(Q^{3f}), NHC(═O)N(Q^{3f})₂, or N(Q³)₂;
         each Q^{3f} is independently H or Q^{3f};
         each Q^{3f} is independently lower alkyl, lower alkoxy, lower haloalkyl, phenyl, benzyl, cycloalkyl, heterocycloalkyl, or heteroaryl, optionally substituted with one or more Q^{3g}; and
         each Q^{3g} is independently halogen, hydroxy, lower alkyl, lower hydroxyalkyl, lower haloalkyl, or lower alkoxy;
Q⁴ is Q^{4a} or Q^{4b};
   Q^{4a} is hydroxy, halogen, or cyano;
   Q^{4b} is lower alkyl, lower alkoxy, lower alkynyl, lower alkenyl, lower hydroxyalkyl, amino, or lower haloalkyl, optionally substituted with one or more Q^{4c};
   each Q^{4c} is independently Q^{4d} or Q^{4e};
      each Q^{4d} is independently halogen, hydroxy, or cyano;
      each Q^{4e} is independently lower alkyl, lower haloalkyl, lower alkoxy, amino, cycloalkyl, phenyl, heterocycloalkyl, or heteroaryl, optionally substituted with one or more Q^{4f};
         each Q^{4f} is independently hydroxy, halogen, lower alkyl, lower alkenyl, oxo, lower haloalkyl, lower alkoxy, lower hydroxyalkyl or amino;
            with the proviso that the compound of Formula I is not 2-Thiophen-2-yl-5H-pyrrolo[2,3-b]pyrazine-7-carboxylic acid isopropylamide; 2-Cyclopropyl-5H-pyrrolo[2,3-b]pyrazine-7-carboxylic acid (4-hydroxy-3,3-dimethyl-butyl)-amide; 2-[1-(7-Isopropylcarbamoyl-5H-pyrrolo[2,3-b]pyrazin-2-yl)-piperidin-3-yl]-propionic acid tert-butyl ester; 2-Cyclopropyl-5H-pyrrolo[2,3-b]pyrazine-7-carboxylic acid tert-butylamide; 2-Cyclohexyl-5H-pyrrolo[2,3-b]pyrazine-7-carboxylic acid isopropylamide; 2-Cyclohex-1-enyl-5H-pyrrolo[2,3-b]pyrazine-7-carboxylic acid isopropylamide; 2-Chloro-5H-pyrrolo[2,3-b]pyrazine-7-carboxylic acid isopropylamide; 2-Isopropyl-5H-pyrrolo[2,3-b]pyrazine-7-carboxylic acid isopropylamide; 2-Isopropenyl-5H-pyrrolo[2,3-b]pyrazine-7-carboxylic acid isopropylamide; 2-(Cyclopentyl-methyl-amino)-5H-pyrrolo[2,3-b]pyrazine-7-carboxylic acid isopropylamide; [1-(7-Isopropylcarbamoyl-5H-pyrrolo[2,3-b]pyrazin-2-yl)-piperidin-3-yl]-methyl-carbamic acid tert-butyl ester; 2-(3-Methylamino-piperidin-1-yl)-5H-pyrrolo[2,3-b]pyrazine-7-carboxylic acid isopropylamide; 2-(Cyclopentyl-methyl-amino)-5H-pyrrolo[2,3-b]pyrazine-7-carboxylic acid isopropylamide, 2-Chloro-5H-pyrrolo[2,3-b]pyrazine-7-carboxylic acid isopropylamide, 2-Isopropenyl-5H-pyrrolo[2,3-b]pyrazine-7-carboxylic acid isopropylamide, 2-Isopropyl-5H-pyrrolo[2,3-b]pyrazine-7-carboxylic acid isopropylamide, 2-Cyclohex-1-enyl-5H-pyrrolo[2,3-b]pyrazine-7-carboxylic acid isopropylamide, 2-Cyclopropyl-5H-pyrrolo[2,3-b]pyrazine-7-carboxylic acid (3-hydroxy-2,2-dimethyl-propyl)-amide, 2-Cyclopropyl-5H-pyrrolo[2,3-b]pyrazine-7-carboxylic acid ((S)-2-hydroxy-1,2-dimethyl-propyl)-amide, 2-Cyclopropyl-5H-pyrrolo[2,3-b]pyrazine-7-carboxylic acid tert-butylamide, 2-Cyclohexyl-5H-pyrrolo[2,3-b]pyrazine-7-carboxylic acid isopropylamide, 2-Thiophen-2-yl-5H-pyrrolo[2,3-b]pyrazine-7-carboxylic acid (3-hydroxy-2,2-dimethyl-propyl)-amide, [1-(7-Isopropylcarbamoyl-5H-pyrrolo[2,3-b]pyrazin-2-yl)-piperidin-3-yl]-methyl-carbamic acid tert-butyl ester, 2-(3-Methylamino-piperidin-1-yl)-5H-pyrrolo[2,3-b]pyrazine-7-carboxylic acid isopropylamide; compound with trifluoro-acetic acid, [1-(7-Isopropylcarbamoyl-5H-pyrrolo[2,3-b]pyrazin-2-yl)-piperidin-3-yl]-methyl-carbamic acid tert-butyl ester, or 2-(3-Methylamino-piperidin-1-yl)-5H-pyrrolo[2,3-b]pyrazine-7-carboxylic acid isopropylamide; compound with trifluoro-acetic acid; or a pharmaceutically acceptable salt thereof.

The application provides a method for treating an inflammatory or autoimmune condition comprising administering to a patient in need thereof a therapeutically effective amount of the compound of formula I.

The application provides a pharmaceutical composition comprising the compound of formula I, admixed with at least one pharmaceutically acceptable carrier, excipient or diluent.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable (e.g., R, R′, or Q) occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “------” drawn through a bond each refer to the point of attachment of a functional group or other chemical moiety to the rest of the molecule of which it is a part. Thus, for example:
MeC(═O)OR⁴ wherein

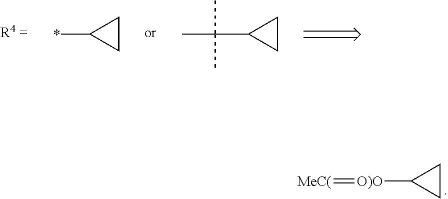

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen or a substituent.

The phrase “come together to form a bicyclic ring system” as used herein means join to form a bicyclic ring system, wherein each ring may be made up of either 4-7 carbon atoms or 4-7 carbon and heteroatoms, and may be saturated or unsaturated.

The phrase “come together to form a spirocyclic ring system” as used herein means two substituents on a singe carbon atom join together to form a spirocyclic ring system, wherein the formed ring may be made up of either 3-7 carbon atoms or 3-7 carbon and heteroatoms, and may be saturated or unsaturated.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” “cycloalkylalkyl” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term—(ar)alkyl refers to either an unsubstituted alkyl or an aralkyl group. The term (hetero)aryl or (het)aryl refers to either an aryl or a heteroaryl group.

Compounds of formula I may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistert with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl. The term “carbonyl” as used herein means a group of formula C(═O). The term “oxo” as used herein means a group of formula (═O), which may be attached to a carbon atom or heteroatom.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁-₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl”, “aryl alkyl”, or “aralkyl” are interpreted similarly except R′ is an aryl radical. The terms “heteroaryl alkyl” or “heteroarylalkyl” are interpreted similarly except R′ is optionally an aryl or a heteroaryl radical.

The term “haloalkyl” as used herein denotes a unbranched or branched chain alkyl group as defined above wherein 1, 2, 3 or more hydrogen atoms are substituted by a halogen. The term “lower haloalkyl” denotes a straight or branched chain hydrocarbon residue containing 1 to 6 carbon atoms, wherein 1, 2, 3 or more hydrogen atoms are substituted by a halogen. Examples are 1-fluoromethyl, 1-chloromethyl, 1-bromomethyl, 1-iodomethyl, difluoromethyl, trifluoromethyl, trichloromethyl, tribromomethyl, triiodomethyl, 1-fluoroethyl, 1-chloroethyl, 1-bromoethyl, 1-iodoethyl, 2-fluoroethyl, 2-chloroethyl, 2-bromoethyl, 2-iodoethyl, 2,2-dichloroethyl, 3-bromopropyl or 2,2,2-trifluoroethyl.

The term “alkylene” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an —O-alkyl wherein alkyl is C₁₋₁₀.

The term “hydroxyalkyl” as used herein denotes an alkyl radical as herein defined wherein one to three hydrogen atoms on different carbon atoms is/are replaced by hydroxyl groups.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term “cycloalkenyl” refers to a partially unsaturated carbocyclic containing 5 to 7 carbon atoms unless otherwise specified and having a carbon-carbon double bond within the ring. For example, C₅₋₆ cycloalkenyl refers to a cycloalkenyl group having from 5 to 6 member atoms. In certain embodiments cycloalkenyl groups have one carbon-carbon double bond within the ring. In other embodiments, cycloalkenyl groups have more than one carbon-carbon double bond within the ring. However, cycloalkenyl rings are not aromatic. Cycloalkenyl groups may be optionally substituted with one or more substituent. Examples of cycloalkenyl include, but are not limited to, cyclopentenyl and cyclohexenyl.

The term “halogen” or “halo” as used herein means fluorine, chlorine, bromine, or iodine.

The term “amino” as used herein encompasses —NR₂, wherein each R group is independently H or lower alkyl, wherein lower alkyl is as defined herein. Examples of amino groups include dimethyl amino, methyl amino and NH₂.

As used herein, the term “aryl” means a monocyclic or bicyclic (also referred to as “biaryl”), substituted or unsubstituted carbocyclic aromatic group. Examples of aryl groups are phenyl, naphthyl and the like.

The term “heteroaryl” as used herein means a monocyclic, or bicyclic (“heterobiaryl”), or tricyclic radical of 5 to 18 ring atoms having at least one aromatic ring containing four to eight atoms per ring, incorporating one or more N, O, or S heteroatoms, the remaining ring atoms being carbon, with the understanding that the attachment point of the heteroaryl radical will be on an aromatic ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, pyrrolyl, pyrazolyl, imidazolyl, indolyl oxazolyl, isoxazolyl, thiazolyl, isothiazolyl, triazolinyl, triazolyl, thiophenyl, furanyl, thiadiazolyl, and oxadiaxolinyl which can optionally be substituted with one or more, preferably one or two substituents selected from hydroxy, cyano, alkyl, alkoxy, thio, lower haloalkoxy, alkylthio, halo, haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties, also referred to as “hetero biaryl”, include, but are not limited to, quinolinyl, indazolyl, isoquinolinyl, benzofuryl, benzothiophenyl, benzoxazole, benzisoxazole, benzothiazole, pyrrolopyridinyl, pyrrolopyrazinyl, 1H-Pyrrolo[2,3-b]pyridine, and benzisothiazole.

The term “heterocycloalkyl”, “heterocyclyl” or “heterocycle” as used herein denotes a monovalent saturated cyclic radical, consisting of one or more rings, preferably one to two rings or three rings, of three to eight atoms per ring, incorporating one or more ring carbon atoms and one or more ring heteroatoms (chosen from N,O or S(═O)₀₋₂), wherein the point of attachment can be through either a carbon atom or a heteroatom, and which can optionally be independently substituted with one or more, preferably one or two or three substituents selected from hydroxy, oxo, cyano, lower alkyl, lower alkoxy, lower haloalkoxy, alkylthio, halo, haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, unless otherwise indicated. Examples of heterocyclic radicals include, but are not limited to, azetidinyl, pyrrolidinyl, hexahydroazepinyl, oxetanyl, tetrahydrofuranyl, tetrahydrothiophenyl, oxazolidinyl, thiazolidinyl, isoxazolidinyl, pyrrolidinyl, morpholinyl, piperazinyl, piperidinyl, isoindolinyl, dihydroisoquinolinyle, tetrahydropyranyl, tetrahydrocarbolinyl, imidazolinyl, thiomorpholinyl, and quinuclidinyl.

The phrase “organ rejection” includes acute allograft or xenograft rejection and chronic allograft or xenograft rejection in the setting of vascularized and/or non-vascularized (e.g. bone marrow, pancreatic islet cells) transplants.

### Inhibitors of JAK and Syk

The application provides a compound of Formula I

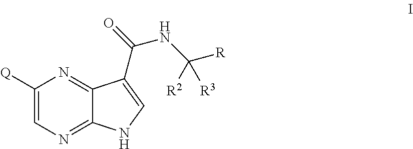

wherein:
R is H, cyano, lower alkyl, R′ or

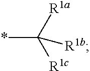

R′ is cycloalkyl, heterocycloalkyl, heteroaryl, or phenyl, wherein each is optionally substituted with one or more R″;
   each R″ is independently halo, hydroxy, cyano, lower alkyl, lower haloalkyl, lower alkoxy, lower hydroxyalkyl, cycloalkyl, C(═O)R′″, or S(═O)₂R′″;
      each R′″ is independently OH or lower alkyl;
R^{1a} and R^{1b} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R′″, S(═O)₂R′″ or CH₂S(═O)₂R′″;
R^{1c} is phenyl, cycloalkyl, heterocycloalkyl, or heteroaryl, optionally substituted with one or more R^{1d};
   each R^{1d} is independently hydroxy, halo, lower alkyl, lower hydroxyalkyl, lower halo alkyl, or lower alkoxy;
      R² is H, hydroxy lower alkyl, lower haloalkyl, or lower alkyl;
      R³ is H, hydroxy, cyano, cyano lower alkyl, or R^{3′};
each R^{3′} is independently lower alkyl, hydroxy lower alkyl, lower alkoxy, lower haloalkyl, lower haloalkoxy, phenyl lower alkyl, cycloalkyl or cycloalkyl lower alkyl, each optionally substituted with one or more R^{3″};
   each R^{3″} is independently lower alkyl, halo, hydroxy, lower alkoxy, lower haloalkyl, lower hydroxy alkyl, oxo, amino, cyano, cyano lower alkyl, S(═O)₂R^{3′″}, C(═O)R^{3′″}, cycloalkyl, heterocycloalkyl, heteroaryl, or heterocycloalkenyl;
   each R^{3′″} is independently H, hydroxy or lower alkyl;

### Q is Q², Q³, or Q⁴;

Q² is heterocycloalkyl, cycloalkyl, cycloalkenyl, heterocycloalkyl phenyl, heteroaryl, biaryl, or heterobiaryl, optionally substituted with one or more Q^{2a};
   Q^{2a} is Q^{2b} or Q^{2c};
      each Q^{2b} is independently halogen, oxo, hydroxy, —CN, —SCH₃, —S(O)₂CH₃, or —S(═O)CH₃;
      each Q^{2a} is independently Q
      or two Q^{2a} come together to form a bicyclic ring system, optionally substituted with one or more Q^{2b} or Q^{2c};
         each Q^{2d} is independently —O(Q^{2e}), —S(═O)₂(Q^{2e}), C(═O)N(Q^{2e})₂, —S(O)₂(Q^{2e}), —C(═O)(Q^{2e}), —C(═O)O(Q^{2e}), —N(Q^{2e})C(═O)(Q^{2e}), —N(Q^{2e})C(═O)O(Q^{2e}), or —N(Q^{2e})C(═O)N(Q^{2e})₂;
         each Q^{2e} is independently H or Q^{2e′};
         each Q^{2e′} is independently lower alkyl, phenyl, benzyl, 5,6,7,8-Tetrahydro-naphthalene, lower haloalkyl, lower alkoxy, cycloalkyl, cycloalkenyl, heterocycloalkyl, spirocyclic heterocycloalkyl, or heteroaryl, optionally substituted with one or more Q^{2f};
         each Q^{2f} is independently Q^{2g} or Q^{2h};
         each Q^{2g} is independently halogen, hydroxy, cyano, oxo, —S(═O)₂(Q^{2i′}), —S(═O)₂N(Q^{2i′})₂, —C(═O)OH, C(═O)N(Q^{2i′})₂, or —C(═O)(Q^{2i′});
         each Q^{2h} is independently lower alkyl, lower alkenyl, lower haloalkyl, lower alkoxy, amino, phenyl, benzyl, cycloalkyl, heterocycloalkyl, or heteroaryl, optionally substituted with one or more Q²ⁱ; and
         each Q²ⁱ is independently halogen, hydroxy, cyano, lower alkyl, lower haloalkyl, or lower alkoxy;
         each Q^{2i′} is independently H or lower alkyl;
Q³ is —O-Q^{3a}, —S-Q^{3a}, —C(═O)(Q^{3a}), —O(CH₂)ₘC(═O)(Q^{3a}), —S(═O)(Q^{3a}), —S(═O)₂(Q^{3a}), —N(Q^{3a})₂, —N(Q^{3a})S(═O)₂(Q^{3a}), —N(Q^{3a})C(═O)(Q^{3a}), —C(═O)N(Q^{3a})₂, N(Q^{3a})C(═O)N(Q^{3a})₂ or —N(Q^{3a})(CH₂)ₘC(═O)N(Q^{3a})₂;
   each Q^{3a} is independently Q^{3b} or Q^{3c};
   each m is independently 0, 1, or 2;
      each Q^{3b} is independently H;
      each Q^{3c} is independently lower alkyl, lower haloalkyl, phenyl, 5,6,7,8-Tetrahydro-naphthalene, naphthalene, 2,2-Dimethyl-2,3-dihydro-benzofuranyl, indanyl, indenyl, indolyl, cycloalkyl, heterocycloalkyl, or heteroaryl, optionally substituted with one or more Q^{3d}; and
         each Q^{3d} is independently Q^{3e} or Q^{3f};
         each Q^{3e} is independently halogen, oxo, cyano, hydroxy, —NHS(═O)₂(Q^{3f}), —NHC(═O)(Q^{3f}), NHC(═O)N(Q^{3f})₂, or N(Q^{3f})₂;
         each Q^{3f} is independently H or Q^{3f};
         each Q^{3f} is independently lower alkyl, lower alkoxy, lower haloalkyl, phenyl, benzyl, cycloalkyl, heterocycloalkyl, or heteroaryl, optionally substituted with one or more Q^{3g}; and
         each Q^{3g} is independently halogen, hydroxy, lower alkyl, lower hydroxyalkyl, lower haloalkyl, or lower alkoxy;
Q⁴ is Q^{4a} or Q^{4b};
   Q^{4a} is hydroxy, halogen, or cyano;
   Q^{4b} is lower alkyl, lower alkoxy, lower alkynyl, lower alkenyl, lower hydroxyalkyl, amino, or lower haloalkyl, optionally substituted with one or more Q^{4c};
   each Q^{4c} is independently Q^{4d} or Q^{4e};
      each Q^{4d} is independently halogen, hydroxy, or cyano;
      each Q^{4e} is independently lower alkyl, lower haloalkyl, lower alkoxy, amino, cycloalkyl, phenyl, heterocycloalkyl, or heteroaryl, optionally substituted with one or more Q^{4f};
         each Q^{4f} is independently hydroxy, halogen, lower alkyl, lower alkenyl, oxo, lower haloalkyl, lower alkoxy, lower hydroxyalkyl or amino;
            with the proviso that the compound of Formula I is not 2-Thiophen-2-yl-5H-pyrrolo[2,3-b]pyrazine-7-carboxylic acid isopropylamide; 2-Cyclopropyl-5H-pyrrolo[2,3-b]pyrazine-7-carboxylic acid (4-hydroxy-3,3-dimethyl-butyl)-amide; 2-[1-(7-Isopropylcarbamoyl-5H-pyrrolo[2,3-b]pyrazin-2-yl)-piperidin-3-yl]-propionic acid tert-butyl ester; 2-Cyclopropyl-5H-pyrrolo[2,3-b]pyrazine-7-carboxylic acid tert-butylamide; 2-Cyclohexyl-5H-pyrrolo[2,3-b]pyrazine-7-carboxylic acid isopropylamide; 2-Cyclohex-1-enyl-5H-pyrrolo[2,3-b]pyrazine-7-carboxylic acid isopropylamide; 2-Chloro-5H-pyrrolo[2,3-b]pyrazine-7-carboxylic acid isopropylamide; 2-Isopropyl-5H-pyrrolo[2,3-b]pyrazine-7-carboxylic acid isopropylamide; 2-Isopropenyl-5H-pyrrolo[2,3-b]pyrazine-7-carboxylic acid isopropylamide; 2-(Cyclopentyl-methyl-amino)-5H-pyrrolo[2,3-b]pyrazine-7-carboxylic acid isopropylamide; [1-(7-Isopropylcarbamoyl-5H-pyrrolo[2,3-b]pyrazin-2-yl)-piperidin-3-yl]-methyl-carbamic acid tert-butyl ester; 2-(3-Methylamino-piperidin-1-yl)-5H-pyrrolo[2,3-b]pyrazine-7-carboxylic acid isopropylamide; 2-(Cyclopentyl-methyl-amino)-5H-pyrrolo[2,3-b]pyrazine-7-carboxylic acid isopropylamide, 2-Chloro-5H-pyrrolo[2,3-b]pyrazine-7-carboxylic acid isopropylamide, 2-Isopropenyl-5H-pyrrolo[2,3-b]pyrazine-7-carboxylic acid isopropylamide, 2-Isopropyl-5H-pyrrolo[2,3-b]pyrazine-7-carboxylic acid isopropylamide, 2-Cyclohex-1-enyl-5H-pyrrolo[2,3-b]pyrazine-7-carboxylic acid isopropylamide, 2-Cyclopropyl-5H-pyrrolo[2,3-b]pyrazine-7-carboxylic acid (3-hydroxy-2,2-dimethyl-propyl)-amide, 2-Cyclopropyl-5H-pyrrolo[2,3-b]pyrazine-7-carboxylic acid ((S)-2-hydroxy-1,2-dimethyl-propyl)-amide, 2-Cyclopropyl-5H-pyrrolo[2,3-b]pyrazine-7-carboxylic acid tert-butylamide, 2-Cyclohexyl-5H-pyrrolo[2,3-b]pyrazine-7-carboxylic acid isopropylamide, 2-Thiophen-2-yl-5H-pyrrolo[2,3-b]pyrazine-7-carboxylic acid (3-hydroxy-2,2-dimethyl-propyl)-amide, [1-(7-Isopropylcarbamoyl-5H-pyrrolo[2,3-b]pyrazin-2-yl)-piperidin-3-yl]-methyl-carbamic acid tert-butyl ester, 2-(3-Methylamino-piperidin-1-yl)-5H-pyrrolo[2,3-b]pyrazine-7-carboxylic acid isopropylamide; compound with trifluoro-acetic acid, [1-(7-Isopropylcarbamoyl-5H-pyrrolo[2,3-b]pyrazin-2-yl)-piperidin-3-yl]-methyl-carbamic acid tert-butyl ester, or 2-(3-Methylamino-piperidin-1-yl)-5H-pyrrolo[2,3-b]pyrazine-7-carboxylic acid isopropylamide; compound with trifluoro-acetic acid; or a pharmaceutically acceptable salt thereof.

In one variation of formula I, either R² or R³ is methyl.

In one variation of formula I, either R² or R³ is lower alkyl and the other is H.

In one variation of formula I, Q is cycloalkyl, heterocycloalkyl, or heteroaryl, each optionally substituted with one or more Q^{2a} and either R² or R³ is methyl.

In one variation of formula I, R^{1a} is lower alkyl, hydroxy, lower haloalkyl, lower alkoxy, cyano, or lower hydroxyalkyl.

In one variation of formula I, R^{1a} is lower alkyl, hydroxy, lower haloalkyl, lower alkoxy, cyano, or lower hydroxyalkyl and Q is cycloalkyl, heterocycloalkyl, or heteroaryl, each optionally substituted with one or more Q^{2a}.

In one variation of formula I, R^{1a} is lower alkyl, hydroxy, lower haloalkyl, lower alkoxy, cyano, or lower hydroxyalkyl and either R² or R³ is methyl.

In one variation of formula I, R^{1a} is lower alkyl, hydroxy, lower haloalkyl, lower alkoxy, cyano, or lower hydroxyalkyl, Q is cycloalkyl, heterocycloalkyl, or heteroaryl, each optionally substituted with one or more Q^{2a} and either R² or R³ is methyl.

In one variation of formula I, R^{1b} is lower alkyl or lower haloalkyl.

In one variation of formula I, R^{1b} is lower alkyl or lower haloalkyl and R^{1a} is lower alkyl, hydroxy, lower haloalkyl, lower alkoxy, cyano, or lower hydroxyalkyl.

In one variation of formula I, R^{1b} is lower alkyl or lower haloalkyl and Q is cycloalkyl, heterocycloalkyl, or heteroaryl, each optionally substituted with one or more Q^{2a}.

In one variation of formula I, R^{1b} is lower alkyl or lower haloalkyl and either R² or R³ is methyl.

In one variation of formula I, R^{1b} is lower alkyl or lower haloalkyl, R^{1a} is lower alkyl, hydroxy, lower haloalkyl, lower alkoxy, cyano, or lower hydroxyalkyl, Q is cycloalkyl, heterocycloalkyl, or heteroaryl, each optionally substituted with one or more Q^{2a} and either R² or R³ is methyl.

In one variation of formula I, R^{1c} is H, hydroxy, or lower alkyl.

In one variation of formula I, R^{1c} is H, hydroxy, or lower alkyl and R^{1a} is lower alkyl, hydroxy, lower haloalkyl, lower alkoxy, cyano, or lower hydroxyalkyl.

In one variation of formula I, R^{1c} is H, hydroxy, or lower alkyl and Q is cycloalkyl, heterocycloalkyl, or heteroaryl, each optionally substituted with one or more Q^{2a}.

In one variation of formula I, R^{1c} is H, hydroxy, or lower alkyl and either R² or R³ is methyl.

In one variation of formula I, R^{1c} is H, hydroxy, or lower alkyl, R^{1a} is lower alkyl, hydroxy, lower haloalkyl, lower alkoxy, cyano, or lower hydroxyalkyl, Q is cycloalkyl, heterocycloalkyl, or heteroaryl, each optionally substituted with one or more Q^{2a} and either R² or R³ is methyl.

In one variation of formula I, R^{1a} and R^{1b} together form spirocycloalkyl or spiroheterocycloalkyl.

In one variation of formula I, Q is cyclopropyl, thienyl, or pyrazolyl.

In one variation of formula I, Q is cyclopropyl, thienyl, or pyrazolyl, each optionally substituted with one or more Q^{2e}.

The application provides the compounds of Formula I′,

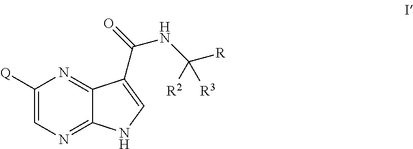

wherein:
R is H, cyano, R′ or

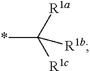

R′ is cycloalkyl, heterocycloalkyl, heteroaryl, or phenyl, wherein each is optionally substituted with one or more R″;
   R″ is halo, hydroxy, cyano, lower alkyl, lower haloalkyl, lower alkoxy, lower hydroxyalkyl, cycloalkyl, C(═O)R′″, or S(═O)₂R′″;
      R′″ is OH or lower alkyl;
R^{1a}, R^{1b}, and R^{1c} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, cycloalkyl, heterocycloalkyl, C(═O)R′″, or S(═O)₂R′″;
   R² is H or lower alkyl;
   R³ is H, hydroxy, cyano, cyano lower alkyl, or R^{3′};
R^{3′} is lower alkyl, hydroxy lower alkyl, lower alkoxy, lower haloalkyl, lower haloalkoxy, phenyl lower alkyl, or cycloalkyl lower alkyl, each optionally substituted with one or more R^{3″};
   each R^{3″} is independently lower alkyl, halo, hydroxy, lower alkoxy, lower haloalkyl, lower hydroxy alkyl, oxo, cyano, cyano lower alkyl, S(═O)₂R^{3′″}, C(═O)R^{3′″}, cycloalkyl, heterocycloalkyl, heteroaryl, or heterocycloalkenyl;
   R^{3′″} is H or lower alkyl;

### Q is Q², Q³, or Q⁴;

Q² is heterocycloalkyl, cycloalkyl, cycloalkenyl, heterocycloalkyl phenyl, heteroaryl, biaryl, or heterobiaryl, optionally substituted with one or more Q^{2a};
   Q^{2a} is Q^{2b} or Q^{2c};
      Q^{2b} is halogen, oxo, hydroxy, —CN, —SCH₃, —S(O)₂CH₃, or —S(═O)CH₃;
      Q^{2c} is Q^{2d} or Q^{2e};
      or two Q^{2a} come together to form a bicyclic ring system, optionally substituted with one or more Q^{2b} or Q^{2c};
         Q^{2d} is —O(Q^{2e}), —S(═O)₂(Q^{2e}), —C(═O)N(Q^{2e})₂, —S(═O)₂(Q^{2e}), or —C(═O)(Q^{2e}), —C(═O)O(Q^{2e}), —N(Q^{2e})C(═O)(Q^{2e}), —N(Q^{2e})C(═O)O(Q^{2e}), or —N(Q^{2e})C(═O)N(Q^{2e})₂;
         each Q^{2e} is independently H or Q^{2e′};
         each Q^{2e′} is independently lower alkyl, phenyl, benzyl, lower haloalkyl, lower alkoxy, cycloalkyl, cycloalkenyl, heterocycloalkyl, or heteroaryl, optionally substituted with one or more Q^{2f};
         Q^{2f} is Q^{2g} or Q^{2h};
         Q^{2g} is halogen, hydroxy, cyano, oxo, or —C(═O)(Q^{2h});
         Q^{2h} is lower alkyl, lower haloalkyl, lower alkoxy, amino, phenyl, benzyl, cycloalkyl, heterocycloalkyl, or heteroaryl, optionally substituted with one or more Q²ⁱ; and
         Q²ⁱ is halogen, hydroxy, cyano, lower alkyl, lower haloalkyl, or lower alkoxy;
Q³ is —O-Q^{3a}, —S-Q^{3a}, —C(═O)(Q^{3a}), —O(CH₂)ₘC(═O)(Q^{3a}), —S(═O)(Q^{3a}), —S(═O)₂(Q^{3a}), —N(Q^{3a})₂, —N(Q^{3a})S(═O)₂(Q^{3a}), —N(Q^{3a})C(═O)(Q^{3a}), —C(═O)N(Q^{3a})₂, N(Q^{3a})C(═O)N(Q^{3a})₂ or —N(Q^{3a})(CH₂)ₘC(═O)N(Q^{3a})₂;
   each Q^{3a} is independently Q^{3b} or Q^{3c};
   m is 0, 1, or 2;
      Q^{3b} is H;
      Q^{3c} is lower alkyl, lower haloalkyl, phenyl, cycloalkyl, heterocycloalkyl, or heteroaryl, optionally substituted with one or more Q^{3d}; and
         each Q^{3d} is independently Q^{3d} is Q^{3e} or Q^{3f};
         Q^{3c} is halogen or hydroxy;
         Q^{3f} is lower alkyl, lower alkoxy, lower haloalkyl, phenyl, cycloalkyl, heterocycloalkyl, or heteroaryl, optionally substituted with one or more Q^{3g}; and
         each Q^{3g} is independently halogen, hydroxy, lower alkyl, lower hydroxyalkyl, lower haloalkyl, or lower alkoxy;
Q⁴ is Q^{4a} or Q^{4b};
   Q^{4a} is hydroxy, halogen, or cyano;
   Q^{4b} is lower alkyl, lower alkoxy, lower alkynyl, lower alkenyl, lower hydroxyalkyl, amino, or lower haloalkyl, optionally substituted with one or more Q^{4c};
   Q^{4c} is Q^{4d} or Q^{4e};
      each Q^{4d} is independently halogen, hydroxy, or cyano;
      each Q^{4c} is independently lower alkyl, lower haloalkyl, lower alkoxy, amino, cycloalkyl, phenyl, heterocycloalkyl, or heteroaryl, optionally substituted with one or more Q^{4f};
         each Q^{4f} is independently hydroxy, halogen, lower alkyl, lower alkenyl, oxo, lower haloalkyl, lower alkoxy, lower hydroxyalkyl or amino;
            with the proviso that when Q is either cyclopropyl or thiophenyl, and R² and R³ are either H or methyl, and any two of R^{1a}, R^{1b}, and R^{1c} are either H or methyl, then the other is not H, hydroxy, or hydroxymethyl; and
            with the proviso that when Q is chloro, isopropyl, isopropenyl, piperidinyl, methyl-piperidin-3-yl-amine, methyl-piperidin-3-yl-carbamic acid tertbutyl ester, cyclohexyl, cyclopentyl-methyl-amino, or cyclohexenyl, and R² and R³ are either H or methyl, then R^{1a}, R^{1b}, and R^{1c} are not all H;
            or a pharmaceutically acceptable salt thereof.

The application provides a compound selected from the group consisting of:
2-Bromo-5H-pyrrolo[2,3-b]pyrazine-7-carboxylic acid (3-hydroxy-2,2-dimethyl-propyl)-amide;
2-Cyclopent-1-enyl-5H-pyrrolo[2,3-b]pyrazine-7-carboxylic acid ((S)-2-hydroxy-1,2-dimethyl-propyl)-amide;
2-Isopropenyl-5H-pyrrolo[2,3-b]pyrazine-7-carboxylic acid ((S)-2-hydroxy-1,2-dimethyl-propyl)-amide;
2-Dimethylamino-5H-pyrrolo[2,3-b]pyrazine-7-carboxylic acid (3-hydroxy-2,2-dimethyl-propyl)-amide;
2-Isopropyl-5H-pyrrolo[2,3-b]pyrazine-7-carboxylic acid ((S)-2-hydroxy-1,2-dimethyl-propyl)-amide;
2-Cyclopentyl-5H-pyrrolo[2,3-b]pyrazine-7-carboxylic acid ((S)-2-hydroxy-1,2-dimethyl-propyl)-amide;
2-Cyclohex-1-enyl-5H-pyrrolo[2,3-b]pyrazine-7-carboxylic acid ((S)-2-hydroxy-1,2-dimethyl-propyl)-amide;
2-Cyclopropyl-5H-pyrrolo[2,3-b]pyrazine-7-carboxylic acid isopropylamide;
2-Cyclopropyl-5H-pyrrolo[2,3-b]pyrazine-7-carboxylic acid (2-methoxy-1-methyl-ethyl)-amide;
2-Pyrrolidin-1-yl-5H-pyrrolo[2,3-b]pyrazine-7-carboxylic acid (3-hydroxy-2,2-dimethyl-propyl)-amide;
2-Cyclohexyl-5H-pyrrolo[2,3-b]pyrazine-7-carboxylic acid ((S)-2-hydroxy-1,2-dimethyl-propyl)-amide;
2-Cyclopropyl-5H-pyrrolo[2,3-b]pyrazine-7-carboxylic acid (3-hydroxy-1,1-dimethyl-butyl)-amide;
2-Cyclopropyl-5H-pyrrolo[2,3-b]pyrazine-7-carboxylic acid (2-cyano-ethyl)-amide;
2-(3,3-Dimethyl-pyrrolidin-1-yl)-5H-pyrrolo[2,3-b]pyrazine-7-carboxylic acid (3-hydroxy-2,2-dimethyl-propyl)-amide;
2-Phenylamino-5H-pyrrolo[2,3-b]pyrazine-7-carboxylic acid (3-hydroxy-2,2-dimethyl-propyl)-amide;
2-(Methylcarbamoylmethyl-amino)-5H-pyrrolo[2,3-b]pyrazine-7-carboxylic acid (3-hydroxy-2,2-dimethyl-propyl)-amide;
2-Cyclopropyl-5H-pyrrolo[2,3-b]pyrazine-7-carboxylic acid [2-hydroxy-1-(2-hydroxy-ethyl)-2-methyl-propyl]-amide;
2-Thiophen-2-yl-5H-pyrrolo[2,3-b]pyrazine-7-carboxylic acid ((S)-2-hydroxy-1,2-dimethyl-propyl)-amide;
2-Cyclopropyl-5H-pyrrolo[2,3-b]pyrazine-7-carboxylic acid (1-cyclopropyl-ethyl)-amide;
2-(2-Methyl-pyridin-4-yl)-5H-pyrrolo[2,3-b]pyrazine-7-carboxylic acid ((S)-2-hydroxy-1,2-dimethyl-propyl)-amide;
2-(6-Methyl-pyridin-3-yl)-5H-pyrrolo[2,3-b]pyrazine-7-carboxylic acid ((S)-2-hydroxy-1,2-dimethyl-propyl)-amide;
2-Cyclopropyl-5H-pyrrolo[2,3-b]pyrazine-7-carboxylic acid ((S)-sec-butyl)-amide;
2-Cyclopropyl-5H-pyrrolo[2,3-b]pyrazine-7-carboxylic acid ((S)-1,2,2-trimethyl-propyl)-amide;
2-Cyclopropyl-5H-pyrrolo[2,3-b]pyrazine-7-carboxylic acid ((S)-2-hydroxy-1-isopropyl-2-methyl-propyl)-amide;
2-Cyclopropyl-5H-pyrrolo[2,3-b]pyrazine-7-carboxylic acid ((S)-1,2-dimethyl-propyl)-amide;
2-Cyclopropyl-5H-pyrrolo[2,3-b]pyrazine-7-carboxylic acid (1-ethyl-propyl)-amide;
2-Cyclopropyl-5H-pyrrolo[2,3-b]pyrazine-7-carboxylic acid (2-dimethylamino-1-methyl-ethyl)-amide;
2-Cyclopropyl-5H-pyrrolo[2,3-b]pyrazine-7-carboxylic acid cyanomethyl-amide;
2-Cyclopropyl-5H-pyrrolo[2,3-b]pyrazine-7-carboxylic acid ((S)-1-ethyl-2-hydroxy-2-methyl-propyl)-amide;
2-Cyclopropyl-5H-pyrrolo[2,3-b]pyrazine-7-carboxylic acid ((R)-2-hydroxy-1,2-dimethyl-propyl)-amide;
2-Cyclopropyl-5H-pyrrolo[2,3-b]pyrazine-7-carboxylic acid ((1S,2S)-2-hydroxy-1,2-dimethyl-butyl)-amide;
2-Cyclopropyl-5H-pyrrolo[2,3-b]pyrazine-7-carboxylic acid ((S)-1-cyclohexyl-ethyl)-amide;
2-Cyclopropyl-5H-pyrrolo[2,3-b]pyrazine-7-carboxylic acid (3-cyano-propyl)-amide;
2-Cyclopropyl-5H-pyrrolo[2,3-b]pyrazine-7-carboxylic acid ((1S,2R)-2-hydroxy-1,2-dimethyl-butyl)-amide;
2-Trifluoromethyl-5H-pyrrolo[2,3-b]pyrazine-7-carboxylic acid (3-hydroxy-2,2-dimethyl-propyl)-amide;
2-Vinyl-5H-pyrrolo[2,3-b]pyrazine-7-carboxylic acid (3-hydroxy-2,2-dimethyl-propyl)-amide;
2-Cyclopropyl-5H-pyrrolo[2,3-b]pyrazine-7-carboxylic acid [(S)-1-(1H-pyrazol-3-yl)-ethyl]-amide;
2-Cyclopropyl-5H-pyrrolo[2,3-b]pyrazine-7-carboxylic acid ((1S,2S)-3-cyclopropyl-2-hydroxy-1,2-dimethyl-propyl)-amide;
2-Ethyl-5H-pyrrolo[2,3-b]pyrazine-7-carboxylic acid (3-hydroxy-2,2-dimethyl-propyl)-amide;
2-Cyclopropyl-5H-pyrrolo[2,3-b]pyrazine-7-carboxylic acid ((S)-1-hydroxymethyl-propyl)-amide;
2-Cyclopropyl-5H-pyrrolo[2,3-b]pyrazine-7-carboxylic acid ((R)-2-hydroxy-1-methyl-ethyl)-amide;
2-Cyclopropyl-5H-pyrrolo[2,3-b]pyrazine-7-carboxylic acid (3-hydroxy-1,1-dimethyl-propyl)-amide;
2-((1R,2R)-2-Methyl-cyclopropyl)-5H-pyrrolo[2,3-b]pyrazine-7-carboxylic acid (3-hydroxy-2,2-dimethyl-propyl)-amide;
2-Cyclopropyl-5H-pyrrolo[2,3-b]pyrazine-7-carboxylic acid ((R)-1-ethyl-2-hydroxy-2-methyl-propyl)-amide;
2-Cyclopropyl-5H-pyrrolo[2,3-b]pyrazine-7-carboxylic acid (2-hydroxy-1,1-dimethyl-ethyl)-amide;
2-((1R,2S)-2-Methyl-cyclopropyl)-5H-pyrrolo[2,3-b]pyrazine-7-carboxylic acid (3-hydroxy-2,2-dimethyl-propyl)-amide;
2-Cyclopropyl-5H-pyrrolo[2,3-b]pyrazine-7-carboxylic acid ((S)-1-hydroxymethyl-2,2-dimethyl-propyl)-amide;
2-Cyclopropyl-5H-pyrrolo[2,3-b]pyrazine-7-carboxylic acid ((1S,2R)-3-cyclopropyl-2-hydroxy-1,2-dimethyl-propyl)-amide;
2-Cyclopropyl-5H-pyrrolo[2,3-b]pyrazine-7-carboxylic acid [(S)-1-(1-hydroxy-1-methyl-ethyl)-pentyl]-amide;
2-Cyclopropyl-5H-pyrrolo[2,3-b]pyrazine-7-carboxylic acid (2-methoxy-2-methyl-propyl)-amide;
2-Cyclopropyl-5H-pyrrolo[2,3-b]pyrazine-7-carboxylic acid (2-hydroxy-1-hydroxymethyl-ethyl)-amide;
2-Cyclopropyl-5H-pyrrolo[2,3-b]pyrazine-7-carboxylic acid ((R)-1-hydroxymethyl-2,2-dimethyl-propyl)-amide;
2-Cyclopropyl-5H-pyrrolo[2,3-b]pyrazine-7-carboxylic acid ((1S,2R)-3,3,3-trifluoro-2-hydroxy-1,2-dimethyl-propyl)-amide;
2-Cyclopropyl-5H-pyrrolo[2,3-b]pyrazine-7-carboxylic acid ((S)-3,3,3-trifluoro-1,2,2-trimethyl-propyl)-amide;
2-Cyclopropyl-5H-pyrrolo[2,3-b]pyrazine-7-carboxylic acid (2,2-dimethyl-propyl)-amide;
2-Cyclopropyl-5H-pyrrolo[2,3-b]pyrazine-7-carboxylic acid ((R)-1-hydroxymethyl-2-methyl-propyl)-amide;
2-Cyclopropyl-5H-pyrrolo[2,3-b]pyrazine-7-carboxylic acid ((S)-2-hydroxy-1-methyl-ethyl)-amide;
2-Cyclopropyl-5H-pyrrolo[2,3-b]pyrazine-7-carboxylic acid ((R)-1-hydroxymethyl-propyl)-amide;
2-Cyclopropyl-5H-pyrrolo[2,3-b]pyrazine-7-carboxylic acid (3-methoxy-2,2-dimethyl-propyl)-amide;
2-Cyclopropyl-5H-pyrrolo[2,3-b]pyrazine-7-carboxylic acid ((R)-1-cyclohexyl-ethyl)-amide;
2-Cyclopropyl-5H-pyrrolo[2,3-b]pyrazine-7-carboxylic acid (2-cyano-1,2,2-trimethyl-ethyl)-amide;
2-Cyclopropyl-5H-pyrrolo[2,3-b]pyrazine-7-carboxylic acid ((R)-1,2,2-trimethyl-propyl)-amide;
2-Cyclopropyl-5H-pyrrolo[2,3-b]pyrazine-7-carboxylic acid ((1S,2S)-3,3,3-trifluoro-2-hydroxy-1,2-dimethyl-propyl)-amide;
2-Cyclopropyl-5H-pyrrolo[2,3-b]pyrazine-7-carboxylic acid ((R)-1-methoxymethyl-2,2-dimethyl-propyl)-amide;
2-Cyclopropyl-5H-pyrrolo[2,3-b]pyrazine-7-carboxylic acid ((S)-1-methoxymethyl-2,2-dimethyl-propyl)-amide;
2-Cyclopropyl-5H-pyrrolo[2,3-b]pyrazine-7-carboxylic acid ((R)-1-phenyl-ethyl)-amide;
2-Cyclopropyl-5H-pyrrolo[2,3-b]pyrazine-7-carboxylic acid ((S)-1-phenyl-ethyl)-amide;
2-Cyclopropyl-5H-pyrrolo[2,3-b]pyrazine-7-carboxylic acid (3-hydroxy-butyl)-amide;
2-Cyclopropyl-5H-pyrrolo[2,3-b]pyrazine-7-carboxylic acid (3-hydroxy-2-methyl-propyl)-amide;
2-Cyclopropyl-5H-pyrrolo[2,3-b]pyrazine-7-carboxylic acid (1-pyridin-2-yl-ethyl)-amide;
2-Cyclopropyl-5H-pyrrolo[2,3-b]pyrazine-7-carboxylic acid (3-hydroxy-1,2,2-trimethyl-propyl)-amide;
2-Pyridin-2-yl-5H-pyrrolo[2,3-b]pyrazine-7-carboxylic acid ((S)-1,2,2-trimethyl-propyl)-amide;
2-Cyclopropyl-5H-pyrrolo[2,3-b]pyrazine-7-carboxylic acid ((S)-1-cyclopropyl-2-hydroxy-2-methyl-propyl)-amide;
2-Cyclopropyl-5H-pyrrolo[2,3-b]pyrazine-7-carboxylic acid ((R)-1-cyclopropyl-2-hydroxy-2-methyl-propyl)-amide;
2-Cyclopropyl-5H-pyrrolo[2,3-b]pyrazine-7-carboxylic acid (1-cyclohexyl-propyl)-amide;
2-Cyclopropyl-5H-pyrrolo[2,3-b]pyrazine-7-carboxylic acid ((R)-1-cyanomethyl-2,2-dimethyl-propyl)-amide;
2-Cyclopropyl-5H-pyrrolo[2,3-b]pyrazine-7-carboxylic acid ((S)-1-cyanomethyl-2,2-dimethyl-propyl)-amide;
2-Cyclopropyl-5H-pyrrolo[2,3-b]pyrazine-7-carboxylic acid (cyclohexyl-cyclopropyl-methyl)-amide;
2-Cyclopropyl-5H-pyrrolo[2,3-b]pyrazine-7-carboxylic acid (2-hydroxy-1,1,2-trimethyl-propyl)-amide;
2-Cyclopropyl-5H-pyrrolo[2,3-b]pyrazine-7-carboxylic acid dicyclopropylmethyl-amide;
2-Cyclopropyl-5H-pyrrolo[2,3-b]pyrazine-7-carboxylic acid (2-cyano-1-cyclopropyl-2,2-dimethyl-ethyl)-amide;
2-Cyclopropyl-5H-pyrrolo[2,3-b]pyrazine-7-carboxylic acid [(R)-1-(1-hydroxy-cyclopentyl)-ethyl]-amide;
2-Cyclopropyl-5H-pyrrolo[2,3-b]pyrazine-7-carboxylic acid ((1R,2R)-2-hydroxy-1,2-dimethyl-butyl)-amide;
2-Cyclopropyl-5H-pyrrolo[2,3-b]pyrazine-7-carboxylic acid ((1R,2R)-2-hydroxy-1,2-dimethyl-pentyl)-amide;
2-Cyclopropyl-5H-pyrrolo[2,3-b]pyrazine-7-carboxylic acid [1-(tetrahydro-pyran-4-yl)-ethyl]-amide;
2-Cyclopropyl-5H-pyrrolo[2,3-b]pyrazine-7-carboxylic acid ((R)-2-cyano-1,2,2-trimethyl-ethyl)-amide;
2-Cyclopropyl-5H-pyrrolo[2,3-b]pyrazine-7-carboxylic acid ((S)-2-cyano-1,2,2-trimethyl-ethyl)-amide;
2-Cyclopropyl-5H-pyrrolo[2,3-b]pyrazine-7-carboxylic acid ((1S,2R,3S)-1-cyclohexylmethyl-3-cyclopropyl-2,3-dihydroxy-propyl)-amide;
2-Cyclopropyl-5H-pyrrolo[2,3-b]pyrazine-7-carboxylic acid (1-cyano-2-methyl-propyl)-amide;
2-Cyclopropyl-5H-pyrrolo[2,3-b]pyrazine-7-carboxylic acid (cyano-cyclopropyl-methyl)-amide;
2-Cyclopropyl-5H-pyrrolo[2,3-b]pyrazine-7-carboxylic acid ((1R,2R)-3-cyano-2-hydroxy-1,2-dimethyl-propyl)-amide;
3-Cyclopropyl-3-[(2-cyclopropyl-5H-pyrrolo[2,3-b]pyrazine-7-carbonyl)-amino]-2,2-dimethyl-propionic acid;
2-Cyclopropyl-5H-pyrrolo[2,3-b]pyrazine-7-carboxylic acid (2-hydroxy-2-methyl-1-trifluoromethyl-propyl)-amide;
2-Cyclopropyl-5H-pyrrolo[2,3-b]pyrazine-7-carboxylic acid ((S)-1-cyclohexyl-2-hydroxy-2-methyl-propyl)-amide;
2-Cyclopropyl-5H-pyrrolo[2,3-b]pyrazine-7-carboxylic acid (1-cyclopentyl-ethyl)-amide;
2-Phenoxy-5H-pyrrolo[2,3-b]pyrazine-7-carboxylic acid (1-cyclopropyl-ethyl)-amide;
2-Cyclopropyl-5H-pyrrolo[2,3-b]pyrazine-7-carboxylic acid [(S)-1-(1-hydroxy-cyclopentyl)-ethyl]-amide;
2-Cyclopropyl-5H-pyrrolo[2,3-b]pyrazine-7-carboxylic acid (3-methanesulfonyl-2,2-dimethyl-propyl)-amide;
2-(1-Ethyl-1H-pyrazol-4-yl)-5H-pyrrolo[2,3-b]pyrazine-7-carboxylic acid ((R)-2-hydroxy-1,2-dimethyl-propyl)-amide;
2-Cyclopropyl-5H-pyrrolo[2,3-b]pyrazine-7-carboxylic acid [(R)-1-(1-cyano-cyclopentyl)-ethyl]-amide;
2-Cyclopropyl-5H-pyrrolo[2,3-b]pyrazine-7-carboxylic acid [(S)-1-(1-cyano-cyclopentyl)-ethyl]-amide;
2-(1-Methyl-1H-pyrazol-4-yl)-5H-pyrrolo[2,3-b]pyrazine-7-carboxylic acid ((R)-2-hydroxy-1,2-dimethyl-propyl)-amide;
2-Thiophen-2-yl-5H-pyrrolo[2,3-b]pyrazine-7-carboxylic acid ((R)-2-hydroxy-1,2-dimethyl-propyl)-amide;
2-Cyclopropyl-5H-pyrrolo[2,3-b]pyrazine-7-carboxylic acid [(R)-cyclopropyl-(1-hydroxy-cyclopentyl)-methyl]-amide;
2-(2,4-Difluoro-phenoxy)-5H-pyrrolo[2,3-b]pyrazine-7-carboxylic acid (1-cyclopropyl-ethyl)-amide;
2-Cyclopropyl-5H-pyrrolo[2,3-b]pyrazine-7-carboxylic acid (2-cyano-1-cyclopropyl-ethyl)-amide;
2-Cyclopropyl-5H-pyrrolo[2,3-b]pyrazine-7-carboxylic acid cyclohexylmethyl-amide;
2-Cyclopropyl-5H-pyrrolo[2,3-b]pyrazine-7-carboxylic acid (1-methanesulfonyl-piperidin-3-ylmethyl)-amide;
2-Cyclopropyl-5H-pyrrolo[2,3-b]pyrazine-7-carboxylic acid (1-methanesulfonyl-pyrrolidin-3-ylmethyl)-amide;
2-(3,6-Dihydro-2H-pyran-4-yl)-5H-pyrrolo[2,3-b]pyrazine-7-carboxylic acid ((R)-2-hydroxy-1,2-dimethyl-propyl)-amide;
2-Thiazol-2-yl-5H-pyrrolo[2,3-b]pyrazine-7-carboxylic acid ((R)-2-hydroxy-1,2-dimethyl-propyl)-amide;
2-Pyridin-2-yl-5H-pyrrolo[2,3-b]pyrazine-7-carboxylic acid ((R)-2-hydroxy-1,2-dimethyl-propyl)-amide;
2-(4-Fluoro-phenoxy)-5H-pyrrolo[2,3-b]pyrazine-7-carboxylic acid (1-cyclopropyl-ethyl)-amide;
2-(2-Fluoro-phenoxy)-5H-pyrrolo[2,3-b]pyrazine-7-carboxylic acid (1-cyclopropyl-ethyl)-amide;
2-Cyano-5H-pyrrolo[2,3-b]pyrazine-7-carboxylic acid ((R)-2-hydroxy-1,2-dimethyl-propyl)-amide;
2-Phenoxy-5H-pyrrolo[2,3-b]pyrazine-7-carboxylic acid ((R)-2-hydroxy-1,2-dimethyl-propyl)-amide;
2-Cyclopropyl-5H-pyrrolo[2,3-b]pyrazine-7-carboxylic acid [(R)-1-(1-cyano-cyclohexyl)-ethyl]-amide;
2-Cyclopropyl-5H-pyrrolo[2,3-b]pyrazine-7-carboxylic acid [(S)-1-(1-cyano-cyclohexyl)-ethyl]-amide;
2-Phenoxy-5H-pyrrolo[2,3-b]pyrazine-7-carboxylic acid isopropylamide;
2-Phenoxy-5H-pyrrolo[2,3-b]pyrazine-7-carboxylic acid ((S)-1,2,2-trimethyl-propyl)-amide;
2-Phenoxy-5H-pyrrolo[2,3-b]pyrazine-7-carboxylic acid ((S)-sec-butyl)-amide;
2-Phenoxy-5H-pyrrolo[2,3-b]pyrazine-7-carboxylic acid ((S)-1,2-dimethyl-propyl)-amide;
2-Phenoxy-5H-pyrrolo[2,3-b]pyrazine-7-carboxylic acid ((S)-1-cyclohexyl-ethyl)-amide;
2-(2,4-Difluoro-phenoxy)-5H-pyrrolo[2,3-b]pyrazine-7-carboxylic acid ((R)-1-cyclohexyl-ethyl)-amide;
2-(2,4-Difluoro-phenoxy)-5H-pyrrolo[2,3-b]pyrazine-7-carboxylic acid ((S)-2-hydroxy-1,2-dimethyl-propyl)-amide;
2-(2,4-Difluoro-phenoxy)-5H-pyrrolo[2,3-b]pyrazine-7-carboxylic acid ((R)-sec-butyl)-amide;
2-(2,4-Difluoro-phenoxy)-5H-pyrrolo[2,3-b]pyrazine-7-carboxylic acid ((R)-1,2-dimethyl-propyl)-amide;
2-Phenoxy-5H-pyrrolo[2,3-b]pyrazine-7-carboxylic acid ((S)-2-hydroxy-1,2-dimethyl-propyl)-amide;
2-Phenoxy-5H-pyrrolo[2,3-b]pyrazine-7-carboxylic acid ((R)-1-cyclohexyl-ethyl)-amide;
2-Phenoxy-5H-pyrrolo[2,3-b]pyrazine-7-carboxylic acid ((R)-1,2,2-trimethyl-propyl)-amide;
2-Phenoxy-5H-pyrrolo[2,3-b]pyrazine-7-carboxylic acid ethylamide;
2-(2,4-Difluoro-phenoxy)-5H-pyrrolo[2,3-b]pyrazine-7-carboxylic acid isopropylamide;
2-(2,4-Difluoro-phenoxy)-5H-pyrrolo[2,3-b]pyrazine-7-carboxylic acid ((S)-1,2,2-trimethyl-propyl)-amide;
2-(2,4-Difluoro-phenoxy)-5H-pyrrolo[2,3-b]pyrazine-7-carboxylic acid ((R)-1,2,2-trimethyl-propyl)-amide;
2-Cyclopropyl-5H-pyrrolo[2,3-b]pyrazine-7-carboxylic acid ((R)-2-cyano-1-cyclopropyl-ethyl)-amide;
2-Cyclopropyl-5H-pyrrolo[2,3-b]pyrazine-7-carboxylic acid (1-acetyl-piperidin-3-ylmethyl)-amide;
2-Cyclopropyl-5H-pyrrolo[2,3-b]pyrazine-7-carboxylic acid (1-acetyl-pyrrolidin-3-ylmethyl)-amide;
2-(1-Ethyl-1H-pyrazol-4-yl)-5H-pyrrolo[2,3-b]pyrazine-7-carboxylic acid [(S)-1-(1-hydroxy-cyclopentyl)-ethyl]-amide;
2-(1-Methyl-1H-pyrazol-4-yl)-5H-pyrrolo[2,3-b]pyrazine-7-carboxylic acid ((S)-2-cyano-1,2,2-trimethyl-ethyl)-amide;
2-(1-Methyl-1H-pyrazol-4-yl)-5H-pyrrolo[2,3-b]pyrazine-7-carboxylic acid ((S)-1-cyclohexyl-ethyl)-amide;
2-(1-Methyl-1H-pyrazol-4-yl)-5H-pyrrolo[2,3-b]pyrazine-7-carboxylic acid ((S)-1,2,2-trimethyl-propyl)-amide;
2-Thiophen-2-yl-5H-pyrrolo[2,3-b]pyrazine-7-carboxylic acid ((S)-1,2,2-trimethyl-propyl)-amide;
2-(4-Trifluoromethyl-phenyl)-5H-pyrrolo[2,3-b]pyrazine-7-carboxylic acid ((S)-1,2,2-trimethyl-propyl)-amide;
2-Cyclopropyl-5H-pyrrolo[2,3-b]pyrazine-7-carboxylic acid ((S)-3-methanesulfonyl-1,2,2-trimethyl-propyl)-amide;
2-[1-(3-Chloro-phenyl)-1H-imidazol-4-yl]-5H-pyrrolo[2,3-b]pyrazine-7-carboxylic acid ((S)-1,2,2-trimethyl-propyl)-amide;
2-[1-(3-Trifluoromethyl-phenyl)-1H-imidazol-4-yl]-5H-pyrrolo[2,3-b]pyrazine-7-carboxylic acid ((S)-1,2,2-trimethyl-propyl)-amide;
2-[1-(5-Chloro-2-fluoro-phenyl)-1H-imidazol-4-yl]-5H-pyrrolo[2,3-b]pyrazine-7-carboxylic acid ((S)-1,2,2-trimethyl-propyl)-amide;
2-[1-(2-Fluoro-5-methyl-phenyl)-1H-imidazol-4-yl]-5H-pyrrolo[2,3-b]pyrazine-7-carboxylic acid ((S)-1,2,2-trimethyl-propyl)-amide;
2-[1-(2-Fluoro-5-trifluoromethyl-phenyl)-1H-imidazol-4-yl]-5H-pyrrolo[2,3-b]pyrazine-7-carboxylic acid ((S)-1,2,2-trimethyl-propyl)-amide;
2-(1-m-Tolyl-1H-imidazol-4-yl)-5H-pyrrolo[2,3-b]pyrazine-7-carboxylic acid ((S)-1,2,2-trimethyl-propyl)-amide;
2-[1-(3-Ethyl-phenyl)-1H-imidazol-4-yl]-5H-pyrrolo[2,3-b]pyrazine-7-carboxylic acid ((S)-1,2,2-trimethyl-propyl)-amide;
2-[1-(3-Isopropyl-phenyl)-1H-imidazol-4-yl]-5H-pyrrolo[2,3-b]pyrazine-7-carboxylic acid ((S)-1,2,2-trimethyl-propyl)-amide;
2-[1-(3-tert-Butyl-phenyl)-1H-imidazol-4-yl]-5H-pyrrolo[2,3-b]pyrazine-7-carboxylic acid ((S)-1,2,2-trimethyl-propyl)-amide;
2-(1,3-Dimethyl-1H-pyrazol-4-yl)-5H-pyrrolo[2,3-b]pyrazine-7-carboxylic acid ((S)-2-methoxy-1-methyl-ethyl)-amide;
2-(5-Ethylcarbamoyl-thiophen-2-yl)-5H-pyrrolo[2,3-b]pyrazine-7-carboxylic acid ((S)-1,2,2-trimethyl-propyl)-amide;
2-(5-Isopropylcarbamoyl-thiophen-2-yl)-5H-pyrrolo[2,3-b]pyrazine-7-carboxylic acid ((S)-1,2,2-trimethyl-propyl)-amide;
2-(5-tert-Butylcarbamoyl-thiophen-2-yl)-5H-pyrrolo[2,3-b]pyrazine-7-carboxylic acid ((S)-1,2,2-trimethyl-propyl)-amide;
2-[5-(1-Methyl-2-pyrazol-1-yl-ethylcarbamoyl)-thiophen-2-yl]-5H-pyrrolo[2,3-b]pyrazine-7-carboxylic acid ((S)-1,2,2-trimethyl-propyl)-amide;
2-{5-[2-(4-Fluoro-phenyl)-1-methyl-ethylcarbamoyl]-thiophen-2-yl}-5H-pyrrolo[2,3-b]pyrazine-7-carboxylic acid ((S)-1,2,2-trimethyl-propyl)-amide;
2-(5-Diethylcarbamoyl-thiophen-2-yl)-5H-pyrrolo[2,3-b]pyrazine-7-carboxylic acid ((S)-1,2,2-trimethyl-propyl)-amide;
2-[5-(4-Methyl-piperazine-1-carbonyl)-thiophen-2-yl]-5H-pyrrolo[2,3-b]pyrazine-7-carboxylic acid ((S)-1,2,2-trimethyl-propyl)-amide;
2-[5-((R)-1-Cyclopropyl-ethylcarbamoyl)-thiophen-2-yl]-5H-pyrrolo[2,3-b]pyrazine-7-carboxylic acid ((S)-1,2,2-trimethyl-propyl)-amide;
2-[1-(3-Vinyl-phenyl)-1H-imidazol-4-yl]-5H-pyrrolo[2,3-b]pyrazine-7-carboxylic acid ((S)-1,2,2-trimethyl-propyl)-amide;
2-{5-[(Pyridin-3-ylmethyl)-carbamoyl]-thiophen-2-yl}-5H-pyrrolo[2,3-b]pyrazine-7-carboxylic acid ((S)-1,2,2-trimethyl-propyl)-amide;
2-{5-[(Pyridin-4-ylmethyl)-carbamoyl]-thiophen-2-yl}-5H-pyrrolo[2,3-b]pyrazine-7-carboxylic acid ((S)-1,2,2-trimethyl-propyl)-amide;
2-{5-[(Pyridin-2-ylmethyl)-carbamoyl]-thiophen-2-yl}-5H-pyrrolo[2,3-b]pyrazine-7-carboxylic acid ((S)-1,2,2-trimethyl-propyl)-amide;
2-[5-(4-Cyano-piperidine-1-carbonyl)-thiophen-2-yl]-5H-pyrrolo[2,3-b]pyrazine-7-carboxylic acid ((S)-1,2,2-trimethyl-propyl)-amide;
2-[5-(Cyclopentylmethyl-carbamoyl)-thiophen-2-yl]-5H-pyrrolo[2,3-b]pyrazine-7-carboxylic acid ((S)-1,2,2-trimethyl-propyl)-amide;
2-[5-((R)-2-Hydroxy-1-methyl-ethylcarbamoyl)-thiophen-2-yl]-5H-pyrrolo[2,3-b]pyrazine-7-carboxylic acid ((S)-1,2,2-trimethyl-propyl)-amide;
2-[5-((R)-1-Methyl-2-phenyl-ethylcarbamoyl)-thiophen-2-yl]-5H-pyrrolo[2,3-b]pyrazine-7-carboxylic acid ((S)-1,2,2-trimethyl-propyl)-amide;
2-[5-(1-Pyridin-3-yl-ethylcarbamoyl)-thiophen-2-yl]-5H-pyrrolo[2,3-b]pyrazine-7-carboxylic acid ((S)-1,2,2-trimethyl-propyl)-amide;
2-[5-(Cyanomethyl-carbamoyl)-thiophen-2-yl]-5H-pyrrolo[2,3-b]pyrazine-7-carboxylic acid ((S)-1,2,2-trimethyl-propyl)-amide;
2-[5-(2-Sulfamoyl-ethylcarbamoyl)-thiophen-2-yl]-5H-pyrrolo[2,3-b]pyrazine-7-carboxylic acid ((S)-1,2,2-trimethyl-propyl)-amide;
2-[5-(2-Imidazol-1-yl-1-methyl-ethylcarbamoyl)-thiophen-2-yl]-5H-pyrrolo[2,3-b]pyrazine-7-carboxylic acid ((S)-1,2,2-trimethyl-propyl)-amide;
2-[5-(4-Hydroxy-4-methyl-piperidine-1-carbonyl)-thiophen-2-yl]-5H-pyrrolo[2,3-b]pyrazine-7-carboxylic acid ((S)-1,2,2-trimethyl-propyl)-amide;
2-[5-(1-Methyl-2-pyridin-2-yl-ethylcarbamoyl)-thiophen-2-yl]-5H-pyrrolo[2,3-b]pyrazine-7-carboxylic acid ((S)-1,2,2-trimethyl-propyl)-amide;
2-[5-(7-Aza-bicyclo[2.2.1]heptane-7-carbonyl)-thiophen-2-yl]-5H-pyrrolo[2,3-b]pyrazine-7-carboxylic acid ((S)-1,2,2-trimethyl-propyl)-amide;
2-[5-(3-Cyano-azetidine-1-carbonyl)-thiophen-2-yl]-5H-pyrrolo[2,3-b]pyrazine-7-carboxylic acid ((S)-1,2,2-trimethyl-propyl)-amide;
2-[5-(3-Carbamoyl-azetidine-1-carbonyl)-thiophen-2-yl]-5H-pyrrolo[2,3-b]pyrazine-7-carboxylic acid ((S)-1,2,2-trimethyl-propyl)-amide;
2-[5-(Azetidine-1-carbonyl)-thiophen-2-yl]-5H-pyrrolo[2,3-b]pyrazine-7-carboxylic acid ((S)-1,2,2-trimethyl-propyl)-amide;
2-[5-(2,6-Dimethyl-piperidine-1-carbonyl)-thiophen-2-yl]-5H-pyrrolo[2,3-b]pyrazine-7-carboxylic acid ((S)-1,2,2-trimethyl-propyl)-amide;
1-{5-[7-((S)-1,2,2-Trimethyl-propylcarbamoyl)-5H-pyrrolo[2,3-b]pyrazin-2-yl]-thiophene-2-carbonyl}-piperidine-4-carboxylic acid;
2-[5-(4-Acetylamino-piperidine-1-carbonyl)-thiophen-2-yl]-5H-pyrrolo[2,3-b]pyrazine-7-carboxylic acid ((S)-1,2,2-trimethyl-propyl)-amide;
2-[5-(4-Methyl-benzylcarbamoyl)-thiophen-2-yl]-5H-pyrrolo[2,3-b]pyrazine-7-carboxylic acid ((S)-1,2,2-trimethyl-propyl)-amide;
2-[5-(4-Fluoro-benzylcarbamoyl)-thiophen-2-yl]-5H-pyrrolo[2,3-b]pyrazine-7-carboxylic acid ((S)-1,2,2-trimethyl-propyl)-amide;
2-[5-(2,3-Dichloro-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Methyl-benzylcarbamoyl)-thiophen-2-yl]-5H-pyrrolo[2,3-b]pyrazine-7-carboxylic acid ((S)-1,2,2-trimethyl-propyl)-amide;
2-[5-(2,6-Difluoro-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Chloro-6-fluoro-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Methyl-cyclohexylcarbamoyl)-thiophen-2-yl]-5H-pyrrolo[2,3-b]pyrazine-7-carboxylic acid ((S)-1,2,2-trimethyl-propyl)-amide;
2-[5-((1S,2R)-2-Phenyl-cyclopropylcarbamoyl)-thiophen-2-yl]-5H-pyrrolo[2,3-b]pyrazine-7-carboxylic acid ((S)-1,2,2-trimethyl-propyl)-amide;
2-{5-[(4-Methyl-thiophen-2-ylmethyl)-carbamoyl]-thiophen-2-yl}-5H-pyrrolo[2,3-b]pyrazine-7-carboxylic acid ((S)-1,2,2-trimethyl-propyl)-amide;
2-{5-[(5-Methyl-furan-2-ylmethyl)-carbamoyl]-thiophen-2-yl}-5H-pyrrolo[2,3-b]pyrazine-7-carboxylic acid ((S)-1,2,2-trimethyl-propyl)-amide;
2-[5-(Adamantan-1-ylcarbamoyl)-thiophen-2-yl]-5H-pyrrolo[2,3-b]pyrazine-7-carboxylic acid ((S)-1,2,2-trimethyl-propyl)-amide;
2-{5-[1-(4-Fluoro-phenyl)-ethylcarbamoyl]-thiophen-2-yl}-5H-pyrrolo[2,3-b]pyrazine-7-carboxylic acid ((S)-1,2,2-trimethyl-propyl)-amide;
2-[5-(Methoxy-methyl-carbamoyl)-thiophen-2-yl]-5H-pyrrolo[2,3-b]pyrazine-7-carboxylic acid ((S)-1,2,2-trimethyl-propyl)-amide;
2-(5-Methoxycarbamoyl-thiophen-2-yl)-5H-pyrrolo[2,3-b]pyrazine-7-carboxylic acid ((S)-1,2,2-trimethyl-propyl)-amide;
2-(5-Prop-2-ynylcarbamoyl-thiophen-2-yl)-5H-pyrrolo[2,3-b]pyrazine-7-carboxylic acid ((S)-1,2,2-trimethyl-propyl)-amide;
2-{5-[(R)-2-(3H-Imidazol-4-yl)-1-methyl-ethylcarbamoyl]-thiophen-2-yl}-5H-pyrrolo[2,3-b]pyrazine-7-carboxylic acid ((S)-1,2,2-trimethyl-propyl)-amide;
2-[5-(5,6,7,8-Tetrahydro-naphthalen-2-ylcarbamoyl)-thiophen-2-yl]-5H-pyrrolo[2,3-b]pyrazine-7-carboxylic acid ((S)-1,2,2-trimethyl-propyl)-amide;
2-(5-Phenylcarbamoyl-thiophen-2-yl)-5H-pyrrolo[2,3-b]pyrazine-7-carboxylic acid ((S)-1,2,2-trimethyl-propyl)-amide;
2-[5-((R)-1-p-Tolyl-ethylcarbamoyl)-thiophen-2-yl]-5H-pyrrolo[2,3-b]pyrazine-7-carboxylic acid ((S)-1,2,2-trimethyl-propyl)-amide;
2-[5-(2-Methoxy-benzylcarbamoyl)-thiophen-2-yl]-5H-pyrrolo[2,3-b]pyrazine-7-carboxylic acid ((S)-1,2,2-trimethyl-propyl)-amide;
2-[5-(2,5-Dimethoxy-benzylcarbamoyl)-thiophen-2-yl]-5H-pyrrolo[2,3-b]pyrazine-7-carboxylic acid ((S)-1,2,2-trimethyl-propyl)-amide;
2-{5-[(4-Fluoro-benzyl)-methyl-carbamoyl]-thiophen-2-yl}-5H-pyrrolo[2,3-b]pyrazine-7-carboxylic acid ((S)-1,2,2-trimethyl-propyl)-amide;
2-[5-(3-Methoxy-benzylcarbamoyl)-thiophen-2-yl]-5H-pyrrolo[2,3-b]pyrazine-7-carboxylic acid ((S)-1,2,2-trimethyl-propyl)-amide;
2-[5-(3-Trifluoromethyl-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Chloro-4-iodo-phenylcarbamoyl)-thiophen-2-yl]-5H-pyrrolo[2,3-b]pyrazine-7-carboxylic acid ((S)-1,2,2-trimethyl-propyl)-amide;
2-[5-((R)-1,2,2-Trimethyl-propylcarbamoyl)-thiophen-2-yl]-5H-pyrrolo[2,3-b]pyrazine-7-carboxylic acid ((S)-1,2,2-trimethyl-propyl)-amide;
2-[5-(2,2-Dimethyl-propylcarbamoyl)-thiophen-2-yl]-5H-pyrrolo[2,3-b]pyrazine-7-carboxylic acid ((S)-1,2,2-trimethyl-propyl)-amide;
2-[5-((R)-2-Methanesulfonyl-1-methyl-ethylcarbamoyl)-thiophen-2-yl]-5H-pyrrolo[2,3-b]pyrazine-7-carboxylic acid ((S)-1,2,2-trimethyl-propyl)-amide;
2-[5-(1,1-Dioxo-hexahydro-1λ⁶-thiopyran-4-ylcarbamoyl)-thiophen-2-yl]-5H-pyrrolo[2,3-b]pyrazine-7-carboxylic acid ((S)-1,2,2-trimethyl-propyl)-amide;
2-[5-(1,1-Dioxo-1λ⁶-thiomorpholine-4-carbonyl)-thiophen-2-yl]-5H-pyrrolo[2,3-b]pyrazine-7-carboxylic acid ((S)-1,2,2-trimethyl-propyl)-amide;
2-[5-(2-Methoxy-1-methyl-ethylcarbamoyl)-thiophen-2-yl]-5H-pyrrolo[2,3-b]pyrazine-7-carboxylic acid ((S)-1,2,2-trimethyl-propyl)-amide;
2-(5-Carbamoyl-thiophen-2-yl)-5H-pyrrolo[2,3-b]pyrazine-7-carboxylic acid ((S)-1,2,2-trimethyl-propyl)-amide;
2-[5-(3,3,3-Trifluoro-propylcarbamoyl)-thiophen-2-yl]-5H-pyrrolo[2,3-b]pyrazine-7-carboxylic acid ((S)-1,2,2-trimethyl-propyl)-amide;
2-[5-(2-Oxa-6-aza-spiro[3.3]heptane-6-carbonyl)-thiophen-2-yl]-5H-pyrrolo[2,3-b]pyrazine-7-carboxylic acid ((S)-1,2,2-trimethyl-propyl)-amide;
2-[5-(3,3-Bis-hydroxymethyl-azetidine-1-carbonyl)-thiophen-2-yl]-5H-pyrrolo[2,3-b]pyrazine-7-carboxylic acid ((S)-1,2,2-trimethyl-propyl)-amide;
2-[4-Methyl-5-(tetrahydro-pyran-4-ylcarbamoyl)-thiophen-2-yl]-5H-pyrrolo[2,3-b]pyrazine-7-carboxylic acid ((S)-1,2,2-trimethyl-propyl)-amide;
2-[5-(1,1-Dioxo-1λ⁶-thiomorpholine-4-carbonyl)-4-methyl-thiophen-2-yl]-5H-pyrrolo[2,3-b]pyrazine-7-carboxylic acid ((S)-1,2,2-trimethyl-propyl)-amide;
2-[4-Methyl-5-(2-oxa-6-aza-spiro[3.3]heptane-6-carbonyl)-thiophen-2-yl]-5H-pyrrolo[2,3-b]pyrazine-7-carboxylic acid ((S)-1,2,2-trimethyl-propyl)-amide;
2-[5-(3,3-Bis-hydroxymethyl-azetidine-1-carbonyl)-4-methyl-thiophen-2-yl]-5H-pyrrolo[2,3-b]pyrazine-7-carboxylic acid ((S)-1,2,2-trimethyl-propyl)-amide;
2-[5-(Tetrahydro-pyran-4-ylcarbamoyl)-thiophen-2-yl]-5H-pyrrolo[2,3-b]pyrazine-7-carboxylic acid ((S)-2-cyano-1,2,2-trimethyl-ethyl)-amide;
2-[5-(Piperidine-1-carbonyl)-thiophen-2-yl]-5H-pyrrolo[2,3-b]pyrazine-7-carboxylic acid ((S)-2-cyano-1,2,2-trimethyl-ethyl)-amide;
2-[5-(Tetrahydro-pyran-4-ylcarbamoyl)-thiophen-2-yl]-5H-pyrrolo[2,3-b]pyrazine-7-carboxylic acid ((S)-1,2,2-trimethyl-propyl)-amide;
2-(5-Benzylcarbamoyl-thiophen-2-yl)-5H-pyrrolo[2,3-b]pyrazine-7-carboxylic acid ((S)-1,2,2-trimethyl-propyl)-amide;
2-[5-(3-Cyano-benzylcarbamoyl)-thiophen-2-yl]-5H-pyrrolo[2,3-b]pyrazine-7-carboxylic acid ((S)-1,2,2-trimethyl-propyl)-amide;
2-(3-Cyano-phenoxy)-5H-pyrrolo[2,3-b]pyrazine-7-carboxylic acid isopropylamide;
2-(3-Methoxy-phenoxy)-5H-pyrrolo[2,3-b]pyrazine-7-carboxylic acid isopropylamide;
2-(3-Trifluoromethoxy-phenoxy)-5H-pyrrolo[2,3-b]pyrazine-7-carboxylic acid isopropylamide;
2-(3-tert-Butyl-phenoxy)-5H-pyrrolo[2,3-b]pyrazine-7-carboxylic acid isopropylamide;
2-m-Tolyloxy-5H-pyrrolo[2,3-b]pyrazine-7-carboxylic acid isopropylamide;
2-(3-Ethyl-phenoxy)-5H-pyrrolo[2,3-b]pyrazine-7-carboxylic acid isopropylamide;
2-(3-Isopropyl-phenoxy)-5H-pyrrolo[2,3-b]pyrazine-7-carboxylic acid isopropylamide;
2-(3-Trifluoromethyl-phenoxy)-5H-pyrrolo[2,3-b]pyrazine-7-carboxylic acid isopropylamide;
2-(2-Trifluoromethyl-phenoxy)-5H-pyrrolo[2,3-b]pyrazine-7-carboxylic acid isopropylamide;
2-(2-Benzyl-phenoxy)-5H-pyrrolo[2,3-b]pyrazine-7-carboxylic acid isopropylamide;
2-(2-Ethyl-phenoxy)-5H-pyrrolo[2,3-b]pyrazine-7-carboxylic acid isopropylamide;
2-(5,6,7,8-Tetrahydro-naphthalen-1-yloxy)-5H-pyrrolo[2,3-b]pyrazine-7-carboxylic acid isopropylamide;
2-(5,6,7,8-Tetrahydro-naphthalen-2-yloxy)-5H-pyrrolo[2,3-b]pyrazine-7-carboxylic acid isopropylamide;
2-(Naphthalen-1-yloxy)-5H-pyrrolo[2,3-b]pyrazine-7-carboxylic acid isopropylamide;
2-(Naphthalen-2-yloxy)-5H-pyrrolo[2,3-b]pyrazine-7-carboxylic acid isopropylamide;
2-(3-Chloro-phenoxy)-5H-pyrrolo[2,3-b]pyrazine-7-carboxylic acid isopropylamide;
2-(3-Chloro-phenoxy)-5H-pyrrolo[2,3-b]pyrazine-7-carboxylic acid ethylamide;
2-(3-Cyano-phenoxy)-5H-pyrrolo[2,3-b]pyrazine-7-carboxylic acid ethylamide;
2-(3-Trifluoromethoxy-phenoxy)-5H-pyrrolo[2,3-b]pyrazine-7-carboxylic acid ethylamide;
2-(3-tert-Butyl-phenoxy)-5H-pyrrolo[2,3-b]pyrazine-7-carboxylic acid ethylamide;
2-m-Tolyloxy-5H-pyrrolo[2,3-b]pyrazine-7-carboxylic acid ethylamide;
2-(3-Ethyl-phenoxy)-5H-pyrrolo[2,3-b]pyrazine-7-carboxylic acid ethylamide;
2-(3-Isopropyl-phenoxy)-5H-pyrrolo[2,3-b]pyrazine-7-carboxylic acid ethylamide;
2-(3-Trifluoromethyl-phenoxy)-5H-pyrrolo[2,3-b]pyrazine-7-carboxylic acid ethylamide;
2-o-Tolyloxy-5H-pyrrolo[2,3-b]pyrazine-7-carboxylic acid isopropylamide;
2-(2-Trifluoromethoxy-phenoxy)-5H-pyrrolo[2,3-b]pyrazine-7-carboxylic acid isopropylamide;
2-(2,2-Dimethyl-2,3-dihydro-benzofuran-7-yloxy)-5H-pyrrolo[2,3-b]pyrazine-7-carboxylic acid isopropylamide;
2-(2-Chloro-phenoxy)-5H-pyrrolo[2,3-b]pyrazine-7-carboxylic acid isopropylamide;
2-(2-Methoxy-phenoxy)-5H-pyrrolo[2,3-b]pyrazine-7-carboxylic acid isopropylamide;
2-o-Tolyloxy-5H-pyrrolo[2,3-b]pyrazine-7-carboxylic acid ethylamide;
2-(3,5-Dimethoxy-phenoxy)-5H-pyrrolo[2,3-b]pyrazine-7-carboxylic acid isopropylamide;
2-(5,6,7,8-Tetrahydro-naphthalen-1-yloxy)-5H-pyrrolo[2,3-b]pyrazine-7-carboxylic acid ethylamide;
2-(5,6,7,8-Tetrahydro-naphthalen-2-yloxy)-5H-pyrrolo[2,3-b]pyrazine-7-carboxylic acid ethylamide;
2-(Naphthalen-1-yloxy)-5H-pyrrolo[2,3-b]pyrazine-7-carboxylic acid ethylamide;
2-(Naphthalen-2-yloxy)-5H-pyrrolo[2,3-b]pyrazine-7-carboxylic acid ethylamide;
2-(3,5-Dimethoxy-phenoxy)-5H-pyrrolo[2,3-b]pyrazine-7-carboxylic acid ethylamide;
2-(3-Methoxy-phenoxy)-5H-pyrrolo[2,3-b]pyrazine-7-carboxylic acid ethylamide;
2-(2-Chloro-phenoxy)-5H-pyrrolo[2,3-b]pyrazine-7-carboxylic acid ethylamide;
2-(4-Cyano-phenoxy)-5H-pyrrolo[2,3-b]pyrazine-7-carboxylic acid isopropylamide;
2-(4-Cyano-phenoxy)-5H-pyrrolo[2,3-b]pyrazine-7-carboxylic acid ethylamide;
2-((R)-3-Methanesulfonylamino-indan-5-yloxy)-5H-pyrrolo[2,3-b]pyrazine-7-carboxylic acid isopropylamide;
2-((R)-3-Acetylamino-indan-5-yloxy)-5H-pyrrolo[2,3-b]pyrazine-7-carboxylic acid isopropylamide;
2-((R)-3-Methanesulfonylamino-indan-5-yloxy)-5H-pyrrolo[2,3-b]pyrazine-7-carboxylic acid ethylamide;
2-((R)-3-Acetylamino-indan-5-yloxy)-5H-pyrrolo[2,3-b]pyrazine-7-carboxylic acid ethylamide;
2-(1H-Indol-6-yloxy)-5H-pyrrolo[2,3-b]pyrazine-7-carboxylic acid isopropylamide;
2-(1H-Indol-6-yloxy)-5H-pyrrolo[2,3-b]pyrazine-7-carboxylic acid ethylamide;
2-(1H-Indol-4-yloxy)-5H-pyrrolo[2,3-b]pyrazine-7-carboxylic acid isopropylamide;
2-(1H-Indol-4-yloxy)-5H-pyrrolo[2,3-b]pyrazine-7-carboxylic acid ethylamide;
2-(1-Methyl-1H-indol-6-yloxy)-5H-pyrrolo[2,3-b]pyrazine-7-carboxylic acid isopropylamide;
2-(1H-Indol-5-yloxy)-5H-pyrrolo[2,3-b]pyrazine-7-carboxylic acid isopropylamide;
2-(6-Methyl-pyridin-2-yloxy)-5H-pyrrolo[2,3-b]pyrazine-7-carboxylic acid isopropylamide;
2-(4,6-Dimethyl-pyridin-2-yloxy)-5H-pyrrolo[2,3-b]pyrazine-7-carboxylic acid isopropylamide;
2-(2-Methyl-pyridin-3-yloxy)-5H-pyrrolo[2,3-b]pyrazine-7-carboxylic acid isopropylamide;
2-((R)-3-Amino-indan-5-yloxy)-5H-pyrrolo[2,3-b]pyrazine-7-carboxylic acid isopropylamide
2-((R)-3-Propionylamino-indan-5-yloxy)-5H-pyrrolo[2,3-b]pyrazine-7-carboxylic acid isopropylamide;
2-{(R)-3-[(Tetrahydro-pyran-4-carbonyl)-amino]-indan-5-yloxy}-5H-pyrrolo[2,3-b]pyrazine-7-carboxylic acid isopropylamide;
2-[(R)-3-(Cyclopropanecarbonyl-amino)-indan-5-yloxy]-5H-pyrrolo[2,3-b]pyrazine-7-carboxylic acid isopropylamide;
2-[(R)-3-(2,2-Dimethyl-propionylamino)-indan-5-yloxy]-5H-pyrrolo[2,3-b]pyrazine-7-carboxylic acid isopropylamide;
2-((R)-3-Benzoylamino-indan-5-yloxy)-5H-pyrrolo[2,3-b]pyrazine-7-carboxylic acid isopropylamide;
2-((R)-3-Acetylamino-indan-5-yloxy)-5H-pyrrolo[2,3-b]pyrazine-7-carboxylic acid ((S)-1,2,2-trimethyl-propyl)-amide;
2-((S)-3-Acetylamino-indan-5-yloxy)-5H-pyrrolo[2,3-b]pyrazine-7-carboxylic acid isopropylamide;
2-((S)-3-Amino-indan-5-yloxy)-5H-pyrrolo[2,3-b]pyrazine-7-carboxylic acid isopropylamide;
2-(Indan-5-yloxy)-5H-pyrrolo[2,3-b]pyrazine-7-carboxylic acid isopropylamide;
2-((R)-1-Acetylamino-indan-5-yloxy)-5H-pyrrolo[2,3-b]pyrazine-7-carboxylic acid ((S)-1,2,2-trimethyl-propyl)-amide;
2-[(R)-3-(3-Methyl-ureido)-indan-5-yloxy]-5H-pyrrolo[2,3-b]pyrazine-7-carboxylic acid isopropylamide;
2-(3-Hydroxy-indan-5-yloxy)-5H-pyrrolo[2,3-b]pyrazine-7-carboxylic acid isopropylamide;
2-((R)-3-Acetylamino-indan-5-yloxy)-5H-pyrrolo[2,3-b]pyrazine-7-carboxylic acid ((R)-1-cyclopropyl-ethyl)-amide;
2-((R)-3-Acetylamino-indan-5-yloxy)-5H-pyrrolo[2,3-b]pyrazine-7-carboxylic acid ((S)-1-cyclopropyl-ethyl)-amide;
2-((R)-3-Acetylamino-indan-5-yloxy)-5H-pyrrolo[2,3-b]pyrazine-7-carboxylic acid ((S)-sec-butyl)-amide;
2-(3-Oxo-indan-5-yloxy)-5H-pyrrolo[2,3-b]pyrazine-7-carboxylic acid isopropylamide;
2-((R)-3-Acetylamino-indan-5-yloxy)-5H-pyrrolo[2,3-b]pyrazine-7-carboxylic acid (cyano-methyl-methyl)-amide;
2-((R)-3-Ureido-indan-5-yloxy)-5H-pyrrolo[2,3-b]pyrazine-7-carboxylic acid isopropylamide;
2-(2-Acetylamino-indan-5-yloxy)-5H-pyrrolo[2,3-b]pyrazine-7-carboxylic acid isopropylamide;
2-((R)-3-Formylamino-indan-5-yloxy)-5H-pyrrolo[2,3-b]pyrazine-7-carboxylic acid isopropylamide;
2-(1H-Inden-5-yloxy)-5H-pyrrolo[2,3-b]pyrazine-7-carboxylic acid isopropylamide;
2-((R)-3-Hydroxy-indan-5-yloxy)-5H-pyrrolo[2,3-b]pyrazine-7-carboxylic acid isopropylamide;
2-((S)-3-Hydroxy-indan-5-yloxy)-5H-pyrrolo[2,3-b]pyrazine-7-carboxylic acid isopropylamide;
2-((R)-1-Amino-indan-5-yloxy)-5H-pyrrolo[2,3-b]pyrazine-7-carboxylic acid ((S)-1,2,2-trimethyl-propyl)-amide;
2-((R)-8-Acetylamino-5,6,7,8-tetrahydro-naphthalen-2-yloxy)-5H-pyrrolo[2,3-b]pyrazine-7-carboxylic acid ((S)-1,2,2-trimethyl-propyl)-amide;
2-((R)-8-Amino-5,6,7,8-tetrahydro-naphthalen-2-yloxy)-5H-pyrrolo[2,3-b]pyrazine-7-carboxylic acid ((S)-1,2,2-trimethyl-propyl)-amide;

2-((R)-8-Acetylamino-5,6,7,8-tetrahydro-naphthalen-2-yloxy)-5H-pyrrolo[2,3-b]pyrazine-7-carboxylic acid ((R)-1-cyclopropyl-ethyl)-amide;
2-((R)-8-Formylamino-5,6,7,8-tetrahydro-naphthalen-2-yloxy)-5H-pyrrolo[2,3-b]pyrazine-7-carboxylic acid ((R)-1-cyclopropyl-ethyl)-amide;
2-((R)-8-Amino-5,6,7,8-tetrahydro-naphthalen-2-yloxy)-5H-pyrrolo[2,3-b]pyrazine-7-carboxylic acid ((R)-1-cyclopropyl-ethyl)-amide;
2-((R)-3-Acetylamino-indan-5-yloxy)-5H-pyrrolo[2,3-b]pyrazine-7-carboxylic acid dicyclopropylmethyl-amide;
2-((R)-1-Acetylamino-indan-5-yloxy)-5H-pyrrolo[2,3-b]pyrazine-7-carboxylic acid ((R)-1-cyclopropyl-ethyl)-amide; and
2-((R)-8-Acetylamino-5,6,7,8-tetrahydro-naphthalen-2-yloxy)-5H-pyrrolo[2,3-b]pyrazine-7-carboxylic acid ((S)-2-methoxy-1-methyl-ethyl)-amide.
The application provides a method for treating an inflammatory or autoimmune condition comprising administering to a patient in need thereof a therapeutically effective amount of the compound of formula I or I′.

The application provides the above method, further comprising administering an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, or an agent for treating immunodeficiency disorders.

The application provides a method for treating an inflammatory condition comprising administering to a patient in need thereof a therapeutically effective amount of the compound of formula I or I′.

The application provides a method for treating rheumatoid arthritis comprising administering to a patient in need thereof a therapeutically effective amount of the compound of Formula I or I′.

The application provides a method for treating asthma comprising administering to a patient in need thereof a therapeutically effective amount of the compound of Formula I or I′.

The application provides a method for inhibiting T-cell proliferative disorder comprising administering to a patient in need thereof a therapeutically effective amount of the compound of formula I or I′.

The application provides a method for inhibiting T-cell proliferative disorder comprising administering to a patient in need thereof a therapeutically effective amount of the compound of formula I or I′.

The application provides the above method, wherein the proliferative disorder is cancer.

The application provides a method for treating a B-cell proliferative disorder comprising administering to a patient in need thereof a therapeutically effective amount of the compound of formula I or I′.

The application provides a method for treating an immune disorder including lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease, and Leukemia, comprising administering to a patient in need thereof a therapeutically effective amount of the compound of formula I or I′.

The application provides a method for preventing or treating all forms of organ rejection, including acute allograft or xenograft rejection and chronic allograft or xenograft rejection, of vascularized or non-vascularized transplants, comprising administering to a patient in need thereof the compound of formula I or I′.

The application provides a method for inhibiting JAK3 activity comprising administering the compound of formula I or I′, wherein the compound exhibits an IC₅₀ of 50 micromolar or less in an in vitro biochemical assay of JAK3 activity.

The application provides the above method, wherein the compound exhibits an IC₅₀ of 100 nanomolar or less in an in vitro biochemical assay of JAK3 activity.

The application provides the above method, wherein the compound exhibits an IC₅₀ of 10 nanomolar or less in an in vitro biochemical assay of JAK3 activity.

The application provides a method for inhibiting SYK activity comprising administering the compound of formula I or I′, wherein the compound exhibits an IC₅₀ of 50 micromolar or less in an in vitro biochemical assay of SYK activity.

The application provides the above method, wherein the compound exhibits an IC₅₀ of 100 nanomolar or less in an in vitro biochemical assay of SYK activity.

The application provides the above method, wherein the compound exhibits an IC₅₀ of 10 nanomolar or less in an in vitro biochemical assay of SYK activity.

The application provides a method for treating an inflammatory condition comprising co-administering to a patient in need thereof a therapeutically effective amount of an anti-inflammatory compound in combination with the compound of formula I or I′.

The application provides a method for treating an immune disorder comprising co-administering to a patient in need thereof a therapeutically effective amount of an immunosuppressant compound in combination with the compound of formula I or I′.

The application provides a pharmaceutical composition comprising the compound of formula I or I′, admixed with at least one pharmaceutically acceptable carrier, excipient or diluent.

The application provides the above pharmaceutical compound of formula I or I′, further comprising an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, and an agent for treating immunodeficiency disorders.

The application provides the compound as described above for use in the treatment of an inflammatory or autoimmune condition. The application provides the compound as described above for use in the treatment of any one of the condition mentioned above.

The application provides a use of the compound of formula I or I′ in the manufacture of a medicament for the treatment of an inflammatory disorder.

The application provides a use of the compound of formula I or I′ in the manufacture of a medicament for the treatment of an autoimmune disorder.

The application provides a compound or method as described herein.

### Compounds and Preparation

Examples of representative compounds encompassed by the present invention and within the scope of the invention are provided in the following Table. These examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOMTM v.4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts exemplified compounds according to Formula I.

TABLE I

| | | SYSTEMATIC | |
|---|---|---|---|
| # | STRUCTURE | NAME | MP |
| I-1 | 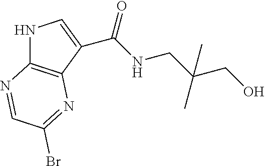 [SMILES: CC(C)(CO)CNC(=O)c1c[nH]c2ncc(Br)nc12] | 2-Bromo-5H- pyrrolo[2,3- b]pyrazine-7- carboxylic acid (3-hydroxy-2,2- dimethyl- propyl)-amide | 248.0- 250.0 |
| I-2 | 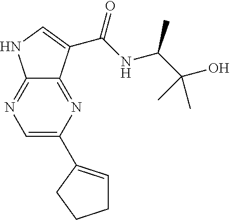 [SMILES: C[C@H](NC(=O)c1c[nH]c2ncc(C3=CCCC3)nc12)C(C)(C)O] | 2-Cyclopent-1- enyl-5H- pyrrolo[2,3- b]pyrazine-7- carboxylic acid ((S)-2-hydroxy- 1,2-dimethyl- propyl)-amide | |
| I-3 | 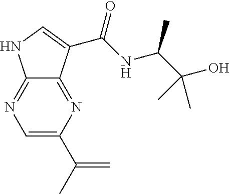 [SMILES: C=C(C)c1cnc2[nH]cc(C(=O)N[C@@H](C)C(C)(C)O)c2n1] | 2-Isopropenyl- 5H-pyrrolo[2,3- b]pyrazine-7- carboxylic acid ((S)-2-hydroxy- 1,2-dimethyl- propyl)-amide | |
| I-4 | 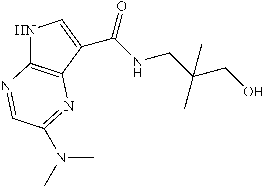 [SMILES: CN(C)c1cnc2[nH]cc(C(=O)NCC(C)(C)CO)c2n1] | 2-Dimethylamino- 5H-pyrrolo[2,3- b]pyrazine-7- carboxylic acid (3-hydroxy-2,2- dimethyl- propyl)-amide | 222.0- 224.0 |
| I-5 | 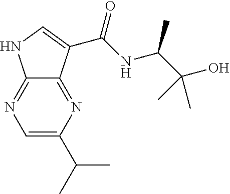 [SMILES: CC(C)c1cnc2[nH]cc(C(=O)N[C@@H](C)C(C)(C)O)c2n1] | 2-Isopropyl-5H- pyrrolo[2,3- b]pyrazine-7- carboxylic acid ((S)-2-hydroxy- 1,2-dimethyl- propyl)-amide | |
| I-6 | 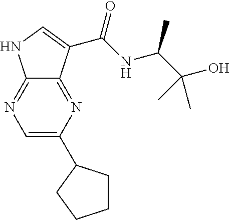 [SMILES: C[C@H](NC(=O)c1c[nH]c2ncc(C3CCCC3)nc12)C(C)(C)O] | 2-Cyclopentyl- 5H-pyrrolo[2,3- b]pyrazine-7- carboxylic acid ((S)-2-hydroxy- 1,2-dimethyl- propyl)-amide | |
| I-7 | 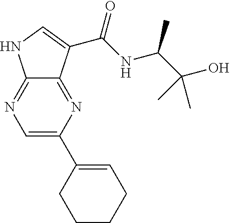 [SMILES: C[C@H](NC(=O)c1c[nH]c2ncc(C3=CCCCC3)nc12)C(C)(C)O] | 2-Cyclohex-1- enyl-5H- pyrrolo[2,3- b]pyrazine-7- carboxylic acid ((S)-2-hydroxy- 1,2-dimethyl- propyl)-amide | |
| I-8 | 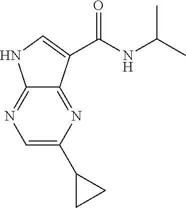 [SMILES: CC(C)NC(=O)c1c[nH]c2ncc(C3CC3)nc12] | 2-Cyclopropyl- 5H-pyrrolo[2,3- b]pyrazine-7- carboxylic acid isopropylamide | >300- |
| I-9 | 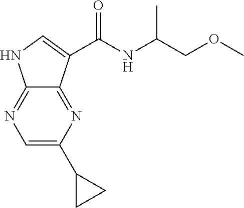 [SMILES: COCC(C)NC(=O)c1c[nH]c2ncc(C3CC3)nc12] | 2-Cyclopropyl- 5H-pyrrolo[2,3- b]pyrazine-7- carboxylic acid (2-methoxy-1- methyl-ethyl)- amide | 238.0- 240.0 |
| I-10 | 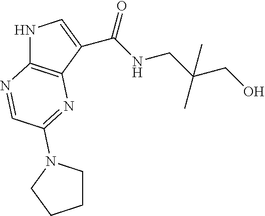 [SMILES: CC(C)(CO)CNC(=O)c1c[nH]c2ncc(N3CCCC3)nc12] | 2-Pyrrolidin-1- yl-5H- pyrrolo[2,3- b]pyrazine-7- carboxylic acid (3-hydroxy-2,2- dimethyl- propyl)-amide | 220.0- 222.0 |
| I-11 | 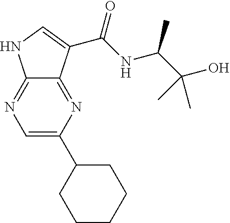 [SMILES: C[C@H](NC(=O)c1c[nH]c2ncc(C3CCCCC3)nc12)C(C)(C)O] | 2-Cyclohexyl- 5H-pyrrolo[2,3- b]pyrazine-7- carboxylic acid ((S)-2-hydroxy- 1,2-dimethyl- propyl)-amide | |
| I-12 | 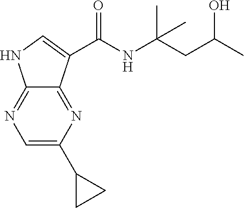 [SMILES: CC(O)CC(C)(C)NC(=O)c1c[nH]c2ncc(C3CC3)nc12] | 2-Cyclopropyl- 5H-pyrrolo[2,3- b]pyrazine-7- carboxylic acid (3-hydroxy-1,1- dimethyl-butyl)- amide | 230.0- 232.0 |
| I-13 | 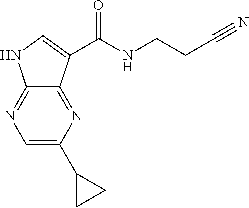 [SMILES: N#CCCNC(=O)c1c[nH]c2ncc(C3CC3)nc12] | 2-Cyclopropyl- 5H-pyrrolo[2,3- b]pyrazine-7- carboxylic acid (2-cyano-ethyl)- amide | 236.0- 238.0 |
| I-14 | 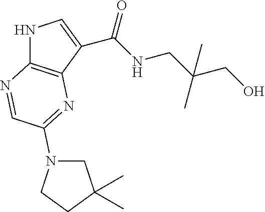 [SMILES: CC(C)(CO)CNC(=O)c1c[nH]c2ncc(N3CCC(C)(C)C3)nc12] | 2-(3,3- Dimethyl- pyrrolidin-1-yl)- 5H-pyrrolo[2,3- b]pyrazine-7- carboxylic acid (3-hydroxy-2,2- dimethyl- propyl)-amide | 223.0- 225.0 |
| I-15 | 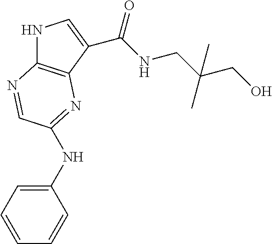 [SMILES: CC(C)(CO)CNC(=O)c1c[nH]c2ncc(Nc3ccccc3)nc12] | 2-Phenylamino- 5H-pyrrolo[2,3- b]pyrazine-7- carboxylic acid (3-hydroxy-2,2- dimethyl- propyl)-amide | 280.0- 282.0 |
| I-16 | 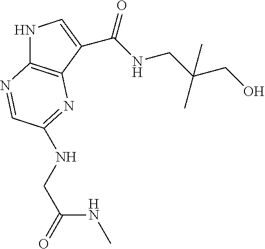 [SMILES: CNC(=O)CNc1cnc2[nH]cc(C(=O)NCC(C)(C)CO)c2n1] | 2-(Methyl- carbamoylmethyl- amino)-5H- pyrrolo[2,3- b]pyrazine-7- carboxylic acid (3-hydroxy-2,2- dimethyl- propyl)-amide | 270- 275 |
| I-17 | 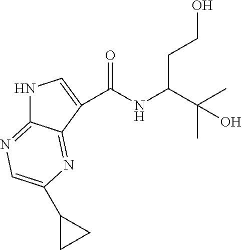 [SMILES: CC(C)(O)C(CCO)NC(=O)c1c[nH]c2ncc(C3CC3)nc12] | 2-Cyclopropyl- 5H-pyrrolo[2,3- b]pyrazine-7- carboxylic acid [2-hydroxy-1-(2- hydroxy-ethyl)- 2-methyl- propyl]-amide | 195- 198 |
| I-18 | 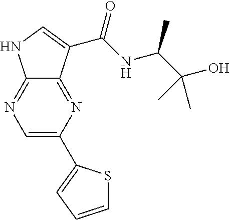 [SMILES: C[C@H](NC(=O)c1c[nH]c2ncc(-c3cccs3)nc12)C(C)(C)O] | 2-Thiophen-2- yl-5H- pyrrolo[2,3- b]pyrazine-7- carboxylic acid ((S)-2-hydroxy- 1,2-dimethyl- propyl)-amide | |
| I-19 | 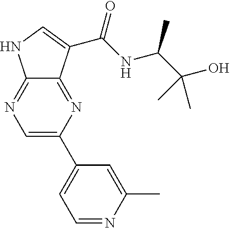 [SMILES: Cc1cc(-c2cnc3[nH]cc(C(=O)N[C@@H](C)C(C)(C)O)c3n2)ccn1] | 2-Cyclopropyl- 5H-pyrrolo[2,3- b]pyrazine-7- carboxylic acid (1-cyclopropyl- ethyl)-amide | 269- 272 |
| I-20 | 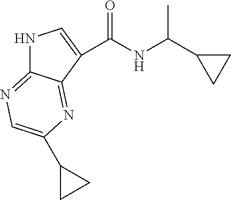 [SMILES: CC(NC(=O)c1c[nH]c2ncc(C3CC3)nc12)C1CC1] | 2-(2-Methyl- pyridin-4-yl)- 5H-pyrrolo[2,3- b]pyrazine-7- carboxylic acid ((S)-2-hydroxy- 1,2-dimethyl- propyl)-amide | |
| I-21 | 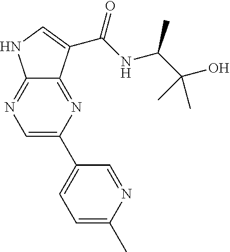 [SMILES: Cc1ccc(-c2cnc3[nH]cc(C(=O)N[C@@H](C)C(C)(C)O)c3n2)cn1] | 2-(6-Methyl- pyridin-3-yl)- 5H-pyrrolo[2,3- b]pyrazine-7- carboxylic acid ((S)-2-hydroxy- 1,2-dimethyl- propyl)-amide | |
| I-22 | 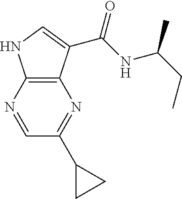 [SMILES: CC[C@H](C)NC(=O)c1c[nH]c2ncc(C3CC3)nc12] | 2-Cyclopropyl- 5H-pyrrolo[2,3- b]pyrazine-7- carboxylic acid ((S)-sec-butyl)- amide | 280.0- 282.0 |
| I-23 | 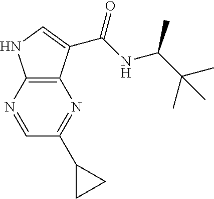 [SMILES: C[C@H](NC(=O)c1c[nH]c2ncc(C3CC3)nc12)C(C)(C)C] | 2-Cyclopropyl- 5H-pyrrolo[2,3- b]pyrazine-7- carboxylic acid ((S)-1,2,2- trimethyl- propyl)-amide | >300- |
| I-24 | 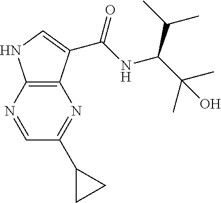 [SMILES: CC(C)[C@H](NC(=O)c1c[nH]c2ncc(C3CC3)nc12)C(C)(C)O] | 2-Cyclopropyl- 5H-pyrrolo[2,3- b]pyrazine-7- carboxylic acid ((S)-2-hydroxy- 1-isopropyl-2- methyl-propyl)- amide | 232.0- 234.0 |
| I-25 | 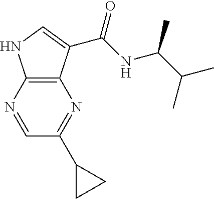 [SMILES: CC(C)[C@H](C)NC(=O)c1c[nH]c2ncc(C3CC3)nc12] | 2-Cyclopropyl- 5H-pyrrolo[2,3- b]pyrazine-7- carboxylic acid ((S)-1,2- dimethyl- propyl)-amide | 281.0- 283.0 |
| I-26 | 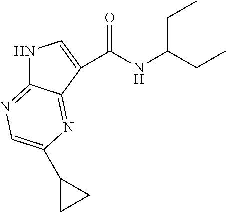 [SMILES: CCC(CC)NC(=O)c1c[nH]c2ncc(C3CC3)nc12] | 2-Cyclopropyl- 5H-pyrrolo[2,3- b]pyrazine-7- carboxylic acid (1-ethyl-propyl)- amide | 245- 246 |
| I-27 | 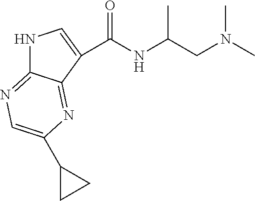 [SMILES: CC(CN(C)C)NC(=O)c1c[nH]c2ncc(C3CC3)nc12] | 2-Cyclopropyl- 5H-pyrrolo[2,3- b]pyrazine-7- carboxylic acid (2-dimethylamino- 1-methyl-ethyl)- amide | 225- 229 |
| I-28 | 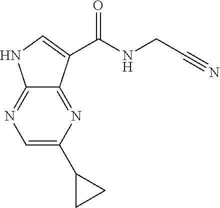 [SMILES: N#CCNC(=O)c1c[nH]c2ncc(C3CC3)nc12] | 2-Cyclopropyl- 5H-pyrrolo[2,3- b]pyrazine-7- carboxylic acid cyanomethyl- amide | 240.0- 242.0 |
| I-29 | 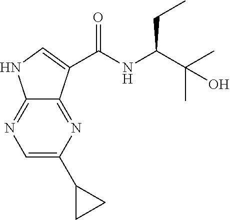 [SMILES: CC[C@H](NC(=O)c1c[nH]c2ncc(C3CC3)nc12)C(C)(C)O] | 2-Cyclopropyl- 5H-pyrrolo[2,3- b]pyrazine-7- carboxylic acid ((S)-1-ethyl-2- hydroxy-2- methyl-propyl)- amide | 229.0- 231.0 |
| I-30 | 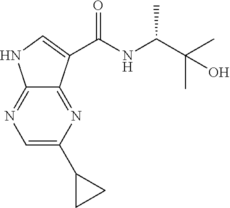 [SMILES: C[C@@H](NC(=O)c1c[nH]c2ncc(C3CC3)nc12)C(C)(C)O] | 2-Cyclopropyl- 5H-pyrrolo[2,3- b]pyrazine-7- carboxylic acid ((R)-2-hydroxy- 1,2-dimethyl- propyl)-amide | 269.0- 271.0 |
| I-31 | 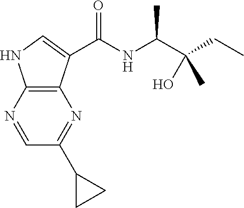 [SMILES: CC[C@](C)(O)[C@H](C)NC(=O)c1c[nH]c2ncc(C3CC3)nc12] | 2-Cyclopropyl- 5H-pyrrolo[2,3- b]pyrazine-7- carboxylic acid ((1S,2S)-2- hydroxy-1,2- dimethyl-butyl)- amide | 243.0- 245.0 |
| I-32 | 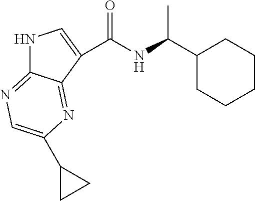 [SMILES: C[C@H](NC(=O)c1c[nH]c2ncc(C3CC3)nc12)C1CCCCC1] | 2-Cyclopropyl- 5H-pyrrolo[2,3- b]pyrazine-7- carboxylic acid ((S)-1-cyclohexyl- ethyl)-amide | 246- 249 |
| I-33 | 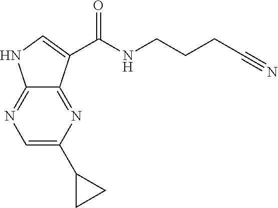 [SMILES: N#CCCCNC(=O)c1c[nH]c2ncc(C3CC3)nc12] | 2-Cyclopropyl- 5H-pyrrolo[2,3- b]pyrazine-7- carboxylic acid (3-cyano- propyl)-amide | 232.0- 234.0 |
| I-34 | 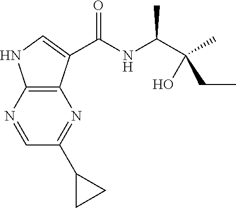 [SMILES: CC[C@@](C)(O)[C@H](C)NC(=O)c1c[nH]c2ncc(C3CC3)nc12] | 2-Cyclopropyl- 5H-pyrrolo[2,3- b]pyrazine-7- carboxylic acid ((1S,2R)-2- hydroxy-1,2- dimethyl-butyl)- amide | 262.0- 264.0 |
| I-35 | 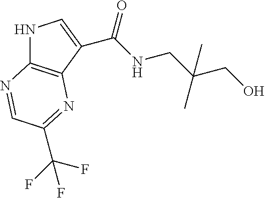 [SMILES: CC(C)(CO)CNC(=O)c1c[nH]c2ncc(C(F)(F)F)nc12] | 2-Trifluoromethyl- 5H-pyrrolo[2,3- b]pyrazine-7- carboxylic acid (3-hydroxy-2,2- dimethyl- propyl)-amide | 221.0- 223.0 |
| I-36 | 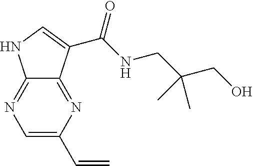 [SMILES: C=Cc1cnc2[nH]cc(C(=O)NCC(C)(C)CO)c2n1] | 2-Vinyl-5H- pyrrolo[2,3- b]pyrazine-7- carboxylic acid (3-hydroxy-2,2- dimethyl- propyl)-amide | |
| I-37 | 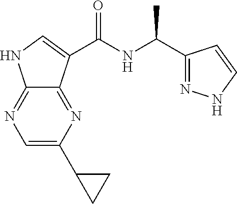 [SMILES: C[C@H](NC(=O)c1c[nH]c2ncc(C3CC3)nc12)c1cc[nH]n1] | 2-Cyclopropyl- 5H-pyrrolo[2,3- b]pyrazine-7- carboxylic acid [(S)-1-(1H- pyrazol-3-yl)- ethyl]-amide | |
| I-38 | 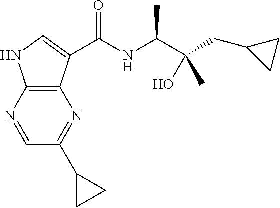 [SMILES: C[C@H](NC(=O)c1c[nH]c2ncc(C3CC3)nc12)[C@@](C)(O)CC1CC1] | 2-Cyclopropyl- 5H-pyrrolo[2,3- b]pyrazine-7- carboxylic acid ((1S,2S)-3- cyclopropyl-2- hydroxy-1,2- dimethyl- propyl)-amide | |
| I-39 | 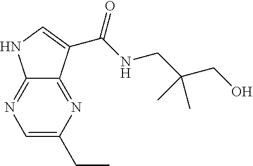 [SMILES: CCc1cnc2[nH]cc(C(=O)NCC(C)(C)CO)c2n1] | 2-Ethyl-5H- pyrrolo[2,3- b]pyrazine-7- carboxylic acid (3-hydroxy-2,2- dimethyl- propyl)-amide | |
| I-40 | 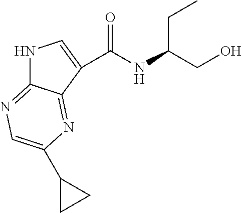 [SMILES: CC[C@@H](CO)NC(=O)c1c[nH]c2ncc(C3CC3)nc12] | 2-Cyclopropyl- 5H-pyrrolo[2,3- b]pyrazine-7- carboxylic acid ((S)-1- hydroxymethyl- propyl)-amide | 250- 252 |
| I-41 | 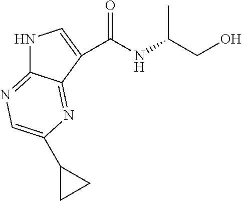 [SMILES: C[C@H](CO)NC(=O)c1c[nH]c2ncc(C3CC3)nc12] | 2-Cyclopropyl- 5H-pyrrolo[2,3- b]pyrazine-7- carboxylic acid ((R)-2-hydroxy- 1-methyl-ethyl)- amide | 265- 268 |
| I-42 | 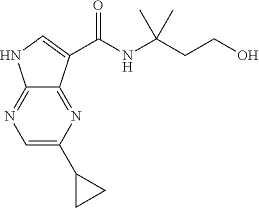 [SMILES: CC(C)(CCO)NC(=O)c1c[nH]c2ncc(C3CC3)nc12] | 2-Cyclopropyl- 5H-pyrrolo[2,3- b]pyrazine-7- carboxylic acid (3-hydroxy-1,1- dimethyl- propyl)-amide | 250.0- 252.0 |
| I-43 | 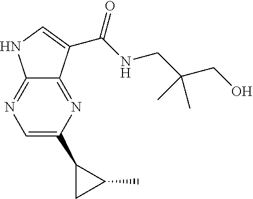 [SMILES: C[C@@H]1C[C@H]1c1cnc2[nH]cc(C(=O)NCC(C)(C)CO)c2n1] | 2-((1R,2R)-2- Methyl- cyclopropyl)- 5H-pyrrolo[2,3- b]pyrazine-7- carboxylic acid (3-hydroxy-2,2- dimethyl- propyl)-amide | |
| I-44 | 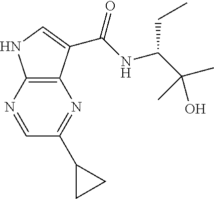 [SMILES: CC[C@@H](NC(=O)c1c[nH]c2ncc(C3CC3)nc12)C(C)(C)O] | 2-Cyclopropyl- 5H-pyrrolo[2,3- b]pyrazine-7- carboxylic acid ((R)-1-ethyl-2- hydroxy-2- methyl-propyl)- amide | 218.0- 220.0 |
| I-45 | 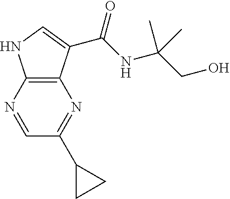 [SMILES: CC(C)(CO)NC(=O)c1c[nH]c2ncc(C3CC3)nc12] | 2-Cyclopropyl- 5H-pyrrolo[2,3- b]pyrazine-7- carboxylic acid (2-hydroxy-1,1- dimethyl-ethyl)- amide | 293.0- 295.0 |
| I-46 | 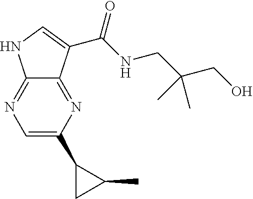 [SMILES: C[C@H]1C[C@H]1c1cnc2[nH]cc(C(=O)NCC(C)(C)CO)c2n1] | 2-((1R,2S)-2- Methyl- cyclopropyl)- 5H-pyrrolo[2,3- b]pyrazine-7- carboxylic acid (3-hydroxy-2,2- dimethyl- propyl)-amide | 244.0- 246.0 |
| I-47 | 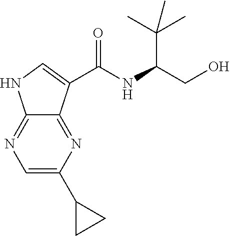 [SMILES: CC(C)(C)[C@@H](CO)NC(=O)c1c[nH]c2ncc(C3CC3)nc12] | 2-Cyclopropyl- 5H-pyrrolo[2,3- b]pyrazine-7- carboxylic acid ((S)-1- hydroxymethyl- 2,2-dimethyl- propyl)-amide | 259.0- 261.0 |
| I-48 | 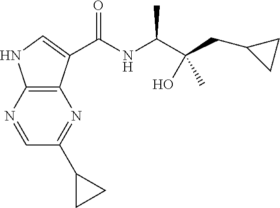 [SMILES: C[C@H](NC(=O)c1c[nH]c2ncc(C3CC3)nc12)[C@](C)(O)CC1CC1] | 2-Cyclopropyl- 5H-pyrrolo[2,3- b]pyrazine-7- carboxylic acid ((1S,2R)-3- cyclopropyl-2- hydroxy-1,2- dimethyl- propyl)-amide | |
| I-49 | 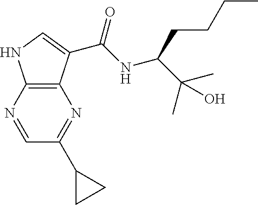 [SMILES: CCCC[C@H](NC(=O)c1c[nH]c2ncc(C3CC3)nc12)C(C)(C)O] | 2-Cyclopropyl- 5H-pyrrolo[2,3- b]pyrazine-7- carboxylic acid [(S)-1-(1- hydroxy-1- methyl-ethyl)- pentyl]-amide | 170.0- 172.0 |
| I-50 | 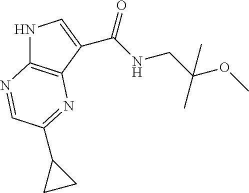 [SMILES: COC(C)(C)CNC(=O)c1c[nH]c2ncc(C3CC3)nc12] | 2-Cyclopropyl- 5H-pyrrolo[2,3- b]pyrazine-7- carboxylic acid (2-methoxy-2- methyl-propyl)- amide | 259.0- 262.0 |
| I-51 | 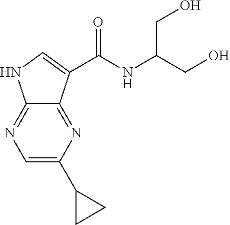 [SMILES: O=C(NC(CO)CO)c1c[nH]c2ncc(C3CC3)nc12] | 2-Cyclopropyl- 5H-pyrrolo[2,3- b]pyrazine-7- carboxylic acid (2-hydroxy-1- hydroxymethyl- ethyl)-amide | 255.0- 256.7 |
| I-52 | 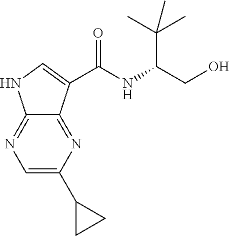 [SMILES: CC(C)(C)[C@H](CO)NC(=O)c1c[nH]c2ncc(C3CC3)nc12] | 2-Cyclopropyl- 5H-pyrrolo[2,3- b]pyrazine-7- carboxylic acid ((R)-1- hydroxymethyl- 2,2-dimethyl- propyl)-amide | 270.0- 273.0 |
| I-53 | 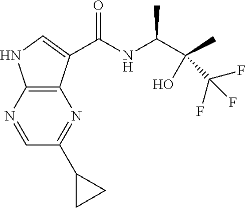 [SMILES: C[C@H](NC(=O)c1c[nH]c2ncc(C3CC3)nc12)[C@@](C)(O)C(F)(F)F] | 2-Cyclopropyl- 5H-pyrrolo[2,3- b]pyrazine-7- carboxylic acid ((1S,2R)-3,3,3- trifluoro-2- hydroxy-1,2- dimethyl- propyl)-amide | 280.0- 283.0 |
| I-54 | 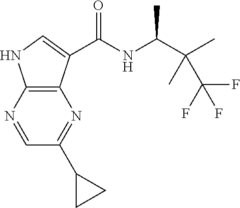 [SMILES: C[C@H](NC(=O)c1c[nH]c2ncc(C3CC3)nc12)C(C)(C)C(F)(F)F] | 2-Cyclopropyl- 5H-pyrrolo[2,3- b]pyrazine-7- carboxylic acid ((S)-3,3,3- trifluoro-1,2,2- trimethyl- propyl)-amide | >300- |
| I-55 | 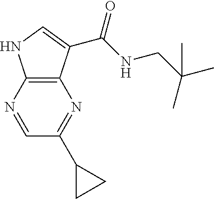 [SMILES: CC(C)(C)CNC(=O)c1c[nH]c2ncc(C3CC3)nc12] | 2-Cyclopropyl- 5H-pyrrolo[2,3- b]pyrazine-7- carboxylic acid (2,2-dimethyl- propyl)-amide | |
| I-56 | 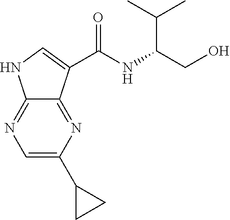 [SMILES: CC(C)[C@H](CO)NC(=O)c1c[nH]c2ncc(C3CC3)nc12] | 2-Cyclopropyl- 5H-pyrrolo[2,3- b]pyrazine-7- carboxylic acid ((R)-1- hydroxymethyl- 2-methyl- propyl)-amide | 250.0- 253.0 |
| I-57 | 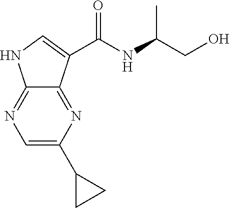 [SMILES: C[C@@H](CO)NC(=O)c1c[nH]c2ncc(C3CC3)nc12] | 2-Cyclopropyl- 5H-pyrrolo[2,3- b]pyrazine-7- carboxylic acid ((S)-2-hydroxy- 1-methyl-ethyl)- amide | 274.0- 276.0 |
| I-58 | 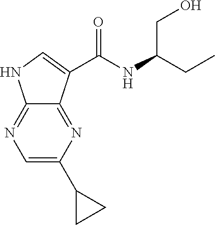 [SMILES: CC[C@H](CO)NC(=O)c1c[nH]c2ncc(C3CC3)nc12] | 2-Cyclopropyl- 5H-pyrrolo[2,3- b]pyrazine-7- carboxylic acid ((R)-1- hydroxymethyl- propyl)-amide | 250.0- 253.0 |
| I-59 | 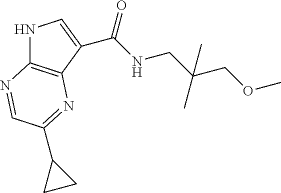 [SMILES: COCC(C)(C)CNC(=O)c1c[nH]c2ncc(C3CC3)nc12] | 2-Cyclopropyl- 5H-pyrrolo[2,3- b]pyrazine-7- carboxylic acid (3-methoxy-2,2- dimethyl- propyl)-amide | 230.0- 232.0 |
| I-60 | 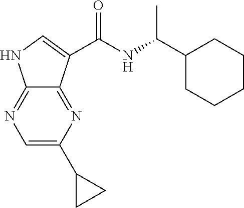 [SMILES: C[C@@H](NC(=O)c1c[nH]c2ncc(C3CC3)nc12)C1CCCCC1] | 2-Cyclopropyl- 5H-pyrrolo[2,3- b]pyrazine-7- carboxylic acid ((R)-1- cyclohexyl- ethyl)-amide | 253.0- 255.0 |
| I-61 | 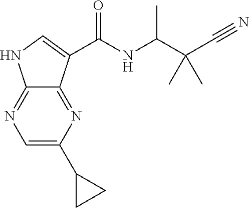 [SMILES: CC(NC(=O)c1c[nH]c2ncc(C3CC3)nc12)C(C)(C)C#N] | 2-Cyclopropyl- 5H-pyrrolo[2,3- b]pyrazine-7- carboxylic acid (2-cyano-1,2,2- trimethyl-ethyl)- amide | 295.0- 297.0 |
| I-62 | 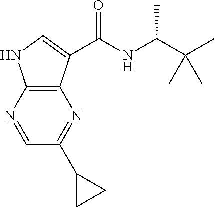 [SMILES: C[C@@H](NC(=O)c1c[nH]c2ncc(C3CC3)nc12)C(C)(C)C] | 2-Cyclopropyl- 5H-pyrrolo[2,3- b]pyrazine-7- carboxylic acid ((R)-1,2,2- trimethyl- propyl)-amide | 298.0- 300.0 |
| I-63 | 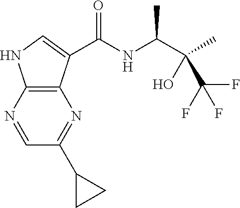 [SMILES: C[C@H](NC(=O)c1c[nH]c2ncc(C3CC3)nc12)[C@](C)(O)C(F)(F)F] | 2-Cyclopropyl- 5H-pyrrolo[2,3- b]pyrazine-7- carboxylic acid ((1S,2S)-3,3,3- trifluoro-2- hydroxy-1,2- dimethyl- propyl)-amide | 290.0- 292.0 |
| I-64 | 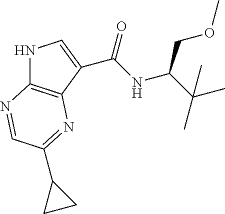 [SMILES: COC[C@H](NC(=O)c1c[nH]c2ncc(C3CC3)nc12)C(C)(C)C] | 2-Cyclopropyl- 5H-pyrrolo[2,3- b]pyrazine-7- carboxylic acid ((R)-1- methoxymethyl- 2,2-dimethyl- propyl)-amide | 265- 270 |
| I-65 | 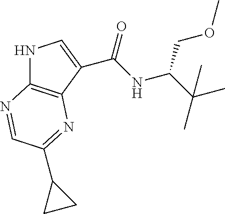 [SMILES: COC[C@@H](NC(=O)c1c[nH]c2ncc(C3CC3)nc12)C(C)(C)C] | 2-Cyclopropyl- 5H-pyrrolo[2,3- b]pyrazine-7- carboxylic acid ((S)-1- methoxymethyl- 2,2-dimethyl- propyl)-amide | 268.0- 270.0 |
| I-66 | 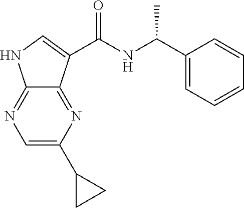 [SMILES: C[C@@H](NC(=O)c1c[nH]c2ncc(C3CC3)nc12)c1ccccc1] | 2-Cyclopropyl- 5H-pyrrolo[2,3- b]pyrazine-7- carboxylic acid ((R)-1-phenyl- ethyl)-amide | 278.0- 280.0 |
| I-67 | 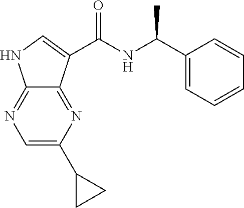 [SMILES: C[C@H](NC(=O)c1c[nH]c2ncc(C3CC3)nc12)c1ccccc1] | 2-Cyclopropyl- 5H-pyrrolo[2,3- b]pyrazine-7- carboxylic acid ((S)-1-phenyl- ethyl)-amide | 272.0- 274.0 |
| I-68 | 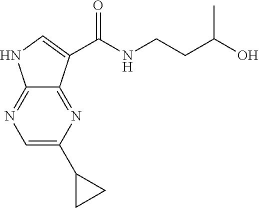 [SMILES: CC(O)CCNC(=O)c1c[nH]c2ncc(C3CC3)nc12] | 2-Cyclopropyl- 5H-pyrrolo[2,3- b]pyrazine-7- carboxylic acid (3-hydroxy- butyl)-amide | 228.0- 230.0 |
| I-69 | 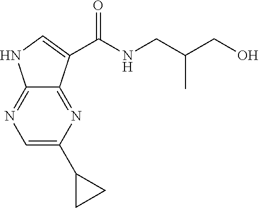 [SMILES: CC(CO)CNC(=O)c1c[nH]c2ncc(C3CC3)nc12] | 2-Cyclopropyl- 5H-pyrrolo[2,3- b]pyrazine-7- carboxylic acid (3-hydroxy-2- methyl-propyl)- amide | 252.0- 254.0 |
| I-70 | 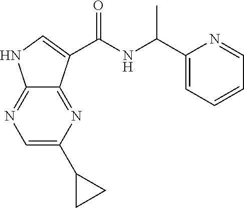 [SMILES: CC(NC(=O)c1c[nH]c2ncc(C3CC3)nc12)c1ccccn1] | 2-Cyclopropyl- 5H-pyrrolo[2,3- b]pyrazine-7- carboxylic acid (1-pyridin-2-yl- ethyl)-amide | 217.0- 219.0 |
| I-71 | 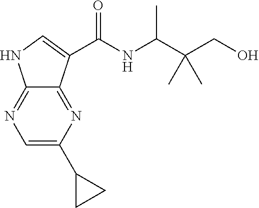 [SMILES: CC(NC(=O)c1c[nH]c2ncc(C3CC3)nc12)C(C)(C)CO] | 2-Cyclopropyl- 5H-pyrrolo[2,3- b]pyrazine-7- carboxylic acid (3-hydroxy- 1,2,2-trimethyl- propyl)-amide | 268.0- 270.0 |
| I-72 | 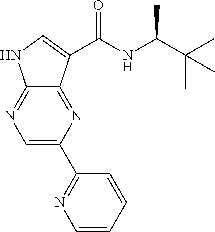 [SMILES: C[C@H](NC(=O)c1c[nH]c2ncc(-c3ccccn3)nc12)C(C)(C)C] | 2-Pyridin-2-yl- 5H-pyrrolo[2,3- b]pyrazine-7- carboxylic acid ((S)-1,2,2- trimethyl- propyl)-amide | >300- |
| I-73 | 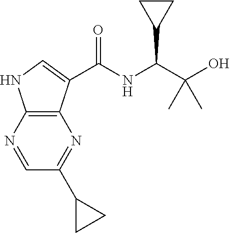 [SMILES: CC(C)(O)[C@@H](NC(=O)c1c[nH]c2ncc(C3CC3)nc12)C1CC1] | 2-Cyclopropyl- 5H-pyrrolo[2,3- b]pyrazine-7- carboxylic acid ((S)-1- cyclopropyl-2- hydroxy-2- methyl-propyl)- amide | 238.0- 240.0 |
| I-74 | 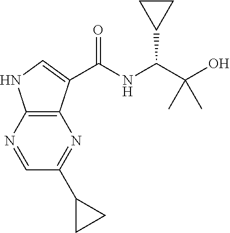 [SMILES: CC(C)(O)[C@H](NC(=O)c1c[nH]c2ncc(C3CC3)nc12)C1CC1] | 2-Cyclopropyl- 5H-pyrrolo[2,3- b]pyrazine-7- carboxylic acid ((R)-1- cyclopropyl-2- hydroxy-2- methyl-propyl)- amide | 235.0- 237.0 |
| I-75 | 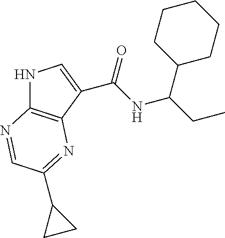 [SMILES: CCC(NC(=O)c1c[nH]c2ncc(C3CC3)nc12)C1CCCCC1] | 2-Cyclopropyl- 5H-pyrrolo[2,3- b]pyrazine-7- carboxylic acid (1-cyclohexyl- propyl)-amide | 208.0- 210.0 |
| I-76 | 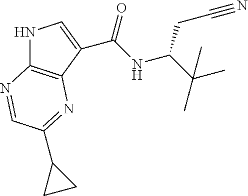 [SMILES: CC(C)(C)[C@@H](CC#N)NC(=O)c1c[nH]c2ncc(C3CC3)nc12] | 2-Cyclopropyl- 5H-pyrrolo[2,3- b]pyrazine-7- carboxylic acid ((R)-1- cyanomethyl- 2,2-dimethyl- propyl)-amide | 259.0- 261.0 |
| I-77 | 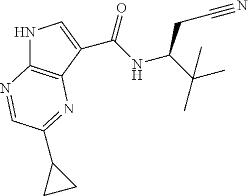 [SMILES: CC(C)(C)[C@H](CC#N)NC(=O)c1c[nH]c2ncc(C3CC3)nc12] | 2-Cyclopropyl- 5H-pyrrolo[2,3- b]pyrazine-7- carboxylic acid ((S)-1- cyanomethyl- 2,2-dimethyl- propyl)-amide | 258.0- 260.0 |
| I-78 | 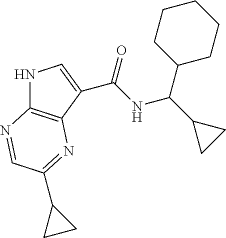 [SMILES: O=C(NC(C1CCCCC1)C1CC1)c1c[nH]c2ncc(C3CC3)nc12] | 2-Cyclopropyl- 5H-pyrrolo[2,3- b]pyrazine-7- carboxylic acid (cyclohexyl- cyclopropyl- methyl)-amide | 174.0- 176.0 |
| I-79 | 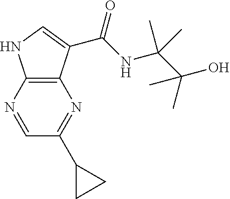 [SMILES: CC(C)(O)C(C)(C)NC(=O)c1c[nH]c2ncc(C3CC3)nc12] | 2-Cyclopropyl- 5H-pyrrolo[2,3- b]pyrazine-7- carboxylic acid (2-hydroxy- 1,1,2-trimethyl- propyl)-amide | 270.0- 273.0 |
| I-80 | 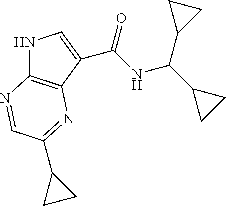 [SMILES: O=C(NC(C1CC1)C1CC1)c1c[nH]c2ncc(C3CC3)nc12] | 2-Cyclopropyl- 5H-pyrrolo[2,3- b]pyrazine-7- carboxylic acid dicyclopropyl- methyl-amide | 224.0- 226.0 |
| I-81 | 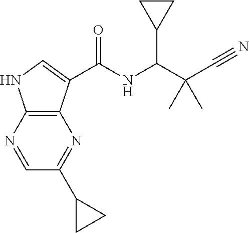 [SMILES: CC(C)(C#N)C(NC(=O)c1c[nH]c2ncc(C3CC3)nc12)C1CC1] | 2-Cyclopropyl- 5H-pyrrolo[2,3- b]pyrazine-7- carboxylic acid (2-cyano-1- cyclopropyl-2,2- dimethyl-ethyl)- amide | 230.0- 232.0 |
| I-82 | 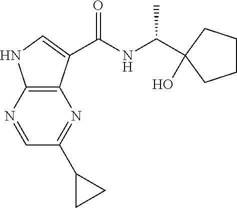 [SMILES: C[C@@H](NC(=O)c1c[nH]c2ncc(C3CC3)nc12)C1(O)CCCC1] | 2-Cyclopropyl- 5H-pyrrolo[2,3- b]pyrazine-7- carboxylic acid [(R)-1-(1- hydroxy- cyclopentyl)- ethyl]-amide | 287.0- 290.0 |
| I-83 | 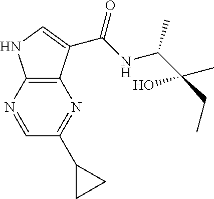 [SMILES: CC[C@@](C)(O)[C@@H](C)NC(=O)c1c[nH]c2ncc(C3CC3)nc12] | 2-Cyclopropyl- 5H-pyrrolo[2,3- b]pyrazine-7- carboxylic acid ((1R,2R)-2- hydroxy-1,2- dimethyl-butyl)- amide | 245.0- 247.0 |
| I-84 | 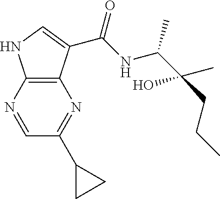 [SMILES: CCC[C@@](C)(O)[C@@H](C)NC(=O)c1c[nH]c2ncc(C3CC3)nc12] | 2-Cyclopropyl- 5H-pyrrolo[2,3- b]pyrazine-7- carboxylic acid ((1R,2R)-2- hydroxy-1,2- dimethyl- pentyl)-amide | 222.0- 224.0 |
| I-85 | 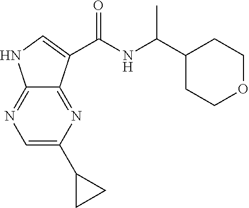 [SMILES: CC(NC(=O)c1c[nH]c2ncc(C3CC3)nc12)C1CCOCC1] | 2-Cyclopropyl- 5H-pyrrolo[2,3- b]pyrazine-7- carboxylic acid [1-(tetrahydro- pyran-4-yl)- ethyl]-amide | 260.0- 262.0 |
| I-86 | 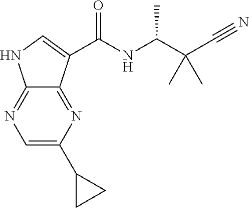 [SMILES: C[C@@H](NC(=O)c1c[nH]c2ncc(C3CC3)nc12)C(C)(C)C#N] | 2-Cyclopropyl- 5H-pyrrolo[2,3- b]pyrazine-7- carboxylic acid ((R)-2-cyano- 1,2,2-trimethyl- ethyl)-amide | 278.0- 281.0 |
| I-87 | 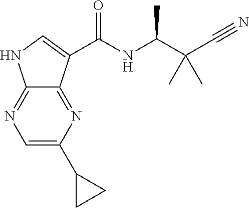 [SMILES: C[C@H](NC(=O)c1c[nH]c2ncc(C3CC3)nc12)C(C)(C)C#N] | 2-Cyclopropyl- 5H-pyrrolo[2,3- b]pyrazine-7- carboxylic acid ((S)-2-cyano- 1,2,2-trimethyl- ethyl)-amide | 277.0- 279.0 |
| I-88 | 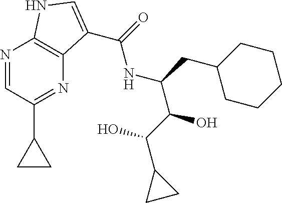 [SMILES: O=C(N[C@@H](CC1CCCCC1)[C@@H](O)[C@@H](O)C1CC1)c1c[nH]c2ncc(C3CC3)nc12] | 2-Cyclopropyl- 5H-pyrrolo[2,3- b]pyrazine-7- carboxylic acid ((1S,2R,3S)-1- cyclohexylmethyl- 3-cyclopropyl- 2,3-dihydroxy- propyl)-amide | |
| I-89 | 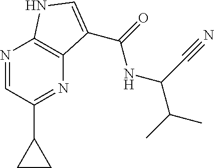 [SMILES: CC(C)C(C#N)NC(=O)c1c[nH]c2ncc(C3CC3)nc12] | 2-Cyclopropyl- 5H-pyrrolo[2,3- b]pyrazine-7- carboxylic acid (1-cyano-2- methyl-propyl)- amide | |
| I-90 | 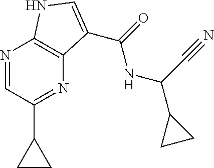 [SMILES: N#CC(NC(=O)c1c[nH]c2ncc(C3CC3)nc12)C1CC1] | 2-Cyclopropyl- 5H-pyrrolo[2,3- b]pyrazine-7- carboxylic acid (cyano- cyclopropyl- methyl)-amide | |
| I-91 | 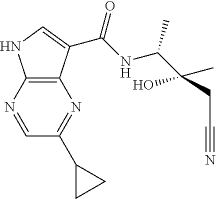 [SMILES: C[C@@H](NC(=O)c1c[nH]c2ncc(C3CC3)nc12)[C@](C)(O)CC#N] | 2-Cyclopropyl- 5H-pyrrolo[2,3- b]pyrazine-7- carboxylic acid ((1R,2R)-3- cyano-2- hydroxy-1,2- dimethyl- propyl)-amide | 234.0- 236.0 |
| I-92 | 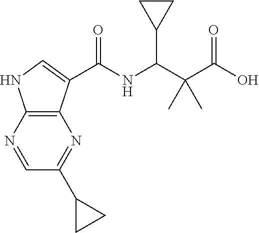 [SMILES: CC(C)(C(=O)O)C(NC(=O)c1c[nH]c2ncc(C3CC3)nc12)C1CC1] | 3-Cyclopropyl- 3-[(2- cyclopropyl-5H- pyrrolo[2,3- b]pyrazine-7- carbonyl)- amino]-2,2- dimethyl- propionic acid | 265.0- 267.0 |
| I-93 | 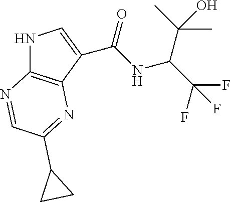 [SMILES: CC(C)(O)C(NC(=O)c1c[nH]c2ncc(C3CC3)nc12)C(F)(F)F] | 2-Cyclopropyl- 5H-pyrrolo[2,3- b]pyrazine-7- carboxylic acid (2-hydroxy-2- methyl-1- trifluoromethyl- propyl)-amide | 258.0- 260.0 |
| I-94 | 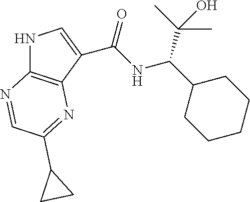 [SMILES: CC(C)(O)[C@@H](NC(=O)c1c[nH]c2ncc(C3CC3)nc12)C1CCCCC1] | 2-Cyclopropyl- 5H-pyrrolo[2,3- b]pyrazine-7- carboxylic acid ((S)-1- cyclohexyl-2- hydroxy-2- methyl-propyl)- amide | 251.0- 253.0 |
| I-95 | 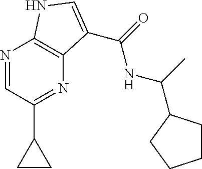 [SMILES: CC(NC(=O)c1c[nH]c2ncc(C3CC3)nc12)C1CCCC1] | 2-Cyclopropyl- 5H-pyrrolo[2,3- b]pyrazine-7- carboxylic acid (1-cyclopentyl- ethyl)-amide | |
| I-96 | 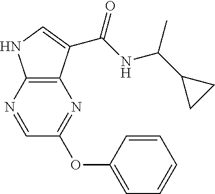 [SMILES: CC(NC(=O)c1c[nH]c2ncc(Oc3ccccc3)nc12)C1CC1] | 2-Phenoxy-5H- pyrrolo[2,3- b]pyrazine-7- carboxylic acid (1-cyclopropyl- ethyl)-amide | 242.0- 245.0 |
| I-97 | 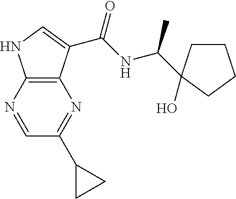 [SMILES: C[C@H](NC(=O)c1c[nH]c2ncc(C3CC3)nc12)C1(O)CCCC1] | 2-Cyclopropyl- 5H-pyrrolo[2,3- b]pyrazine-7- carboxylic acid [(S)-1-(1- hydroxy- cyclopentyl)- ethyl]-amide | 292.0- 294.0 |
| I-98 | 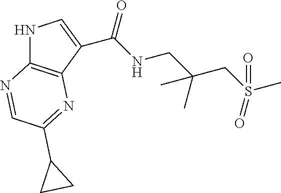 [SMILES: CC(C)(CNC(=O)c1c[nH]c2ncc(C3CC3)nc12)CS(C)(=O)=O] | 2-Cyclopropyl- 5H-pyrrolo[2,3- b]pyrazine-7- carboxylic acid (3- methanesulfonyl- 2,2-dimethyl- propyl)-amide | 206.0- 208.0 |
| I-99 | 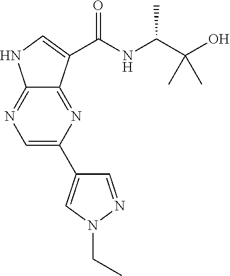 [SMILES: CCn1cc(-c2cnc3[nH]cc(C(=O)N[C@H](C)C(C)(C)O)c3n2)cn1] | 2-(1-Ethyl-1H- pyrazol-4-yl)- 5H-pyrrolo[2,3- b]pyrazine-7- carboxylic acid ((R)-2-hydroxy- 1,2-dimethyl- propyl)-amide | 270.0- 272.0 |
| I-100 | 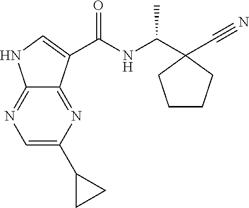 [SMILES: C[C@@H](NC(=O)c1c[nH]c2ncc(C3CC3)nc12)C1(C#N)CCCC1] | 2-Cyclopropyl- 5H-pyrrolo[2,3- b]pyrazine-7- carboxylic acid [(R)-1-(1-cyano- cyclopentyl)- ethyl]-amide | 220.0- 223.0 |
| I-101 | 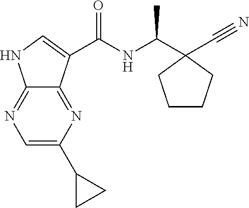 [SMILES: C[C@H](NC(=O)c1c[nH]c2ncc(C3CC3)nc12)C1(C#N)CCCC1] | 2-Cyclopropyl- 5H-pyrrolo[2,3- b]pyrazine-7- carboxylic acid [(S)-1-(1-cyano- cyclopentyl)- ethyl]-amide | 220.0- 223.0 |
| I-102 | 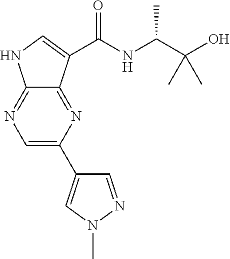 [SMILES: C[C@@H](NC(=O)c1c[nH]c2ncc(-c3cnn(C)c3)nc12)C(C)(C)O] | 2-(1-Methyl- 1H-pyrazol-4- yl)-5H- pyrrolo[2,3- b]pyrazine-7- carboxylic acid ((R)-2-hydroxy- 1,2-dimethyl- propyl)-amide | 285.0- 288.0 |
| I-103 | 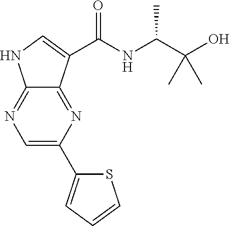 [SMILES: C[C@@H](NC(=O)c1c[nH]c2ncc(-c3cccs3)nc12)C(C)(C)O] | 2-Thiophen-2- yl-5H- pyrrolo[2,3- b]pyrazine-7- carboxylic acid ((R)-2-hydroxy- 1,2-dimethyl- propyl)-amide | 272.0- 275.0 |
| I-104 | 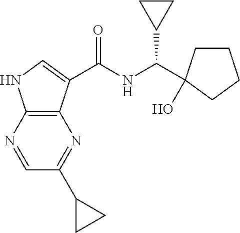 [SMILES: O=C(N[C@H](C1CC1)C1(O)CCCC1)c1c[nH]c2ncc(C3CC3)nc12] | 2-Cyclopropyl- 5H-pyrrolo[2,3- b]pyrazine-7- carboxylic acid [(R)- cyclopropyl-(1- hydroxy- cyclopentyl)- methyl]-amide | 195.0- 197.0 |
| I-105 | 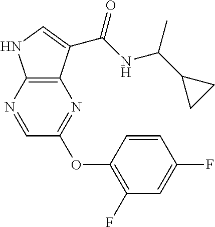 [SMILES: CC(NC(=O)c1c[nH]c2ncc(Oc3ccc(F)cc3F)nc12)C1CC1] | 2-(2,4-Difluoro- phenoxy)-5H- pyrrolo[2,3- b]pyrazine-7- carboxylic acid (1-cyclopropyl- ethyl)-amide | |
| I-106 | 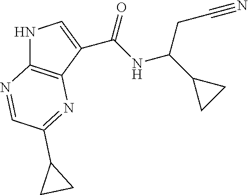 [SMILES: N#CCC(NC(=O)c1c[nH]c2ncc(C3CC3)nc12)C1CC1] | 2-Cyclopropyl- 5H-pyrrolo[2,3- b]pyrazine-7- carboxylic acid (2-cyano-1- cyclopropyl- ethyl)-amide | 230.0- 232.0 |
| I-107 | 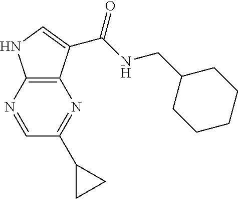 [SMILES: O=C(NCC1CCCCC1)c1c[nH]c2ncc(C3CC3)nc12] | 2-Cyclopropyl- 5H-pyrrolo[2,3- b]pyrazine-7- carboxylic acid cyclohexylmethyl- amide | 284.2- 284.7 |
| I-108 | 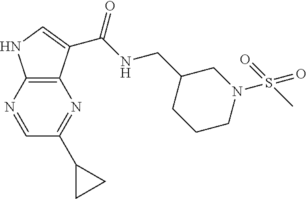 [SMILES: CS(=O)(=O)N1CCCC(CNC(=O)c2c[nH]c3ncc(C4CC4)nc23)C1] | 2-Cyclopropyl- 5H-pyrrolo[2,3- b]pyrazine-7- carboxylic acid (1- methanesulfonyl- piperidin-3- ylmethyl)-amide | 247.6- 248.4° C. |
| I-109 | 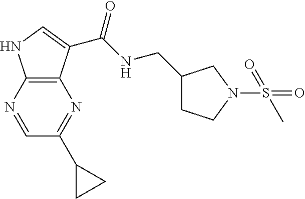 [SMILES: CS(=O)(=O)N1CCC(CNC(=O)c2c[nH]c3ncc(C4CC4)nc23)C1] | 2-Cyclopropyl- 5H-pyrrolo[2,3- b]pyrazine-7- carboxylic acid (1- methanesulfonyl- pyrrolidin-3- ylmethyl)-amide | 248.0- 249.0 |
| I-110 | 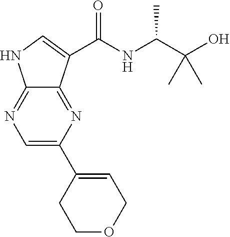 [SMILES: C[C@@H](NC(=O)c1c[nH]c2ncc(C3=CCOCC3)nc12)C(C)(C)O] | 2-(3,6-Dihydro- 2H-pyran-4-yl)- 5H-pyrrolo[2,3- b]pyrazine-7- carboxylic acid ((R)-2-hydroxy- 1,2-dimethyl- propyl)-amide | |
| I-111 | 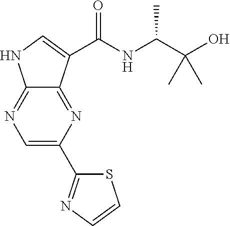 [SMILES: C[C@@H](NC(=O)c1c[nH]c2ncc(-c3nccs3)nc12)C(C)(C)O] | 2-Thiazol-2-yl- 5H-pyrrolo[2,3- b]pyrazine-7- carboxylic acid ((R)-2-hydroxy- 1,2-dimethyl- propyl)-amide | |
| I-112 | 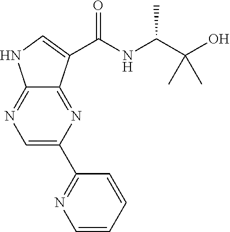 [SMILES: C[C@@H](NC(=O)c1c[nH]c2ncc(-c3ccccn3)nc12)C(C)(C)O] | 2-Pyridin-2-yl- 5H-pyrrolo[2,3- b]pyrazine-7- carboxylic acid ((R)-2-hydroxy- 1,2-dimethyl- propyl)-amide | |
| I-113 | 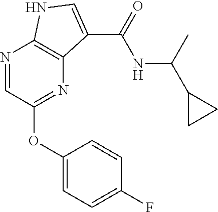 [SMILES: CC(NC(=O)c1c[nH]c2ncc(Oc3ccc(F)cc3)nc12)C1CC1] | 2-(4-Fluoro- phenoxy)-5H- pyrrolo[2,3- b]pyrazine-7- carboxylic acid (1-cyclopropyl- ethyl)-amide | |
| I-114 | 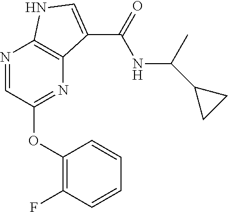 [SMILES: CC(NC(=O)c1c[nH]c2ncc(Oc3ccccc3F)nc12)C1CC1] | 2-(2-Fluoro- phenoxy)-5H- pyrrolo[2,3- b]pyrazine-7- carboxylic acid (1-cyclopropyl- ethyl)-amide | |
| I-115 | 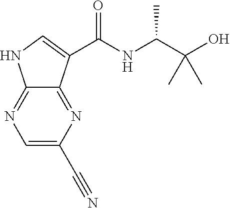 [SMILES: C[C@@H](NC(=O)c1c[nH]c2ncc(C#N)nc12)C(C)(C)O] | 2-Cyano-5H- pyrrolo[2,3- b]pyrazine-7- carboxylic acid ((R)-2-hydroxy- 1,2-dimethyl- propyl)-amide | |
| I-116 | 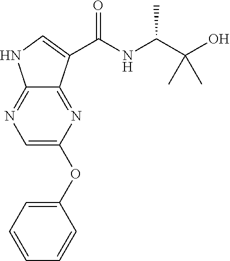 [SMILES: C[C@@H](NC(=O)c1c[nH]c2ncc(Oc3ccccc3)nc12)C(C)(C)O] | 2-Phenoxy-5H- pyrrolo[2,3- b]pyrazine-7- carboxylic acid ((R)-2-hydroxy- 1,2-dimethyl- propyl)-amide | |
| I-117 | 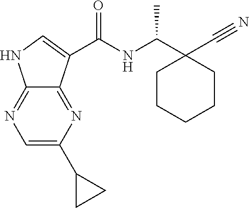 [SMILES: C[C@@H](NC(=O)c1c[nH]c2ncc(C3CC3)nc12)C1(C#N)CCCCC1] | 2-Cyclopropyl- 5H-pyrrolo[2,3- b]pyrazine-7- carboxylic acid [(R)-1-(1-cyano- cyclohexyl)- ethyl]-amide | |
| I-118 | 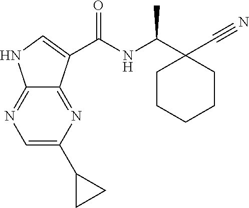 [SMILES: C[C@H](NC(=O)c1c[nH]c2ncc(C3CC3)nc12)C1(C#N)CCCCC1] | 2-Cyclopropyl- 5H-pyrrolo[2,3- b]pyrazine-7- carboxylic acid [(S)-1-(1-cyano- cyclohexyl)- ethyl]-amide | |
| I-119 | 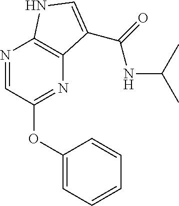 [SMILES: CC(C)NC(=O)c1c[nH]c2ncc(Oc3ccccc3)nc12] | 2-Phenoxy-5H- pyrrolo[2,3- b]pyrazine-7- carboxylic acid isopropylamide | 263- 265 |
| I-120 | 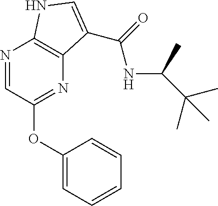 [SMILES: C[C@H](NC(=O)c1c[nH]c2ncc(Oc3ccccc3)nc12)C(C)(C)C] | 2-Phenoxy-5H- pyrrolo[2,3- b]pyrazine-7- carboxylic acid ((S)-1,2,2- trimethyl- propyl)-amide | 270- 273 |
| I-121 | 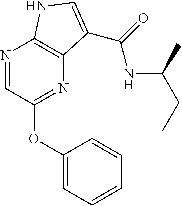 [SMILES: CC[C@H](C)NC(=O)c1c[nH]c2ncc(Oc3ccccc3)nc12] | 2-Phenoxy-5H- pyrrolo[2,3- b]pyrazine-7- carboxylic acid ((S)-sec-butyl)- amide | 227- 229 |
| I-122 | 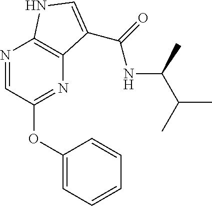 [SMILES: CC(C)[C@H](C)NC(=O)c1c[nH]c2ncc(Oc3ccccc3)nc12] | 2-Phenoxy-5H- pyrrolo[2,3- b]pyrazine-7- carboxylic acid ((S)-1,2- dimethyl- propyl)-amide | 234- 235 |
| I-123 | 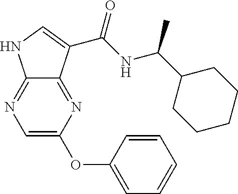 [SMILES: C[C@H](NC(=O)c1c[nH]c2ncc(Oc3ccccc3)nc12)C1CCCCC1] | 2-Phenoxy-5H- pyrrolo[2,3- b]pyrazine-7- carboxylic acid ((S)-1- cyclohexyl- ethyl)-amide | 227- 230 |
| I-124 | 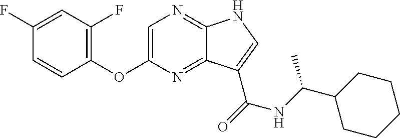 [SMILES: C[C@@H](NC(=O)c1c[nH]c2ncc(Oc3ccc(F)cc3F)nc12)C1CCCCC1] | 2-(2,4-Difluoro- phenoxy)-5H- pyrrolo[2,3- b]pyrazine-7- carboxylic acid ((R)-1- cyclohexyl- ethyl)-amide | 233.0- 235.0 |
| I-125 | 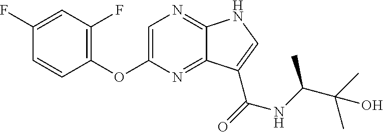 [SMILES: C[C@H](NC(=O)c1c[nH]c2ncc(Oc3ccc(F)cc3F)nc12)C(C)(C)O] | 2-(2,4-Difluoro- phenoxy)-5H- pyrrolo[2,3- b]pyrazine-7- carboxylic acid ((S)-2-hydroxy- 1,2-dimethyl- propyl)-amide | 224.0- 226.0 |
| I-126 | 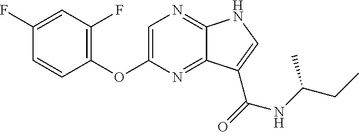 [SMILES: CC[C@@H](C)NC(=O)c1c[nH]c2ncc(Oc3ccc(F)cc3F)nc12] | 2-(2,4-Difluoro- phenoxy)-5H- pyrrolo[2,3- b]pyrazine-7- carboxylic acid ((R)-sec-butyl)- amide | 246.0- 248.0 |
| I-127 | 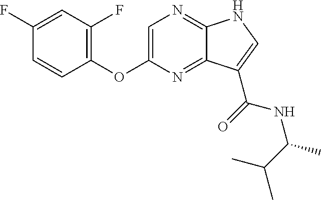 [SMILES: CC(C)[C@@H](C)NC(=O)c1c[nH]c2ncc(Oc3ccc(F)cc3F)nc12] | 2-(2,4-Difluoro- phenoxy)-5H- pyrrolo[2,3- b]pyrazine-7- carboxylic acid ((R)-1,2- dimethyl- propyl)-amide | 235.0- 237.0 |
| I-128 | 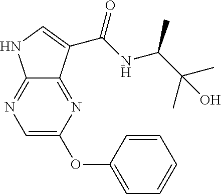 [SMILES: C[C@H](NC(=O)c1c[nH]c2ncc(Oc3ccccc3)nc12)C(C)(C)O] | 2-Phenoxy-5H- pyrrolo[2,3- b]pyrazine-7- carboxylic acid ((S)-2-hydroxy- 1,2-dimethyl- propyl)-amide | 232- 233 |
| I-129 | 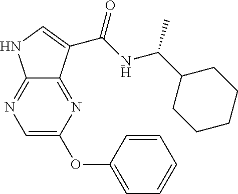 [SMILES: C[C@@H](NC(=O)c1c[nH]c2ncc(Oc3ccccc3)nc12)C1CCCCC1] | 2-Phenoxy-5H- pyrrolo[2,3- b]pyrazine-7- carboxylic acid ((R)-1- cyclohexyl- ethyl)-amide | 231- 232 |
| I-130 | 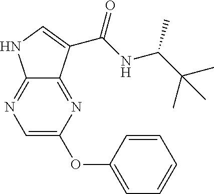 [SMILES: C[C@@H](NC(=O)c1c[nH]c2ncc(Oc3ccccc3)nc12)C(C)(C)C] | 2-Phenoxy-5H- pyrrolo[2,3- b]pyrazine-7- carboxylic acid ((R)-1,2,2- trimethyl- propyl)-amide | 273- 274 |
| I-131 | 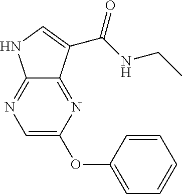 [SMILES: CCNC(=O)c1c[nH]c2ncc(Oc3ccccc3)nc12] | 2-Phenoxy-5H- pyrrolo[2,3- b]pyrazine-7- carboxylic acid ethylamide | 230- 232 |
| I-132 | 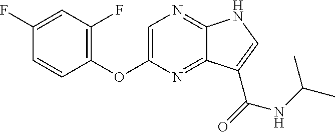 [SMILES: CC(C)NC(=O)c1c[nH]c2ncc(Oc3ccc(F)cc3F)nc12] | 2-(2,4-Difluoro- phenoxy)-5H- pyrrolo[2,3- b]pyrazine-7- carboxylic acid isopropylamide | |
| I-133 | 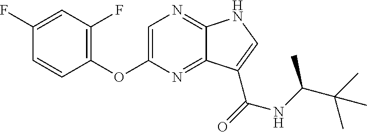 [SMILES: C[C@H](NC(=O)c1c[nH]c2ncc(Oc3ccc(F)cc3F)nc12)C(C)(C)C] | 2-(2,4-Difluoro- phenoxy)-5H- pyrrolo[2,3- b]pyrazine-7- carboxylic acid ((S)-1,2,2- trimethyl- propyl)-amide | |
| I-134 | 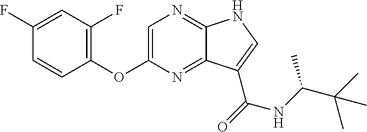 [SMILES: C[C@@H](NC(=O)c1c[nH]c2ncc(Oc3ccc(F)cc3F)nc12)C(C)(C)C] | 2-(2,4-Difluoro- phenoxy)-5H- pyrrolo[2,3- b]pyrazine-7- carboxylic acid ((R)-1,2,2- trimethyl- propyl)-amide | |
| I-135 | 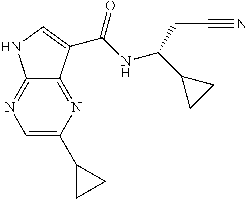 [SMILES: N#CC[C@@H](NC(=O)c1c[nH]c2ncc(C3CC3)nc12)C1CC1] | 2-Cyclopropyl- 5H-pyrrolo[2,3- b]pyrazine-7- carboxylic acid ((R)-2-cyano-1- cyclopropyl- ethyl)-amide | |
| I-136 | 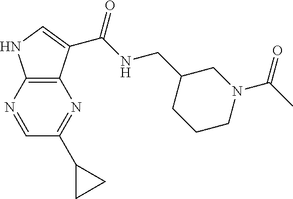 [SMILES: CC(=O)N1CCCC(CNC(=O)c2c[nH]c3ncc(C4CC4)nc23)C1] | 2-Cyclopropyl- 5H-pyrrolo[2,3- b]pyrazine-7- carboxylic acid (1-acetyl- piperidin-3- ylmethyl)-amide | 198.4- 199.1 |
| I-137 | 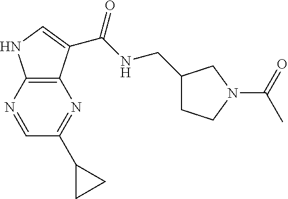 [SMILES: CC(=O)N1CCC(CNC(=O)c2c[nH]c3ncc(C4CC4)nc23)C1] | 2-Cyclopropyl- 5H-pyrrolo[2,3- b]pyrazine-7- carboxylic acid (1-acetyl- pyrrolidin-3- ylmethyl)-amide | 233.8- 235.0 |
| I-138 | 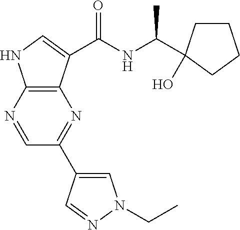 [SMILES: CCn1cc(-c2cnc3[nH]cc(C(=O)N[C@@H](C)C4(O)CCCC4)c3n2)cn1] | 2-(1-Ethyl-1H- pyrazol-4-yl)- 5H-pyrrolo[2,3- b]pyrazine-7- carboxylic acid [(S)-1-(1- hydroxy- cyclopentyl)- ethyl]-amide | |
| I-139 | 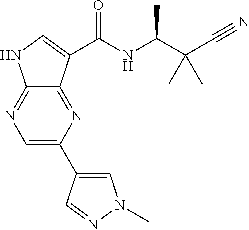 [SMILES: C[C@H](NC(=O)c1c[nH]c2ncc(-c3cnn(C)c3)nc12)C(C)(C)C#N] | 2-(1-Methyl- 1H-pyrazol-4- yl)-5H- pyrrolo[2,3- b]pyrazine-7- carboxylic acid ((S)-2-cyano- 1,2,2-trimethyl- ethyl)-amide | |
| I-140 | 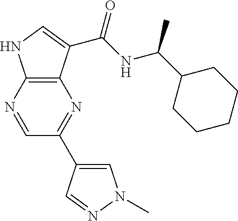 [SMILES: C[C@H](NC(=O)c1c[nH]c2ncc(-c3cnn(C)c3)nc12)C1CCCCC1] | 2-(1-Methyl- 1H-pyrazol-4- yl)-5H- pyrrolo[2,3- b]pyrazine-7- carboxylic acid ((S)-1- cyclohexyl- ethyl)-amide | |
| I-141 | 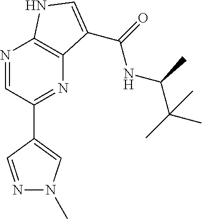 [SMILES: C[C@H](NC(=O)c1c[nH]c2ncc(-c3cnn(C)c3)nc12)C(C)(C)C] | 2-(1-Methyl- 1H-pyrazol-4- yl)-5H- pyrrolo[2,3- b]pyrazine-7- carboxylic acid ((S)-1,2,2- trimethyl- propyl)-amide | 296- 297 |
| I-142 | 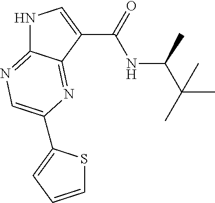 [SMILES: C[C@H](NC(=O)c1c[nH]c2ncc(-c3cccs3)nc12)C(C)(C)C] | 2-Thiophen-2- yl-5H- pyrrolo[2,3- b]pyrazine-7- carboxylic acid ((S)-1,2,2- trimethyl- propyl)-amide | 311- 312 |
| I-143 | 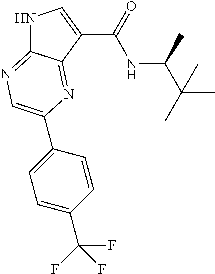 [SMILES: C[C@H](NC(=O)c1c[nH]c2ncc(-c3ccc(C(F)(F)F)cc3)nc12)C(C)(C)C] | 2-(4- Trifluoromethyl- phenyl)-5H- pyrrolo[2,3- b]pyrazine-7- carboxylic acid ((S)-1,2,2- trimethyl- propyl)-amide | >300 |
| I-144 | 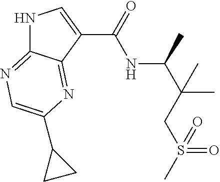 [SMILES: C[C@H](NC(=O)c1c[nH]c2ncc(C3CC3)nc12)C(C)(C)CS(C)(=O)=O] | 2-Cyclopropyl- 5H-pyrrolo[2,3- b]pyrazine-7- carboxylic acid ((S)-3- methanesulfonyl- 1,2,2-trimethyl- propyl)-amide | 232- 234 |
| I-145 | 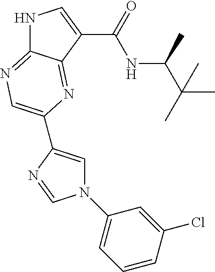 [SMILES: C[C@H](NC(=O)c1c[nH]c2ncc(-c3cn(-c4cccc(Cl)c4)cn3)nc12)C(C)(C)C] | 2-[1-(3-Chloro- phenyl)-1H- imidazol-4-yl]- 5H-pyrrolo[2,3- b]pyrazine-7- carboxylic acid ((S)-1,2,2- trimethyl- propyl)-amide | 337- 339 |
| I-146 | 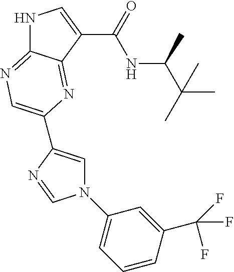 [SMILES: C[C@H](NC(=O)c1c[nH]c2ncc(-c3cn(-c4cccc(C(F)(F)F)c4)cn3)nc12)C(C)(C)C] | 2-[1-(3- Trifluoromethyl- phenyl)-1H- imidazol-4-yl]- 5H-pyrrolo[2,3- b]pyrazine-7- carboxylic acid ((S)-1,2,2- trimethyl- propyl)-amide | 332- 333 |
| I-147 | 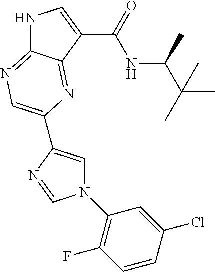 [SMILES: C[C@H](NC(=O)c1c[nH]c2ncc(-c3cn(-c4cc(Cl)ccc4F)cn3)nc12)C(C)(C)C] | 2-[1-(5-Chloro- 2-fluoro- phenyl)-1H- imidazol-4-yl]- 5H-pyrrolo[2,3- b]pyrazine-7- carboxylic acid ((S)-1,2,2- trimethyl- propyl)-amide | 337- 339 |
| I-148 | 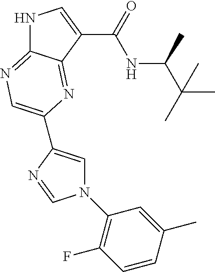 [SMILES: Cc1ccc(F)c(-n2cnc(-c3cnc4[nH]cc(C(=O)N[C@@H](C)C(C)(C)C)c4n3)c2)c1] | 2-[1-(2-Fluoro- 5-methyl- phenyl)-1H- imidazol-4-yl]- 5H-pyrrolo[2,3- b]pyrazine-7- carboxylic acid ((S)-1,2,2- trimethyl- propyl)-amide | 331- 332 |
| I-149 | 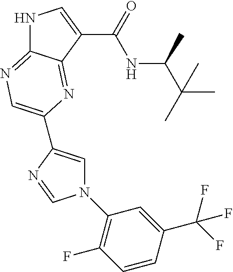 [SMILES: C[C@H](NC(=O)c1c[nH]c2ncc(-c3cn(-c4cc(C(F)(F)F)ccc4F)cn3)nc12)C(C)(C)C] | 2-[1-(2-Fluoro- 5- trifluoromethyl- phenyl)-1H- imidazol-4-yl]- 5H-pyrrolo[2,3- b]pyrazine-7- carboxylic acid ((S)-1,2,2- trimethyl- propyl)-amide | >300 |
| I-150 | 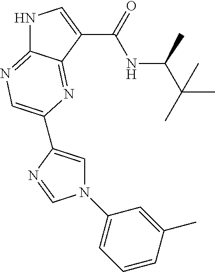 [SMILES: Cc1cccc(-n2cnc(-c3cnc4[nH]cc(C(=O)N[C@@H](C)C(C)(C)C)c4n3)c2)c1] | 2-(1-m-Tolyl- 1H-imidazol-4- yl)-5H- pyrrolo[2,3- b]pyrazine-7- carboxylic acid ((S)-1,2,2- trimethyl- propyl)-amide | 314- 316 |
| I-151 | 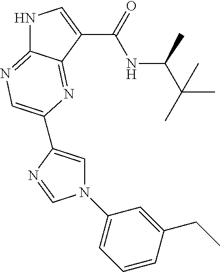 [SMILES: CCc1cccc(-n2cnc(-c3cnc4[nH]cc(C(=O)N[C@@H](C)C(C)(C)C)c4n3)c2)c1] | 2-[1-(3-Ethyl- phenyl)-1H- imidazol-4-yl]- 5H-pyrrolo[2,3- b]pyrazine-7- carboxylic acid ((S)-1,2,2- trimethyl- propyl)-amide | 284- 287 |
| I-152 | 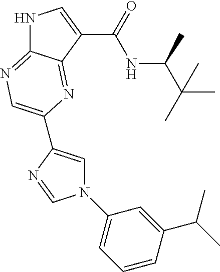 [SMILES: CC(C)c1cccc(-n2cnc(-c3cnc4[nH]cc(C(=O)N[C@@H](C)C(C)(C)C)c4n3)c2)c1] | 2-[1-(3- Isopropyl- phenyl)-1H- imidazol-4-yl]- 5H-pyrrolo[2,3- b]pyrazine-7- carboxylic acid ((S)-1,2,2- trimethyl- propyl)-amide | 242- 245 |
| I-153 | 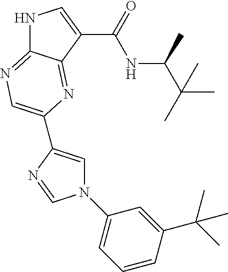 [SMILES: C[C@H](NC(=O)c1c[nH]c2ncc(-c3cn(-c4cccc(C(C)(C)C)c4)cn3)nc12)C(C)(C)C] | 2-[1-(3-tert- Butyl-phenyl)- 1H-imidazol-4- yl]-5H- pyrrolo[2,3- b]pyrazine-7- carboxylic acid ((S)-1,2,2- trimethyl- propyl)-amide | 226- 228 |
| I-154 | 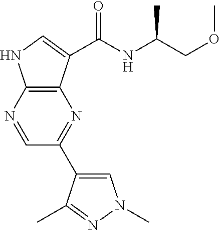 [SMILES: COC[C@H](C)NC(=O)c1c[nH]c2ncc(-c3cn(C)nc3C)nc12] | 2-(1,3- Dimethyl-1H- pyrazol-4-yl)- 5H-pyrrolo[2,3- b]pyrazine-7- carboxylic acid ((S)-2-methoxy- 1-methyl-ethyl)- amide | |
| I-155 | 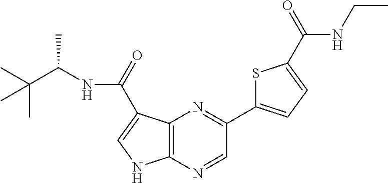 [SMILES: CCNC(=O)c1ccc(-c2cnc3[nH]cc(C(=O)N[C@@H](C)C(C)(C)C)c3n2)s1] | 2-(5- Ethylcarbamoyl- thiophen-2-yl)- 5H-pyrrolo[2,3- b]pyrazine-7- carboxylic acid ((S)-1,2,2- trimethyl- propyl)-amide | |
| I-156 | 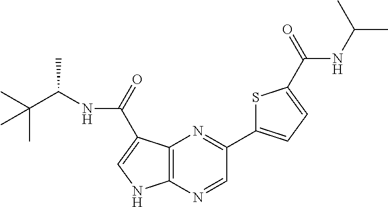 [SMILES: CC(C)NC(=O)c1ccc(-c2cnc3[nH]cc(C(=O)N[C@@H](C)C(C)(C)C)c3n2)s1] | 2-(5-Isopropyl- carbamoyl- thiophen-2- yl)-5H- pyrrolo[2,3- b]pyrazine-7- carboxylic acid ((S)-1,2,2- trimethyl- propyl)-amide | |
| I-157 | 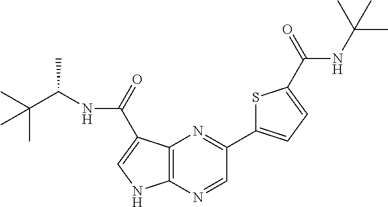 [SMILES: C[C@H](NC(=O)c1c[nH]c2ncc(-c3ccc(C(=O)NC(C)(C)C)s3)nc12)C(C)(C)C] | 2-(5-tert- Butylcarbamoyl- thiophen-2-yl)- 5H-pyrrolo[2,3- b]pyrazine-7- carboxylic acid ((S)-1,2,2- trimethyl- propyl)-amide | |
| I-158 | 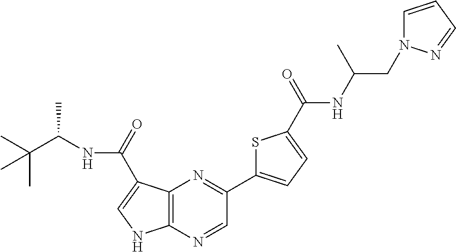 [SMILES: CC(Cn1cccn1)NC(=O)c1ccc(-c2cnc3[nH]cc(C(=O)N[C@@H](C)C(C)(C)C)c3n2)s1] | 2-[5-(1-Methyl- 2-pyrazol-1-yl- ethylcarbamoyl)- thiophen-2-yl]- 5H-pyrrolo[2,3- b]pyrazine-7- carboxylic acid ((S)-1,2,2- trimethyl- propyl)-amide | |
| I-159 | 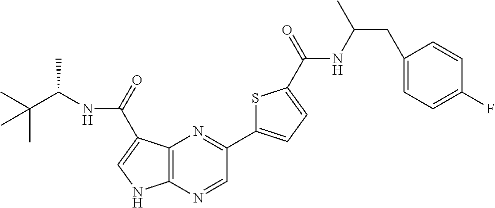 [SMILES: CC(Cc1ccc(F)cc1)NC(=O)c1ccc(-c2cnc3[nH]cc(C(=O)N[C@@H](C)C(C)(C)C)c3n2)s1] | 2-{5-[2-(4- Fluoro-phenyl)- 1-methyl- ethylcarbamoyl]- thiophen-2-yl}- 5H-pyrrolo[2,3- b]pyrazine-7- carboxylic acid ((S)-1,2,2- trimethyl- propyl)-amide | |
| I-160 | 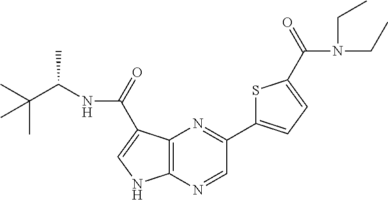 [SMILES: CCN(CC)C(=O)c1ccc(-c2cnc3[nH]cc(C(=O)N[C@@H](C)C(C)(C)C)c3n2)s1] | 2-(5- Diethylcarbamoyl- thiophen-2- yl)-5H- pyrrolo[2,3- b]pyrazine-7- carboxylic acid ((S)-1,2,2- trimethyl- propyl)-amide | |
| I-161 | 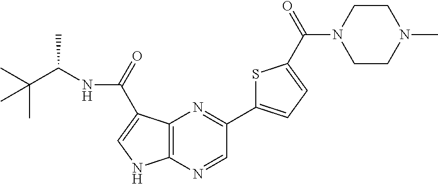 [SMILES: C[C@H](NC(=O)c1c[nH]c2ncc(-c3ccc(C(=O)N4CCN(C)CC4)s3)nc12)C(C)(C)C] | 2-[5-(4-Methyl- piperazine-1- carbonyl)- thiophen-2-yl]- 5H-pyrrolo[2,3- b]pyrazine-7- carboxylic acid ((S)-1,2,2- trimethyl- propyl)-amide | |
| I-162 | 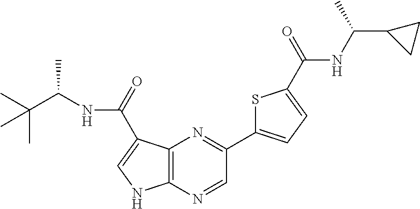 [SMILES: C[C@H](NC(=O)c1c[nH]c2ncc(-c3ccc(C(=O)N[C@H](C)C4CC4)s3)nc12)C(C)(C)C] | 2-[5-((R)-1- Cyclopropyl- ethylcarbamoyl)- thiophen-2-yl]- 5H-pyrrolo[2,3- b]pyrazine-7- carboxylic acid ((S)-1,2,2- trimethyl- propyl)-amide | |
| I-163 | 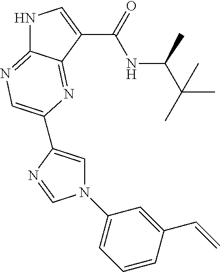 [SMILES: C=Cc1cccc(-n2cnc(-c3cnc4[nH]cc(C(=O)N[C@@H](C)C(C)(C)C)c4n3)c2)c1] | 2-[1-(3-Vinyl- phenyl)-1H- imidazol-4-yl]- 5H-pyrrolo[2,3- b]pyrazine-7- carboxylic acid ((S)-1,2,2- trimethyl- propyl)-amide | 253- 257 |
| I-164 | 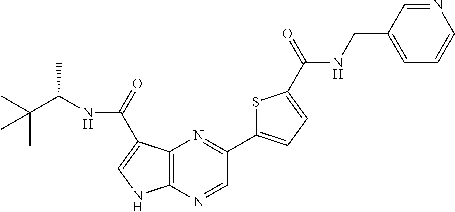 [SMILES: C[C@H](NC(=O)c1c[nH]c2ncc(-c3ccc(C(=O)NCc4cccnc4)s3)nc12)C(C)(C)C] | 2-{5-[(Pyridin- 3-ylmethyl)- carbamoyl]- thiophen-2-yl}- 5H-pyrrolo[2,3- b]pyrazine-7- carboxylic acid ((S)-1,2,2- trimethyl- propyl)-amide | |
| I-165 | 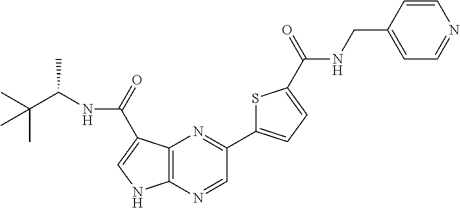 [SMILES: C[C@H](NC(=O)c1c[nH]c2ncc(-c3ccc(C(=O)NCc4ccncc4)s3)nc12)C(C)(C)C] | 2-{5-[(Pyridin- 4-ylmethyl)- carbamoyl]- thiophen-2-yl}- 5H-pyrrolo[2,3- b]pyrazine-7- carboxylic acid ((S)-1,2,2- trimethyl- propyl)-amide | |
| I-166 | 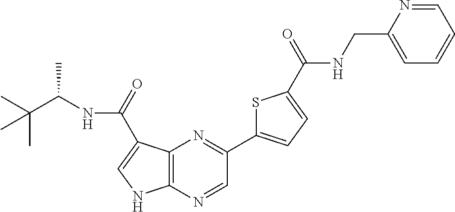 [SMILES: C[C@H](NC(=O)c1c[nH]c2ncc(-c3ccc(C(=O)NCc4ccccn4)s3)nc12)C(C)(C)C] | 2-{5-[(Pyridin- 2-ylmethyl)- carbamoyl]- thiophen-2-yl}- 5H-pyrrolo[2,3- b]pyrazine-7- carboxylic acid ((S)-1,2,2- trimethyl- propyl)-amide | |
| I-167 | 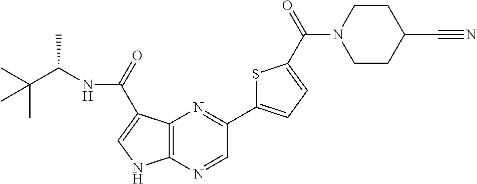 [SMILES: C[C@H](NC(=O)c1c[nH]c2ncc(-c3ccc(C(=O)N4CCC(C#N)CC4)s3)nc12)C(C)(C)C] | 2-[5-(4-Cyano- piperidine-1- carbonyl)- thiophen-2-yl]- 5H-pyrrolo[2,3- b]pyrazine-7- carboxylic acid ((S)-1,2,2- trimethyl- propyl)-amide | |
| I-168 | 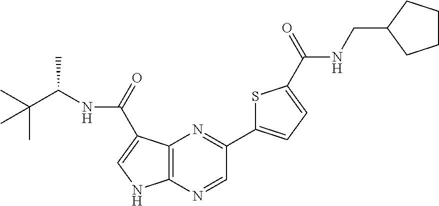 [SMILES: C[C@H](NC(=O)c1c[nH]c2ncc(-c3ccc(C(=O)NCC4CCCC4)s3)nc12)C(C)(C)C] | 2-[5-(Cyclo- pentylmethyl- carbamoyl)- thiophen-2-yl]- 5H-pyrrolo[2,3- b]pyrazine-7- carboxylic acid ((S)-1,2,2- trimethyl- propyl)-amide | |
| I-169 | 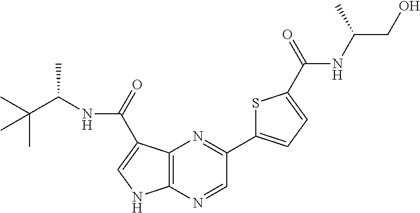 [SMILES: C[C@H](CO)NC(=O)c1ccc(-c2cnc3[nH]cc(C(=O)N[C@@H](C)C(C)(C)C)c3n2)s1] | 2-[5-((R)-2- Hydroxy-1- methyl- ethylcarbamoyl)- thiophen-2-yl]- 5H-pyrrolo[2,3- b]pyrazine-7- carboxylic acid ((S)-1,2,2- trimethyl- propyl)-amide | |
| I-170 | 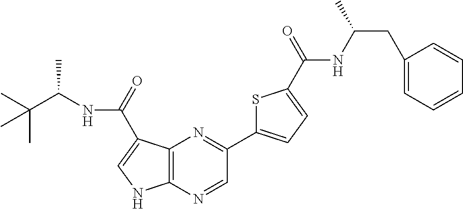 [SMILES: C[C@H](Cc1ccccc1)NC(=O)c1ccc(-c2cnc3[nH]cc(C(=O)N[C@@H](C)C(C)(C)C)c3n2)s1] | 2-[5-((R)-1- Methyl-2- phenyl- ethylcarbamoyl)- thiophen-2-yl]- 5H-pyrrolo[2,3- b]pyrazine-7- carboxylic acid ((S)-1,2,2- trimethyl- propyl)-amide | |
| I-171 | 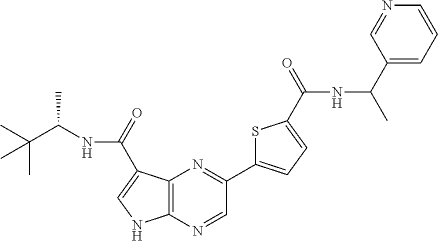 [SMILES: CC(NC(=O)c1ccc(-c2cnc3[nH]cc(C(=O)N[C@@H](C)C(C)(C)C)c3n2)s1)c1cccnc1] | 2-[5-(1-Pyridin- 3-yl- ethylcarbamoyl)- thiophen-2-yl]- 5H-pyrrolo[2,3- b]pyrazine-7- carboxylic acid ((S)-1,2,2- trimethyl- propyl)-amide | |
| I-172 | 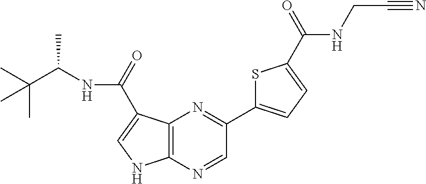 [SMILES: C[C@H](NC(=O)c1c[nH]c2ncc(-c3ccc(C(=O)NCC#N)s3)nc12)C(C)(C)C] | 2-[5- (Cyanomethyl- carbamoyl)- thiophen-2-yl]- 5H-pyrrolo[2,3- b]pyrazine-7- carboxylic acid ((S)-1,2,2- trimethyl- propyl)-amide | |
| I-173 | 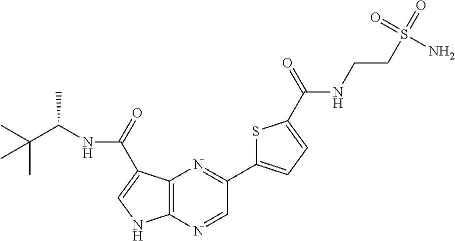 [SMILES: C[C@H](NC(=O)c1c[nH]c2ncc(-c3ccc(C(=O)NCCS(N)(=O)=O)s3)nc12)C(C)(C)C] | 2-[5-(2- Sulfamoyl- ethylcarbamoyl)- thiophen-2-yl]- 5H-pyrrolo[2,3- b]pyrazine-7- carboxylic acid ((S)-1,2,2- trimethyl- propyl)-amide | |
| I-174 | 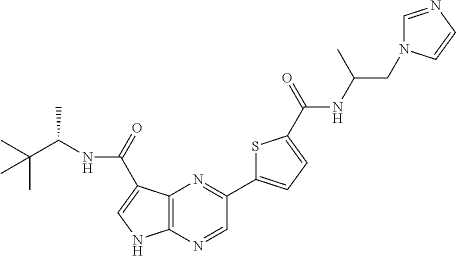 [SMILES: CC(Cn1ccnc1)NC(=O)c1ccc(-c2cnc3[nH]cc(C(=O)N[C@@H](C)C(C)(C)C)c3n2)s1] | 2-[5-(2- Imidazol-1-yl-1- methyl- ethylcarbamoyl)- thiophen-2-yl]- 5H-pyrrolo[2,3- b]pyrazine-7- carboxylic acid ((S)-1,2,2- trimethyl- propyl)-amide | |
| I-175 | 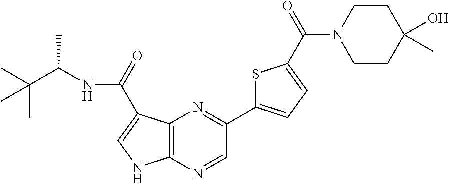 [SMILES: C[C@H](NC(=O)c1c[nH]c2ncc(-c3ccc(C(=O)N4CCC(C)(O)CC4)s3)nc12)C(C)(C)C] | 2-[5-(4-Hydroxy- 4-methyl- piperidine-1- carbonyl)- thiophen-2-yl]- 5H-pyrrolo[2,3- b]pyrazine-7- carboxylic acid ((S)-1,2,2- trimethyl- propyl)-amide | |
| I-176 | 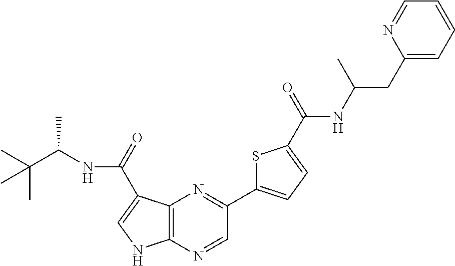 [SMILES: CC(Cc1ccccn1)NC(=O)c1ccc(-c2cnc3[nH]cc(C(=O)N[C@@H](C)C(C)(C)C)c3n2)s1] | 2-[5-(1-Methyl- 2-pyridin-2-yl- ethylcarbamoyl)- thiophen-2-yl]- 5H-pyrrolo[2,3- b]pyrazine-7- carboxylic acid ((S)-1,2,2- trimethyl- propyl)-amide | |
| I-177 | 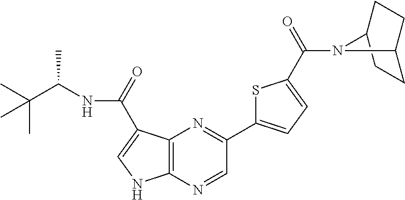 [SMILES: C[C@H](NC(=O)c1c[nH]c2ncc(-c3ccc(C(=O)N4C5CCC4CC5)s3)nc12)C(C)(C)C] | 2-[5-(7-Aza- bicyclo[2.2.1] heptane-7- carbonyl)- thiophen-2-yl]- 5H-pyrrolo[2,3- b]pyrazine-7- carboxylic acid ((S)-1,2,2- trimethyl- propyl)-amide | |
| I-178 | 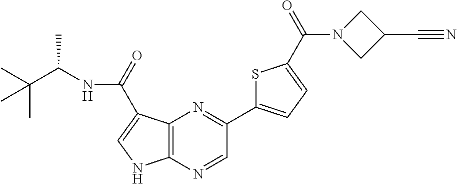 [SMILES: C[C@H](NC(=O)c1c[nH]c2ncc(-c3ccc(C(=O)N4CC(C#N)C4)s3)nc12)C(C)(C)C] | 2-[5-(3-Cyano- azetidine-1- carbonyl)- thiophen-2-yl]- 5H-pyrrolo[2,3- b]pyrazine-7- carboxylic acid ((S)-1,2,2- trimethyl- propyl)-amide | |
| I-179 | 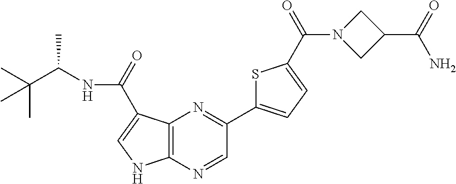 [SMILES: C[C@H](NC(=O)c1c[nH]c2ncc(-c3ccc(C(=O)N4CC(C(N)=O)C4)s3)nc12)C(C)(C)C] | 2-[5-(3- Carbamoyl- azetidine-1- carbonyl)- thiophen-2-yl]- 5H-pyrrolo[2,3- b]pyrazine-7- carboxylic acid ((S)-1,2,2- trimethyl- propyl)-amide | |
| I-180 | 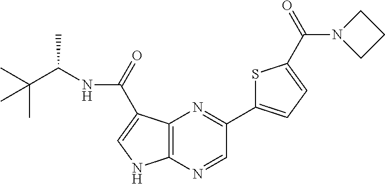 [SMILES: C[C@H](NC(=O)c1c[nH]c2ncc(-c3ccc(C(=O)N4CCC4)s3)nc12)C(C)(C)C] | 2-[5-(Azetidine- 1-carbonyl)- thiophen-2-yl]- 5H-pyrrolo[2,3- b]pyrazine-7- carboxylic acid ((S)-1,2,2- trimethyl- propyl)-amide | |
| I-181 | 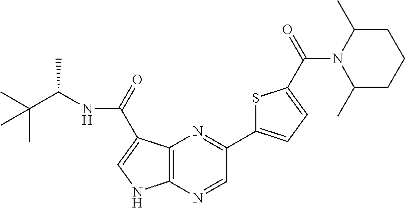 [SMILES: CC1CCCC(C)N1C(=O)c1ccc(-c2cnc3[nH]cc(C(=O)N[C@@H](C)C(C)(C)C)c3n2)s1] | 2-[5-(2,6- Dimethyl- piperidine-1- carbonyl)- thiophen-2-yl]- 5H-pyrrolo[2,3- b]pyrazine-7- carboxylic acid ((S)-1,2,2- trimethyl- propyl)-amide | |
| I-182 | 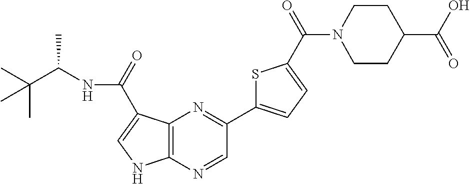 [SMILES: C[C@H](NC(=O)c1c[nH]c2ncc(-c3ccc(C(=O)N4CCC(C(=O)O)CC4)s3)nc12)C(C)(C)C] | 1-{5-[7-((S)- 1,2,2-Trimethyl- propylcarbamoyl)- 5H-pyrrolo[2,3- b]pyrazin-2-yl]- thiophene-2- carbonyl}- piperidine-4- carboxylic acid | |
| I-183 | 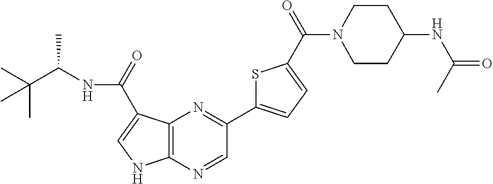 [SMILES: CC(=O)NC1CCN(C(=O)c2ccc(-c3cnc4[nH]cc(C(=O)N[C@@H](C)C(C)(C)C)c4n3)s2)CC1] | 2-[5-[4- Acetylamino- piperidine-1- carbonyl)- thiophen-2-yl]- 5H-pyrrolo[2,3- b]pyrazine-7- carboxylic acid ((S)-1,2,2- trimethyl- propyl)-amide | |
| I-184 | 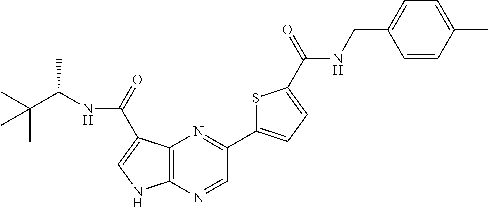 [SMILES: Cc1ccc(CNC(=O)c2ccc(-c3cnc4[nH]cc(C(=O)N[C@@H](C)C(C)(C)C)c4n3)s2)cc1] | 2-[5-(4-Methyl- benzylcarbamoyl)- thiophen-2-yl]- 5H-pyrrolo[2,3- b]pyrazine-7- carboxylic acid ((S)-1,2,2- trimethyl- propyl)-amide | |
| I-185 | 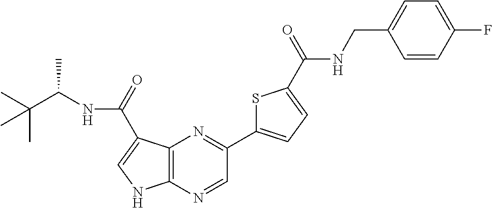 [SMILES: C[C@H](NC(=O)c1c[nH]c2ncc(-c3ccc(C(=O)NCc4ccc(F)cc4)s3)nc12)C(C)(C)C] | 2-[5-(4-Fluoro- benzylcarbamoyl)- thiophen-2-yl]- 5H-pyrrolo[2,3- b]pyrazine-7- carboxylic acid ((S)-1,2,2- trimethyl- propyl)-amide | |
| I-186 | 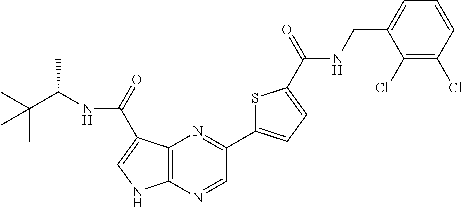 [SMILES: C[C@H](NC(=O)c1c[nH]c2ncc(-c3ccc(C(=O)NCc4cccc(Cl)c4Cl)s3)nc12)C(C)(C)C] | 2-[5-(2,3- Dichloro- benzylcarbamoyl)- thiophen-2-yl]- 5H-pyrrolo[2,3- b]pyrazine-7- carboxylic acid ((S)-1,2,2- trimethyl- propyl)-amide | |
| I-187 | 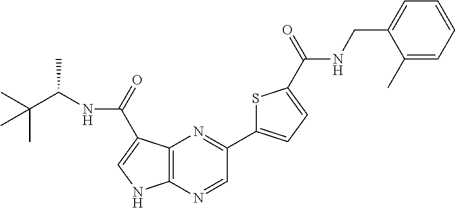 [SMILES: Cc1ccccc1CNC(=O)c1ccc(-c2cnc3[nH]cc(C(=O)N[C@@H](C)C(C)(C)C)c3n2)s1] | 2-[5-(2-Methyl- benzylcarbamoyl)- thiophen-2-yl]- 5H-pyrrolo[2,3- b]pyrazine-7- carboxylic acid ((S)-1,2,2- trimethyl- propyl)-amide | |
| I-188 | 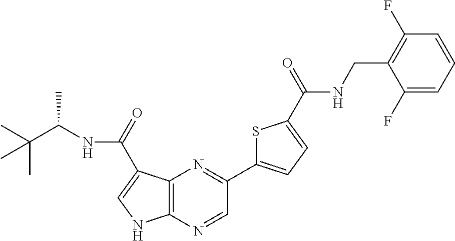 [SMILES: C[C@H](NC(=O)c1c[nH]c2ncc(-c3ccc(C(=O)NCc4c(F)cccc4F)s3)nc12)C(C)(C)C] | 2-[5-(2,6- Difluoro- benzylcarbamoyl)- thiophen-2-yl]- 5H-pyrrolo[2,3- b]pyrazine-7- carboxylic acid ((S)-1,2,2- trimethyl- propyl)-amide | |
| I-189 | 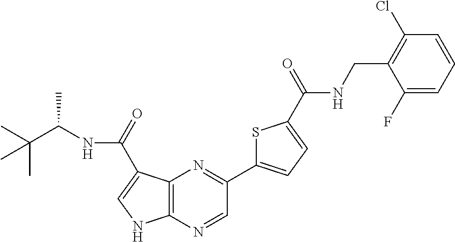 [SMILES: C[C@H](NC(=O)c1c[nH]c2ncc(-c3ccc(C(=O)NCc4c(F)cccc4Cl)s3)nc12)C(C)(C)C] | 2-[5-(2-Chloro- 6-fluoro- benzylcarbamoyl)- thiophen-2-yl]- 5H-pyrrolo[2,3- b]pyrazine-7- carboxylic acid ((S)-1,2,2- trimethyl- propyl)-amide | |
| I-190 | 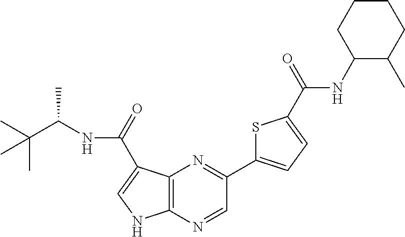 [SMILES: CC1CCCCC1NC(=O)c1ccc(-c2cnc3[nH]cc(C(=O)N[C@@H](C)C(C)(C)C)c3n2)s1] | 2-[5-(2-Methyl- cyclohexyl- carbamoyl)- thiophen-2-yl]- 5H-pyrrolo[2,3- b]pyrazine-7- carboxylic acid ((S)-1,2,2- trimethyl- propyl)-amide | |
| I-191 | 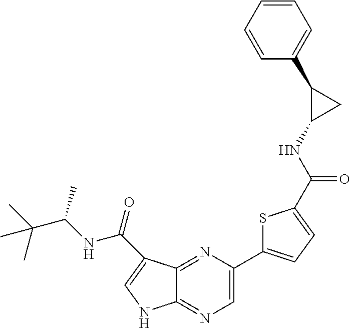 [SMILES: C[C@H](NC(=O)c1c[nH]c2ncc(-c3ccc(C(=O)N[C@H]4C[C@@H]4c4ccccc4)s3)nc12)C(C)(C)C] | 2-[5-((1S,2R)-2- Phenyl- cyclopropyl- carbamoyl)- thiophen-2-yl]- 5H-pyrrolo[2,3- b]pyrazine-7- carboxylic acid ((S)-1,2,2- trimethyl- propyl)-amide | |
| I-192 | 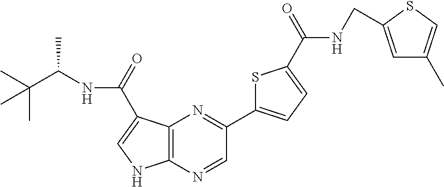 [SMILES: Cc1csc(CNC(=O)c2ccc(-c3cnc4[nH]cc(C(=O)N[C@@H](C)C(C)(C)C)c4n3)s2)c1] | 2-{5-[(4- Methyl- thiophen-2- ylmethyl)- carbamoyl]- thiophen-2-yl}- 5H-pyrrolo[2,3- b]pyrazine-7- carboxylic acid ((S)-1,2,2- trimethyl- propyl)-amide | |
| I-193 | 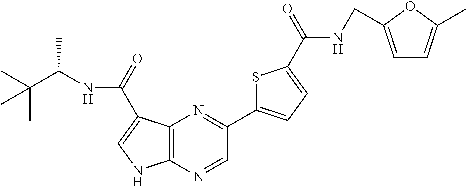 [SMILES: Cc1ccc(CNC(=O)c2ccc(-c3cnc4[nH]cc(C(=O)N[C@@H](C)C(C)(C)C)c4n3)s2)o1] | 2-{5-[(5- Methyl-furan-2- ylmethyl)- carbamoyl]- thiophen-2-yl}- 5H-pyrrolo[2,3- b]pyrazine-7- carboxylic acid ((S)-1,2,2- trimethyl- propyl)-amide | |
| I-194 | 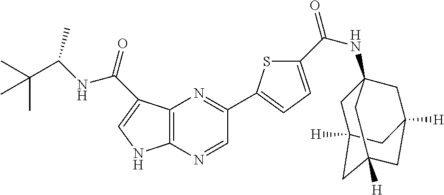 [SMILES: C[C@H](NC(=O)c1c[nH]c2ncc(-c3ccc(C(=O)N[C@]45C[C@H]6C[C@H](C[C@H](C6)C4)C5)s3)nc12)C(C)(C)C] | 2-[5- (Adamantan-1- ylcarbamoyl)- thiophen-2-yl]- 5H-pyrrolo[2,3- b]pyrazine-7- carboxylic acid ((S)-1,2,2- trimethyl- propyl)-amide | |
| I-195 | 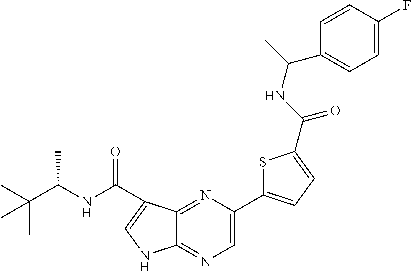 [SMILES: CC(NC(=O)c1ccc(-c2cnc3[nH]cc(C(=O)N[C@@H](C)C(C)(C)C)c3n2)s1)c1ccc(F)cc1] | 2-{5-[1-(4- Fluoro-phenyl)- ethylcarbamoyl]- thiophen-2-yl}- 5H-pyrrolo[2,3- b]pyrazine-7- carboxylic acid ((S)-1,2,2- trimethyl- propyl)-amide | |
| I-196 | 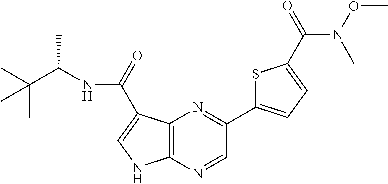 [SMILES: CON(C)C(=O)c1ccc(-c2cnc3[nH]cc(C(=O)N[C@@H](C)C(C)(C)C)c3n2)s1] | 2-[5-(Methoxy- methyl- carbamoyl)- thiophen-2-yl]- 5H-pyrrolo[2,3- b]pyrazine-7- carboxylic acid ((S)-1,2,2- trimethyl- propyl)-amide | |
| I-197 | 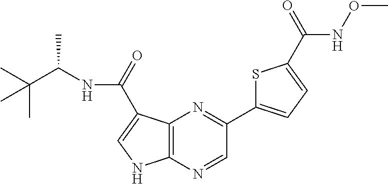 [SMILES: CONC(=O)c1ccc(-c2cnc3[nH]cc(C(=O)N[C@@H](C)C(C)(C)C)c3n2)s1] | 2-(5-Methoxy- carbamoyl- thiophen-2- yl)-5H- pyrrolo[2,3- b]pyrazine-7- carboxylic acid ((S)-1,2,2- trimethyl- propyl)-amide | |
| I-198 | 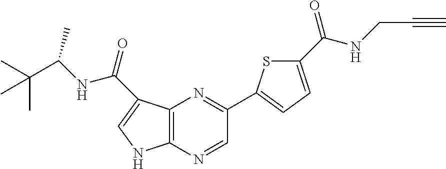 [SMILES: C#CCNC(=O)c1ccc(-c2cnc3[nH]cc(C(=O)N[C@@H](C)C(C)(C)C)c3n2)s1] | 2-(5-Prop-2- ynylcarbamoyl- thiophen-2-yl)- 5H-pyrrolo[2,3- b]pyrazine-7- carboxylic acid ((S)-1,2,2- trimethyl- propyl)-amide | |
| I-199 | 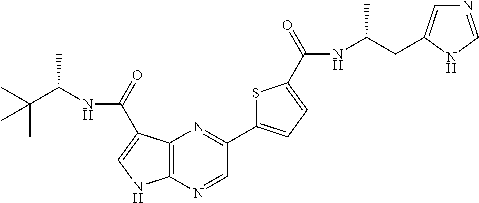 [SMILES: C[C@H](Cc1cnc[nH]1)NC(=O)c1ccc(-c2cnc3[nH]cc(C(=O)N[C@@H](C)C(C)(C)C)c3n2)s1] | 2-{5-[(R)-2- (3H-Imidazol-4- yl)-1-methyl- ethylcarbamoyl]- thiophen-2-yl}- 5H-pyrrolo[2,3- b]pyrazine-7- carboxylic acid ((S)-1,2,2- trimethyl- propyl)-amide | |
| I-200 | 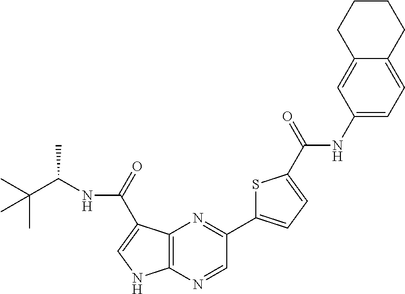 [SMILES: C[C@H](NC(=O)c1c[nH]c2ncc(-c3ccc(C(=O)Nc4ccc5c(c4)CCCC5)s3)nc12)C(C)(C)C] | 2-[5-(5,6,7,8- Tetrahydro- naphthalen-2- ylcarbamoyl)- thiophen-2-yl]- 5H-pyrrolo[2,3- b]pyrazine-7- carboxylic acid ((S)-1,2,2- trimethyl- propyl)-amide | |
| I-201 | 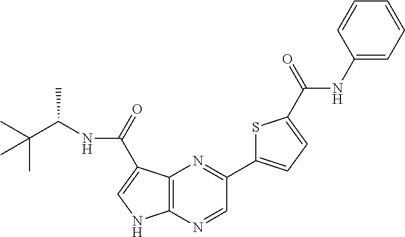 [SMILES: C[C@H](NC(=O)c1c[nH]c2ncc(-c3ccc(C(=O)Nc4ccccc4)s3)nc12)C(C)(C)C] | 2-(5- Phenylcarbamoyl- thiophen-2-yl)- 5H-pyrrolo[2,3- b]pyrazine-7- carboxylic acid ((S)-1,2,2- trimethyl- propyl)-amide | |
| I-202 | 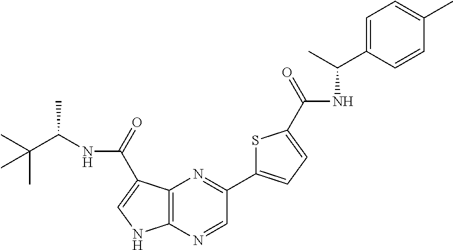 [SMILES: Cc1ccc([C@@H](C)NC(=O)c2ccc(-c3cnc4[nH]cc(C(=O)N[C@@H](C)C(C)(C)C)c4n3)s2)cc1] | 2-[5-((R)-1-p- Tolyl- ethylcarbamoyl)- thiophen-2-yl]- 5H-pyrrolo[2,3- b]pyrazine-7- carboxylic acid ((S)-1,2,2- trimethyl- propyl)-amide | |
| I-203 | 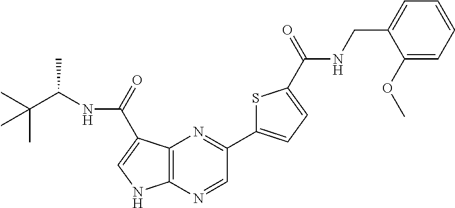 [SMILES: COc1ccccc1CNC(=O)c1ccc(-c2cnc3[nH]cc(C(=O)N[C@@H](C)C(C)(C)C)c3n2)s1] | 2-[5-(2- Methoxy- benzylcarbamoyl)- thiophen-2-yl]- 5H-pyrrolo[2,3- b]pyrazine-7- carboxylic acid ((S)-1,2,2- trimethyl- propyl)-amide | |
| I-204 | 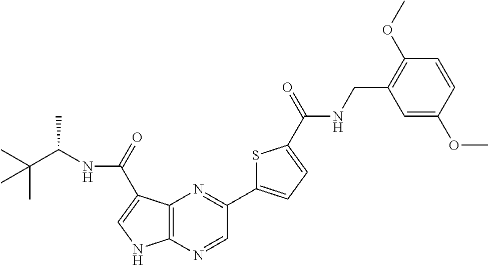 [SMILES: COc1ccc(OC)c(CNC(=O)c2ccc(-c3cnc4[nH]cc(C(=O)N[C@@H](C)C(C)(C)C)c4n3)s2)c1] | 2-[5-(2,5- Dimethoxy- benzylcarbamoyl)- thiophen-2-yl]- 5H-pyrrolo[2,3- b]pyrazine-7- carboxylic acid ((S)-1,2,2- trimethyl- propyl)-amide | |
| I-205 | 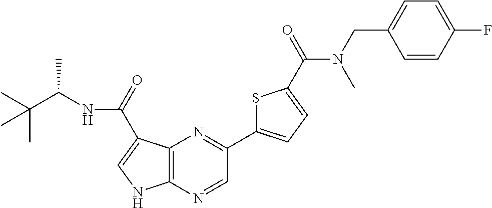 [SMILES: C[C@H](NC(=O)c1c[nH]c2ncc(-c3ccc(C(=O)N(C)Cc4ccc(F)cc4)s3)nc12)C(C)(C)C] | 2-{5-[(4-Fluoro- benzyl)-methyl- carbamoyl]- thiophen-2-yl}- 5H-pyrrolo[2,3- b]pyrazine-7- carboxylic acid ((S)-1,2,2- trimethyl- propyl)-amide | |
| I-206 | 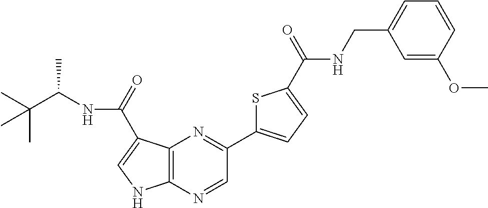 [SMILES: COc1cccc(CNC(=O)c2ccc(-c3cnc4[nH]cc(C(=O)N[C@@H](C)C(C)(C)C)c4n3)s2)c1] | 2-[5-(3- Methoxy- benzylcarbamoyl)- thiophen-2-yl]- 5H-pyrrolo[2,3- b]pyrazine-7- carboxylic acid ((S)-1,2,2- trimethyl- propyl)-amide | |
| I-207 | 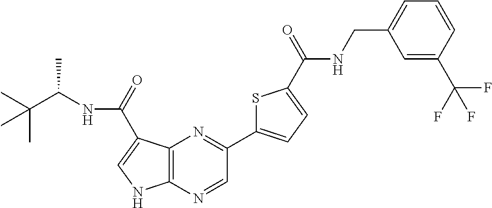 [SMILES: C[C@H](NC(=O)c1c[nH]c2ncc(-c3ccc(C(=O)NCc4cccc(C(F)(F)F)c4)s3)nc12)C(C)(C)C] | 2-[5-(3- Trifluoromethyl- benzylcarbamoyl)- thiophen-2-yl]- 5H-pyrrolo[2,3- b]pyrazine-7- carboxylic acid ((S)-1,2,2- trimethyl- propyl)-amide | |
| I-208 | 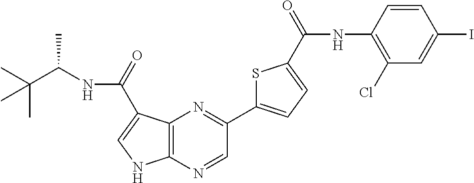 [SMILES: C[C@H](NC(=O)c1c[nH]c2ncc(-c3ccc(C(=O)Nc4ccc(I)cc4Cl)s3)nc12)C(C)(C)C] | 2-[5-(2-Chloro- 4-iodo- phenylcarbamoyl)- thiophen-2- yl]-5H- pyrrolo[2,3- b]pyrazine-7- carboxylic acid ((S)-1,2,2- trimethyl- propyl)-amide | |
| I-209 | 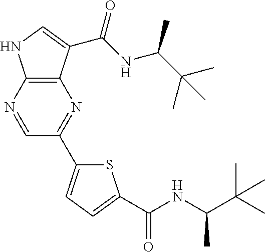 [SMILES: C[C@H](NC(=O)c1c[nH]c2ncc(-c3ccc(C(=O)N[C@H](C)C(C)(C)C)s3)nc12)C(C)(C)C] | 2-[5-((R)-1,2,2- Trimethyl- propylcarbamoyl)- thiophen-2-yl]- 5H-pyrrolo[2,3- b]pyrazine-7- carboxylic acid ((S)-1,2,2- trimethyl- propyl)-amide | |
| I-210 | 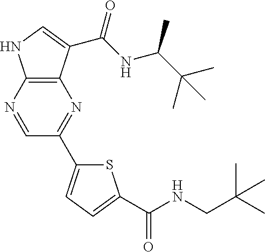 [SMILES: C[C@H](NC(=O)c1c[nH]c2ncc(-c3ccc(C(=O)NCC(C)(C)C)s3)nc12)C(C)(C)C] | 2-[5-(2,2- Dimethyl- propylcarbamoyl)- thiophen-2-yl]- 5H-pyrrolo[2,3- b]pyrazine-7- carboxylic acid ((S)-1,2,2- trimethyl- propyl)-amide | |
| I-211 | 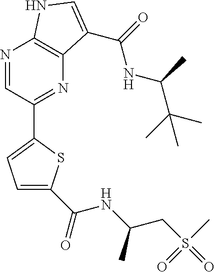 [SMILES: C[C@H](CS(C)(=O)=O)NC(=O)c1ccc(-c2cnc3[nH]cc(C(=O)N[C@@H](C)C(C)(C)C)c3n2)s1] | 2-[5-((R)-2- Methanesulfonyl- 1-methyl- ethylcarbamoyl)- thiophen-2-yl]- 5H-pyrrolo[2,3- b]pyrazine-7- carboxylic acid ((S)-1,2,2- trimethyl- propyl)-amide | |
| I-212 | 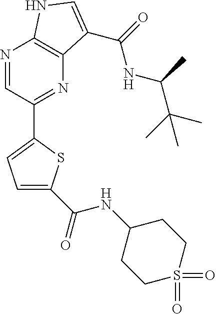 [SMILES: C[C@H](NC(=O)c1c[nH]c2ncc(-c3ccc(C(=O)NC4CCS(=O)(=O)CC4)s3)nc12)C(C)(C)C] | 2-[5-(1,1- Dioxo- hexahydro-1λ⁶- thiopyran-4- ylcarbamoyl)- thiophen-2-yl]- 5H-pyrrolo[2,3- b]pyrazine-7- carboxylic acid ((S)-1,2,2- trimethyl- propyl)-amide | |
| I-213 | 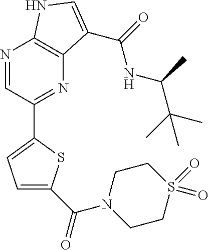 [SMILES: C[C@H](NC(=O)c1c[nH]c2ncc(-c3ccc(C(=O)N4CCS(=O)(=O)CC4)s3)nc12)C(C)(C)C] | 2-[5-(1,1- Dioxo-1λ⁶- thiomorpholine- 4-carbonyl)- thiophen-2-yl]- 5H-pyrrolo[2,3- b]pyrazine-7- carboxylic acid ((S)-1,2,2- trimethyl- propyl)-amide | |
| I-214 | 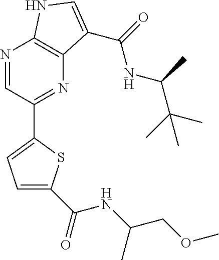 [SMILES: COCC(C)NC(=O)c1ccc(-c2cnc3[nH]cc(C(=O)N[C@@H](C)C(C)(C)C)c3n2)s1] | 2-[5-(2- Methoxy-1- methyl- ethylcarbamoyl)- thiophen-2-yl]- 5H-pyrrolo[2,3- b]pyrazine-7- carboxylic acid ((S)-1,2,2- trimethyl- propyl)-amide | |
| I-215 | 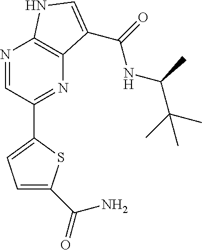 [SMILES: C[C@H](NC(=O)c1c[nH]c2ncc(-c3ccc(C(N)=O)s3)nc12)C(C)(C)C] | 2-(5- Carbamoyl- thiophen-2-yl)- 5H-pyrrolo[2,3- b]pyrazine-7- carboxylic acid ((S)-1,2,2- trimethyl- propyl)-amide | |
| I-216 | 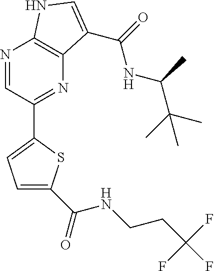 [SMILES: C[C@H](NC(=O)c1c[nH]c2ncc(-c3ccc(C(=O)NCCC(F)(F)F)s3)nc12)C(C)(C)C] | 2-[5-(3,3,3- Trifluoro- propylcarbamoyl)- thiophen-2-yl]- 5H-pyrrolo[2,3- b]pyrazine-7- carboxylic acid ((S)-1,2,2- trimethyl- propyl)-amide | |
| I-217 | 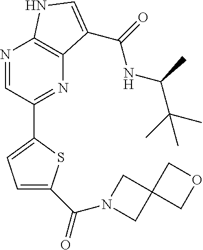 [SMILES: C[C@H](NC(=O)c1c[nH]c2ncc(-c3ccc(C(=O)N4CC5(COC5)C4)s3)nc12)C(C)(C)C] | 2-[5-(2-Oxa-6- aza- spiro[3.3]heptane- 6-carbonyl)- thiophen-2-yl]- 5H-pyrrolo[2,3- b]pyrazine-7- carboxylic acid ((S)-1,2,2- trimethyl- propyl)-amide | |
| I-218 | 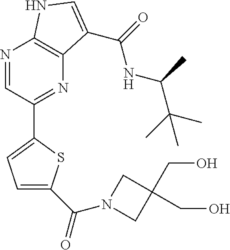 [SMILES: C[C@H](NC(=O)c1c[nH]c2ncc(-c3ccc(C(=O)N4CC(CO)(CO)C4)s3)nc12)C(C)(C)C] | 2-[5-(3,3-Bis- hydroxymethyl- azetidine-1- carbonyl)- thiophen-2-yl]- 5H-pyrrolo[2,3- b]pyrazine-7- carboxylic acid ((S)-1,2,2- trimethyl- propyl)-amide | |
| I-219 | 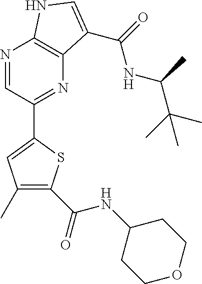 [SMILES: Cc1cc(-c2cnc3[nH]cc(C(=O)N[C@@H](C)C(C)(C)C)c3n2)sc1C(=O)NC1CCOCC1] | 2-[4-Methyl-5- (tetrahydro- pyran-4- ylcarbamoyl)- thiophen-2-yl]- 5H-pyrrolo[2,3- b]pyrazine-7- carboxylic acid ((S)-1,2,2- trimethyl- propyl)-amide | |
| I-220 | 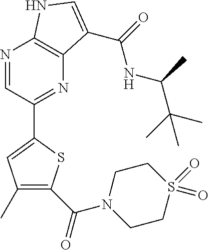 [SMILES: Cc1cc(-c2cnc3[nH]cc(C(=O)N[C@@H](C)C(C)(C)C)c3n2)sc1C(=O)N1CCS(=O)(=O)CC1] | 2-[5-(1,1- Dioxo-1λ⁶- thiomorpholine- 4-carbonyl)-4- methyl- thiophen-2-yl]- 5H-pyrrolo[2,3- b]pyrazine-7- carboxylic acid ((S)-1,2,2- trimethyl- propyl)-amide | |
| I-221 | 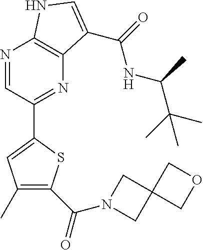 [SMILES: Cc1cc(-c2cnc3[nH]cc(C(=O)N[C@@H](C)C(C)(C)C)c3n2)sc1C(=O)N1CC2(COC2)C1] | 2-[4-Methyl-5- (2-oxa-6-aza- spiro[3.3]heptane- 6-carbonyl)- thiophen-2-yl]- 5H-pyrrolo[2,3- b]pyrazine-7- carboxylic acid ((S)-1,2,2- trimethyl- propyl)-amide | |
| I-222 | 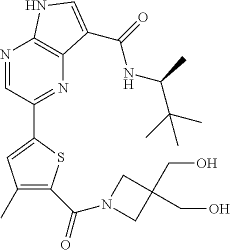 [SMILES: Cc1cc(-c2cnc3[nH]cc(C(=O)N[C@@H](C)C(C)(C)C)c3n2)sc1C(=O)N1CC(CO)(CO)C1] | 2-[5-(3,3-Bis- hydroxymethyl- azetidine-1- carbonyl)-4- methyl- thiophen-2-yl]- 5H-pyrrolo[2,3- b]pyrazine-7- carboxylic acid ((S)-1,2,2- trimethyl- propyl)-amide | |
| I-223 | 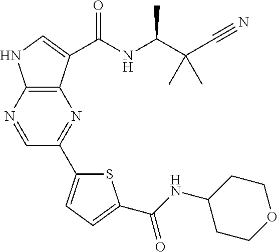 [SMILES: C[C@H](NC(=O)c1c[nH]c2ncc(-c3ccc(C(=O)NC4CCOCC4)s3)nc12)C(C)(C)C#N] | 2-[5- (Tetrahydro- pyran-4- ylcarbamoyl)- thiophen-2-yl]- 5H-pyrrolo[2,3- b]pyrazine-7- carboxylic acid ((S)-2-cyano- 1,2,2-trimethyl- ethyl)-amide | |
| I-224 | 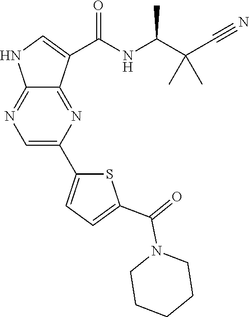 [SMILES: C[C@H](NC(=O)c1c[nH]c2ncc(-c3ccc(C(=O)N4CCCCC4)s3)nc12)C(C)(C)C#N] | 2-[5- (Piperidine-1- carbonyl)- thiophen-2-yl]- 5H-pyrrolo[2,3- b]pyrazine-7- carboxylic acid ((S)-2-cyano- 1,2,2-trimethyl- ethyl)-amide | 253- 257 |
| I-225 | 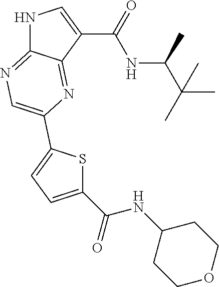 [SMILES: C[C@H](NC(=O)c1c[nH]c2ncc(-c3ccc(C(=O)NC4CCOCC4)s3)nc12)C(C)(C)C] | 2-[5- (Tetrahydro- pyran-4- ylcarbamoyl)- thiophen-2-yl]- 5H-pyrrolo[2,3- b]pyrazine-7- carboxylic acid ((S)-1,2,2- trimethyl- propyl)-amide | 333- 334 |
| I-226 | 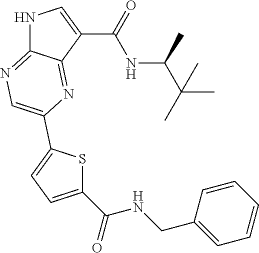 [SMILES: C[C@H](NC(=O)c1c[nH]c2ncc(-c3ccc(C(=O)NCc4ccccc4)s3)nc12)C(C)(C)C] | 2-(5- Benzylcarbamoyl- thiophen-2-yl)- 5H-pyrrolo[2,3- b]pyrazine-7- carboxylic acid ((S)-1,2,2- trimethyl- propyl)-amide | 225- 226 |
| I-227 | 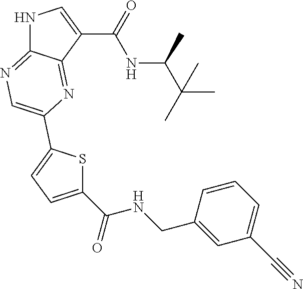 [SMILES: C[C@H](NC(=O)c1c[nH]c2ncc(-c3ccc(C(=O)NCc4cccc(C#N)c4)s3)nc12)C(C)(C)C] | 2-[5-(3-Cyano- benzylcarbamoyl)- thiophen-2-yl]- 5H-pyrrolo[2,3- b]pyrazine-7- carboxylic acid ((S)-1,2,2- trimethyl- propyl)-amide | 171- 174 |
| I-228 | 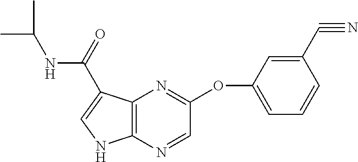 [SMILES: CC(C)NC(=O)c1c[nH]c2ncc(Oc3cccc(C#N)c3)nc12] | 2-(3-Cyano- phenoxy)-5H- pyrrolo[2,3- b]pyrazine-7- carboxylic acid isopropylamide | |
| I-229 | 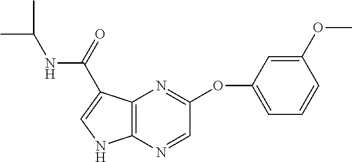 [SMILES: COc1cccc(Oc2cnc3[nH]cc(C(=O)NC(C)C)c3n2)c1] | 2-(3-Methoxy- phenoxy)-5H- pyrrolo[2,3- b]pyrazine-7- carboxylic acid isopropylamide | |
| I-230 | 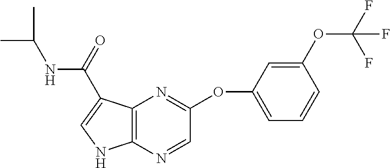 [SMILES: CC(C)NC(=O)c1c[nH]c2ncc(Oc3cccc(OC(F)(F)F)c3)nc12] | 2-(3- Trifluoromethoxy- phenoxy)-5H- pyrrolo[2,3- b]pyrazine-7- carboxylic acid isopropylamide | |
| I-231 | 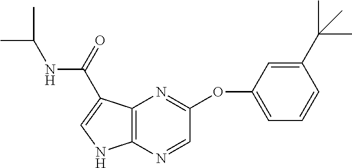 [SMILES: CC(C)NC(=O)c1c[nH]c2ncc(Oc3cccc(C(C)(C)C)c3)nc12] | 2-(3-tert-Butyl- phenoxy)-5H- pyrrolo[2,3- b]pyrazine-7- carboxylic acid isopropylamide | |
| I-232 | 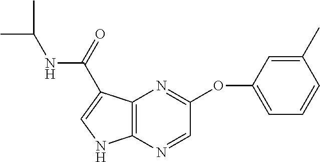 [SMILES: Cc1cccc(Oc2cnc3[nH]cc(C(=O)NC(C)C)c3n2)c1] | 2-m-Tolyloxy- 5H-pyrrolo[2,3- b]pyrazine-7- carboxylic acid isopropylamide | |
| I-233 | 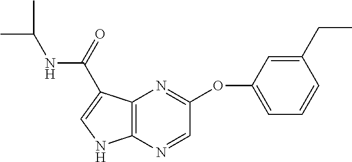 [SMILES: CCc1cccc(Oc2cnc3[nH]cc(C(=O)NC(C)C)c3n2)c1] | 2-(3-Ethyl- phenoxy)-5H- pyrrolo[2,3- b]pyrazine-7- carboxylic acid isopropylamide | |
| I-234 | 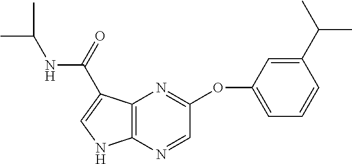 [SMILES: CC(C)NC(=O)c1c[nH]c2ncc(Oc3cccc(C(C)C)c3)nc12] | 2-(3-Isopropyl- phenoxy)-5H- pyrrolo[2,3- b]pyrazine-7- carboxylic acid isopropylamide | |
| I-235 | 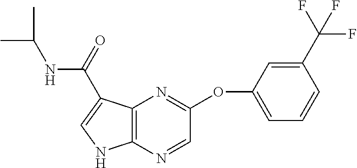 [SMILES: CC(C)NC(=O)c1c[nH]c2ncc(Oc3cccc(C(F)(F)F)c3)nc12] | 2-(3- Trifluoromethyl- phenoxy)-5H- pyrrolo[2,3- b]pyrazine-7- carboxylic acid isopropylamide | |
| I-236 | 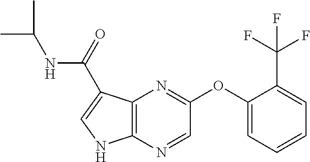 [SMILES: CC(C)NC(=O)c1c[nH]c2ncc(Oc3ccccc3C(F)(F)F)nc12] | 2-(2- Trifluoromethyl- phenoxy)-5H- pyrrolo[2,3- b]pyrazine-7- carboxylic acid isopropylamide | |
| I-237 | 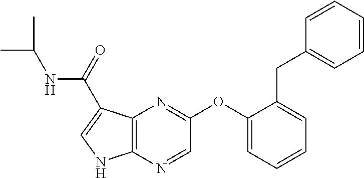 [SMILES: CC(C)NC(=O)c1c[nH]c2ncc(Oc3ccccc3Cc3ccccc3)nc12] | 2-(2-Benzyl- phenoxy)-5H- pyrrolo[2,3- b]pyrazine-7- carboxylic acid isopropylamide | |
| I-238 | 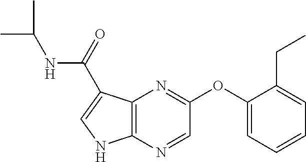 [SMILES: CCc1ccccc1Oc1cnc2[nH]cc(C(=O)NC(C)C)c2n1] | 2-(2-Ethyl- phenoxy)-5H- pyrrolo[2,3- b]pyrazine-7- carboxylic acid isopropylamide | |
| I-239 | 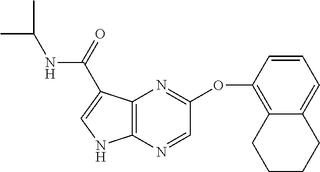 [SMILES: CC(C)NC(=O)c1c[nH]c2ncc(Oc3cccc4c3CCCC4)nc12] | 2-(5,6,7,8- Tetrahydro- naphthalen-1- yloxy)-5H- pyrrolo[2,3- b]pyrazine-7- carboxylic acid isopropylamide | |
| I-240 | 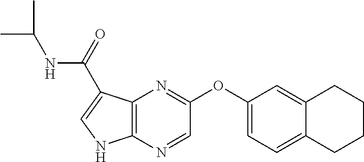 [SMILES: CC(C)NC(=O)c1c[nH]c2ncc(Oc3ccc4c(c3)CCCC4)nc12] | 2-(5,6,7,8- Tetrahydro- naphthalen-2- yloxy)-5H- pyrrolo[2,3- b]pyrazine-7- carboxylic acid isopropylamide | |
| I-241 | 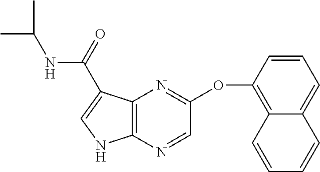 [SMILES: CC(C)NC(=O)c1c[nH]c2ncc(Oc3cccc4ccccc34)nc12] | 2-(Naphthalen- 1-yloxy)-5H- pyrrolo[2,3- b]pyrazine-7- carboxylic acid isopropylamide | |
| I-242 | 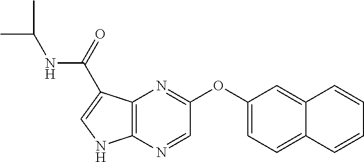 [SMILES: CC(C)NC(=O)c1c[nH]c2ncc(Oc3ccc4ccccc4c3)nc12] | 2-(Naphthalen- 2-yloxy)-5H- pyrrolo[2,3- b]pyrazine-7- carboxylic acid isopropylamide | |
| I-243 | 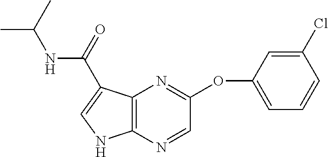 [SMILES: CC(C)NC(=O)c1c[nH]c2ncc(Oc3cccc(Cl)c3)nc12] | 2-(3-Chloro- phenoxy)-5H- pyrrolo[2,3- b]pyrazine-7- carboxylic acid isopropylamide | |
| I-244 | 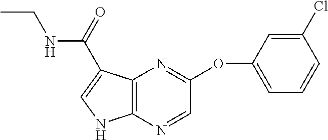 [SMILES: CCNC(=O)c1c[nH]c2ncc(Oc3cccc(Cl)c3)nc12] | 2-(3-Chloro- phenoxy)-5H- pyrrolo[2,3- b]pyrazine-7- carboxylic acid ethylamide | |
| I-245 | 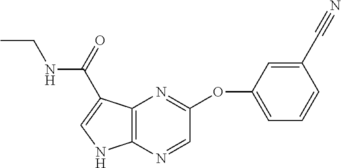 [SMILES: CCNC(=O)c1c[nH]c2ncc(Oc3cccc(C#N)c3)nc12] | 2-(3-Cyano- phenoxy)-5H- pyrrolo[2,3- b]pyrazine-7- carboxylic acid ethylamide | |
| I-246 | 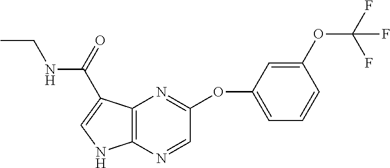 [SMILES: CCNC(=O)c1c[nH]c2ncc(Oc3cccc(OC(F)(F)F)c3)nc12] | 2-(3- Trifluoromethoxy- phenoxy)-5H- pyrrolo[2,3- b]pyrazine-7- carboxylic acid ethylamide | |
| I-247 | 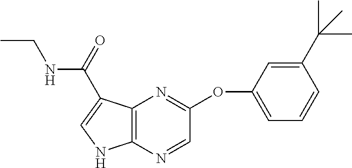 [SMILES: CCNC(=O)c1c[nH]c2ncc(Oc3cccc(C(C)(C)C)c3)nc12] | 2-(3-tert-Butyl- phenoxy)-5H- pyrrolo[2,3- b]pyrazine-7- carboxylic acid ethylamide | |
| I-248 | 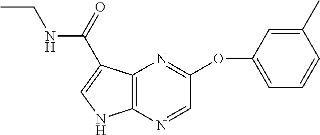 [SMILES: CCNC(=O)c1c[nH]c2ncc(Oc3cccc(C)c3)nc12] | 2-m-Tolyloxy- 5H-pyrrolo[2,3- b]pyrazine-7- carboxylic acid ethylamide | |
| I-249 | 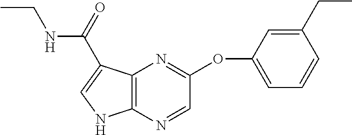 [SMILES: CCNC(=O)c1c[nH]c2ncc(Oc3cccc(CC)c3)nc12] | 2-(3-Ethyl- phenoxy)-5H- pyrrolo[2,3- b]pyrazine-7- carboxylic acid ethylamide | |
| I-250 | 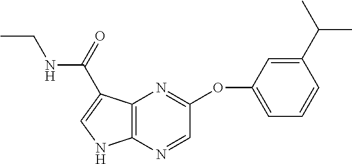 [SMILES: CCNC(=O)c1c[nH]c2ncc(Oc3cccc(C(C)C)c3)nc12] | 2-(3-Isopropyl- phenoxy)-5H- pyrrolo[2,3- b]pyrazine-7- carboxylic acid ethylamide | |
| I-251 | 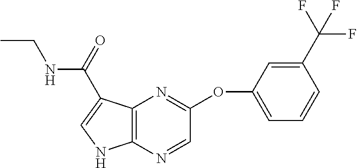 [SMILES: CCNC(=O)c1c[nH]c2ncc(Oc3cccc(C(F)(F)F)c3)nc12] | 2-(3- Trifluoromethyl- phenoxy)-5H- pyrrolo[2,3- b]pyrazine-7- carboxylic acid ethylamide | |
| I-252 | 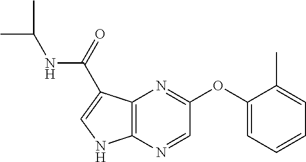 [SMILES: Cc1ccccc1Oc1cnc2[nH]cc(C(=O)NC(C)C)c2n1] | 2-o-Tolyloxy- 5H-pyrrolo[2,3- b]pyrazine-7- carboxylic acid isopropylamide | |
| I-253 | 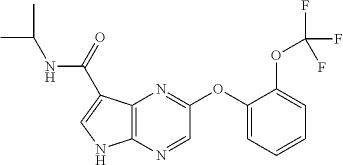 [SMILES: CC(C)NC(=O)c1c[nH]c2ncc(Oc3ccccc3OC(F)(F)F)nc12] | 2-(2- Trifluoromethoxy- phenoxy)-5H- pyrrolo[2,3- b]pyrazine-7- carboxylic acid isopropylamide | |
| I-254 | 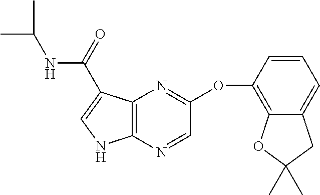 [SMILES: CC(C)NC(=O)c1c[nH]c2ncc(Oc3cccc4c3OC(C)(C)C4)nc12] | 2-(2,2- Dimethyl-2,3- dihydro- benzofuran-7- yloxy)-5H- pyrrolo[2,3- b]pyrazine-7- carboxylic acid isopropylamide | |
| I-255 | 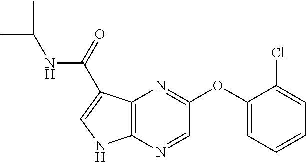 [SMILES: CC(C)NC(=O)c1c[nH]c2ncc(Oc3ccccc3Cl)nc12] | 2-(2-Chloro- phenoxy)-5H- pyrrolo[2,3- b]pyrazine-7- carboxylic acid isopropylamide | |
| I-256 | 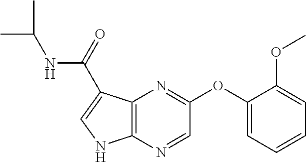 [SMILES: COc1ccccc1Oc1cnc2[nH]cc(C(=O)NC(C)C)c2n1] | 2-(2-Methoxy- phenoxy)-5H- pyrrolo[2,3- b]pyrazine-7- carboxylic acid isopropylamide | |
| I-257 | 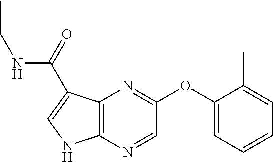 [SMILES: CCNC(=O)c1c[nH]c2ncc(Oc3ccccc3C)nc12] | 2-o-Tolyloxy- 5H-pyrrolo[2,3- b]pyrazine-7- carboxylic acid ethylamide | |
| I-258 | 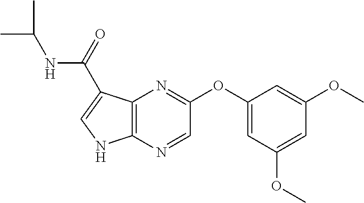 [SMILES: COc1cc(OC)cc(Oc2cnc3[nH]cc(C(=O)NC(C)C)c3n2)c1] | 2-(3,5- Dimethoxy- phenoxy)-5H- pyrrolo[2,3- b]pyrazine-7- carboxylic acid isopropylamide | |
| I-259 | 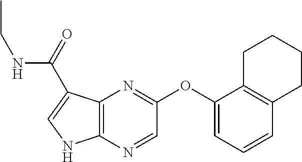 [SMILES: CCNC(=O)c1c[nH]c2ncc(Oc3cccc4c3CCCC4)nc12] | 2-(5,6,7,8- Tetrahydro- naphthalen-1- yloxy)-5H- pyrrolo[2,3- pyrazine-7- carboxylic acid ethylamide | |
| I-260 | 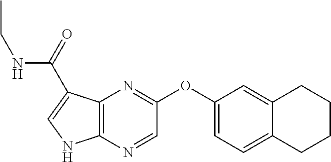 [SMILES: CCNC(=O)c1c[nH]c2ncc(Oc3ccc4c(c3)CCCC4)nc12] | 2-(5,6,7,8- Tetrahydro- naphthalen-2- yloxy)-5H- pyrrolo[2,3- b]pyrazine-7- carboxylic acid ethylamide | |
| I-261 | 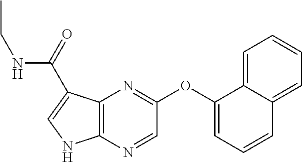 [SMILES: CCNC(=O)c1c[nH]c2ncc(Oc3cccc4ccccc34)nc12] | 2-(Naphthalen- 1-yloxy)-5H- pyrrolo[2,3- b]pyrazine-7- carboxylic acid ethylamide | |
| I-262 | 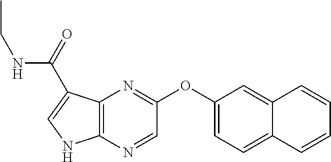 [SMILES: CCNC(=O)c1c[nH]c2ncc(Oc3ccc4ccccc4c3)nc12] | 2-(Naphthalen- 2-yloxy)-5H- pyrrolo[2,3- b]pyrazine-7- carboxylic acid ethylamide | |
| I-263 | 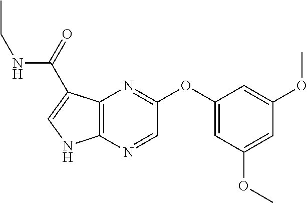 [SMILES: CCNC(=O)c1c[nH]c2ncc(Oc3cc(OC)cc(OC)c3)nc12] | 2-(3,5- Dimethoxy- phenoxy)-5H- pyrrolo[2,3- b]pyrazine-7- carboxylic acid ethylamide | |
| I-264 | 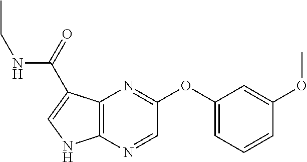 [SMILES: CCNC(=O)c1c[nH]c2ncc(Oc3cccc(OC)c3)nc12] | 2-(3-Methoxy- phenoxy)-5H- pyrrolo[2,3- b]pyrazine-7- carboxylic acid ethylamide | |
| I-265 | 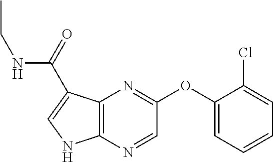 [SMILES: CCNC(=O)c1c[nH]c2ncc(Oc3ccccc3Cl)nc12] | 2-(2-Chloro- phenoxy)-5H- pyrrolo[2,3- b]pyrazine-7- carboxylic acid ethylamide | |
| I-266 | 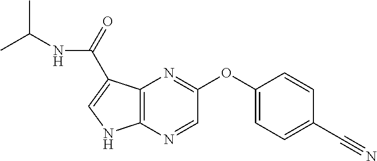 [SMILES: CC(C)NC(=O)c1c[nH]c2ncc(Oc3ccc(C#N)cc3)nc12] | 2-(4-Cyano- phenoxy)-5H- pyrrolo[2,3- b]pyrazine-7- carboxylic acid isopropylamide | |
| I-267 | 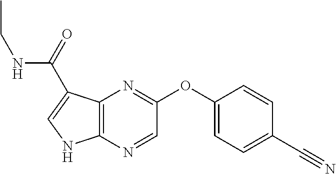 [SMILES: CCNC(=O)c1c[nH]c2ncc(Oc3ccc(C#N)cc3)nc12] | 2-(4-Cyano- phenoxy)-5H- pyrrolo[2,3- b]pyrazine-7- carboxylic acid ethylamide | |
| I-268 | 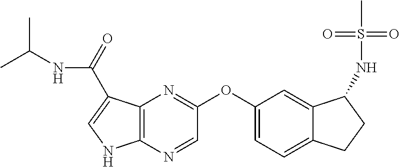 [SMILES: CC(C)NC(=O)c1c[nH]c2ncc(Oc3ccc4c(c3)[C@H](NS(C)(=O)=O)CC4)nc12] | 2-((R)-3- Methanesulfonyl amino-indan-5- yloxy)-5H- pyrrolo[2,3- b]pyrazine-7- carboxylic acid isopropylamide | |
| I-269 | 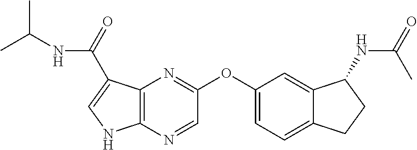 [SMILES: CC(=O)N[C@@H]1CCc2ccc(Oc3cnc4[nH]cc(C(=O)NC(C)C)c4n3)cc21] | 2-((R)-3- Acetylamino- indan-5-yloxy)- 5H-pyrrolo[2,3- b]pyrazine-7- carboxylic acid isopropylamide | |
| I-270 | 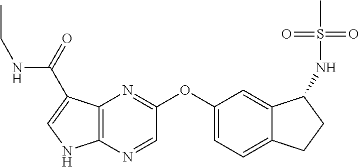 [SMILES: CCNC(=O)c1c[nH]c2ncc(Oc3ccc4c(c3)[C@H](NS(C)(=O)=O)CC4)nc12] | 2-((R)-3- Methanesulfonyl amino-indan-5- yloxy)-5H- pyrrolo[2,3- b]pyrazine-7- carboxylic acid ethylamide | |
| I-271 | 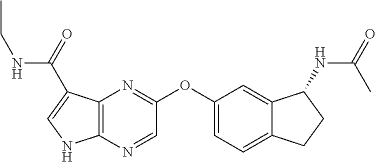 [SMILES: CCNC(=O)c1c[nH]c2ncc(Oc3ccc4c(c3)[C@H](NC(C)=O)CC4)nc12] | 2-((R)-3- Acetylamino- indan-5-yloxy)- 5H-pyrrolo[2,3- b]pyrazine-7- carboxylic acid ethylamide | |
| I-272 | 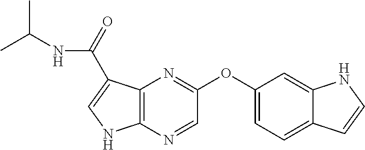 [SMILES: CC(C)NC(=O)c1c[nH]c2ncc(Oc3ccc4cc[nH]c4c3)nc12] | 2-(1H-Indol-6- yloxy)-5H- pyrrolo[2,3- b]pyrazine-7- carboxylic acid isopropylamide | |
| I-273 | 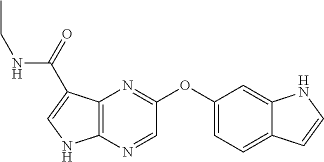 [SMILES: CCNC(=O)c1c[nH]c2ncc(Oc3ccc4cc[nH]c4c3)nc12] | 2-(1H-Indol-6- yloxy)-5H- pyrrolo[2,3- b]pyrazine-7- carboxylic acid ethylamide | |
| I-274 | 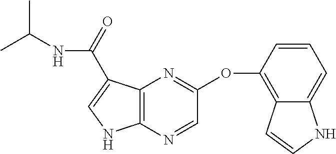 [SMILES: CC(C)NC(=O)c1c[nH]c2ncc(Oc3cccc4[nH]ccc34)nc12] | 2-(1H-Indol-4- yloxy)-5H- pyrrolo[2,3- b]pyrazine-7- carboxylic acid isopropylamide | |
| I-275 | 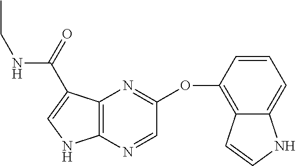 [SMILES: CCNC(=O)c1c[nH]c2ncc(Oc3cccc4[nH]ccc34)nc12] | 2-(1H-Indol-4- yloxy)-5H- pyrrolo[2,3- b]pyrazine-7- carboxylic acid ethylamide | |
| I-276 | 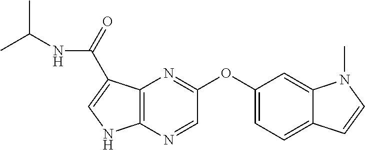 [SMILES: CC(C)NC(=O)c1c[nH]c2ncc(Oc3ccc4ccn(C)c4c3)nc12] | 2-(1-Methyl- 1H-indol-6- yloxy)-5H- pyrrolo[2,3- b]pyrazine-7- carboxylic acid isopropylamide | |
| I-277 | 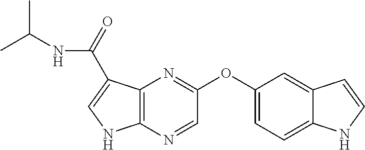 [SMILES: CC(C)NC(=O)c1c[nH]c2ncc(Oc3ccc4[nH]ccc4c3)nc12] | 2-(1H-Indol-5- yloxy)-5H- pyrrolo[2,3- b]pyrazine-7- carboxylic acid isopropylamide | |
| I-278 | 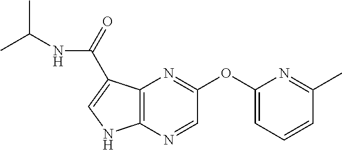 [SMILES: Cc1cccc(Oc2cnc3[nH]cc(C(=O)NC(C)C)c3n2)n1] | 2-(6-Methyl- pyridin-2- yloxy)-5H- pyrrolo[2,3- b]pyrazine-7- carboxylic acid isopropylamide | |
| I-279 | 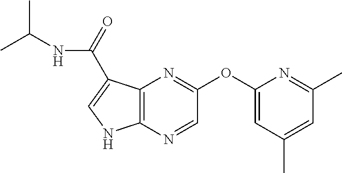 [SMILES: Cc1cc(C)nc(Oc2cnc3[nH]cc(C(=O)NC(C)C)c3n2)c1] | 2-(4,6- Dimethyl- pyridin-2- yloxy)-5H- pyrrolo[2,3- b]pyrazine-7- carboxylic acid isopropylamide | |
| I-280 | 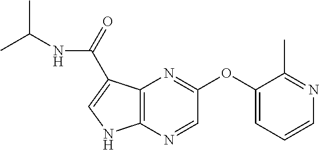 [SMILES: Cc1ncccc1Oc1cnc2[nH]cc(C(=O)NC(C)C)c2n1] | 2-(2-Methyl- pyridin-3- yloxy)-5H- pyrrolo[2,3- b]pyrazine-7- carboxylic acid isopropylamide | |
| I-281 | 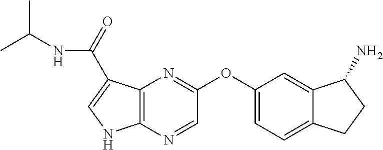 [SMILES: CC(C)NC(=O)c1c[nH]c2ncc(Oc3ccc4c(c3)[C@H](N)CC4)nc12] | 2-((R)-3- Amino-indan-5- yloxy)-5H- pyrrolo[2,3- b]pyrazine-7- carboxylic acid isopropylamide | |
| I-282 | 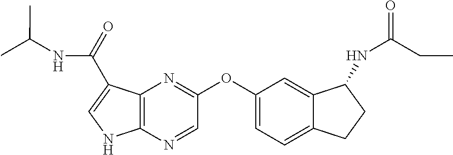 [SMILES: CCC(=O)N[C@@H]1CCc2ccc(Oc3cnc4[nH]cc(C(=O)NC(C)C)c4n3)cc21] | 2-((R)-3- Propionylamino- indan-5-yloxy)- 5H-pyrrolo[2,3- b]pyrazine-7- carboxylic acid isopropylamide | |
| I-283 | 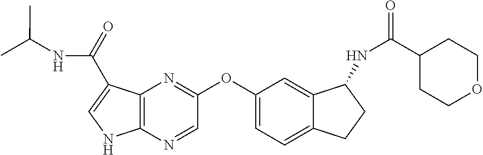 [SMILES: CC(C)NC(=O)c1c[nH]c2ncc(Oc3ccc4c(c3)[C@H](NC(=O)C3CCOCC3)CC4)nc12] | 2-{(R)-3- [(Tetrahydro- pyran-4- carbonyl)- amino]-indan-5- yloxy}-5H- pyrrolo[2,3- b]pyrazine-7- carboxylic acid isopropylamide | |
| I-284 | 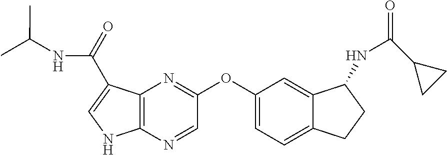 [SMILES: CC(C)NC(=O)c1c[nH]c2ncc(Oc3ccc4c(c3)[C@H](NC(=O)C3CC3)CC4)nc12] | 2-[(R)-3- (Cyclopropane- carbonyl-amino)- indan-5-yloxy]- 5H-pyrrolo[2,3- b]pyrazine-7- carboxylic acid isopropylamide | |
| I-285 | 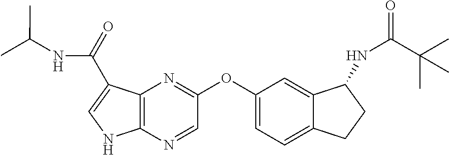 [SMILES: CC(C)NC(=O)c1c[nH]c2ncc(Oc3ccc4c(c3)[C@H](NC(=O)C(C)(C)C)CC4)nc12] | 2-[(R)-3-(2,2- Dimethyl- propionylamino)- indan-5-yloxy]- 5H-pyrrolo[2,3- b]pyrazine-7- carboxylic acid isopropylamide | |
| I-286 | 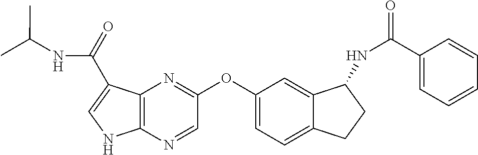 [SMILES: CC(C)NC(=O)c1c[nH]c2ncc(Oc3ccc4c(c3)[C@H](NC(=O)c3ccccc3)CC4)nc12] | 2-((R)-3- Benzoylamino- indan-5-yloxy)- 5H-pyrrolo[2,3- b]pyrazine-7- carboxylic acid isopropylamide | |
| I-287 | 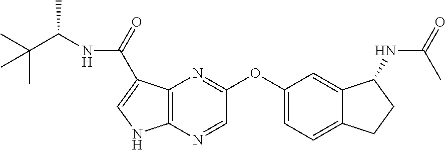 [SMILES: CC(=O)N[C@@H]1CCc2ccc(Oc3cnc4[nH]cc(C(=O)N[C@@H](C)C(C)(C)C)c4n3)cc21] | 2-((R)-3- Acetylamino- indan-5-yloxy)- 5H-pyrrolo[2,3- b]pyrazine-7- carboxylic acid ((S)-1,2,2- trimethyl- propyl)-amide | |
| I-288 | 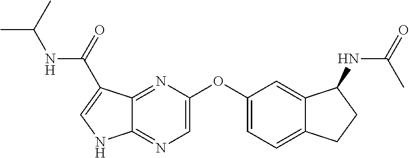 [SMILES: CC(=O)N[C@H]1CCc2ccc(Oc3cnc4[nH]cc(C(=O)NC(C)C)c4n3)cc21] | 2-((S)-3- Acetylamino- indan-5-yloxy)- 5H-pyrrolo[2,3- b]pyrazine-7- carboxylic acid isopropylamide | |
| I-289 | 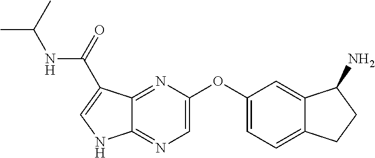 [SMILES: CC(C)NC(=O)c1c[nH]c2ncc(Oc3ccc4c(c3)[C@@H](N)CC4)nc12] | 2-((S)-3-Amino- indan-5-yloxy)- 5H-pyrrolo[2,3- b]pyrazine-7- carboxylic acid isopropylamide | |
| I-290 | 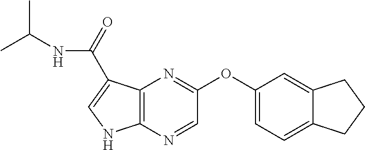 [SMILES: CC(C)NC(=O)c1c[nH]c2ncc(Oc3ccc4c(c3)CCC4)nc12] | 2-(Indan-5- yloxy)-5H- pyrrolo[2,3- b]pyrazine-7- carboxylic acid isopropylamide | |
| I-291 | 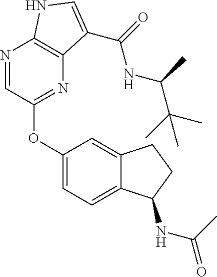 [SMILES: CC(=O)N[C@@H]1CCc2cc(Oc3cnc4[nH]cc(C(=O)N[C@@H](C)C(C)(C)C)c4n3)ccc21] | 2-((R)-1- Acetylamino- indan-5-yloxy)- 5H-pyrrolo[2,3- b]pyrazine-7- carboxylic acid ((S)-1,2,2- trimethyl- propyl)-amide | |
| I-292 | 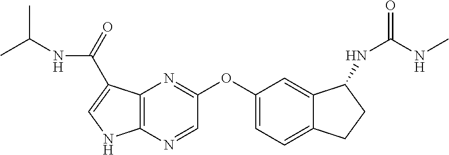 [SMILES: CNC(=O)N[C@@H]1CCc2ccc(Oc3cnc4[nH]cc(C(=O)NC(C)C)c4n3)cc21] | 2-[(R)-3-(3- Methyl-ureido)- indan-5-yloxy]- 5H-pyrrolo[2,3- b]pyrazine-7- carboxylic acid isopropylamide | |
| I-293 | 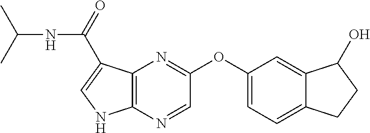 [SMILES: CC(C)NC(=O)c1c[nH]c2ncc(Oc3ccc4c(c3)C(O)CC4)nc12] | 2-(3-Hydroxy- indan-5-yloxy)- 5H-pyrrolo[2,3- b]pyrazine-7- carboxylic acid isopropylamide | |
| I-294 | 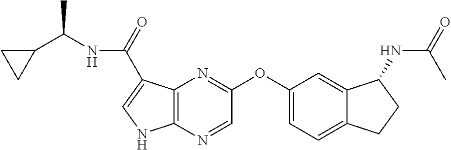 [SMILES: CC(=O)N[C@@H]1CCc2ccc(Oc3cnc4[nH]cc(C(=O)N[C@H](C)C5CC5)c4n3)cc21] | 2-((R)-3- Acetylamino- indan-5-yloxy)- 5H-pyrrolo[2,3- b]pyrazine-7- carboxylic acid ((R)-1- cyclopropyl- ethyl)-amide | |
| I-295 | 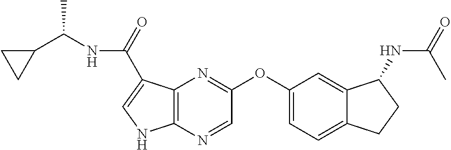 [SMILES: CC(=O)N[C@@H]1CCc2ccc(Oc3cnc4[nH]cc(C(=O)N[C@@H](C)C5CC5)c4n3)cc21] | 2-((R)-3- Acetylamino- indan-5-yloxy)- 5H-pyrrolo[2,3- b]pyrazine-7- carboxylic acid ((S)-1- cyclopropyl- ethyl)-amide | |
| I-296 | 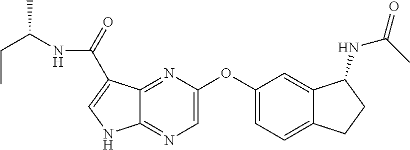 [SMILES: CC[C@H](C)NC(=O)c1c[nH]c2ncc(Oc3ccc4c(c3)[C@H](NC(C)=O)CC4)nc12] | 2-((R)-3- Acetylamino- indan-5-yloxy)- 5H-pyrrolo[2,3- b]pyrazine-7- carboxylic acid ((S)-sec-butyl)- amide | |
| I-297 | 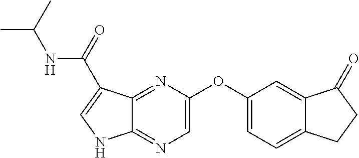 [SMILES: CC(C)NC(=O)c1c[nH]c2ncc(Oc3ccc4c(c3)C(=O)CC4)nc12] | 2-(3-Oxo-indan- 5-yloxy)-5H- pyrrolo[2,3- b]pyrazine-7- carboxylic acid isopropylamide | |
| I-298 | 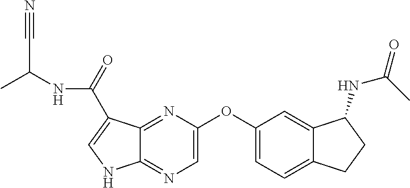 [SMILES: CC(=O)N[C@@H]1CCc2ccc(Oc3cnc4[nH]cc(C(=O)NC(C)C#N)c4n3)cc21] | 2-((R)-3- Acetylamino- indan-5-yloxy)- 5H-pyrrolo[2,3- b]pyrazine-7- carboxylic acid (cyano-methyl- methyl)-amide | |
| I-299 | 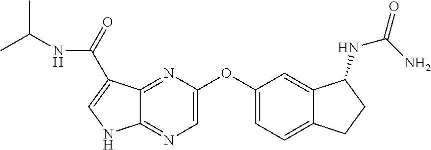 [SMILES: CC(C)NC(=O)c1c[nH]c2ncc(Oc3ccc4c(c3)[C@H](NC(N)=O)CC4)nc12] | 2-((R)-3- Ureido-indan-5- yloxy)-5H- pyrrolo[2,3- b]pyrazine-7- carboxylic acid isopropylamide | |
| I-300 | 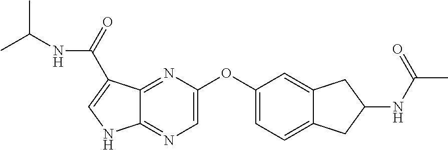 [SMILES: CC(=O)NC1Cc2ccc(Oc3cnc4[nH]cc(C(=O)NC(C)C)c4n3)cc2C1] | 2-(2- Acetylamino- indan-5-yloxy)- 5H-pyrrolo[2,3- b]pyrazine-7- carboxylic acid isopropylamide | |
| I-301 | 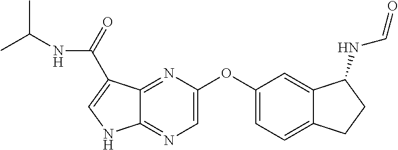 [SMILES: CC(C)NC(=O)c1c[nH]c2ncc(Oc3ccc4c(c3)[C@H](NC=O)CC4)nc12] | 2-((R)-3- Formylamino- indan-5-yloxy)- 5H-pyrrolo[2,3- b]pyrazine-7- carboxylic acid isopropylamide | |
| I-302 | 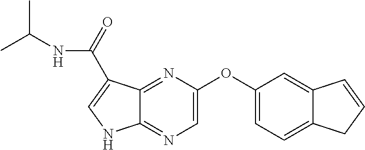 [SMILES: CC(C)NC(=O)c1c[nH]c2ncc(Oc3ccc4c(c3)C=CC4)nc12] | 2-(1H-Inden-5- yloxy)-5H- pyrrolo[2,3- b]pyrazine-7- carboxylic acid isopropylamide | |
| I-303 | 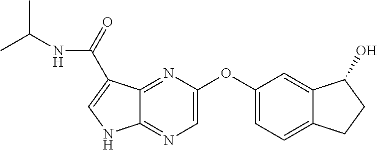 [SMILES: CC(C)NC(=O)c1c[nH]c2ncc(Oc3ccc4c(c3)[C@H](O)CC4)nc12] | 2-((R)-3- Hydroxy-indan- 5-yloxy)-5H- pyrrolo[2,3- b]pyrazine-7- carboxylic acid isopropylamide | |
| I-304 | 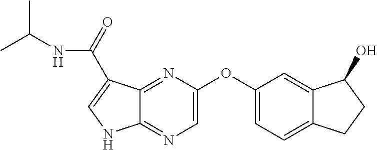 [SMILES: CC(C)NC(=O)c1c[nH]c2ncc(Oc3ccc4c(c3)[C@@H](O)CC4)nc12] | 2-((S)-3- Hydroxy-indan- 5-yloxy)-5H- pyrrolo[2,3- b]pyrazine-7- carboxylic acid isopropylamide | |
| I-305 | 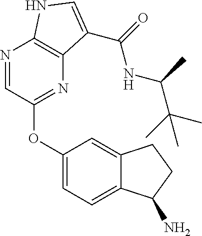 [SMILES: C[C@H](NC(=O)c1c[nH]c2ncc(Oc3ccc4c(c3)CC[C@H]4N)nc12)C(C)(C)C] | 2-((R)-1- Amino-indan-5- yloxy)-5H- pyrrolo[2,3- b]pyrazine-7- carboxylic acid ((S)-1,2,2- trimethyl- propyl)-amide | |
| I-306 | 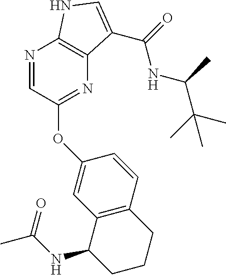 [SMILES: CC(=O)N[C@@H]1CCCc2ccc(Oc3cnc4[nH]cc(C(=O)N[C@@H](C)C(C)(C)C)c4n3)cc21] | 2-((R)-8- Acetylamino- 5,6,7,8- tetrahydro- naphthalen-2- yloxy)-5H- pyrrolo[2,3- b]pyrazine-7- carboxylic acid ((S)-1,2,2- trimethyl- propyl)-amide | |
| I-307 | 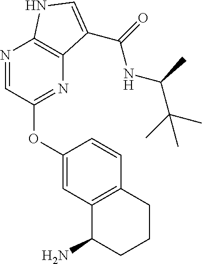 [SMILES: C[C@H](NC(=O)c1c[nH]c2ncc(Oc3ccc4c(c3)[C@H](N)CCC4)nc12)C(C)(C)C] | 2-((R)-8- Amino-5,6,7,8- tetrahydro- naphthalen-2- yloxy)-5H- pyrrolo[2,3- b]pyrazine-7- carboxylic acid ((S)-1,2,2- trimethyl- propyl)-amide | |
| I-308 | 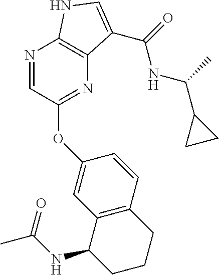 [SMILES: CC(=O)N[C@@H]1CCCc2ccc(Oc3cnc4[nH]cc(C(=O)N[C@H](C)C5CC5)c4n3)cc21] | 2-((R)-8- Acetylamino- 5,6,7,8- tetrahydro- naphthalen-2- yloxy)-5H- pyrrolo[2,3- b]pyrazine-7- carboxylic acid ((R)-1- cyclopropyl- ethyl)-amide | |
| I-309 | 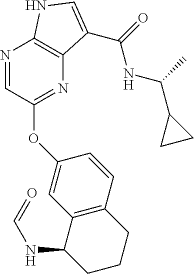 [SMILES: C[C@@H](NC(=O)c1c[nH]c2ncc(Oc3ccc4c(c3)[C@H](NC=O)CCC4)nc12)C1CC1] | 2-((R)-8- Formylamino- 5,6,7,8- tetrahydro- naphthalen-2- yloxy)-5H- pyrrolo[2,3- b]pyrazine-7- carboxylic acid ((R)-1- cyclopropyl- ethyl)-amide | |
| I-310 | 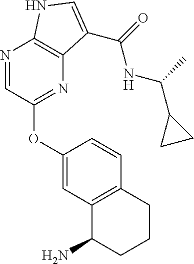 [SMILES: C[C@@H](NC(=O)c1c[nH]c2ncc(Oc3ccc4c(c3)[C@H](N)CCC4)nc12)C1CC1] | 2-((R)-8- Amino-5,6,7,8- tetrahydro- naphthalen-2- yloxy)-5H- pyrrolo[2,3- b]pyrazine-7- carboxylic acid ((R)-1- cyclopropyl- ethyl)-amide | |
| I-311 | 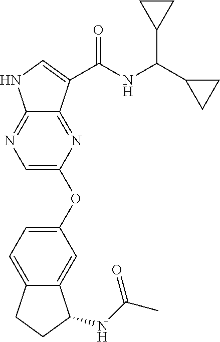 [SMILES: CC(=O)N[C@@H]1CCc2ccc(Oc3cnc4[nH]cc(C(=O)NC(C5CC5)C5CC5)c4n3)cc21] | 2-((R)-3- Acetylamino- indan-5-yloxy)- 5H-pyrrolo[2,3- b]pyrazine-7- carboxylic acid dicyclopropyl- methyl-amide | 134- 143 |
| I-312 | 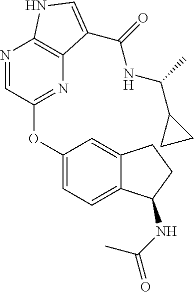 [SMILES: CC(=O)N[C@@H]1CCc2cc(Oc3cnc4[nH]cc(C(=O)N[C@H](C)C5CC5)c4n3)ccc21] | 2-((R)-1- Acetylamino- indan-5-yloxy)- 5H-pyrrolo[2,3- b]pyrazine-7- carboxylic acid ((R)-1- cyclopropyl- ethyl)-amide | |
| I-313 | 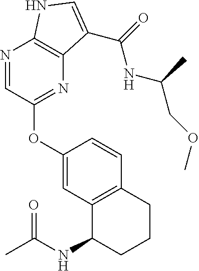 [SMILES: COC[C@H](C)NC(=O)c1c[nH]c2ncc(Oc3ccc4c(c3)[C@H](NC(C)=O)CCC4)nc12] | 2-((R)-8- Acetylamino- 5,6,7,8- tetrahydro- naphthalen-2- yloxy)-5H- pyrrolo[2,3- b]pyrazine-7- carboxylic acid ((S)-2-methoxy- 1-methyl-ethyl)- amide | |

The following Scheme, Preparations, and Examples illustrate the preparation and biological evaluation of compounds within the scope of the invention. These preparations and examples which follow are provided to enable those skilled in the art to more clearly understand and to practice the present invention. They should not be considered as limiting the scope of the invention, but merely as being illustrative and representative thereof.

### Synthesis

Incorporation of a variety of moieties on pyrrolopyrazines is disclosed in U.S. application Ser. No. 12/378,837, filed on Feb. 20, 2009, Ser. No. 12/378,869, filed on Feb. 20, 2009, Ser. No. 12/378,971, filed on Feb. 20, 2009, Ser. No. 12/378,977, filed on Feb. 20, 2009, and Ser. No. 12/378,978, filed on Feb. 20, 2009, each of which is expressly incorporated herein by reference.

In particular, the synthetic disclosures in the aforementioned applications, as well as that presented in Scheme 1, and those of the Procedures and the Examples presented below, describe synthetic details to enable the incorporation of the variety of moieties included in the below generic structure Formula I at position Q:

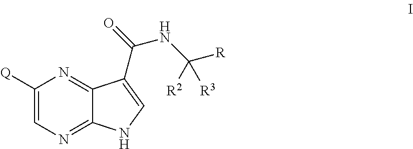

For example, U.S. application Ser. No. 12/378,837 discloses pyrrolopyrazine compounds wherein Q can be H, hydroxy, cyano, or halogen; or lower alkyl, lower alkenyl, lower alkynyl, lower hydroxyalkyl, amino, or lower haloalkyl, each optionally substituted.

For example, U.S. application Ser. No. 12/378,869 discloses pyrrolopyrazine compounds wherein Q can be phenyl substituted with two substituents which come together to form a heterocyclic or heteroaryl ring system, each optionally substituted.

For example, U.S. application Ser. No. 12/378,971 discloses pyrrolopyrazine compounds wherein Q can be —O-Q^{3a}, —S-Q^{3a}, —C(═O)(Q^{3a}), —O(CH₂)ₘC(═O)(Q^{3a}), —S(═O)(Q^{3a}), —S(═O)₂(Q^{3a}), —N(Q^{3a})₂, —N(Q^{3a})S(═O)₂(Q^{3a}), —N(Q^{3a})C(═O)(Q^{3a}), —C(═O)N(Q^{3a})₂, or —N(Q^{3a})C(═O)N(Q^{3a})₂, wherein m is 0, 1, or 2 and each Q^{3′} independently can be lower alkyl, lower haloalkyl, phenyl, cycloalkyl, heterocycloalkyl, or heteroaryl, each optionally substituted, or H.

For example, U.S. application Ser. No. 12/378,977 discloses pyrrolopyrazine compounds wherein Q can be phenyl or indolyl, each optionally substituted.

For example, U.S. application Ser. No. 12/378,978 pyrrolopyrazine compounds wherein Q can be cycloalkyl, cycloalkenyl, heterocycloalkyl, or heteroaryl, each optionally substituted.

The synthetic details in Scheme 1, as well as those of the Procedures and the Examples presented below, describe the synthetic preparations enabling the incorporation of moieties included in the above generic structure at positions R, R² and R³.

A representative method for the preparation of the compounds of the present invention is outlined in Scheme 1 below:

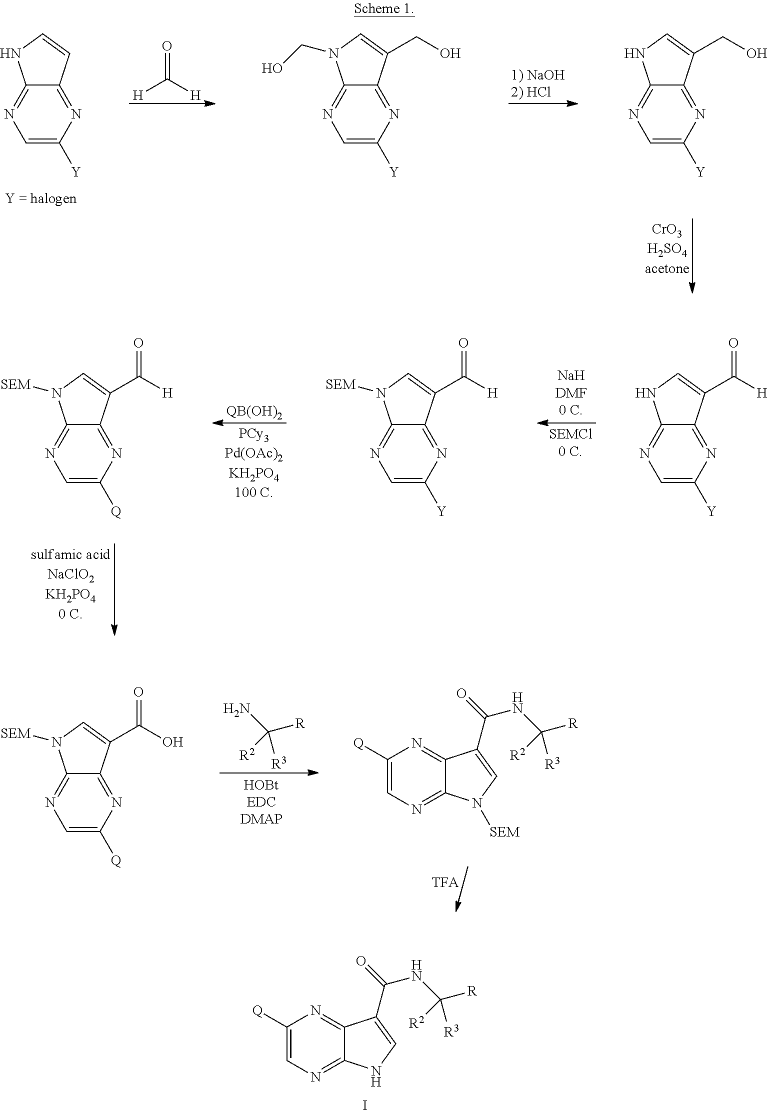

As shown in Scheme 1 above, R can be H, cyano, R′ or

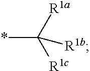

R′ can be cycloalkyl, heterocycloalkyl, or phenyl, wherein each can be optionally substituted with one or more R″; R″ can be halo, hydroxy, cyano, lower alkyl, lower haloalkyl, lower alkoxy, lower hydroxyalkyl, cycloalkyl, C(═O)R′″, or S(═O)₂R′″; R′″ can be OH, lower alkyl, lower alkoxy, lower haloalkyl, lower hydroxyalkyl, cycloalkyl, or amino; R^{la}, R^{1b}, and R^{1c} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, cycloalkyl, heterocycloalkyl, C(═O)R′″, or S(═O)₂R′″; or R^{1a} and R^{1b} together form spirocycloalkyl or spiroheterocycloalkyl, each of which can be optionally substituted with one or more R^{3′}; R² can be H or lower alkyl; R³ can be H, lower alkyl, hydroxy, hydroxy lower alkyl, lower alkoxy, lower haloalkyl, lower haloalkoxy, phenyl, phenyl lower alkyl, cycloalkyl, cycloalkyl lower alkyl, cyano, cyano lower alkyl, or heterocycloalkyl; or R³ and R′ come together to form a spirocyclic ring system, each of which can be optionally substituted with one or more R^{3′}; each R^{3′} can be independently lower alkyl, halo, hydroxy, lower alkoxy, lower haloalkyl, lower hydroxy alkyl, oxo, cyano, cyano lower alkyl, S(═O)₂R^{3″}, C(═O)R^{3′}, cycloalkyl, heterocycloalkyl, heteroaryl, or heterocycloalkenyl; R^{3″} can be H or lower alkyl; Q can be Q², Q³, or Q⁴; Q² can be heterocycloalkyl, cycloalkyl, cycloalkenyl, heterocycloalkyl phenyl, heteroaryl, biaryl, or heterobiaryl, optionally substituted with one or more Q^{2a}; Q^{2a} can be Q^{2b} or Q^{2c}; Q^{2b} can be halogen, oxo, hydroxy, —CN, —SCH₃, —S(O)₂CH₃, or —S(═O)CH₃; Q^{2c} can be Q^{2d} or Q^{2e}; or two Q^{2a} come together to form a bicyclic ring system, optionally substituted with one or more Q^{2b} or Q^{2c}; Q^{2d} can be —O(Q^{2e}), —S(═O)₂(Q^{2e}), —C(═O)N(Q^{2e})₂, —S(O)₂(Q^{2e}), —C(═O)(Q^{2e}), —C(═O)O(Q^{2e}), —N(Q^{2e})C(═O)(Q^{2e}), —N(Q^{2e})C(═O)O(Q^{2e}), or —N(Q^{2e})C(═O)N(Q^{2e})₂; each Q^{2e} can be independently H or Q^{2e′}; each Q^{2e′} can be independently lower alkyl, phenyl, benzyl, lower haloalkyl, lower alkoxy, cycloalkyl, cycloalkenyl, heterocycloalkyl, or heteroaryl, optionally substituted with one or more Q^{2f}; Q^{2f} can be Q^{2g} or Q^{2h}; Q^{2g} can be halogen, hydroxy, cyano, oxo, or —C(═O)Q^{2h}); Q^{2h} can be lower alkyl, lower haloalkyl, lower alkoxy, amino, phenyl, benzyl, cycloalkyl, heterocycloalkyl, or heteroaryl, optionally substituted with one or more Q²ⁱ; and Q²ⁱ can be halogen, hydroxy, cyano, lower alkyl, lower haloalkyl, or lower alkoxy; Q³ can be —O-Q^{3a}, —S-Q^{3a}, —C(═O)(Q^{3a}), —O(CH₂)ₘC(═O)(Q^{3a}), —S(═O)(Q^{3a}), —S(═O)₂(Q^{3a}), —N(Q^{3a})₂, —N(Q^{3a})S(═O)₂(Q^{3a}), —N(Q^{3a})C(═O)(Q^{3a}), —C(═O)N(Q^{3a})_{2,} N(Q^{3a})C(═O)N(Q^{3a})₂ or —N(Q^{3a})(CH₂)ₘC(═O)N(Q^{3a})₂; each Q^{3a} can be independently Q^{3b} or Q^{3c}; m can be 0, 1, or 2; Q^{3b} can be H; Q^{3c} can be lower alkyl, lower haloalkyl, phenyl, cycloalkyl, heterocycloalkyl, or heteroaryl, optionally substituted with one or more Q^{3d}; and each Q^{3d} can be independently Q^{3d} can be Q^{3e} or Q^{3f}; Q^{3e} can be halogen or hydroxy; Q^{3f} can be lower alkyl, lower alkoxy, lower haloalkyl, phenyl, cycloalkyl, heterocycloalkyl, or heteroaryl, optionally substituted with one or more Q^{3g}; and each Q^{3g} can be independently halogen, hydroxy, lower alkyl, lower hydroxyalkyl, lower haloalkyl, or lower alkoxy; Q⁴ can be Q^{4a} or Q^{4b}; Q^{4a} can be hydroxy, halogen, or cyano Q^{4b} can be lower alkyl, lower alkoxy, lower alkynyl, lower alkenyl, lower hydroxyalkyl, amino, or lower haloalkyl, optionally substituted with one or more Q^{4c}; Q^{4c} can be Q^{4d} or Q^{4e}; each Q^{4d} can be independently halogen, hydroxy, or cyano; each Q^{4e} can be independently lower alkyl, lower haloalkyl, lower alkoxy, amino, cycloalkyl, phenyl, heterocycloalkyl, or heteroaryl, optionally substituted with one or more Q^{4f}; each Q^{4f} can be independently hydroxy, halogen, lower alkyl, lower alkenyl, oxo, lower haloalkyl, lower alkoxy, lower hydroxyalkyl or amino; with the proviso that when Q is either cyclopropyl or thiophenyl, and R² and R³ are either H or methyl, and any two of R^{1a}, R^{1b}, and R^{1c} are either H or methyl, then the other can not be H, hydroxy, or hydroxymethyl; and with the proviso that when Q is chloro, isopropyl, isopropenyl, piperidinyl, cyclohexyl, or cyclohexenyl, and R² and R³ are either H or methyl, then R^{1a}, R^{1b} and R^{1c} can not all be H.

### Procedures

The following Procedures detail chemical syntheses of intermediates used to provided the final compounds prepared as disclosed in the Examples.

### Procedure 1.

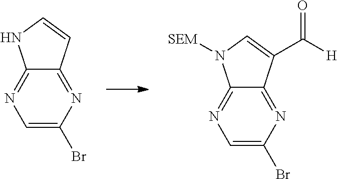 [SMILES: Brc1cnc2[nH]ccc2n1 ; C[Si](C)(C)CCOCn1cc(C=O)c2nc(Br)cnc21]

### Step 1

To a partial suspension of 2-bromo-5H-pyrrolo[2,3-b]pyrazine (5.0 g, 25.2 mmol) in 1,4-dioxane (100 mL) was added 2.0 M aqueous NaOH (25 mL, 50.0 mmol) and 37% aqueous formaldehyde (19 mL, 252 mmol). The dark homogenous reaction mixture was stirred at room temperature overnight. The organics were evaporated under reduced pressure. The aqueous layer was neutralized with 1.0 M HCl and extracted with EtOAc (2×). The combined organics were concentrated to afford 2.6 g of an orange solid. Upon standing, a thick brown precipitate formed in the aqueous layer. The precipitate was collected by filtration and dried. The brown solid was extracted with hot 10% MeOH/EtOAC (3×200 mL). The extracts were combined and evaporated to provide an additional 3.05 g of orange solid. Overall yield was 5.65 g (87%) of (2-bromo-7-hydroxymethyl-pyrrolo[2,3-b]pyrazin-5-yl)-methanol.

### Step 2

To a suspension of (2-bromo-7-hydroxymethyl-pyrrolo[2,3-b]pyrazin-5-yl)-methanol (5.65 g, 21.9 mmol) in THF (150 mL) was added a solution of 2.0 M aqueous NaOH (33 mL, 66 mmol). The homogeneous reaction mixture was stirred overnight then the organics were removed under reduced pressure. The aqueous residue was brought to pH 4 with 1.0 M aqueous HCl. The resulting precipitate was collected via filtration and rinsed with H₂O to afford 3.68 g of a yellow solid. The filtrate was extracted with EtOAc (2×) and the organics were concentrated under reduced pressure to provide an additional 0.92 g of yellow solid. Overall yield was 4.60 g (92%) of (2-bromo-5H-pyrrolo[2,3-b]pyrazin-7-yl)-methanol.

### Step 3

A stock solution of Jones reagent (2.67 M) was prepared by carefully adding concentrated H₂SO₄ (2.3 mL) to CrO₃ (2.67 g) then diluting to 10 mL with H₂O. To a partial suspension of (2-bromo-5H-pyrrolo[2,3-b]pyrazin-7-yl)-methanol (4.6 g, 20.1 mmol) in acetone (300 mL) was slowly added Jones reagent (9 mL, 24.0 mmol). During the addition the starting material gradually dissolved and a thick green precipitate was formed. The reaction mixture was stirred for 15 min then quenched with i-PrOH (2 mL) and filtered over Celite, rinsing with acetone. The filtrate was concentrated to provide 4.76 g of 2-bromo-5H-pyrrolo[2,3-b]pyrazine-7-carbaldehyde as a yellow-orange solid that was used without further purification. To a solution of this solid in DMF (50 mL) at 0° C. was added NaH (60% in mineral oil, 1.2 g, 30.1 mmol). The reaction mixture was stirred at room temperature for 30 min then cooled back to 0° C. and 2-(trimethylsilyl)ethoxymethyl chloride (4.3 mL, 24.1 mmol) was slowly added. The reaction mixture was warmed to room temperature and stirred for 1 h then quenched with H₂O and extracted with EtOAc (3×). The combined organics were washed with H₂O (3×) and brine then dried over MgSO₄ and concentrated. The residue was purified by SiO₂ chromatography (20% to 30% EtOAc/hexanes) to isolate 3.82 g (53%) of 2-bromo-5-(2-trimethylsilanyl-ethoxymethyl)-5H-pyrrolo[2,3-b]pyrazine-7-carbaldehyde as a yellow solid.

### Procedure 2.

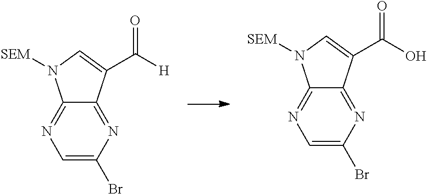 [SMILES: C[Si](C)(C)CCOCn1cc(C=O)c2nc(Br)cnc21 ; C[Si](C)(C)CCOCn1cc(C(=O)O)c2nc(Br)cnc21]

In a flask 2-bromo-5-(2-trimethylsilanylethoxymethyl)-5H-pyrrolo[2,3-b]pyrazine-7-carbaldehyde (3.11 g, 8.74 mmol) was dissolved in dioxane (120 mL) and H₂O (30 mL) and the mixture cooled at 0° C. Sulfamic acid (5.09 g, 52.4 mmol) was added, followed by a solution of sodium chlorite (1.28 g, 11.4 mmol) and potassium dihydrogen phosphate (14.3 g, 104.9 mmol) in H₂O (75 mL) via an addition funnel over 15 min. The mixture was allowed to warm to room temperature over 2 h. The resulting yellow solid was filtered off, washed with H₂O and hexane and dried. The filtrate was then extracted with EtOAc, and the combined organics washed with brine, dried over MgSO₄ and concentrated to give additional product. In total 3.71 g of 2-bromo-5-(2-trimethylsilanylethoxymethyl)-5H-pyrrolo[2,3-b]pyrazine-7-carboxylic acid was obtained as a yellow solid.

### Procedure 3.

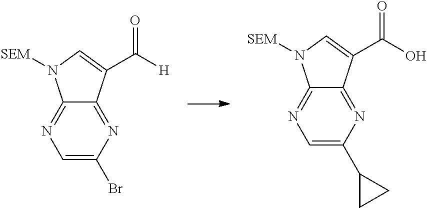 [SMILES: C[Si](C)(C)CCOCn1cc(C=O)c2nc(Br)cnc21 ; C[Si](C)(C)CCOCn1cc(C(=O)O)c2nc(C3CC3)cnc21]

### Step 1

A mixture of 2-bromo-5-(2-trimethylsilanyl-ethoxymethyl)-5H-pyrrolo[2,3-b]pyrazine-7-carbaldehyde (0.33 g, 0.93 mmol), cyclopropyl boronic acid (0.12 g, 1.39 mmol), tricyclohexyl phosphine (0.026 g, 0.09 mmol), palladium(II) acetate (0.01 g, 0.046 mmol) and potassium phosphate tribasic (0.63 g, 2.97 mmol) in 4 mL of toluene and 0.5 mL of water was flushed with Argon for 5 min then heated at 100° C. for 18 h. The cooled mixture was filtered through a pad of Celite, washed with EtOAc, and concentrated under reduced pressure. The residue was purified by silica gel chromatography eluting with 10% EtOAc/hexanes to afford 0.24 g (81%) of 2-cyclopropyl-5-(2-trimethylsilanyl-ethoxymethyl)-5H-pyrrolo[2,3-b]pyrazine-7-carbaldehyde as a yellow powder.

### Step 2

To a solution of 2-cyclopropyl-5-(2-trimethylsilanyl-ethoxymethyl)-5H-pyrrolo[2,3-b]pyrazine-7-carbaldehyde (0.24 g, 0.75 mmol) in 1,4-dioxane (10 mL) and water (2 mL) at 0° C. was added sulfamic acid (0.44 g, 4.54 mmol). Then added dropwise a solution of sodium chlorite (0.09 g, 0.98 mmol) and potassium dihydrogen phosphate (1.22 g, 9.0 mmol) in 6 mL of water. After the addition, the reaction mixture was warmed to room temperature and stirred for 2 h then partitioned between water and ethyl acetate. The organic layer was washed with brine, dried over sodium sulfate and concentrated under reduced pressure. The residue was triturated with hexanes to obtain 0.22 g (87%) of 2-cyclopropyl-5-(2-trimethylsilanyl-ethoxymethyl)-5H-pyrrolo[2,3-b]pyrazine-7-carboxylic acid as a light yellow powder.

### Procedure 4.

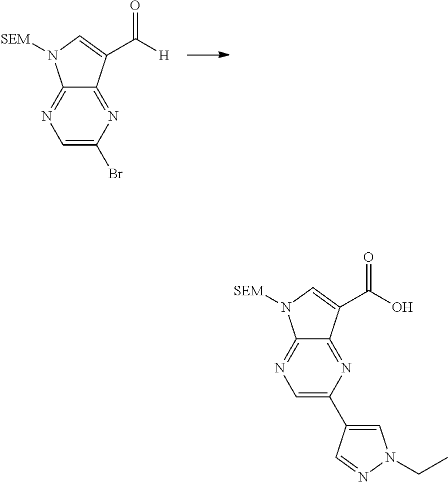 [SMILES: C[Si](C)(C)CCOCn1cc(C=O)c2nc(Br)cnc21 ; CCn1cc(-c2cnc3c(n2)c(C(=O)O)cn3COCC[Si](C)(C)C)cn1]

### Step 1

To a solution of 2-bromo-5-((2-(trimethylsilyl)ethoxy)methyl)-5H-pyrrolo[2,3-b]pyrazine-7-carbaldehyde (1.33 g, 3.73 mmol) and 1-ethyl-4-(4,4,5,5-tetramethyl-1,3,2-dioxaborolan-2-yl)-1H-pyrazole (995 mg, 4.48 mmol) in 1,2-DME (20 mL) were added Pd(Ph₃P)₄ (0.22 g, 0.19 mmol) and 2.0 M aqueous K₂CO₃ (5.6 ml, 11.2 mmol). The reaction mixture was degassed by bubbling N₂ for 15 min then heated at 100° C. overnight. The resultant maroon reaction mixture was cooled and diluted with H₂O then extracted with EtOAc (2×). The combined organics were dried over MgSO₄ and concentrated. The crude residue was purified by SiO₂ chromatography (30% to 80% EtOAc/hexanes) to afford 1.12 g (81%) of 2-(1-ethyl-1H-pyrazol-4-yl)-5-(2-(trimethylsilyl)ethoxy)methyl)-5H-pyrrolo[2,3-b]pyrazine-7-carbaldehyde as a light orange-brown solid.

### Step 2

To a solution of 2-(1-ethyl-1H-pyrazol-4-yl)-5-(2-(trimethylsilyl)ethoxy)methyl)-5H-pyrrolo[2,3-b]pyrazine-7-carbaldehyde (1.12 g, 3.01 mmol) in 1,4-dioxane (50 mL) and H₂O (10 mL) at 0° C. was added sulfamic acid (1.76 g, 18.1 mmol). Then added a solution of NaClO₂ (0.44 g, 3.92 mmol) and KH₂PO₄ (4.92 g, 36.2 mmol) in H₂O (30 mL) via dropping funnel over 15 min. The ice bath was removed and the yellow cloudy reaction mixture was stirred at room temperature for 2.5 h. The reaction mixture was diluted with H₂O and extracted with EtOAc (2×). The combined organic layers were dried over MgSO₄ and concentrated to an oily yellow solid which was triturated with 5% EtOAc/hexanes to afford 1.05 g (90%) of 2-(1-ethyl-1H-pyrazol-4-yl)-5-(2-(trimethylsilyl)ethoxy)methyl)-5H-pyrrolo[3,2-b]pyrazine-7-carboxylic acid as a light yellow solid.

### Pharmaceutical Compositions and Administration

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polyactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Indications and Methods of Treatment

The novel pyrrolopyrazine derivatives provided herein selectively inhibit JAK3 and are useful for the treatment of auto-immune and inflammatory diseases. The compounds of the invention modulate the JAK and/or SYK pathways and are useful novel pyrrolopyrazine derivatives for the treatment of auto-immune and inflammatory diseases, wherein preferred compounds selectively inhibit JAK3. For example, the compounds of the invention may inhibit JAK3 and SYK, wherein preferred compounds are selective for JAK3 of the JAK kinases and are useful novel pyrrolopyrazine derivatives for the treatment of auto-immune and inflammatory diseases. The amide linker at the 7-position of the 5H-pyrrolo[2,3-b]pyrazines affords the compounds of formula I and I′ unexpected increased potency in inhibition of JAK and Syk kinases compared to 5H-pyrrolo[2,3-b]pyrazines with other moieties at that position. Furthermore, the compounds of the invention may inhibit JAK3 and JAK2, wherein preferred compounds are selective for JAK3 of the JAK kinases, and are useful novel pyrrolopyrazine derivatives for the treatment of auto-immune and inflammatory diseases. Similarly, the compounds of the invention may inhibit JAK3 and JAK1, wherein preferred compounds are selective for JAK3 of the JAK kinases, and are useful novel pyrrolopyrazine derivatives for the treatment of auto-immune and inflammatory diseases.

The application provides a method for treating an inflammatory or autoimmune condition comprising administering to a patient in need thereof a therapeutically effective amount of the compound of formula I or I′.

The application provides the above method, further comprising administering an additional therapeutic agent selected from a chemotherapeutic or anti-proliferative agent, an anti-inflammatory agent, an immunomodulatory or immunosuppressive agent, a neurotrophic factor, an agent for treating cardiovascular disease, an agent for treating diabetes, or an agent for treating immunodeficiency disorders.

The application provides a method for treating an inflammatory condition comprising administering to a patient in need thereof a therapeutically effective amount of the compound of formula I or I′.

The application provides a method for inhibiting T-cell proliferative disorder comprising administering to a patient in need thereof a therapeutically effective amount of the compound of formula I or I′.

The application provides a method for inhibiting T-cell proliferative disorder comprising administering to a patient in need thereof a therapeutically effective amount of the compound of formula I or I′.

The application provides the above method, wherein the proliferative disorder is cancer.

The application provides a method for treating a B-cell proliferative disorder comprising administering to a patient in need thereof a therapeutically effective amount of the compound of formula I or I′.

The application provides a method for treating an immune disorder including lupus, multiple sclerosis, rheumatoid arthritis, psoriasis, Type I diabetes, complications from organ transplants, xeno transplantation, diabetes, cancer, asthma, atopic dermatitis, autoimmune thyroid disorders, ulcerative colitis, Crohn's disease, Alzheimer's disease, and Leukemia, comprising administering to a patient in need thereof a therapeutically effective amount of the compound of formula I or I′.

The application provides a method for preventing or treating all forms of organ rejection, including acute allograft or xenograft rejection and chronic allograft or xenograft rejection, of vascularized or non-vascularized transplants, comprising administering to a patient in need thereof the compound of formula I or I′.

The application provides a method for inhibiting JAK3 activity comprising administering the compound of formula I or I′, wherein the compound exhibits an IC₅₀ of 50 micromolar or less in an in vitro biochemical assay of JAK3 activity.

The application provides the above method, wherein the compound exhibits an IC₅₀ of 100 nanomolar or less in an in vitro biochemical assay of JAK3 activity.

The application provides the above method, wherein the compound exhibits an IC₅₀ of 10 nanomolar or less in an in vitro biochemical assay of JAK3 activity.

The application provides a method for inhibiting SYK activity comprising administering the compound of formula I or I′, wherein the compound exhibits an IC₅₀ of 50 micromolar or less in an in vitro biochemical assay of SYK activity.

The application provides the above method, wherein the compound exhibits an IC₅₀ of 100 nanomolar or less in an in vitro biochemical assay of SYK activity.

The application provides the above method, wherein the compound exhibits an IC₅₀ of 10 nanomolar or less in an in vitro biochemical assay of SYK activity.

The application provides a method for treating an inflammatory condition comprising co-administering to a patient in need thereof a therapeutically effective amount of an anti-inflammatory compound in combination with the compound of formula I or I′.

The application provides a method for treating an immune disorder comprising co-administering to a patient in need thereof a therapeutically effective amount of an immunosuppressant compound in combination with the compound of formula I or I′.

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), ethyl (Et), ethyl acetate (EtOAc), ethanol (EtOH), 2-ethoxy-2H-quinoline-1-carboxylic acid ethyl ester (EEDQ), diethyl ether (Et₂O), O-(7-azabenzotriazole-1-yl)-N,N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), lithium hexamethyl disilazane (LiHMDS), methanol (MeOH), melting point (mp or MP), MeSO₂— (mesyl or Ms), methyl (Me), acetonitrile (MeCN), m-chloroperbenzoic acid (MCPBA), mass spectrum (ms or MS), methyl t-butyl ether (MTBE), N-bromosuccinimide (NBS), N-carboxyanhydride (NCA), N-chlorosuccinimide (NCS), N-methylmorpholine (NMM), N-methylpyrrolidone (NMP), pyridinium chlorochromate (PCC), pyridinium dichromate (PDC), phenyl (Ph), propyl (Pr), iso-propyl (i-Pr), pounds per square inch (psi), pyridine (pyr), room temperature (rt or RT), 2-(trimethylsilyl)ethoxymethyl chloride (SEMCl), tert-butyldimethylsilyl or t-BuMe₂Si (TBDMS), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), N-urethane-N-carboxyanhydride (UNCA), Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### Example 1

### 2-Cyclopropyl-5H-pyrrolo[2,3-b]pyrazine-7-carboxylic acid [(R)-1-(1-hydroxy-cyclopentyl)-ethyl]-amide

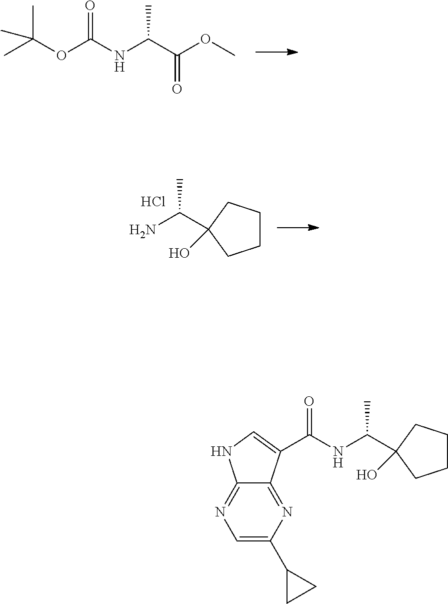

### Step 1

To a solution of Boc-D-alanine methyl ester (2.03 g, 10.0 mmol) in THF (20 mL) at 0° C. was slowly added allyl magnesium bromide (1.0 M in Et₂O, 35 mL, 35.0 mmol). The resultant white slurry was stirred at 0° C. for 1 h then at room temperature for 2 h. The reaction mixture was cooled to 0° C. and quenched with saturated aqueous NH₄Cl then diluted with H₂O and extracted with EtOAc. The combined organics were washed with H₂O, dried over MgSO₄ and concentrated to afford a viscous colorless oil. This oil was dissolved in CH₂Cl₂ (200 mL) and Grubbs 2^{nd} generation catalyst (0.17 g, 0.2 mmol) was added. The maroon reaction mixture was heated at reflux overnight. An additional amount of catalyst (0.085 g, 0.1 mmol) was added and heating was continued for 6 h. The reaction mixture was concentrated and purified by SiO₂ chromatography (10% to 40% EtOAc/hexanes) to afford 1.46 g (64%) of [(R)-1-(1-hydroxycyclopent-3-enyl)-ethyl]-carbamic acid tert-butyl ester as a light brown oil.

### Step 2

To a solution of [(R)-1-(1-hydroxycyclopent-3-enyl)-ethyl]-carbamic acid tert-butyl ester (0.62 g, 2.7 mmol) in MeOH (20 mL) was added 10% Pd on carbon (65 mg). The reaction mixture was stirred under an atmosphere of H₂ (1 atm) overnight then filtered over Celite, rinsing with EtOAc. The filtrate was concentrated and purified by SiO₂ chromatography (10% to 25% EtOAc/hexanes) to afford 336 mg of [(R)-1-(1-hydroxycyclopentyl)-ethyl]-carbamic acid tert-butyl ester as a colorless oil.

### Step 3

The above oil was dissolved in 1.0 M HCl in MeOH (10 mL) and stirred at room temperature overnight. The reaction mixture was concentrated to afford 218 mg (50%) of 1-((R)-1-amino-ethyl)-cyclopentanol hydrochloride as a hydroscopic white solid.

### Step 4

In a flask were combined 2-cyclopropyl-5-(2-trimethylsilanyl-ethoxymethyl)-5H-pyrrolo[2,3-b]pyrazine-7-carboxylic acid (120 mg, 0.36 mmol), 1-((R)-1-amino-ethyl)-cyclopentanol hydrochloride (70 mg, 0.43 mmol), EDC (77 mg, 0.40 mmol), and HOBt (54 mg, 0.40 mmol). Then added DMF (2 mL) followed by i-Pr₂NEt (0.16 mL, 0.90 mmol). The reaction mixture was stirred at room temperature for 4 h then quenched with H₂O and extracted with EtOAc (3×). The combined organics were washed with H₂O (3×) then dried over MgSO₄ and concentrated to afford 153 mg (96%) of 2-cyclopropyl-5-(2-trimethylsilanylethoxymethyl)-5H-pyrrolo[2,3-b]pyrazine-7-carboxylic acid [(R)-1-(1-hydroxy-cyclopentyl)-ethyl]-amide as a pale yellow foam.

### Step 5

To a solution of 2-cyclopropyl-5-(2-trimethylsilanylethoxymethyl)-5H-pyrrolo[2,3-b]pyrazine-7-carboxylic acid [(R)-1-(1-hydroxy-cyclopentyl)-ethyl]-amide (153 mg, 0.34 mmol) in CH₂Cl₂ (3 mL) was added TFA (1 mL). The reaction mixture was stirred for 3 h then concentrated. The residue was dissolved in CH₂Cl₂ (5 mL) and ethylene diamine (1 mL) was added. The reaction mixture was stirred for 1 h then concentrated. The residue was triturated with 10% MeOH/EtOAc. The resultant white solid was collected by filtration to afford 73 mg (68%) of 2-cyclopropyl-5H-pyrrolo[2,3-b]pyrazine-7-carboxylic acid [(R)-1-(1-hydroxy-cyclopentyl)-ethyl]-amide. MS: (M+H)⁺=315; mp=287.0-290.0.

### Example 2

### 2-Cyclopropyl-5H-pyrrolo[2,3-b]pyrazine-7-carboxylic acid [(S)-1-(1-hydroxy-cyclopentyl)-ethyl]-amide

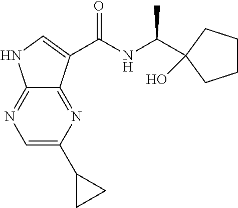 [SMILES: C[C@H](NC(=O)c1c[nH]c2ncc(C3CC3)nc12)C1(O)CCCC1]

Prepared according to the procedure outlined in Example 1, substituting Boc-L-alanine methyl ester for Boc-D-alanine methyl ester in step 1. MS: (M+H)⁺=315; mp 292.0-294.0.

### Example 3

### 2-Cyclopropyl-5H-pyrrolo[2,3-b]pyrazine-7-carboxylic acid ((S)-1,2,2-trimethylpropyl)-amide

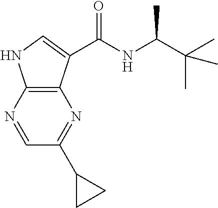 [SMILES: C[C@H](NC(=O)c1c[nH]c2ncc(C3CC3)nc12)C(C)(C)C]

Prepared according to the procedure outlined in Example 1, steps 4-5 substituting (S)-1,2,2-trimethylpropylamine for 1-((R)-1-amino-ethyl)-cyclopentanol hydrochloride. MS: (M+H)⁺=287; mp>300.

### Example 4

### 2-Cyclopropyl-5H-pyrrolo[2,3-b]pyrazine-7-carboxylic acid ((S)-3,3,3-trifluoro-1,2,2-trimethyl-propyl)-amide

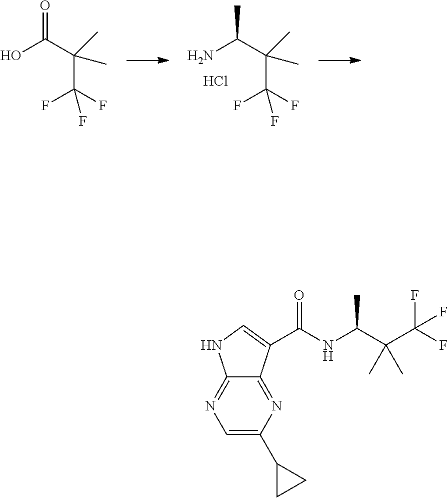

### Step 1

To a solution of 3,3,3-trifluoro-2,2-dimethylpropionic acid (2.5 g, 16.0 mmol) in dichloromethane (35 mL) was added N,O-dimethylhydroxylamine hydrochloride (2.34 g, 24 mmol), N-methylmorpholine (4.9 mL, 45 mmol) and 1-hydroxybenzotriazole hydrate (2.45 g, 16 mmol). The mixture was stirred vigorously for 5 minutes and then 1-ethyl-3-(3-dimethylaminopropyl)carbodiimide hydrochloride (5.22 g, 27.2 mmol) was added in one portion. The mixture was stirred 72 hours. The crude was taken up in 4% aqueous HCl solution (150 mL) and dichloromethane (150 mL) and transferred to a separatory funnel. The dichloromethane phase was collected and washed consecutively with equal volumes of aqueous 5% sodium bicarbonate, and then brine solution. The aqueous phases were back extracted with methylene chloride (2×80 mL). The organic phases were combined, dried (magnesium sulfate), filtered and the volume reduced, carefully on a rotary evaporator. The crude remainder, in dichloromethane (20 mL) was filtered through a short plug of silica and the solvent carefully removed under partial vacuum to provide the desired product as a light yellow oil (2.25 g), which was used directly in the next step.

### Step 2

To a cold (ice bath, 0° C.) solution of 3,3,3-trifluoro-N-methoxy-2,2,N-trimethyl-propionamide (1.25 g, 6.3 mmol) in tetrahydrofuran (10 mL) under argon (balloon) was added a 3 M solution of methylmagnesium bromide (4.2 ml, 12.6 mmol) in ether, via slow dropwise addition. The material was stirred to ambient over night and then quenched via addition of a saturated solution of ammonium chloride (15 mL). Water (20 mL) and ether (25 mL) were added and the material was shaken in a separatory funnel. The ether phase was collected and washed with brine (25 mL). The aqueous phases were back extracted with ether (2×25 mL). The organic phases were combined, dried over magnesium sulfate and filtered. The solvent was removed by careful distillation. The remainder was taken up in dry dichloromethane (20 mL) and the solvent was distilled off (repeat one more time). A clear mobile oil was obtained (assume 6 mmol) which was dried over molecular sieves and used directly in the next step.

### Step 3

To a mixture of titanium(IV) ethoxide (1.06 mL, 5.1 mmol) and (R)-(+)-2-methyl-2-propanesulfinamide (303 mg, 2.5 mmol) in dry tetrahydrofuran (5 mL) under argon atmosphere was added 4,4,4-trifluoro-3,3-dimethyl-butan-2-one (½ of material from step 2, assume 3 mmol). The material was heated to 75° C. for 18 h. The mixture was cooled to −45° C. and L-selectride (1 M in THF, 8 mL, 8 mmol) was added via dropwise addition. After 5 minutes at −45° C. the cooling bath was removed and the material was stirred for 3 hours. The mixture was cooled in an ice bath and methanol was added, drop-wise until frothing ceased. The material was stirred vigorously and brine (10 mL), was added, providing a solid suspension. This was filtered through a plug of Celite, washing well with ethyl acetate. The filtrate was collected and washed with an equal volume of brine. The aqueous phase was back extracted with ethyl acetate (2×30 mL). The organic phases were combined, dried with magnesium sulfate, filtered and evaporated. The remainder was purified via column chromatography on flash silica (30 g) eluting with 25-75% ethyl acetate/hexanes to provide 2-methyl-propane-2-sulfinic acid ((S)-3,3,3-trifluoro-1,2,2-trimethyl-propyl)-amide as a white crystalline solid (70 mg).

### Step 4

2-Methyl-propane-2-sulfinic acid ((S)-3,3,3-trifluoro-1,2,2-trimethyl-propyl)-amide (70 mg, 0.27 mmol) was dissolved in a 30% solution of hydrochloric acid in ethanol (1 mL) and the capped solution was stirred for 2 hours. The volatiles were evaporated and the remainder was taken up in dichloromethane (15 mL). The solvent was again evaporated and the material was placed under high vacuum for 30 minutes to afford (S)-3,3,3-trifluoro-1,2,2-trimethylpropylamine hydrochloride which was used without further purification.

### Step 5

2-Cyclopropyl-5H-pyrrolo[2,3-b]pyrazine-7-carboxylic acid ((S)-3,3,3-trifluoro-1,2,2-trimethyl-propyl)-amide. Prepared according to the procedure outlined in Example 1, steps 4-5 substituting (S)-3,3,3-trifluoro-1,2,2-trimethylpropylamine hydrochloride for 1-((R)-1-amino-ethyl)-cyclopentanol hydrochloride. MS: (M+H)⁺=341; mp>300.

### Example 5

### 2-Cyclopropyl-5H-pyrrolo[2,3-b]pyrazine-7-carboxylic acid (2-hydroxy-1,1,2-trimethyl-propyl)-amide

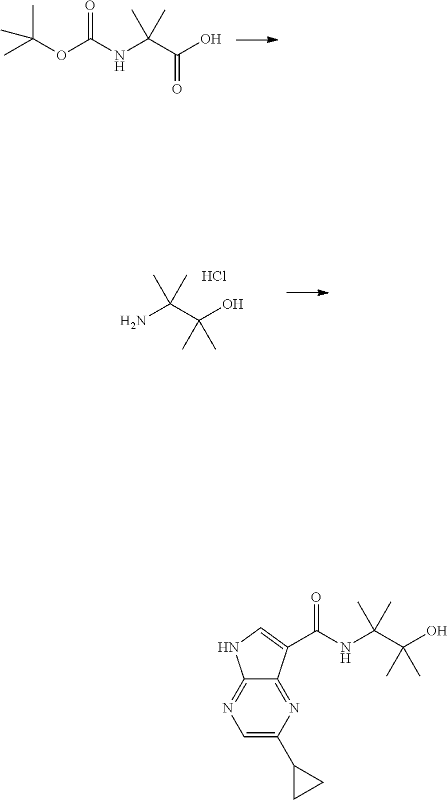

### Step 1

In a round-bottomed flask N-Boc-aminoisobutyric acid (1.20 g, 5.90 mmol) was dissolved in dichloromethane (22 mL) and MeOH (11 mL). (Trimethylsilyl)diazomethane (2.0M in hexanes, 5.0 mL, 10.0 mmol) was added dropwise and the reaction mixture was stirred at room temperature for 2.5 h. The reaction mixture was quenched with a small portion of acetic acid and concentrated under reduced pressure. The residue was dissolved in dichloromethane and washed with saturated aqueous Na₂CO₃. The aqueous layer was extracted with dichloromethane and the combined organics were dried over Na₂SO₄ and concentrated to afford 1.3 g (99%) of N-Boc-aminoisobutryric acid methyl ester as an off-white solid.

### Step 2

To a solution of N-Boc-aminoisobutryric acid methyl ester (0.60 g, 2.76 mmol) in THF (20 mL) at 0° C. was slowly added methylmagnesium bromide (3.0 M in diethyl ether, 3.6 mL, 10.8 mmol). The reaction mixture was stirred at 0° C. for 1 h then at room temperature for 5 h. The reaction was cooled back to 0° C. and quenched with saturated aqueous NH₄Cl then extracted with EtOAc (2×). The combined organics were washed with water and brine then dried over Na₂SO₄ and concentrated. The residue was purified by chromatography over 24 g SiO₂ eluting with 0% to 20% EtOAc/hexanes to afford 0.41 g (68%) of (2-hydroxy-1,1,2-trimethyl-propyl)-carbamic acid tert-butyl ester as a white solid.

### Step 3

In a round-bottomed flask, (2-hydroxy-1,1,2-trimethyl-propyl)-carbamic acid tert-butyl ester (100 mg, 0.46 mmol) was dissolved in 1.0 M HCl in MeOH (3.0 mL, 3.0 mmol). The reaction mixture was stirred at 50° C. for 4 h then cooled to room temperature and concentrated to afford 70 mg (99%) of 3-amino-2,3-dimethyl-butan-2-ol hydrochloride as an off-white solid.

### Step 4

2-Cyclopropyl-5H-pyrrolo[2,3-b]pyrazine-7-carboxylic acid (2-hydroxy-1,1,2-trimethyl-propyl)-amide. Prepared according to the procedure outlined in Example 1, steps 4-5 substituting 3-amino-2,3-dimethyl-butan-2-ol hydrochloride for 1-((R)-1-amino-ethyl)-cyclopentanol hydrochloride. MS: (M+H)⁺=303; mp=270.0-273.0.

### Example 6

### 2-Cyclopropyl-5H-pyrrolo[2,3-b]pyrazine-7-carboxylic acid ((S)-2-cyano-1,2,2-trimethyl-ethyl)-amide

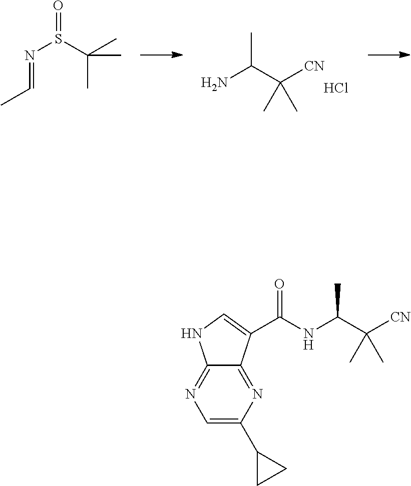

### Step 1

In a flask 2-methyl-propane-2-sulfinic acid amide (2.00 g, 16.5 mmol) was dissolved in CH₂Cl₂ (7.0 mL). Acetaldehyde (6.70 mL, 119 mmol), MgSO₄ (4.79 g, 39.8 mmol) and pyridinium tosylate (100 mg, 0.398 mmol) were added. The reaction mixture was stirred overnight at room temperature, filtered and concentrated to give 2.48 g of 2-methyl-propane-2-sulfinic acid (E)-ethylideneamide as a brown oil which was used without further purification.

### Step 2

In a flask, isobutyronitrile (0.91 mL, 10.2 mmol) was dissolved in THF (20 mL) and cooled at −78° C. LiHMDS (1.0 M in THF, 11.2 mL, 11.2 mmol) was added and the mixture stirred for 30 min at −78° C. A solution of 2-methyl-propane-2-sulfinic acid (E)-ethylideneamide (1.00 g, 6.8 mmol) in THF (5.0 mL) was slowly added. The mixture was stirred at −78° C. for 2 h and at 0° C. for 2 h then allowed to warm to room temperature overnight. The reaction mixture was quenched with saturated aqueous ammonium chloride and extracted with EtOAc. The combined organics were washed with brine, dried over MgSO₄ and concentrated. The residue was purified by SiO₂ chromatography (20-100% EtOAc/hexane) to afford 714 mg (49%) 2-methyl-propane-2-sulfinic acid (2-cyano-1,2,2-trimethyl-ethyl)-amide as a yellow viscous oil.

### Step 3

2-Methyl-propane-2-sulfinic acid (2-cyano-1,2,2-trimethyl-ethyl)-amide (714 mg, 3.30 mmol) was dissolved in 0.70 M HCl (10.0 mL) and stirred at room temperature for 2 h. Concentration gave 525 mg of 3-amino-2,2-dimethyl-butyronitrile hydrochloride as a pale brown solid which was used without further purification.

### Step 4

In a flask were combined 2-cyclopropyl-5-(2-trimethylsilanylethoxymethyl)-5H-pyrrolo[2,3-b]pyrazine-7-carboxylic acid (200 mg, 0.60 mmol), 3-amino-2,2-dimethyl-butyronitrile hydrochloride (223 mg, 1.50 mmol), EDC (264 mg, 1.38 mmol) and HOBt (186 mg, 1.38 mmol). DMF (4.0 mL) was added followed by i-Pr₂NEt (0.33 mL, 1.92 mmol). The reaction mixture was stirred at room temperature for 1 h and then concentrated. The residue was purified by SiO₂ chromatography (20-100% EtOAc/hexane) and then the enantiomers separated by preparative chiral HPLC (Chiralcel OJ-H, Hexanes/EtOH) to give 63 mg (24%) of 2-cyclopropyl-5-(2-trimethylsilanyl-ethoxymethyl)-5H-pyrrolo[2,3-b]pyrazine-7-carboxylic acid ((S)-2-cyano-1,2,2-trimethyl-ethyl)-amide as a colorless viscous oil and 67 mg (26%) 2-cyclopropyl-5-(2-trimethylsilanyl-ethoxymethyl)-5H-pyrrolo[2,3-b]pyrazine-7-carboxylic acid ((R)-2-cyano-1,2,2-trimethyl-ethyl)-amide as a colorless viscous oil.

### Step 5

In a flask 2-cyclopropyl-5-(2-trimethylsilanyl-ethoxymethyl)-5H-pyrrolo[2,3-b]pyrazine-7-carboxylic acid ((S)-2-cyano-1,2,2-trimethyl-ethyl)-amide (63 mg, 0.146 mmol) was dissolved in CH₂Cl₂ (1.5 mL) and TFA (0.50 mL) added. The reaction mixture was stirred for 2 h and concentrated. The residue was dissolved in CH₂Cl₂ (2.5 mL) then ethylene diamine (0.50 mL, 7.48 mmol) was added and the mixture stirred at room temperature overnight. The reaction mixture was then concentrated and the residue purified by SiO₂ chromatography (20-100%% EtOAc/hexane) to afford 32.5 mg (75%) 2-cyclopropyl-5H-pyrrolo[2,3-b]pyrazine-7-carboxylic acid ((S)-2-cyano-1,2,2-trimethyl-ethyl)-amide as a white powder. MS: (M+H)⁺=298.

### Example 7

### 2-Cyclopropyl-5H-pyrrolo[2,3-b]pyrazine-7-carboxylic acid ((R)-2-cyano-1,2,2-trimethyl-ethyl)-amide

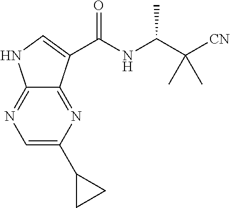 [SMILES: C[C@@H](NC(=O)c1c[nH]c2ncc(C3CC3)nc12)C(C)(C)C#N]

Prepared according to the procedure outlined in Example 6, step 5 substituting 2-cyclopropyl-5-(2-trimethylsilanyl-ethoxymethyl)-5H-pyrrolo[2,3-b]pyrazine-7-carboxylic acid ((R)-2-cyano-1,2,2-trimethyl-ethyl)-amide for 2-cyclopropyl-5-(2-trimethylsilanyl-ethoxymethyl)-5H-pyrrolo[2,3-b]pyrazine-7-carboxylic acid ((S)-2-cyano-1,2,2-trimethyl-ethyl)-amide. MS: (M+H)⁺=298.

### Example 8

### 2-Cyclopropyl-5H-pyrrolo[2,3-b]pyrazine-7-carboxylic acid [(S)-1-(1-cyano-cyclopentyl)-ethyl]-amide and 2-cyclopropyl-5H-pyrrolo[2,3-b]pyrazine-7-carboxylic acid [(R)-1-(1-cyano-cyclopentyl)-ethyl]-amide

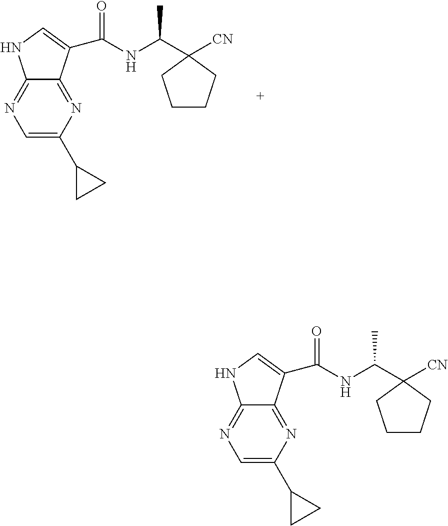 [SMILES: C[C@H](NC(=O)c1c[nH]c2ncc(C3CC3)nc12)C1(C#N)CCCC1 ; C[C@@H](NC(=O)c1c[nH]c2ncc(C3CC3)nc12)C1(C#N)CCCC1]

Prepared according to the procedure outlined in Example 6 substituting cyclopentanecarbonitrile for isobutyronitrile. Enantiomers were separated by preparative chiral HPLC at step 4. (S)-enantiomer MS: (M+H)⁺=324; mp 220.0-223.0. (R)-enantiomer MS: (M+H)⁺=324; mp 220.0-223.0.

### Example 9

### 2-Cyclopropyl-5H-pyrrolo[2,3-b]pyrazine-7-carboxylic acid [(S)-1-(1-cyano-cyclohexyl)-ethyl]-amide and 2-cyclopropyl-5H-pyrrolo[2,3-b]pyrazine-7-carboxylic acid [(R)-1-(1-cyano-cyclohexyl)-ethyl]-amide

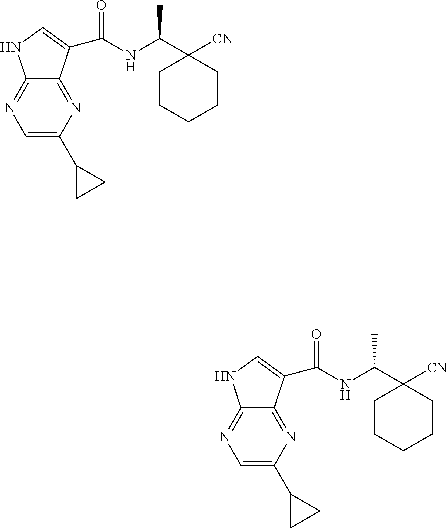 [SMILES: C[C@H](NC(=O)c1c[nH]c2ncc(C3CC3)nc12)C1(C#N)CCCCC1 ; C[C@@H](NC(=O)c1c[nH]c2ncc(C3CC3)nc12)C1(C#N)CCCCC1]

Prepared according to the procedure outlined in Example 6 substituting cyclohexanecarbonitrile for isobutyronitrile. Enantiomers were separated by preparative chiral HPLC at step 4. (S)-enantiomer MS: (M+H)⁺=338. (R)-enantiomer MS: (M+H)⁺=338.

### Example 10

### 2-Cyclopropyl-5H-pyrrolo[2,3-b]pyrazine-7-carboxylic acid [1-(tetrahydro-pyran-4-yl)-ethyl]-amide

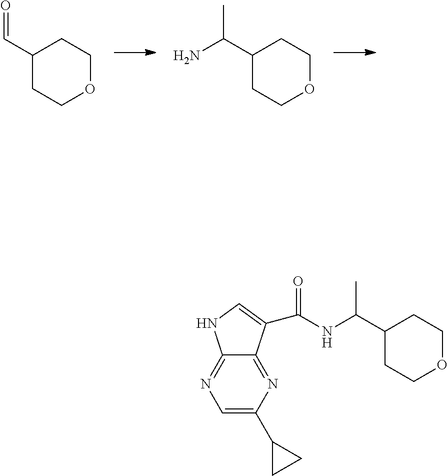

### Step 1

To a solution of tetrahydropyran-4-carbaldehyde (5.00 g, 43.8 mmol) in Et₂O (100 mL) at 0° C. was added dropwise methyl magnesium bromide (3.0 M in Et₂O, 18.9 mL, 56.9 mmol). The reaction mixture was allowed to warm to room temperature and stirred overnight. The mixture was quenched with 50% sat NH₄Cl and extracted with EtOAc. The organic extracts were washed with saturated aqueous NaCl, dried over MgSO₄ and evaporated to give 4.34 g of 1-(tetrahydropyran-4-yl)-ethanol as a colorless oil.

### Step 2

The oil from step 1 was dissolved in CH₂Cl₂ (50 mL) and triethylamine (9.8 mL, 70 mmol) was added. The mixture was cooled to 0° C. and methanesulfonyl chloride (4.07 mL, 52.6 mmol) in CH₂Cl₂ (25 mL) was added dropwise. The reaction mixture was allowed to warm to room temperature and stirred overnight. The mixture was quenched with H₂O and the aqueous layer was extracted with CH₂Cl₂. The combined organics were washed with 1M HCl, 50% satd NaHCO₃, and satd NaCl then dried over MgSO₄ and evaporated to give 6.38 g (70%) of methanesulfonic acid 1-(tetrahydro-pyran-4-yl)-ethyl ester as a colorless oil.

### Step 3

To a solution of methanesulfonic acid 1-(tetrahydro-pyran-4-yl)-ethyl ester (1.0 g, 4.80 mmol) in DMF (10.0 mL) was added sodium azide (624 mg, 9.60 mmol) and the mixture was stirred overnight at 70° C. The reaction mixture was cooled to room temperature and H₂O was added. The aqueous layer extracted with EtOAc then the combined organics were washed with sat LiCl, sat NaCl, dried over MgSO₄ and evaporated to give 0.77 g of 4-(1-azido-ethyl)-tetrahydro-pyran as a pale yellow oil.

### Step 4

The oil from step 3 was dissolved in MeOH (10 mL) and 10% Pd on carbon (40 mg) was added. The mixture was stirred under an atmosphere of H₂ (1 atm) for 1.5 h then filtered and evaporated to give 412 mg (66%) 1-(tetrahydropyran-4-yl)-ethylamine as a pale yellow oil.

### Step 5

2-Cyclopropyl-5H-pyrrolo[2,3-b]pyrazine-7-carboxylic acid [1-(tetrahydro-pyran-4-yl)-ethyl]-amide. Prepared according to the procedure outlined in Example 1, step 4 substituting 2-cyclopropyl-5H-pyrrolo[2,3-b]pyrazine-7-carboxylic acid for 2-cyclopropyl-5-(2-trimethylsilanyl-ethoxymethyl)-5H-pyrrolo[2,3-b]pyrazine-7-carboxylic acid and 1-(tetrahydropyran-4-yl)-ethylamine for 1-((R)-1-amino-ethyl)-cyclopentanol hydrochloride. MS: (M+H)⁺=315; mp 260.0-262.0.

### Example 11

### 2-Bromo-5H-pyrrolo[2,3-b]pyrazine-7-carboxylic acid (3-hydroxy-2,2-dimethyl-propyl)-amide

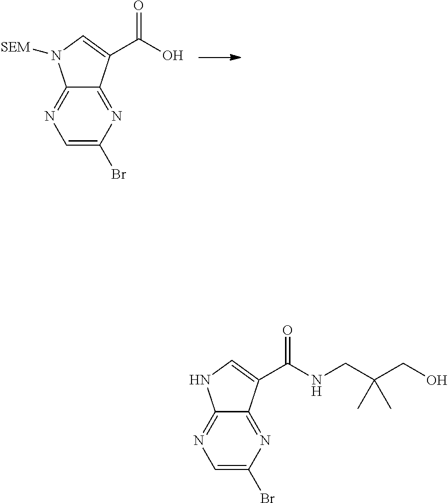 [SMILES: C[Si](C)(C)CCOCn1cc(C(=O)O)c2nc(Br)cnc21 ; CC(C)(CO)CNC(=O)c1c[nH]c2ncc(Br)nc12]

### Step 1

2-Bromo-5-(2-trimethylsilanyl-ethoxymethyl)-5H-pyrrolo[2,3-b]pyrazine-7-carboxylic acid (2.0 g, 5.39 mmol) was suspended in 36 mL of acetonitrile. N,N-diisopropylethylamine (2.8 mL, 16.2 mmol), O-benzotriazol-1-yl-N,N,N′,N′-tetramethyluronium tetrafluoroborate (1.9 g, 5.93 mmol) and 3-amino-2,2-dimethyl-propan-1-ol (0.56 g, 5.39 mmol) were added and the reaction mixture was stirred for 1.5 h. Water and ethyl acetate were added and the layers were separated. The aqueous layer was extracted once more with ethyl acetate and the combined organic layers were washed with sodium chloride solution, dried over sodium sulfate and concentrated. The residue was purified by silica gel chromatography (EtOAc/hexanes) to give 2.0 g (81%) of 2-bromo-5-(2-trimethylsilanyl-ethoxymethyl)-5H-pyrrolo[2,3-b]pyrazine-7-carboxylic acid (3-hydroxy-2,2-dimethyl-propyl)-amide.

### Step 2

2-Bromo-5H-pyrrolo[2,3-b]pyrazine-7-carboxylic acid (3-hydroxy-2,2-dimethyl-propyl)-amide was prepared according to the procedure outlined in Example 1, step 5 substituting 2-bromo-5-(2-trimethylsilanyl-ethoxymethyl)-5H-pyrrolo[2,3-b]pyrazine-7-carboxylic acid (3-hydroxy-2,2-dimethyl-propyl)-amide for 2-cyclopropyl-5-(2-trimethylsilanylethoxymethyl)-5H-pyrrolo[2,3-b]pyrazine-7-carboxylic acid [(R)-1-(1-hydroxy-cyclopentyl)-ethyl]-amide. MS: (M+H)⁺=328; mp=248.0-250.0.

### Example 12

### 2-Cyclopropyl-5H-pyrrolo[2,3b]pyrazine-7-carboxylic acid (3-methanesulfonyl-2,2-dimethyl-propyl)-amide

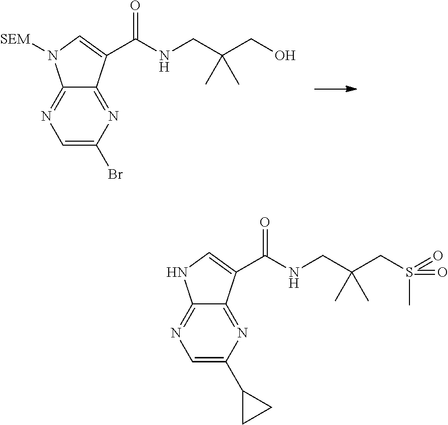 [SMILES: CC(C)(CO)CNC(=O)c1cn(COCC[Si](C)(C)C)c2ncc(Br)nc12 ; CC(C)(CNC(=O)c1c[nH]c2ncc(C3CC3)nc12)CS(C)(=O)=O]

### Step 1

2-Bromo-5-(2-trimethylsilanyl-ethoxymethyl)-5H-pyrrolo[2,3-b]pyrazine-7-carboxylic acid (2.0 g, 5.39 mmol) was suspended in 36 mL of acetonitrile. N,N-diisopropylethylamine (2.8 mL, 16.2 mmol), O-benzotriazol-1-yl-N,N,N′,N′-tetramethyluronium tetrafluoroborate (1.9 g, 5.93 mmol) and 3-amino-2,2-dimethyl-propan-1-ol (0.56 g, 5.39 mmol) were added and the reaction mixture was stirred for 1.5 h. Water and ethyl acetate were added and the layers were separated. The aqueous layer was extracted once more with ethyl acetate and the combined organic layers were washed with sodium chloride solution, dried over sodium sulfate and concentrated. The residue was purified by silica gel chromatography (EtOAc/hexanes) to give 2.0 g (81%) of 2-bromo-5-(2-trimethylsilanyl-ethoxymethyl)-5H-pyrrolo[2,3-b]pyrazine-7-carboxylic acid (3-hydroxy-2,2-dimethyl-propyl)-amide.

### Step 2

2-Bromo-5-(2-trimethylsilanyl-ethoxymethyl)-5H-pyrrolo[2,3-b]pyrazine-7-carboxylic acid (3-hydroxy-2,2-dimethyl-propyl)-amide (0.47 g, 1.02 mmol) was dissolved in 4.9 mL of toluene and 0.25 mL of water. The solution was purged with argon gas and palladium acetate (12 mg, 0.05 mmol), tricyclohexylphosphine (29 mg, 0.102 mmol), cyclopropylboronic acid (0.114 g, 1.33 mmol) and potassium phosphate tribasic (0.76 g, 3.57 mmol) were added. The reaction was stirred at 100° C. for 16 h then cooled to room temperature. Aqueous sodium bicarbonate and ethyl acetate were added and the layers were separated. The aqueous layer was extracted with ethyl acetate and the combined organic layers were washed with sodium chloride solution, dried over sodium sulfate and evaporated. The residue was purified by silica gel chromatography (MeOH/CH₂Cl₂) to give 0.34 g (79%) of 2-cyclopropyl-5-(2-trimethylsilanyl-ethoxymethyl)-5H-pyrrolo[2,3-b]pyrazine-7-carboxylic acid (3-hydroxy-2,2-dimethyl-propyl)-amide.

### Step 3

2-Cyclopropyl-5-(2-trimethylsilanyl-ethoxymethyl)-5H-pyrrolo[2,3-b]pyrazine-7-carboxylic acid (3-hydroxy-2,2-dimethyl-propyl)-amide (0.34 g, 0.81 mmol) was dissolved in 4 mL of CH₂Cl₂ and cooled in an ice bath. N,N-diisopropylethylamine (0.21 mL, 1.2 mmol) was added, followed by slow addition of methanesulfonyl chloride (0.076 mL, 0.97 mmol). The reaction mixture was warmed to room temperature over 16 h. Ethyl acetate and aqueous hydrochloric acid were added and the layers were separated. The aqueous layer was extracted with ethyl acetate and the combined organic layers were washed with sodium bicarbonate solution, dried over sodium sulfate and evaporated to afford 0.38 g of methanesulfonic acid 3-{[2-cyclopropyl-5-(2-trimethylsilanyl-ethoxymethyl)-5H-pyrrolo[2,3-b]pyrazine-7-carbonyl]-amino}-2,2-dimethyl-propyl ester.

### Step 4

Methanesulfonic acid 3-{[2-cyclopropyl-5-(2-trimethylsilanyl-ethoxymethyl)-5H-pyrrolo[2,3-b]pyrazine-7-carbonyl]-amino}-2,2-dimethyl-propyl ester (0.28 g, 0.56 mmol) was dissolved in 8 mL of DMF. Sodium thiomethoxide (157 mg, 2.24 mmol) was added and the reaction vessel was sealed and stirred in a microwave reactor for 30 min at 100° C. Aqueous sodium bicarbonate solution and dichloromethane were added and the layers were separated. The aqueous layer was extracted with dichloromethane (2×) and the combined organic layers were washed with water and sodium chloride solution, then dried over sodium sulfate and evaporated. The residue was purified by silica gel chromatography (ethyl acetate/hexanes) to give 94 mg (37%) of 2-cyclopropyl-5-(2-trimethylsilanyl-ethoxymethyl)-5H-pyrrolo[2,3-b]pyrazine-7-carboxylic acid (2,2-dimethyl-3-methylsulfanyl-propyl)-amide.

### Step 5

2-cyclopropyl-5-(2-trimethylsilanyl-ethoxymethyl)-5H-pyrrolo[2,3-b]pyrazine-7-carboxylic acid (2,2-dimethyl-3-methylsulfanyl-propyl)-amide (102 mg, 0.226 mmol) was dissolved in 0.9 mL of THF. A solution of Oxone (0.418 g, 0.682 mmol) dissolved in 0.9 mL of H₂O was slowly added and the mixture was stirred at room temperature for 16 h. Ethyl acetate and water were added to the reaction. The layers were separated and the aqueous layer was extracted with ethyl acetate (3×). The combined organic layers were washed with sodium chloride solution, dried over sodium sulfate and evaporated. The residue was purified by silica gel chromatography (ethyl acetate/hexanes) to give 80 mg (73%) of 2-cyclopropyl-5-(2-trimethylsilanyl-ethoxymethyl)-5H-pyrrolo[2,3-b]pyrazine-7-carboxylic acid (3-methanesulfonyl-2,2-dimethyl-propyl)-amide.

### Step 6

2-Cyclopropyl-5H-pyrrolo[2,3b]pyrazine-7-carboxylic acid (3-methanesulfonyl-2,2-dimethyl-propyl)-amide. Prepared according to the procedure outlined in Example 1, step 5 substituting 2-cyclopropyl-5-(2-trimethylsilanyl-ethoxymethyl)-5H-pyrrolo[2,3-b]pyrazine-7-carboxylic acid (3-methanesulfonyl-2,2-dimethyl-propyl)-amide for 2-cyclopropyl-5-(2-trimethylsilanylethoxymethyl)-5H-pyrrolo[2,3-b]pyrazine-7-carboxylic acid [(R)-1-(1-hydroxy-cyclopentyl)-ethyl]-amide. MS: (M+H)⁺ 351, mp=206.0-208.0.

### Example 13

### 2-(3,3-Dimethyl-pyrrolidin-1-yl)-5H-pyrrolo[2,3-b]pyrazine-7-carboxylic acid (3-hydroxy-2,2-dimethyl-propyl)-amide

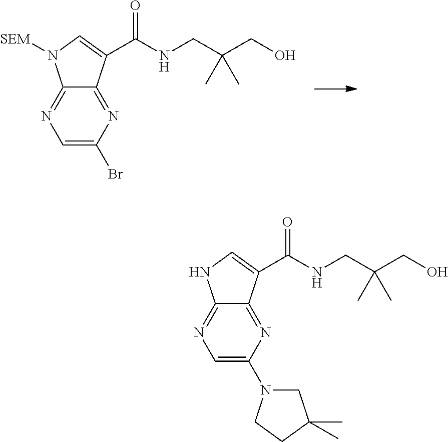 [SMILES: CC(C)(CO)CNC(=O)c1cn(COCC[Si](C)(C)C)c2ncc(Br)nc12 ; CC(C)(CO)CNC(=O)c1c[nH]c2ncc(N3CCC(C)(C)C3)nc12]

### Step 1

2-Bromo-5-(2-trimethylsilanyl-ethoxymethyl)-5H-pyrrolo[2,3-b]pyrazine-7-carboxylic acid (3-hydroxy-2,2-dimethyl-propyl)-amide (0.15 g, 0.33 mmol) was dissolved in 3.3 ml of dimethylsulfoxide and then purged with argon gas. Potassium carbonate (0.113 g, 0.82 mmol), 3,3-dimethylpyrrolidine (0.16 g, 1.64 mmol), DL-proline (11 mg, 0.098 mmol) and then copper(I) iodide (9 mg, 0.049 mmol) were added. The reaction was sealed and stirred in a 100° C. oil bath for 16 h. The reaction was cooled and water and ethyl acetate were added. The layers were separated and the organic layer was extracted once more with ethyl acetate. The combined organic layers were then washed with water and saturated sodium chloride solution, dried over sodium sulfate, and evaporated. The resulting residue was purified by silica gel chromatography (methanol/dichloromethane) to give 130 mg (83%) of 2-(3,3-dimethyl-pyrrolidin-1-yl)-5-(2-trimethylsilanyl-ethoxymethyl)-5H-pyrrolo[2,3-b]pyrazine-7-carboxylic acid (3-hydroxy-2,2-dimethyl-propyl)-amide. (M+H)⁺=476.

### Step 2

2-(3,3-Dimethyl-pyrrolidin-1-yl)-5-(2-trimethylsilanyl-ethoxymethyl)-5H-pyrrolo[2,3-b]pyrazine-7-carboxylic acid (3-hydroxy-2,2-dimethyl-propyl)-amide (0.13 g, 0.27 mmol) was dissolved in 1.3 ml of methanol. Then 1.7 ml of 6M aqueous HCl was added slowly and the reaction was then stirred at 90° C. in a heat block for 30 min. The reaction was cooled, sodium bicarbonate solution was added and then extracted twice with ethyl acetate. The combined organic layers were washed with sodium chloride solution, dried over sodium sulfate and concentrated. The resulting residue was redissolved in 10 ml of ethanol, and sodium acetate (0.73 g, 5.4 mmol) was added. The reaction was stirred at 60° C. for 16 h. After cooling, water was added and the solution extracted with ethyl acetate three times. The combined organic layers were washed with sodium chloride solution, dried over sodium sulfate and evaporated. The residue was purified by silica gel chromatography (methanol/dichloromethane) to yield 51 mg (54%) of 2-(3,3-dimethyl-pyrrolidin-1-yl)-5H-pyrrolo[2,3-b]pyrazine-7-carboxylic acid (3-hydroxy-2,2-dimethyl-propyl)-amide. MS: (M+H)⁺=346; mp=223.0-225.0; elemental analysis: calculated C, 69.59; H, 7.88; N, 20.27. found C, 69.22; H, 7.70; N, 20.07.

### Example 14

### 2-Dimethylamino-5H-pyrrolo[2,3-b]pyrazine-7-carboxylic acid (3-hydroxy-2,2-dimethyl-propyl)-amide

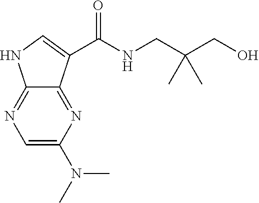 [SMILES: CN(C)c1cnc2[nH]cc(C(=O)NCC(C)(C)CO)c2n1]

Prepared according to the procedure outlined in Example 13 substituting dimethylamine hydrochloride for 3,3-dimethylpyrrolidine. MS: (M+H)⁺=292; mp=222.0-224.0.

### Example 15

### 2-Pyrrolidin-1-yl-5H-pyrrolo[2,3-b]pyrazine-7-carboxylic acid (3-hydroxy-2,2-dimethyl-propyl)-amide

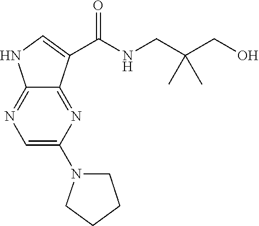 [SMILES: CC(C)(CO)CNC(=O)c1c[nH]c2ncc(N3CCCC3)nc12]

Prepared according to the procedure outlined in Example 13 substituting pyrrolidine for 3,3-dimethylpyrrolidine. MS: (M+H)⁺=318; mp=220.0-222.0.

### Example 16

### 2-Phenylamino-5H-pyrrolo[2,3-b]pyrazine-7-carboxylic acid (3-hydroxy-2,2-dimethyl-propyl)-amide

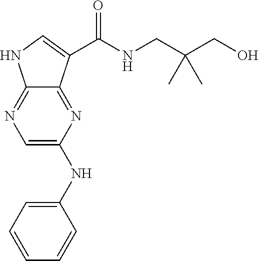 [SMILES: CC(C)(CO)CNC(=O)c1c[nH]c2ncc(Nc3ccccc3)nc12]

Prepared according to the procedure outlined in Example 13 substituting aniline for 3,3-dimethylpyrrolidine. MS: (M+H)⁺=340; mp=280.0-282.0.

### Example 17

### 2-(Methylcarbamoylmethyl-amino)-5H-pyrrolo[2,3-b]pyrazine-7-carboxylic acid (3-hydroxy-2,2-dimethyl-propyl)-amide

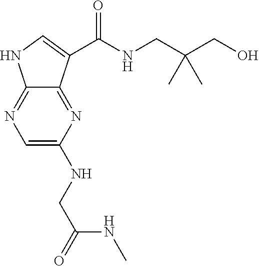 [SMILES: CNC(=O)CNc1cnc2[nH]cc(C(=O)NCC(C)(C)CO)c2n1]

### Step 1

2-(Methylcarbamoylmethyl-amino)-5-(2-trimethylsilanyl-ethoxymethyl)-5H-pyrrolo[2,3-b]pyrazine-7-carboxylic acid (3-hydroxy-2,2-dimethyl-propyl)-amide was prepared according to the procedure outlined in Example 13, step 1 substituting 2-amino-N-methylacetamide for 3,3-dimethylpyrrolidine.

### Step 2

2-(Methylcarbamoylmethyl-amino)-5-(2-trimethylsilanyl-ethoxymethyl)-5H-pyrrolo[2,3-b]pyrazine-7-carboxylic acid (3-hydroxy-2,2-dimethyl-propyl)-amide (90 mg, 0.193 mmol) was dissolved in 2 ml of 1M tetrabutylammonium fluoride in THF. The solution was stirred at 60° C. for 24 h. After cooling the reaction, sodium bicarbonate solution was added and the reaction extracted three times with ethyl acetate. The combined organic layers were washed with sodium chloride solution, dried over sodium sulfate and concentrated. The residue was purified by silica gel chromatography (ammonia/methanol/dichloromethane) to give 15 mg (21%) of 2-(methylcarbamoylmethyl-amino)-5H-pyrrolo[2,3-b]pyrazine-7-carboxylic acid (3-hydroxy-2,2-dimethyl-propyl)-amide. MS: (M+H)⁺=335; mp=270.0-275.0.

### Example 18

### 2-Trifluoromethyl-5H-pyrrolo[2,3-b]pyrazine-7-carboxylic acid (3-hydroxy-2,2-dimethyl-propyl)-amide

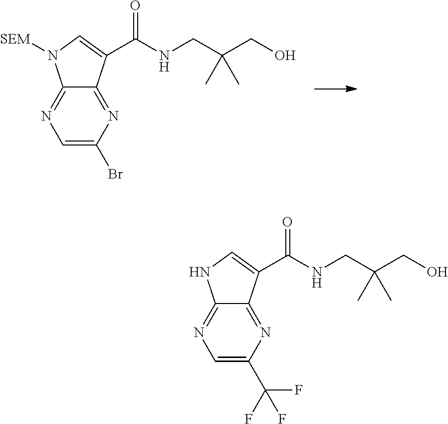 [SMILES: CC(C)(CO)CNC(=O)c1cn(COCC[Si](C)(C)C)c2ncc(Br)nc12 ; CC(C)(CO)CNC(=O)c1c[nH]c2ncc(C(F)(F)F)nc12]

### Step 1

2-Bromo-5-(2-trimethylsilanyl-ethoxymethyl)-5H-pyrrolo[2,3-b]pyrazine-7-carboxylic acid (3-hydroxy-2,2-dimethyl-propyl)-amide (0.5 g, 1.08 mmol) was dissolved in 5 ml of dichloromethane. N,N-diisopropylethylamine (1.5 ml, 8.7 mmol) was added and the reaction was cooled in an ice bath. 2-Trimethylsilylethoxymethyl chloride (0.39 ml, 2.18 mmol) was added slowly and the reaction was stirred for 16 h at room temperature. Dilute aqueous HCl and ethyl acetate were added. The layers were separated and the aqueous layer was extracted once more with ethyl acetate. The combined organic layers were washed with sodium chloride solution and dried over sodium sulfate. After evaporation, the residue was purified by silica gel chromatography (ethyl acetate/hexanes) to give 0.6 g (93%) of 2-bromo-5-(2-trimethylsilanyl-ethoxymethyl)-5H-pyrrolo[2,3-b]pyrazine-7-carboxylic acid [2,2-dimethyl-3-(2-trimethylsilanyl-ethoxymethoxy)-propyl]-amide.

### Step 2

2-Bromo-5-(2-trimethylsilanyl-ethoxymethyl)-5H-pyrrolo[2,3-b]pyrazine-7-carboxylic acid [2,2-dimethyl-3-(2-trimethylsilanyl-ethoxymethoxy)-propyl]-amide (0.18 g, 0.306 mmol) was dissolved in 0.6 ml of N,N-dimethylacetamide. The solution was purged with argon gas and then cooled in an ice bath. Copper (116 mg, 1.83 mmol) and dibromodifluoromethane (0.113 ml, 1.22 mmol) were added, and the reaction vessel was sealed and stirred at 100° C. for 16 h. After cooling, sodium bicarbonate solution and ethyl acetate were added to the reaction. The layers were separated and the aqueous layer was extracted once more with ethyl acetate. The combined organic layers were washed with sodium chloride solution and dried over sodium sulfate. After concentration, the residue was purified by silica gel chromatography (ethyl acetate/hexanes) to give 41 mg (23%) of 2-trifluoromethyl-5-(2-trimethylsilanyl-ethoxymethyl)-5H-pyrrolo[2,3-b]pyrazine-7-carboxylic acid [2,2-dimethyl-3-(2-trimethylsilanyl-ethoxymethoxy)-propyl]-amide. (M+H)⁺=577.

### Step 3

2-Trifluoromethyl-5-(2-trimethylsilanyl-ethoxymethyl)-5H-pyrrolo[2,3-b]pyrazine-7-carboxylic acid [2,2-dimethyl-3-(2-trimethylsilanyl-ethoxymethoxy)-propyl]-amide (41 mg, 0.071 mmol) was dissolved in 0.4 ml of methanol. Then 0.5 ml of 6M aqueous HCl was added slowly and the reaction was stirred in a heat block at 90° C. for 45 min. The reaction was cooled, sodium bicarbonate solution was added and then extracted twice with ethyl acetate. The combined organic layers were washed with sodium chloride solution, dried over sodium sulfate and concentrated. The residue was purified by silica gel chromatography (methanol/dichloromethane) to give 15.7 mg (70%) of 2-trifluoromethyl-5H-pyrrolo[2,3-b]pyrazine-7-carboxylic acid (3-hydroxy-2,2-dimethyl-propyl)-amide. MS: (M+H)⁺=317; mp=221.0-223.0.

### Example 19

### 2-Cyclopropyl-5H-pyrrolo[2,3-b]pyrazine-7-carboxylic acid (2-methoxy-2-methyl-propyl)-amide

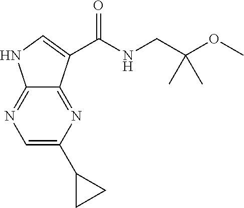 [SMILES: COC(C)(C)CNC(=O)c1c[nH]c2ncc(C3CC3)nc12]

Prepared according to the procedure outlined in Example 12, steps 1-2 and 6 substituting 2-methoxy-2-methylpropylamine for 3-amino-2,2-dimethyl-propan-1-ol in step 1. MS: (M+H)'=289; mp=259.0-262.0.

### Example 20

### 2-Cyclopropyl-5H-pyrrolo[2,3-b]pyrazine-7-carboxylic acid (3-methoxy-2,2-dimethyl-propyl)-amide

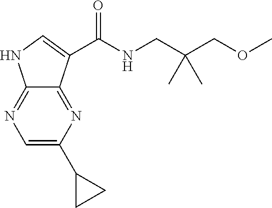 [SMILES: COCC(C)(C)CNC(=O)c1c[nH]c2ncc(C3CC3)nc12]

Prepared according to the procedure outlined in Example 1, steps 4-5 substituting 2,2-dimethyl-3-methoxypropylamine for 1-((R)-1-amino-ethyl)-cyclopentanol hydrochloride. (M+H)⁺=303; mp=230.0-232.0.

### Example 21

### 2-Cyclopropyl-5H-pyrrolo[2,3-b]pyrazine-7-carboxylic acid dicyclopropylmethyl-amide

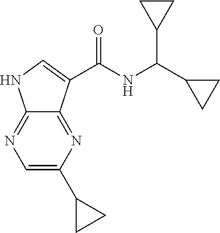 [SMILES: O=C(NC(C1CC1)C1CC1)c1c[nH]c2ncc(C3CC3)nc12]

Prepared according to the procedure outlined in Example 12, steps 1-2 and 6 substituting dicyclopropylmethylamine hydrochloride for 3-amino-2,2-dimethyl-propan-1-ol in step 1. MS: (M+H)⁺=297; mp=224.0-226.0.

### Example 22

### 2-Cyclopropyl-5H-pyrrolo[2,3-b]pyrazine-7-carboxylic acid ((R)-1-methoxymethyl-2,2-dimethyl-propyl)-amide

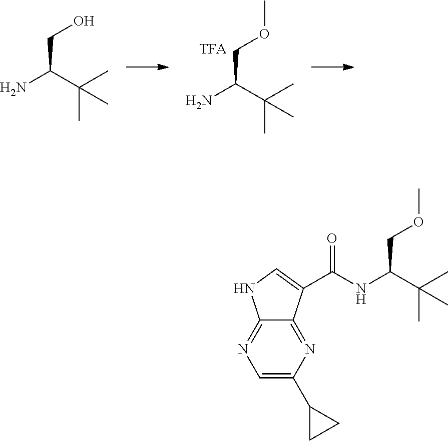

### Step 1

R-tert-leucinol (0.23 g, 1.96 mmol) and di-tert-butyldicarbonate (0.85 g, 3.9 mmol) were dissolved in 10 ml of dichloromethane and stirred for 3 days. Aqueous HCl and ethyl acetate were then added, the layers were separated and the aqueous layer was extracted once more with ethyl acetate. The combined organic layers were washed with sodium chloride solution and dried over sodium sulfate. After evaporation the residue was purified by silica gel chromatography (ethyl acetate/hexanes) to give 0.38 g (88%) of ((R)-1-hydroxymethyl-2,2-dimethyl-propyl)-carbamic acid tert-butyl ester.

### Step 2

((R)-1-hydroxymethyl-2,2-dimethyl-propyl)-carbamic acid tert-butyl ester (0.38 g, 1.74 mmol) was dissolved in 17 ml of acetonitrile and iodomethane (1.6 ml, 26.1 mmol), and then silver oxide (0.65 g, 2.78 mmol; prepared as in Org. Syn. Coll. Vol. VII, p. 386) were added. The reaction flask was covered to block light and the reaction was heated at reflux for 24 h. More iodomethane (6.4 ml) and silver oxide (0.65 g) were added in portions followed by further heating until the reaction was judged to be complete by standard reverse phase LC/MS. The reaction mixture was filtered through diatomaceous earth, rinsing with ethyl acetate. After evaporation, the residue was purified by silica gel chromatography (ethyl acetate/hexanes) to give 0.28 g (69%) of ((R)-1-methoxymethyl-2,2-dimethyl-propyl)-carbamic acid tert-butyl ester.

### Step 3

((R)-1-Methoxymethyl-2,2-dimethyl-propyl)-carbamic acid tert-butyl ester (0.28 g, 1.2 mmol) was dissolved in 6 ml of dichloromethane and then cooled in an ice bath. Added 4 ml of trifluoroacetic acid and the reaction was stirred to room temperature. The reaction solution was evaporated to afford (R)-1-methoxymethyl-2,2-dimethyl-propylamine trifluoroacetate which was used without further purification.

### Step 4

2-Cyclopropyl-5H-pyrrolo[2,3-b]pyrazine-7-carboxylic acid ((R)-1-methoxymethyl-2,2-dimethyl-propyl)-amide was prepared according to the procedure outlined in Example 1, steps 4-5 substituting (R)-1-methoxymethyl-2,2-dimethyl-propylamine trifluoroacetate for 1-((R)-1-amino-ethyl)-cyclopentanol hydrochloride. MS: (M+H)⁺=317; mp=265.0-270.0.

### Example 23

### 2-Cyclopropyl-5H-pyrrolo[2,3-b]pyrazine-7-carboxylic acid ((S)-1-methoxymethyl-2,2-dimethyl-propyl)-amide

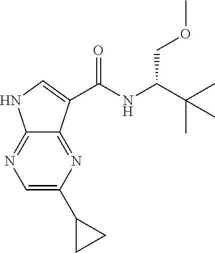 [SMILES: COC[C@@H](NC(=O)c1c[nH]c2ncc(C3CC3)nc12)C(C)(C)C]

Prepared according to the procedure outlined in Example 22 substituting S-tert-leucinol for R-tert-leucinol. MS: (M+H)⁺=317; mp=268.0-270.0.

### Example 24

### 2-Cyclopropyl-5H-pyrrolo[2,3-b]pyrazine-7-carboxylic acid (1-cyclohexyl-propyl)-amide

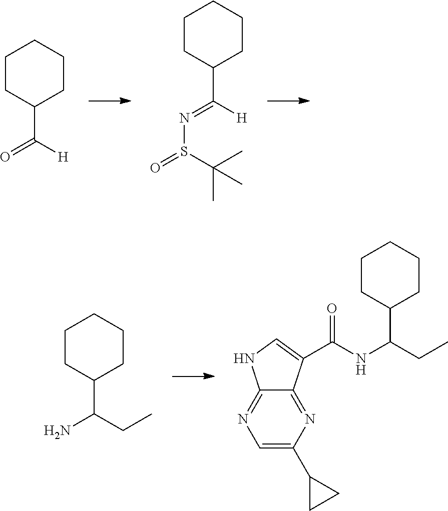

### Step 1

2-Methyl-2-propanesulfinamide (5.0 g, 41.2 mmol), cyclohexane carboxaldehyde (9.9 ml, 82.5 mmol), pyridinium p-toluenesulfonate (0.52 g, 2.06 mmol) and 25 g of magnesium sulfate were combined in a flask with 70 ml of dichloromethane. The reaction mixture was stirred for 16 h and then filtered through diatomaceous earth. After evaporation, the residue was purified by silica gel chromatography (diethyl ether/hexanes) to give 7.79 g (87%) of 2-methyl-propane-2-sulfinic acid 1-cyclohexyl-methylideneamide.

### Step 2

2-Methyl-propane-2-sulfinic acid 1-cyclohexyl-methylideneamide (0.5 g, 2.3 mmol) was dissolved in 12 ml of diethyl ether. The reaction solution was cooled to −40° C. and ethyl magnesium bromide (3M in ether, 1.5 ml, 4.5 mmol) was added dropwise, and the reaction was stirred to 25° C. Ammonium chloride solution and then ethyl acetate were added, the layers separated and the aqueous layer was extracted with ethyl acetate twice more. The combined organic layers were washed with sodium chloride solution, dried over sodium sulfate and concentrated to afford 0.45 g (85%) of 2-methyl-propane-2-sulfinic acid (1-cyclohexyl-propyl)-amide.

### Step 3

2-Methyl-propane-2-sulfinic acid (1-cyclohexyl-propyl)-amide (0.45 g, 1.95 mmol) was dissolved in 1 ml of methanol and 1 ml of 4M HCl in 1,4-dioxane was added. The reaction solution was stirred for 30 min. Diethyl ether was added to the solution and the reaction solvents were partially evaporated resulting in the formation of a precipitate. The solid was filtered, rinsed with hexanes and dried to give 200 mg (57%) of 1-cyclohexyl-propyl-amine hydrochloride.

### Step 4

2-Cyclopropyl-5H-pyrrolo[2,3-b]pyrazine-7-carboxylic acid (1-cyclohexyl-propyl)-amide was prepared according to the procedure outlined in Example 1, steps 4-5 substituting 1-cyclohexyl-propyl-amine hydrochloride for 1-((R)-1-amino-ethyl)-cyclopentanol hydrochloride. MS: (M+H)⁺=327; mp=208.0-210.0; elemental analysis: calculated C, 69.91; H, 8.03; N, 17.16. found C, 69.57; H, 7.96; N, 16.97.

### Example 25

### 2-Cyclopropyl-5H-pyrrolo[2,3-b]pyrazine-7-carboxylic acid (cyclohexyl-cyclopropyl-methyl)-amide

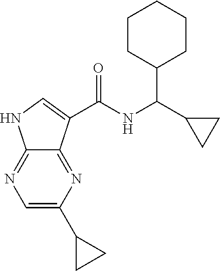 [SMILES: O=C(NC(C1CCCCC1)C1CC1)c1c[nH]c2ncc(C3CC3)nc12]

Prepared according to the procedure outlined in Example 24 substituting cyclopropyl magnesium bromide for ethyl magnesium bromide in step 2. MS: (M+H)⁺=339; mp=174.0-176.0; elemental analysis: calculated C, 70.98; H, 7.74; N, 16.55. found C, 70.68; H, 7.54; N, 16.46.

### Example 26

### 2-Cyclopropyl-5H-pyrrolo[2,3-b]pyrazine-7-carboxylic acid ((S)-1-cyanomethyl-2,2-dimethyl-propyl)-amide

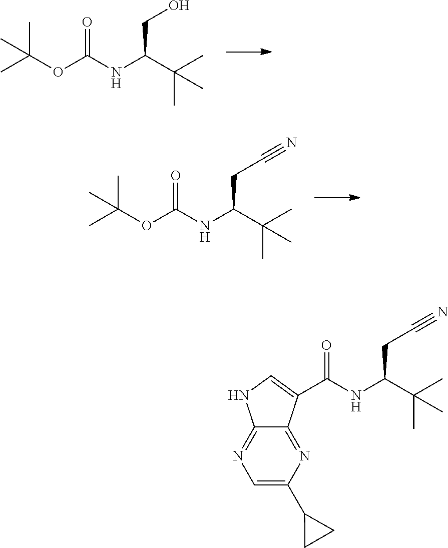 [SMILES: CC(C)(C)OC(=O)N[C@@H](CO)C(C)(C)C ; CC(C)(C)OC(=O)N[C@@H](CC#N)C(C)(C)C ; CC(C)(C)[C@H](CC#N)NC(=O)c1c[nH]c2ncc(C3CC3)nc12]

### Step 1

((R)-1-hydroxymethyl-2,2-dimethyl-propyl)-carbamic acid tert-butyl ester (Example 22, step 1; 0.157 g, 7.2 mmol) was dissolved in 2 ml of tetrahydrofuran. Triethylamine (0.13 ml, 0.935 mmol) was added and the reaction cooled in an ice bath. Methanesulfonyl chloride (0.073 ml, 0.935 mmol) was added slowly and the reaction was stirred to 25° C. over 16 h. Dichloromethane and water were added and the layers were separated. The aqueous layer was extracted once more with dichloromethane, and the combined organic layers were then washed with sodium chloride solution and dried over sodium sulfate. After evaporation, 0.21 g (83%) of methanesulfonic acid (R)-2-tert-butoxycarbonylamino-3,3-dimethyl-butyl ester was obtained.

### Step 2

Methanesulfonic acid (R)-2-tert-butoxycarbonylamino-3,3-dimethyl-butyl ester (0.21 g, 0.71 mmol) was dissolved in 2 ml of N,N-dimethylformamide. Crushed sodium cyanide (104 mg, 2.13 mmol) was added and the mixture was stirred for 4 days at 35° C. Water and ethyl acetate were added and the layers separated. The aqueous layer was extracted twice more with ethyl acetate, the combined organic layers were washed with water and sodium chloride solution, and then dried over sodium sulfate. After evaporation the residue was purified by silica gel chromatography (ethyl acetate/hexanes) to give 0.1 g (62%) of ((S)-1-cyanomethyl-2,2-dimethyl-propyl)-carbamic acid tert-butyl ester.

### Step 3

((S)-1-cyanomethyl-2,2-dimethyl-propyl)-carbamic acid tert-butyl ester (0.1 g, 0.44 mmol) was cooled in an ice bath and cold 4M HCl in 1,4-dioxane was added to dissolve the ester. After 1 h the reaction solution was carefully evaporated to afford (S)-3-amino-4,4-dimethyl-pentanenitrile hydrochloride which was used without further purification.

### Step 4

2-Cyclopropyl-5H-pyrrolo[2,3-b]pyrazine-7-carboxylic acid ((S)-1-cyanomethyl-2,2-dimethyl-propyl)-amide was prepared according to the procedure outlined in Example 1, steps 4-5 substituting (S)-3-amino-4,4-dimethyl-pentanenitrile hydrochloride for 1-((R)-1-amino-ethyl)-cyclopentanol hydrochloride. MS: (M+H)⁺=312; mp=258.0-260.0.

### Example 27

### 2-Cyclopropyl-5H-pyrrolo[2,3-b]pyrazine-7-carboxylic acid ((R)-1-cyanomethyl-2,2-dimethyl-propyl)-amide

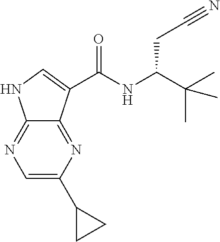 [SMILES: CC(C)(C)[C@@H](CC#N)NC(=O)c1c[nH]c2ncc(C3CC3)nc12]

Prepared according to the procedure outlined in Example 26 substituting ((S)-1-hydroxymethyl-2,2-dimethyl-propyl)-carbamic acid tert-butyl ester for ((R)-1-hydroxymethyl-2,2-dimethyl-propyl)-carbamic acid tert-butyl ester. (M+H)⁺=312; mp=259.0-261.0.

### Example 28

### 2-Cyclopropyl-5H-pyrrolo[2,3b]pyrazine-7-carboxylic acid (2-hydroxy-2-methyl-1-trifluoromethyl-propyl)-amide

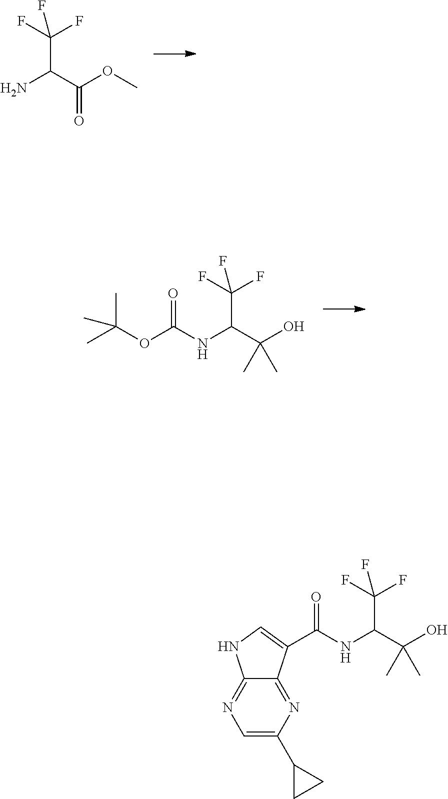

### Step 1

Methyl 3,3,3-trifluoroalaninate hydrochloride (1.0 g, 5.16 mmol) was dissolved in 26 ml of dichloromethane. Triethylamine (0.72 ml, 5.16 mmol) was added and the reaction was cooled in an ice bath. Di-tert-butyldicarbonate (2.2 g, 10.3 mmol) was added slowly and the reaction was stirred for 18 h. Ethyl acetate and ammonium chloride solution were added, the layers were separated and the aqueous layer was extracted once more with ethyl acetate. The combined organic layers were washed with sodium chloride solution and dried over sodium sulfate. After evaporation the residue was purified by silica gel chromatography to give 2-tert-butoxycarbonylamino-3,3,3-trifluoro-propionic acid methyl ester.

### Step 2

2-tert-Butoxycarbonylamino-3,3,3-trifluoro-propionic acid methyl ester (0.16 g, 0.55 mmol) was dissolved in 5 ml of tetrahydrofuran and then cooled in an ice bath. Methyl magnesium chloride (3.0 M in ether, 0.73 ml, 2.18 mmol) was added dropwise to the solution and then stirred for 16 h. Ammonium chloride solution and ethyl acetate were added to the reaction and the layers were separated. The aqueous layer was extracted once more with ethyl acetate and the combined organic layers were washed with sodium chloride solution. Drying over sodium sulfate and evaporation provided 0.11 g of (2-hydroxy-2-methyl-1-trifluoromethyl-propyl)-carbamic acid tert-butyl ester.

### Step 3

(2-Hydroxy-2-methyl-1-trifluoromethyl-propyl)-carbamic acid tert-butyl ester (0.11 g, 0.43 mmol) was cooled in an ice bath and cold 4M HCl in 1,4-dioxane was added to dissolve the ester. After 1 h the reaction solution was carefully evaporated to afford 3-amino-4,4,4-trifluoro-2-methyl-butan-2-ol hydrochloride which was used without further purification.

### Step 4

2-Cyclopropyl-5H-pyrrolo[2,3b]pyrazine-7-carboxylic acid (2-hydroxy-2-methyl-1-trifluoromethyl-propyl)-amide was prepared according to the procedure outlined in Example 1, steps 4-5 substituting 3-amino-4,4,4-trifluoro-2-methyl-butan-2-ol hydrochloride for 1-((R)-1-amino-ethyl)-cyclopentanol hydrochloride. MS: (M+H)⁺=343; mp=258.0-260.0.

### Example 29

### 2-Cyclopropyl-5H-pyrrolo[2,3-b]pyrazine-7-carboxylic acid ((S)-1-cyclohexyl-2-hydroxy-2-methyl-propyl)-amide

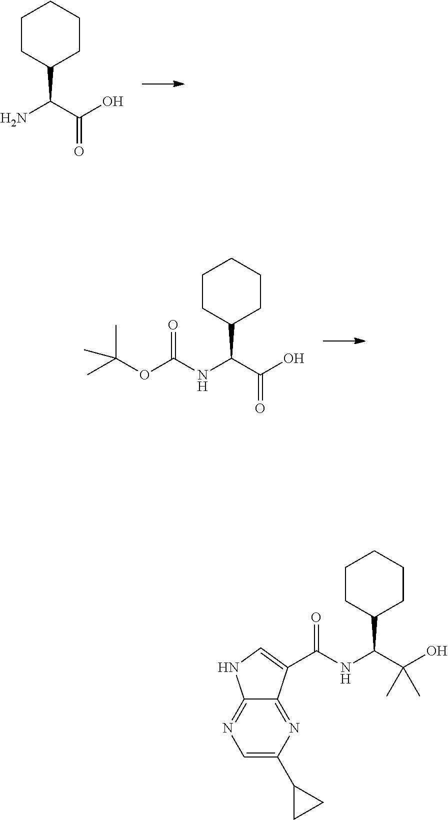 [SMILES: N[C@H](C(=O)O)C1CCCCC1 ; CC(C)(C)OC(=O)N[C@H](C(=O)O)C1CCCCC1 ; CC(C)(O)[C@@H](NC(=O)c1c[nH]c2ncc(C3CC3)nc12)C1CCCCC1]

### Step 1

(S)-Amino-cyclohexyl-acetic acid, hydrochloride salt (1.0 g, 5.16 mmol) was dissolved in 17 ml of 2:1 1,4-dioxane:water and cooled in an ice bath. Sodium hydroxide solution (10.4 ml of 1M aqueous solution) was added slowly to the reaction solution followed by solid sodium bicarbonate (0.43 g, 5.16 mmol). Di-tert-butyldicarbonate (1.68 g, 7.74 mmol) was added and the reaction mixture was stirred for 16 h. The reaction mixture was partially evaporated, then taken up in ethyl acetate and water, and acidified to pH 2 with potassium bisulfate solution. The layers were separated and the aqueous layer was extracted with ethyl acetate twice more. The combined ethyl acetate layers were washed with sodium chloride solution, dried over sodium sulfate and evaporated to 1.52 g of crude (S)-tert-butoxycarbonylamino-cyclohexyl-acetic acid.

### Step 2

(S)-tert-Butoxycarbonylamino-cyclohexyl-acetic acid (1.52 g, 5.16 mmol) was dissolved in 39 ml of toluene and 11 ml of methanol. Trimethylsilyldiazomethane (2.0 M in hexane, 12.9 ml, 25.8 mmol) was added slowly and the reaction mixture was stirred for 16 h. The reaction was evaporated to a solid and purified by silica gel chromatography (ethyl acetate/hexanes) to give 1.26 g (79%) of (S)-tert-butoxycarbonylamino-cyclohexyl-acetic acid methyl ester.

### Step 3

2-Cyclopropyl-5H-pyrrolo[2,3-b]pyrazine-7-carboxylic acid ((S)-1-cyclohexyl-2-hydroxy-2-methyl-propyl)-amide was prepared according to the procedure outlined in Example 28, steps 2-4 substituting (S)-tert-butoxycarbonylamino-cyclohexyl-acetic acid methyl ester for 2-tert-butoxycarbonylamino-3,3,3-trifluoro-propionic acid methyl ester. MS: (M+H)⁺=357; mp=251.0-253.0.

### Example 30

### 2-Cyclopropyl-5H-pyrrolo[2,3-b]pyrazine-7-carboxylic acid ((R)-2-cyano-cyclopropyl-ethyl)-amide

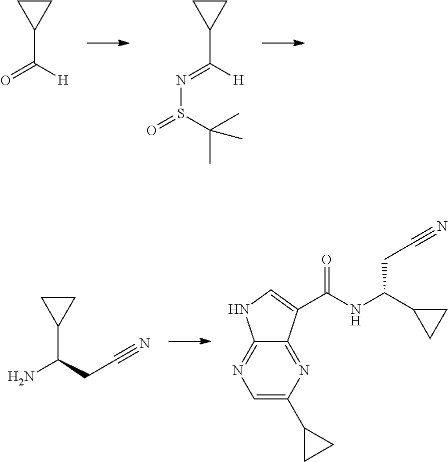 [SMILES: O=CC1CC1 ; CC(C)(C)S(=O)/N=C/C1CC1 ; N#CC[C@@H](N)C1CC1 ; N#CC[C@@H](NC(=O)c1c[nH]c2ncc(C3CC3)nc12)C1CC1]

### Step 1

(R)-2-Methyl-propane-2-sulfinic acid 1-cyclopropyl-methylideneamide was prepared as in Example 24, step 1 substituting (R)-2-methylpropane-2-sulfinamide for 2-methyl-2-propanesulfinamide and cyclopropanecarbaldehyde for cyclohexane carboxaldehyde.

### Step 2

(R)-2-Methyl-propane-2-sulfinic acid 1-cyclopropyl-methylideneamide (0.3 g, 1.73 mmol) was dissolved in 17 ml of tetrahydrofuran. Tetrabutylammonium phenolate (0.58 g, 1.73 mmol, prepared as in Bull. Chem. Soc. Jpn. 2003, 76(11), 2191) was added and the reaction solution was cooled in a dry ice/acetone bath. Trimethylsilylacetonitrile (0.356 ml, 2.6 mmol) was added dropwise and the reaction was stirred in the bath for 2 h. Ammonium chloride solution was added to the reaction solution at approximately 0° C. Ethyl acetate and more water were added, the layers were separated, and the aqueous layer was extracted twice more with ethyl acetate. The combined organic layers were washed with brine and dried over sodium sulfate. After evaporation, the residue was purified by silica gel chromatography (ethyl acetate/hexanes) to give 0.14 g (38%) of (R)—N—((R)-2-cyano-1-cyclopropylethyl)-2-methylpropane-2-sulfinamide.

### Step 3

2-Cyclopropyl-5H-pyrrolo[2,3-b]pyrazine-7-carboxylic acid ((R)-2-cyano-cyclopropyl-ethyl)-amide was prepared as in Example 24, steps 3-4 substituting (R)—N—((R)-2-cyano-1-cyclopropylethyl)-2-methylpropane-2-sulfinamide for 2-methyl-propane-2-sulfinic acid (1-cyclohexyl-propyl)-amide. MS: (M+H)⁺=296; [α]_{D}=−23.

### Example 31

### 2-Cyclopropyl-5H-pyrrolo[2,3-b]pyrazine-7-carboxylic acid ((S)-2-cyano-1-cyclopropyl-ethyl)-amide

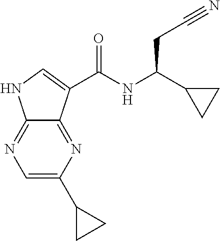 [SMILES: N#CC[C@H](NC(=O)c1c[nH]c2ncc(C3CC3)nc12)C1CC1]

Prepared according to the procedure outlined in Example 30 substituting (S)-(−)-t-butylsulfinamide for (R)-2-methylpropane-2-sulfinamide. MS: (M+H)⁺=296; [α]_{D}=23.7; mp=230.0-232.0.

### Example 32

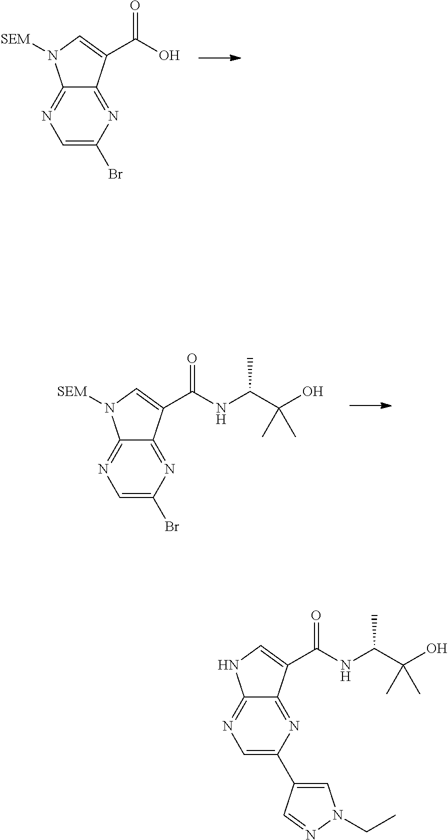 [SMILES: C[Si](C)(C)CCOCn1cc(C(=O)O)c2nc(Br)cnc21 ; C[C@@H](NC(=O)c1cn(COCC[Si](C)(C)C)c2ncc(Br)nc12)C(C)(C)O ; CCn1cc(-c2cnc3[nH]cc(C(=O)N[C@H](C)C(C)(C)O)c3n2)cn1]

### Step 1

To a solution of Boc-D-alanine methyl ester (5.00 g, 24.6 mmol) in THF (100 mL) at 0° C. was slowly added methyl magnesium bromide (3.0 M in Et₂O, 28.7 mL, 86.1 mmol). The resultant white slurry was stirred at 0° C. for 1 h then at room temperature for 2 h. The reaction mixture was quenched with saturated aqueous NH₄Cl, diluted with H₂O and extracted with EtOAc. The combined organics were washed with brine, dried over MgSO₄ and concentrated to give 4.93 g (99%) ((R)-2-hydroxy-1,2-dimethyl-propyl)-carbamic acid tert-butyl ester as a colorless viscous oil.

### Step 2

((R)-2-Hydroxy-1,2-dimethyl-propyl)-carbamic acid tert-butyl ester (4.93 g, 24.2 mmol) was dissolved in 1.0 M HCl (150 mL) and stirred at 50° C. for 4 h. Concentration gave 4.01 g (R)-3-amino-2-methyl-butan-2-ol hydrochloride as a pale brown solid which was used without further purification.

### Step 3

In a flask were combined 2-bromo-5-(2-trimethylsilanylethoxymethyl)-5H-pyrrolo[2,3-b]pyrazine-7-carboxylic acid (3.25 g, 8.74 mmol), (R)-3-amino-2-methyl-butan-2-ol hydrochloride (3.05 g, 21.9 mmol), EDC (3.85 g, 20.1 mmol) and HOBt (2.72 g, 20.1 mmol). Then added DMF (50 mL) followed by i-Pr₂NEt (4.87 mL, 28.0 mmol). The mixture was stirred at room temperature overnight then concentrated under reduced pressure. The residue purified by SiO₂ chromatography (20-100% EtOAc/hexane) to afford 2.40 g (60%) 2-bromo-5-(2-trimethylsilanyl-ethoxymethyl)-5H-pyrrolo[2,3-b]pyrazine-7-carboxylic acid ((R)-2-hydroxy-1,2-dimethyl-propyl)-amide as a yellow solid.

### Step 4

In a pressure tube, 2-bromo-5-(2-trimethylsilanyl-ethoxymethyl)-5H-pyrrolo[2,3-b]pyrazine-7-carboxylic acid ((R)-2-hydroxy-1,2-dimethyl-propyl)-amide (120 mg, 0.26 mmol) and 1-ethyl-1H-pyrazole-4-boronic acid pinacol ester (70 mg, 0.32 mmol) were dissolved in DME (2.0 mL). Aqueous K₂CO₃ (2.0 M, 0.39 mL, 0.78 mmol) and Pd(PPh₃)₄ (15 mg, 0.013 mmol) were added and the mixture was degassed with a gentle stream of N₂ for 15 min. The tube was then sealed and heated at 90° C. for 3 h. The reaction mixture was cooled to room temperature, quenched with H₂O and extracted with EtOAc. The organic extracts were washed with brine, dried over MgSO₄, and concentrated. The residue was purified by SiO₂ chromatography (20-100% EtOAc/hexane) to afford 111 mg (90%) 2-(1-ethyl-1H-pyrazol-4-yl)-5-(2-trimethylsilanyl-ethoxymethyl)-5H-pyrrolo[2,3-b]pyrazine-7-carboxylic acid ((R)-2-hydroxy-1,2-dimethyl-propyl)amide as a pale yellow foam.

### Step 5

To a solution of 2-(1-ethyl-1H-pyrazol-4-yl)-5-(2-trimethylsilanyl-ethoxymethyl)-5H-pyrrolo[2,3-b]pyrazine-7-carboxylic acid ((R)-2-hydroxy-1,2-dimethyl-propyl)-amide in CH₂Cl₂ (2.25 mL) was added TFA (0.75 mL). The reaction mixture was stirred for 2.5 h and concentrated. The residue was dissolved in CH₂Cl₂ (3.75 mL), ethylene diamine (0.75 mL, 11.2 mmol) added and the mixture stirred at room temperature overnight. The reaction mixture was concentrated and the residue was purified by SiO₂ chromatography (0-10% MeOH/CH₂Cl₂) to afford 59 mg (74%) 2-(1-ethyl-1H-pyrazol-4-yl)-5H-pyrrolo[2,3-b]pyrazine-7-carboxylic acid ((R)-2-hydroxy-1,2-dimethyl-propyl)-amide as a pale yellow powder. MS: 343 (M+H)⁺; mp=270.0-272.0.

### Example 33

### 2-(1-Methyl-1H-pyrazol-4-yl)-5H-pyrrolo[2,3-b]pyrazine-7-carboxylic acid ((R)-2-hydroxy-1,2-dimethyl-propyl)-amide

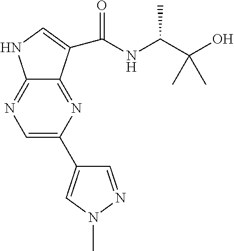 [SMILES: C[C@@H](NC(=O)c1c[nH]c2ncc(-c3cnn(C)c3)nc12)C(C)(C)O]

Prepared according to the procedure outlined in Example 32, steps 4-5 substituting 1-methyl-1H-pyrazole-4-boronic acid pinacol ester for 1-ethyl-1H-pyrazole-4-boronic acid pinacol ester. MS: (M+H)⁺=329; mp 285.0-288.0.

### Example 34

### 2-Thiophen-2-yl-5H-pyrrolo[2,3-b]pyrazine-7-carboxylic acid ((R)-2-hydroxy-1,2-dimethyl-propyl)-amide

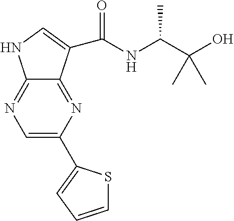 [SMILES: C[C@@H](NC(=O)c1c[nH]c2ncc(-c3cccs3)nc12)C(C)(C)O]

Prepared according to the procedure outlined in Example 32, steps 4-5 substituting thiophene-2-boronic acid pinacol ester for 1-ethyl-1H-pyrazole-4-boronic acid pinacol ester. MS: (M+H)⁺=331; mp 272.0-275.0.

### Example 35

### 2-(3,6-Dihydro-2H-pyran-4-yl)-5H-pyrrolo[2,3-b]pyrazine-7-carboxylic acid ((R)-2-hydroxy-1,2-dimethyl-propyl)-amide

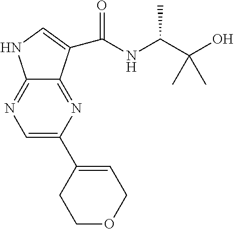 [SMILES: C[C@@H](NC(=O)c1c[nH]c2ncc(C3=CCOCC3)nc12)C(C)(C)O]

Prepared according to the procedure outlined in Example 32, steps 4-5 substituting 3,6-dihydro-2H-pyran-4-ylboronic acid pinacol ester for 1-ethyl-1H-pyrazole-4-boronic acid pinacol ester. MS: (M+H)⁺=331.

### Example 36

### 2-Thiazol-2-yl-5H-pyrrolo[2,3-b]pyrazine-7-carboxylic acid ((R)-2-hydroxy-1,2-dimethyl-propyl)-amide

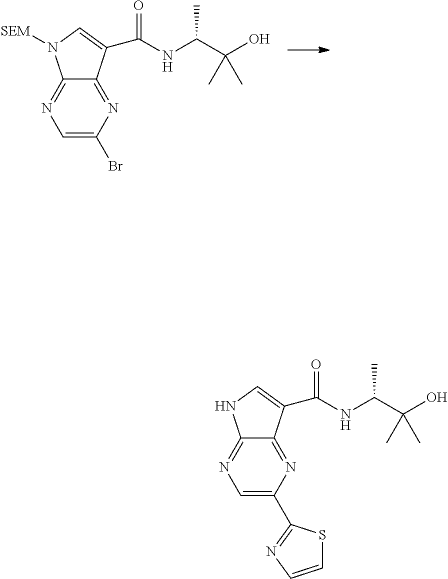 [SMILES: C[C@@H](NC(=O)c1cn(COCC[Si](C)(C)C)c2ncc(Br)nc12)C(C)(C)O ; C[C@@H](NC(=O)c1c[nH]c2ncc(-c3nccs3)nc12)C(C)(C)O]

### Step 1

In a pressure tube 2-bromo-5-(2-trimethylsilanyl-ethoxymethyl)-5H-pyrrolo[2,3b]pyrazine-7-carboxylic acid ((R)-2-hydroxy-1,2-dimethyl-propyl)-amide (120 mg, 0.26 mmol) and 2-tributylstannylthiazole (0.10 mL, 0.32 mmol) were dissolved in DMF (2.0 mL). Pd(PPh₃)₄ (15.2 mg, 0.013 mmol) and copper (I) iodide (10.0 mg, 0.052 mmol) were added and the tube was sealed and heated at 80° C. for 1.5 h. The reaction mixture was cooled and concentrated. The residue was purified by SiO₂ chromatography (0-10% MeOH/CH₂Cl₂) to give 125 mg of 2-thiazol-2-yl-5-(2-trimethylsilanyl-ethoxymethyl)-5H-pyrrolo[2,3-b]pyrazine-7-carboxylic acid ((R)-2-hydroxy-1,2-dimethyl-propyl)-amide as a brown viscous oil.

### Step 2

2-Thiazol-2-yl-5H-pyrrolo[2,3-b]pyrazine-7-carboxylic acid ((R)-2-hydroxy-1,2-dimethyl-propyl)-amide. Prepared according to the procedure outlined in Example 32, step 5 substituting 2-thiazol-2-yl-5-(2-trimethylsilanyl-ethoxymethyl)-5H-pyrrolo[2,3-b]pyrazine-7-carboxylic acid ((R)-2-hydroxy-1,2-dimethyl-propyl)-amide for 2-(1-ethyl-1H-pyrazol-4-yl)-5-(2-trimethylsilanyl-ethoxymethyl)-5H-pyrrolo[2,3-b]pyrazine-7-carboxylic acid ((R)-2-hydroxy-1,2-dimethyl-propyl)-amide. MS: (M+H)⁺=332.

### Example 37

### 2-Pyridin-2-yl-5H-pyrrolo[2,3-b]pyrazine-7-carboxylic acid ((R)-2-hydroxy-1,2-dimethyl-propyl)-amide

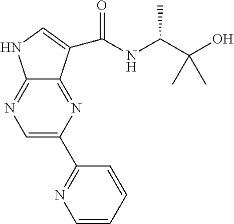 [SMILES: C[C@@H](NC(=O)c1c[nH]c2ncc(-c3ccccn3)nc12)C(C)(C)O]

Prepared according to the procedure outlined in Example 36 substituting 2-(tributylstannyl)pyridine for 2-tributylstannylthiazole. MS: (M+H)⁺=326.

### Example 38

### 2-Cyano-5H-pyrrolo[2,3-b]pyrazine-7-carboxylic acid ((R)-2-hydroxy-1,2-dimethyl-propyl)-amide

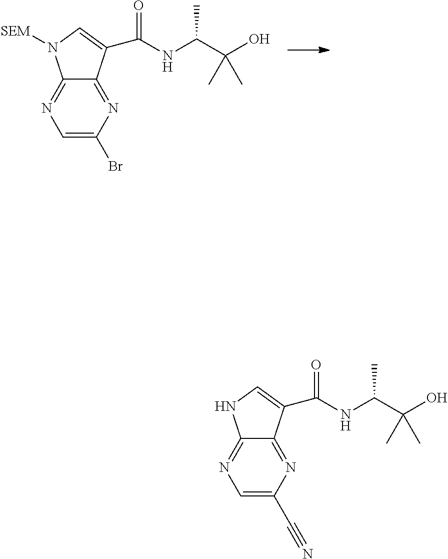 [SMILES: C[C@@H](NC(=O)c1cn(COCC[Si](C)(C)C)c2ncc(Br)nc12)C(C)(C)O ; C[C@@H](NC(=O)c1c[nH]c2ncc(C#N)nc12)C(C)(C)O]

### Step 1

In a microwave tube 2-bromo-5-(2-trimethylsilanyl-ethoxymethyl)-5H-pyrrolo[2,3b]pyrazine-7-carboxylic acid ((R)-2-hydroxy-1,2-dimethyl-propyl)-amide (250 mg, 0.55 mmol), zinc cyanide (97 mg, 0.82 mmol) and Pd(PPh₃)₄ (191 mg, 0.165 mmol) were combined in DMF (5.0 mL) and heated at 140° C. for 15 min. The reaction mixture was evaporated and directly purified by SiO₂ chromatography (20-100% EtOAc/Heptane) to give 186 mg (84%) of 2-cyano-5-(2-trimethylsilanyl-ethoxymethyl)-5H-pyrrolo[2,3-b]pyrazine-7-carboxylic acid ((R)-2-hydroxy-1,2-dimethyl-propyl)-amide as a yellow paste.

### Step 2

2-Cyano-5H-pyrrolo[2,3-b]pyrazine-7-carboxylic acid ((R)-2-hydroxy-1,2-dimethyl-propyl)-amide. Prepared according to the procedure outlined in Example 32, step 5 substituting 2-cyano-5-(2-trimethylsilanyl-ethoxymethyl)-5H-pyrrolo[2,3-b]pyrazine-7-carboxylic acid ((R)-2-hydroxy-1,2-dimethyl-propyl)-amide for 2-(1-ethyl-1H-pyrazol-4-yl)-5-(2-trimethylsilanyl-ethoxymethyl)-5H-pyrrolo[2,3-b]pyrazine-7-carboxylic acid ((R)-2-hydroxy-1,2-dimethyl-propyl)-amide. MS: (M+H)⁺=274.

### Example 39

### 2-Cyclopent-1-enyl-5H-pyrrolo[2,3-b]pyrazine-7-carboxylic acid ((S)-2-hydroxy-1,2-dimethyl-propyl)-amide

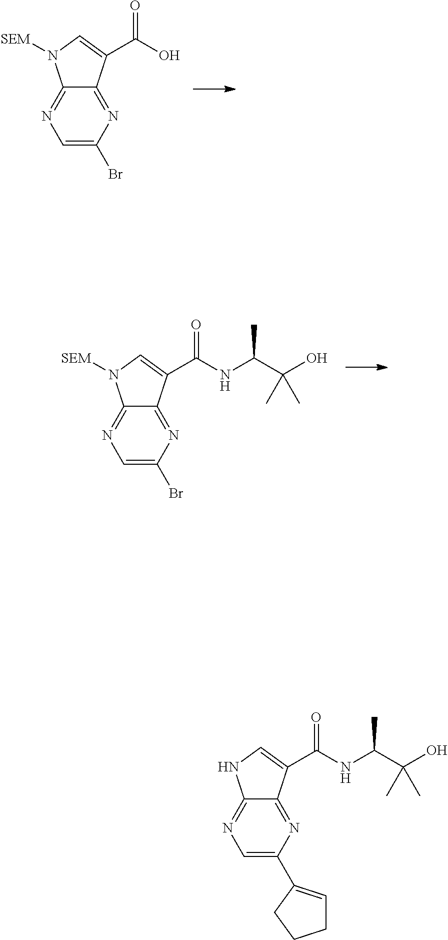 [SMILES: C[Si](C)(C)CCOCn1cc(C(=O)O)c2nc(Br)cnc21 ; C[C@H](NC(=O)c1cn(COCC[Si](C)(C)C)c2ncc(Br)nc12)C(C)(C)O ; C[C@H](NC(=O)c1c[nH]c2ncc(C3=CCCC3)nc12)C(C)(C)O]

Prepared according to the procedure outlined in Example 32, steps 3-5 substituting (S)-3-amino-2-methyl-butan-2-ol hydrochloride (Tetrahedron: Asymmetry 1995, 6, 671) for (R)-3-amino-2-methyl-butan-2-ol hydrochloride in step 3 and substituting cyclopenten-1-ylboronic acid for 1-ethyl-1H-pyrazole-4-boronic acid pinacol ester in step 4. MS: (M+H)⁺=315.

### Example 40

### 2-Cyclopentyl-5H-pyrrolo[2,3-b]pyrazine-7-carboxylic acid ((S)-2-hydroxy-1,2-dimethyl-propyl)-amide

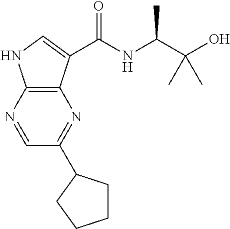 [SMILES: C[C@H](NC(=O)c1c[nH]c2ncc(C3CCCC3)nc12)C(C)(C)O]

Prepared from 2-cyclopent-1-enyl-5H-pyrrolo[2,3-b]pyrazine-7-carboxylic acid ((S)-2-hydroxy-1,2-dimethyl-propyl)-amide (Example 39) by treatment with 10% palladium on carbon under a hydrogen atmosphere of 40 psi for 24 hours. The reaction mixture was filtered through celite and a Whatman syringe filter and the product was purified by trituration with ethyl acetate. MS: (M+H)⁺=317.

### Example 41

### 2-Isopropenyl-5H-pyrrolo[2,3-b]pyrazine-7-carboxylic acid ((S)-2-hydroxy-1,2-dimethyl-propyl)-amide

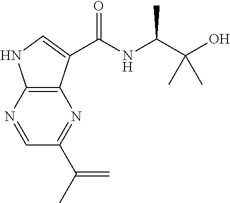 [SMILES: C=C(C)c1cnc2[nH]cc(C(=O)N[C@@H](C)C(C)(C)O)c2n1]

Prepared according to the procedure outlined in Example 32, steps 3-5 substituting (S)-3-amino-2-methyl-butan-2-ol hydrochloride (Tetrahedron: Asymmetry 1995, 6, 671) for (R)-3-amino-2-methyl-butan-2-ol hydrochloride in step 3 and substituting 2-isopropenyl-4,4,5,5-tetramethyl-[1,3,2]dioxaborolane for 1-ethyl-1H-pyrazole-4-boronic acid pinacol ester in step 4. MS: (M+H)⁺=289.

### Example 42

### 2-Isopropyl-5H-pyrrolo[2,3-b]pyrazine-7-carboxylic acid ((S)-2-hydroxy-1,2-dimethyl-propyl)-amide

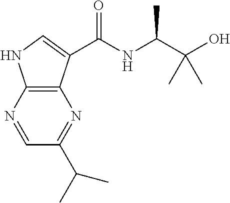 [SMILES: CC(C)c1cnc2[nH]cc(C(=O)N[C@@H](C)C(C)(C)O)c2n1]

Prepared from 2-isopropenyl-5H-pyrrolo[2,3-b]pyrazine-7-carboxylic acid ((S)-2-hydroxy-1,2-dimethyl-propyl)-amide (Example 41) by treatment with 10% palladium on carbon under a hydrogen atmosphere of 40 psi overnight. The reaction mixture was filtered through celite and a Whatman syringe filter and the product was purified by crystallization from ethyl acetate. MS: (M+H)⁺=291.

### Example 43

### 2-Cyclohex-1-enyl-5H-pyrrolo[2,3-b]pyrazine-7-carboxylic acid ((S)-2-hydroxy-1,2-dimethyl-propyl)-amide

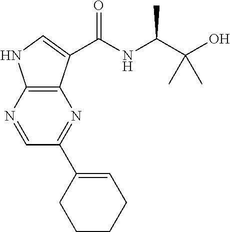 [SMILES: C[C@H](NC(=O)c1c[nH]c2ncc(C3=CCCCC3)nc12)C(C)(C)O]

Prepared according to the procedure outlined in Example 32, steps 3-5 substituting (S)-3-amino-2-methyl-butan-2-ol hydrochloride (Tetrahedron: Asymmetry 1995, 6, 671) for (R)-3-amino-2-methyl-butan-2-ol hydrochloride in step 3 and substituting cyclohexen-1-ylboronic acid for 1-ethyl-1H-pyrazole-4-boronic acid pinacol ester in step 4. MS: (M+H)⁺=329

### Example 44

### 2-Cyclohexyl-5H-pyrrolo[2,3-b]pyrazine-7-carboxylic acid ((S)-2-hydroxy-1,2-dimethyl-propyl)-amide

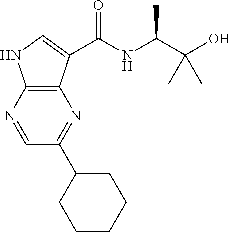 [SMILES: C[C@H](NC(=O)c1c[nH]c2ncc(C3CCCCC3)nc12)C(C)(C)O]

Prepared from 2-cyclohex-1-enyl-5H-pyrrolo[2,3-b]pyrazine-7-carboxylic acid ((S)-2-hydroxy-1,2-dimethyl-propyl)-amide by treatment with 10% palladium on carbon under a hydrogen atmosphere of 50 psi for 48 hours. The reaction mixture was filtered through celite and a Whatman syringe filter and the product was purified by crystallization from ethyl acetate. MS: (M+H)⁺=331.

### Example 45

### 2-Thiophen-2-yl-5H-pyrrolo[2,3-b]pyrazine-7-carboxylic acid ((S)-2-hydroxy-1,2-dimethyl-propyl)-amide

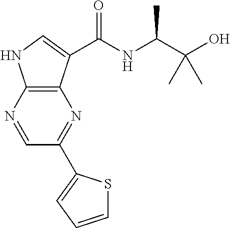 [SMILES: C[C@H](NC(=O)c1c[nH]c2ncc(-c3cccs3)nc12)C(C)(C)O]

Prepared according to the procedure outlined in Example 32, steps 3-5 substituting (S)-3-amino-2-methyl-butan-2-ol hydrochloride (Tetrahedron: Asymmetry 1995, 6, 671) for (R)-3-amino-2-methyl-butan-2-ol hydrochloride in step 3 and substituting 4,4,5,5-tetramethyl-2-thiophen-2-yl-[1,3,2]dioxaborolane for 1-ethyl-1H-pyrazole-4-boronic acid pinacol ester in step 4. The catalyst used in step 4 was Pd(dppf)Cl₂ and the solvent was toluene. MS: (M+H)⁺=331.

### Example 46

### 2-(2-Methyl-pyridin-4-yl)-5H-pyrrolo[2,3-b]pyrazine-7-carboxylic acid ((S)-2-hydroxy-1,2-dimethyl-propyl)-amide hydrochloride

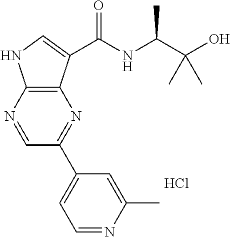 [SMILES: Cc1cc(-c2cnc3[nH]cc(C(=O)N[C@@H](C)C(C)(C)O)c3n2)ccn1 ; Cl]

Prepared according to the procedure outlined in Example 32, steps 3-5 substituting (S)-3-amino-2-methyl-butan-2-ol hydrochloride (Tetrahedron: Asymmetry 1995, 6, 671) for (R)-3-amino-2-methyl-butan-2-ol hydrochloride in step 3 and substituting 2-methyl-4-(4,4,5,5-tetramethyl-[1,3,2]dioxaborolan-2-yl)-pyridine for 1-ethyl-1H-pyrazole-4-boronic acid pinacol ester in step 4. The catalyst used in step 4 was Pd₂(dba)₃ and the solvent was toluene. The hydrochloride salt was prepared by dissolving the free base in boiling dioxane and treating it with 4M HCl in dioxane. MS: (M+H)⁺=340.

### Example 47

### 2-(6-Methyl-pyridin-3-yl)-5H-pyrrolo[2,3-b]pyrazine-7-carboxylic acid ((S)-2-hydroxy-1,2-dimethyl-propyl)-amide hydrochloride

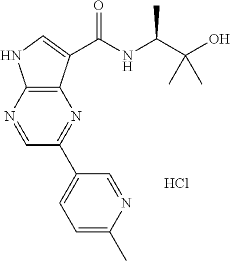 [SMILES: Cc1ccc(-c2cnc3[nH]cc(C(=O)N[C@@H](C)C(C)(C)O)c3n2)cn1 ; Cl]

Prepared according to the procedure outlined in Example 32, steps 3-5 substituting (S)-3-amino-2-methyl-butan-2-ol hydrochloride (Tetrahedron: Asymmetry 1995, 6, 671) for (R)-3-amino-2-methyl-butan-2-ol hydrochloride in step 3 and substituting 2-methyl-5-(4,4,5,5-tetramethyl-[1,3,2]dioxaborolan-2-yl)-pyridine for 1-ethyl-1H-pyrazole-4-boronic acid pinacol ester in step 4. The catalyst used in step 4 was Pd₂(dba)₃ and the solvent was toluene. The hydrochloride salt was prepared by dissolving the free base in boiling dioxane and treating it with 4M HCl in dioxane. MS: (M+H)⁺=340.

### Example 48

### 2-Vinyl-5H-pyrrolo[2,3-b]pyrazine-7-carboxylic acid (3-hydroxy-2,2-dimethyl-propyl)-amide

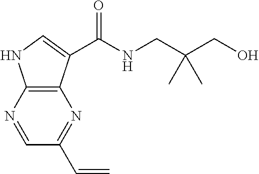 [SMILES: C=Cc1cnc2[nH]cc(C(=O)NCC(C)(C)CO)c2n1]

### Step 1

2-Bromo-5-(2-trimethylsilanyl-ethoxymethyl)-5H-pyrrolo[2,3-b]pyrazine-7-carboxylic acid (3-hydroxy-2,2-dimethyl-propyl)-amide was prepared according to the procedure outlined in Example 32, step 3 substituting 3-amino-2,2-dimethyl-propan-1-ol for (R)-3-amino-2-methyl-butan-2-ol hydrochloride.

### Step 2

In a pressure tube were combined 2-bromo-5-(2-trimethylsilanyl-ethoxymethyl)-5H-pyrrolo[2,3-b]pyrazine-7-carboxylic acid (3-hydroxy-2,2-dimethyl-propyl)-amide (250 mg, 0.55 mmol), potassium vinyltrifluoroborate (110 mg, 0.83 mmol), cesium carbonate (627 mg, 1.90 mmol), Pd(dppf)Cl₂ (22 mg, 0.03 mmol), THF (1.8 mL), and water (0.2 mL). The tube was purged with argon, sealed and heated at 85° C. overnight. The solvents were evaporated and the crude residue was purified by SiO₂ chromatography eluting with 25% to 50% EtOAc/hexanes to afford 157 mg (71%) of 5-(2-trimethylsilanyl-ethoxymethyl)-2-vinyl-5H-pyrrolo[2,3-b]pyrazine-7-carboxylic acid (3-hydroxy-2,2-dimethyl-propyl)-amide.

### Step 3

2-Vinyl-5H-pyrrolo[2,3-b]pyrazine-7-carboxylic acid (3-hydroxy-2,2-dimethyl-propyl)-amide was prepared according to the procedure outlined in Example 32, step 5 substituting 542-trimethylsilanyl-ethoxymethyl)-2-vinyl-5H-pyrrolo[2,3-b]pyrazine-7-carboxylic acid (3-hydroxy-2,2-dimethyl-propyl)-amide for 2-(1-ethyl-1H-pyrazol-4-yl)-5-(2-trimethylsilanyl-ethoxymethyl)-5H-pyrrolo[2,3-b]pyrazine-7-carboxylic acid ((R)-2-hydroxy-1,2-dimethyl-propyl)-amide. MS: (M+H)⁺=275

### Example 49

### 2-Ethyl-5H-pyrrolo[2,3-b]pyrazine-7-carboxylic acid (3-hydroxy-2,2-dimethyl-propyl)-amide

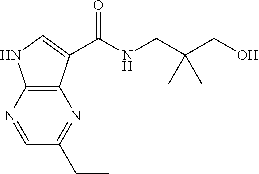 [SMILES: CCc1cnc2[nH]cc(C(=O)NCC(C)(C)CO)c2n1]

Prepared from 2-vinyl-5H-pyrrolo[2,3-b]pyrazine-7-carboxylic acid (3-hydroxy-2,2-dimethyl-propyl)-amide by treatment with 10% palladium on carbon under a hydrogen atmosphere of 50 psi overnight. The reaction mixture was filtered through celite and a Whatman syringe filter and the product was purified by trituration with ethyl acetate. MS: (M+H)⁺=277.

### Example 50

### 2-(2,2-Dimethyl-cyclopropyl)-5H-pyrrolo[2,3-b]pyrazine-7-carboxylic acid (3-hydroxy-2,2-dimethyl-propyl)-amide

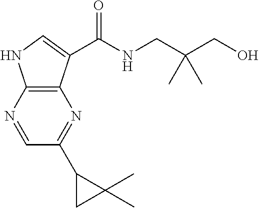 [SMILES: CC(C)(CO)CNC(=O)c1c[nH]c2ncc(C3CC3(C)C)nc12]

### Step 1

2-Bromo-5-(2-trimethylsilanyl-ethoxymethyl)-5H-pyrrolo[2,3-b]pyrazine-7-carboxylic acid (3-hydroxy-2,2-dimethyl-propyl)-amide was prepared according to the procedure outlined in Example 32, step 3 substituting 3-amino-2,2-dimethyl-propan-1-ol for (R)-3-amino-2-methyl-butan-2-ol hydrochloride.

### Step 2

In a pressure tube were combined 2-bromo-5-(2-trimethylsilanyl-ethoxymethyl)-5H-pyrrolo[2,3-b]pyrazine-7-carboxylic acid (3-hydroxy-2,2-dimethyl-propyl)-amide (100 mg, 0.22 mmol), potassium (2,2-dimethyl-cyclopropyl)-trifluoroborate (58 mg, 0.33 mmol), cesium carbonate (251 mg, 0.77 mmol), Pd(dppf)Cl₂ (18 mg, 0.02 mmol), THF (0.75 mL), and water (0.25 mL). The tube was purged with argon, sealed and heated at 100° C. overnight. The solvents were evaporated and the crude residue was purified by SiO₂ chromatography eluting with 25% to 50% EtOAc/hexanes to afford 63 mg (64%) of 2-(2,2-dimethyl-cyclopropyl)-5-(2-trimethylsilanyl-ethoxymethyl)-5H-pyrrolo[2,3-b]pyrazine-7-carboxylic acid (3-hydroxy-2,2-dimethyl-propyl)-amide.

### Step 3

2-(2,2-Dimethyl-cyclopropyl)-5H-pyrrolo[2,3-b]pyrazine-7-carboxylic acid (3-hydroxy-2,2-dimethyl-propyl)-amide was prepared according to the procedure outlined in Example 32, step 5 substituting 1N sodium hydroxide for ethylenediamine. MS: (M+H)⁺=317; mp=261.0-263.0.

### Example 51

### 2-((trans)-2-Methyl-cyclopropyl)-5H-pyrrolo[2,3-b]pyrazine-7-carboxylic acid (3-hydroxy-2,2-dimethyl-propyl)-amide

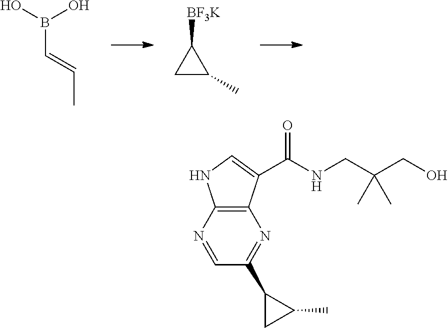

### Step 1

A solution of trans-1-propen-1-ylboronic acid (1.0 g, 11.6 mmol), pinacol (1.5 g, 12.8 mmol) and magnesium sulfate (0.7 g, 5.8 mmol) in diethyl ether (23 mL) was stirred at room temperature for 1 h then concentrated to afford 4,4,5,5-tetramethyl-2-((E)-propenyl)-[1,3,2]dioxaborolane which was used without further purification.

### Step 2

To a solution of 4,4,5,5-tetramethyl-2-((E)-propenyl)-[1,3,2]dioxaborolane (1.9 g, 11.6 mmol, crude from step 1) in toluene (11.6 mL) under nitrogen was carefully added diethyl zinc (1.1 M in toluene, 10.5 mL, 11.6 mmol) followed by diiodomethane (1.3 mL, 16.2 mmol). The reaction mixture was stirred at 50° C. for 4 h. Additional diethyl zinc (1.1 M in toluene, 10.5 mL, 11.6 mmol) and diiodomethane (1.3 mL, 16.2 mmol) were added and heating was continued overnight. The reaction was cooled and 1.0 M HCl (25 mL) was added followed by saturated NaHCO₃ (100 mL). The reaction was filtered and the filtrate was extracted with diethyl ether (2×). The combined organics were washed with water, dried over sodium sulfate and concentrated to provide 4,4,5,5-tetramethyl-2-((trans)-2-methyl-cyclopropyl)-[1,3,2]dioxaborolane. The purity was judged to be 80% by NMR analysis and the isolated product was used without further purification.

### Step 3

A solution of KHF₂ (6.0 g, 77 mmol) in water (7.7 mmol) was added to a solution of 4,4,5,5-tetramethyl-2-((trans)-2-methyl-cyclopropyl)-[1,3,2]dioxaborolane (2.0 g, 11 mmol, crude from step 2) in MeOH (40 mL). The reaction mixture was stirred at room temperature overnight then concentrated. The residue was extracted with acetonitrile (3×). The combined organics were concentrated and residue was triturated with diethyl ether. The resulting solid was collected via filtration, rinsing with diethyl ether. Isolated 787 mg (44%, 3 steps) of potassium trans-1-trifluoroborate-2-methylcyclopropane which was judged to be 80% pure by NMR analysis. Major contaminant was the analogous alkene. Used without further purification.

### Step 4

2-((trans)-2-Methyl-cyclopropyl)-5-(2-trimethylsilanyl-ethoxymethyl)-5H-pyrrolo[2,3-b]pyrazine-7-carboxylic acid (3-hydroxy-2,2-dimethyl-propyl)-amide was prepared according to the procedure outlined in Example 50, step 2 substituting potassium trans-1-trifluoroborate-2-methylcyclopropane for potassium (2,2-dimethyl-cyclopropyl)-trifluoroborate.

### Step 5

2-((trans)-2-Methyl-cyclopropyl)-5H-pyrrolo[2,3-b]pyrazine-7-carboxylic acid (3-hydroxy-2,2-dimethyl-propyl)-amide was prepared according to the procedure outlined in Example 32, step 5 substituting 1N sodium hydroxide for ethylenediamine. MS: (M+H)⁺=303.

### Example 52

### 2-((cis)-2-Methyl-cyclopropyl)-5H-pyrrolo[2,3-b]pyrazine-7-carboxylic acid (3-hydroxy-2,2-dimethyl-propyl)-amide

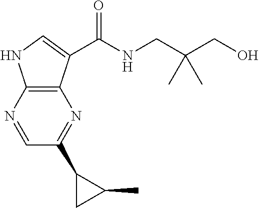 [SMILES: C[C@H]1C[C@H]1c1cnc2[nH]cc(C(=O)NCC(C)(C)CO)c2n1]

Prepared according to the procedure outlined in Example 51 substituting cis-1-propen-1-ylboronic acid for trans-1-propen-1-ylboronic acid. MS: (M+H)⁺=303.

### Example 53

### 2-Cyclopropyl-5H-pyrrolo[2,3-b]pyrazine-7-carboxylic acid ((R)-1,2,2-trimethyl-propyl)-amide

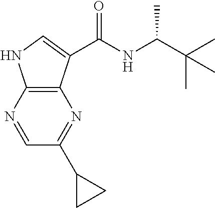 [SMILES: C[C@@H](NC(=O)c1c[nH]c2ncc(C3CC3)nc12)C(C)(C)C]

Prepared according to the procedure outlined in Example 1, steps 4-5 substituting (R)-1,2,2-trimethylpropylamine for 1-((R)-1-amino-ethyl)-cyclopentanol hydrochloride. MS: (M+H)⁺=287; mp=298.0-300.0.

### Example 54

### 2-Cyclopropyl-5H-pyrrolo[2,3-b]pyrazine-7-carboxylic acid isopropylamide

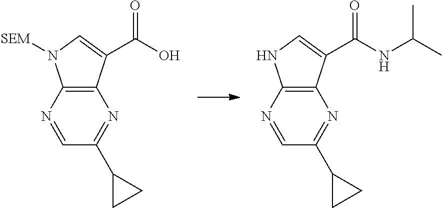 [SMILES: C[Si](C)(C)CCOCn1cc(C(=O)O)c2nc(C3CC3)cnc21 ; CC(C)NC(=O)c1c[nH]c2ncc(C3CC3)nc12]

### Step 1

To a solution of 2-cyclopropyl-5-(2-trimethylsilanyl-ethoxymethyl)-5H-pyrrolo[2,3-b]pyrazine-7-carboxylic acid (0.20 g, 0.59 mmol) in CH₂Cl₂ (5 mL) was added EDC (0.14 g, 0.72 mmol), 4-(dimethylamino)pyridine (0.088 g, 0.72 mmol), and isopropylamine (0.042 g, 0.72 mmol). The reaction mixture was stirred at room temperature overnight then diluted with H₂O and extracted with CH₂Cl₂. The combined organics were washed with brine, dried over Na₂SO₄ and concentrated. The residue was purified by SiO₂ chromatography (30% EtOAc/hexanes) to obtain 0.18 g (81%) of 2-cyclopropyl-5-(2-trimethylsilanyl-ethoxymethyl)-5H-pyrrolo[2,3-b]pyrazine-7-carboxylic acid isopropylamide as an oil.

### Step 2

To a solution of 2-cyclopropyl-5-(2-trimethylsilanyl-ethoxymethyl)-5H-pyrrolo[2,3-b]pyrazine-7-carboxylic acid isopropylamide (0.18 g, 0.48 mmol) in CH₂Cl₂ (5 mL) was added trifluoroacetic acid (1.0 mL). The reaction mixture was stirred at room temperature overnight then concentrated. The residue was dissolved in MeOH (10 mL) and H₂O (2 mL) and Et₃N (2 mL) were added. The reaction mixture was stirred at room temperature overnight then concentrated. The residue was purified by SiO₂ chromatography (2% MeOH/CH₂Cl₂) to afford 0.0.75 g (64%) of 2-cyclopropyl-5H-pyrrolo[2,3-b]pyrazine-7-carboxylic acid isopropylamide as a white solid. MS: (M+H)⁺=245; mp>300.0.

### Example 55

### 2-Cyclopropyl-5H-pyrrolo[2,3-b]pyrazine-7-carboxylic acid (2-methoxy-1-methyl-ethyl)-amide

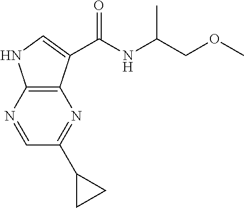 [SMILES: COCC(C)NC(=O)c1c[nH]c2ncc(C3CC3)nc12]

Prepared according to the procedure outlined in Example 54 substituting 2-amino-1-methoxypropane for isopropylamine. MS: (M+H)⁺=275; mp=238.0-240.0.

### Example 56

### 2-Cyclopropyl-5H-pyrrolo[2,3-b]pyrazine-7-carboxylic acid (3-hydroxy-1,1-dimethyl-butyl)-amide

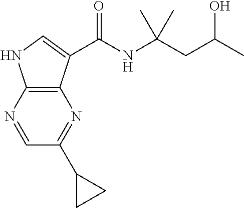 [SMILES: CC(O)CC(C)(C)NC(=O)c1c[nH]c2ncc(C3CC3)nc12]

Prepared according to the procedure outlined in Example 54 substituting 4-amino-4-methyl-pentan-2-ol for isopropylamine. MS: (M+H)⁺=303; mp=230.0-232.0.

### Example 57

### 2-Cyclopropyl-5H-pyrrolo[2,3-b]pyrazine-7-carboxylic acid (2-cyanoethyl)-amide

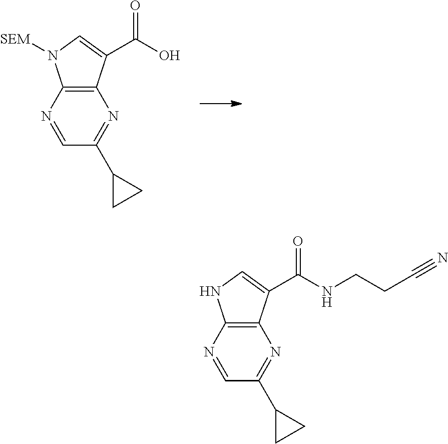 [SMILES: C[Si](C)(C)CCOCn1cc(C(=O)O)c2nc(C3CC3)cnc21 ; N#CCCNC(=O)c1c[nH]c2ncc(C3CC3)nc12]

### Step 1

To a solution of 2-cyclopropyl-5-(2-trimethylsilanyl-ethoxymethyl)-5H-pyrrolo[2,3-b]pyrazine-7-carboxylic acid (0.26 g, 0.77 mmol) in CH₂Cl₂ (10 mL) was added trifluoroacetic acid (1.5 mL). The reaction mixture was stirred at room temperature overnight then concentrated. The residue was dissolved in MeOH (10 mL) and H₂O (1 mL) and Et₃N (2 mL) were added. The reaction mixture was stirred at room temperature overnight then concentrated and dried under high vacuum to afford of 2-cyclopropyl-5H-pyrrolo[2,3-b]pyrazine-7-carboxylic acid which was used without further purification.

### Step 2

To a solution of 2-cyclopropyl-5H-pyrrolo[2,3-b]pyrazine-7-carboxylic acid (0.156 g, 0.77 mmol, crude from step 1) in CH₂Cl₂ (10 mL) was added EDC (0.176 g, 0.92 mmol), 4-(dimethylamino)pyridine (0.11 g, 0.92 mmol), and 3-aminopropionitrile (0.065 g, 0.92 mmol). The reaction mixture was stirred at room temperature overnight then diluted with H₂O and extracted with CH₂Cl₂. The combined organics were washed with brine, dried over Na₂SO₄ and concentrated. The residue was triturated with 50% EtOH/Et₂O to afford 0.059 g (30%) of 2-cyclopropyl-5H-pyrrolo[2,3-b]pyrazine-7-carboxylic acid (2-cyano-ethyl)-amide as an off-white solid. MS: (M+H)⁺=256; mp=236.0-238.0

### Example 58

### 2-Cyclopropyl-5H-pyrrolo[2,3-b]pyrazine-7-carboxylic acid cyanomethyl-amide

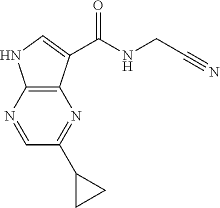 [SMILES: N#CCNC(=O)c1c[nH]c2ncc(C3CC3)nc12]

Prepared according to the procedure outlined in Example 57 substituting aminoacetonitrile for 3-aminopropionitrile. MS: (M+H)⁺=242; mp=240.0-242.0.

### Example 59

### 2-Cyclopropyl-5H-pyrrolo[2,3-b]pyrazine-7-carboxylic acid (3-cyanopropyl)-amide

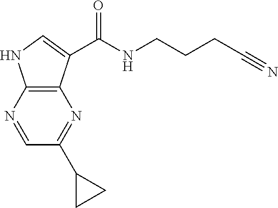 [SMILES: N#CCCCNC(=O)c1c[nH]c2ncc(C3CC3)nc12]

Prepared according to the procedure outlined in Example 57 substituting 4-aminobutanenitrile for 3-aminopropionitrile. MS: (M+H)⁺=270; mp=232.0-234.0.

### Example 60

### 2-Cyclopropyl-5H-pyrrolo[2,3-b]pyrazine-7-carboxylic acid ((S)-1-ethyl-2-hydroxy-2-methyl-propyl)-amide

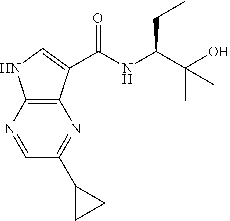 [SMILES: CC[C@H](NC(=O)c1c[nH]c2ncc(C3CC3)nc12)C(C)(C)O]

Prepared according to the procedure outlined in Example 54 substituting (S)-3-amino-2-methyl-pentan-2-ol hydrochloride for isopropylamine. MS: (M+H)⁺=303; mp=229.0-231.0.

### Example 61

### 2-Cyclopropyl-5H-pyrrolo[2,3-b]pyrazine-7-carboxylic acid (3-hydroxy-1,1-dimethyl-propyl)-amide

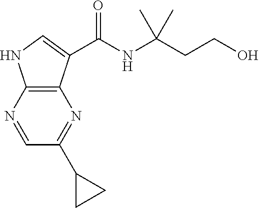 [SMILES: CC(C)(CCO)NC(=O)c1c[nH]c2ncc(C3CC3)nc12]

Prepared according to the procedure outlined in Example 54 substituting 3-amino-3-methyl-butan-1-ol for isopropylamine. MS: (M+H)⁺=289; mp=250.0-252.0.

### Example 62

### 2-Cyclopropyl-5H-pyrrolo[2,3-b]pyrazine-7-carboxylic acid ((S)-1-hydroxymethyl-2,2-dimethyl-propyl)-amide

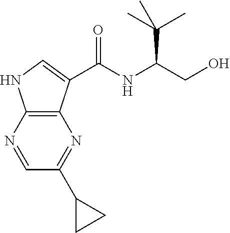 [SMILES: CC(C)(C)[C@@H](CO)NC(=O)c1c[nH]c2ncc(C3CC3)nc12]

Prepared according to the procedure outlined in Example 54 substituting (S)-tert-leucinol for isopropylamine. MS: (M+H)⁺=303; mp=259.0-261.0.

### Example 63

### 2-Cyclopropyl-5H-pyrrolo[2,3-b]pyrazine-7-carboxylic acid (2-hydroxy-1-hydroxymethyl-ethyl)-amide

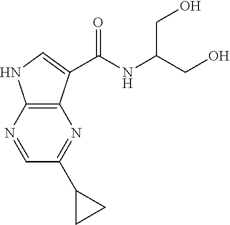 [SMILES: O=C(NC(CO)CO)c1c[nH]c2ncc(C3CC3)nc12]

Prepared according to the procedure outlined in Example 57 substituting 2-amino-1,3-propanediol for 3-aminopropionitrile. MS: (M+H)⁺=277; mp=255.0-256.7.

### Example 64

### 2-Cyclopropyl-5H-pyrrolo[2,3-b]pyrazine-7-carboxylic acid ((R)-1-hydroxymethyl-2,2-dimethyl-propyl)-amide

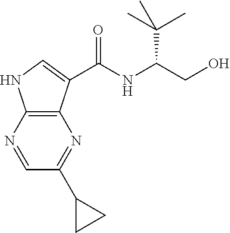 [SMILES: CC(C)(C)[C@H](CO)NC(=O)c1c[nH]c2ncc(C3CC3)nc12]

Prepared according to the procedure outlined in Example 54 substituting (R)-tert-leucinol for isopropylamine. MS: (M+H)⁺=303; mp=270.0-273.0.

### Example 65

### 2-Cyclopropyl-5H-pyrrolo[2,3-b]pyrazine-7-carboxylic acid ((R)-1-hydroxymethyl-2-methyl-propyl)-amide

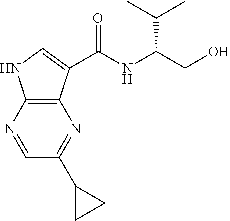 [SMILES: CC(C)[C@H](CO)NC(=O)c1c[nH]c2ncc(C3CC3)nc12]

Prepared according to the procedure outlined in Example 54 substituting D-valinol for isopropylamine. MS: (M+H)⁺=289; mp=250.0-253.0.

### Example 66

### 2-Cyclopropyl-5H-pyrrolo[2,3-b]pyrazine-7-carboxylic acid ((S)-2-hydroxy-1-methyl-ethyl)-amide

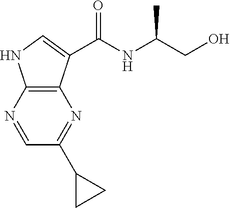 [SMILES: C[C@@H](CO)NC(=O)c1c[nH]c2ncc(C3CC3)nc12]

Prepared according to the procedure outlined in Example 54 substituting L-alaninol for isopropylamine. MS: (M+H)⁺=261; mp=274.0-276.0.

### Example 67

### 2-Cyclopropyl-5H-pyrrolo[2,3-b]pyrazine-7-carboxylic acid ((R)-1-hydroxymethyl-propyl)-amide

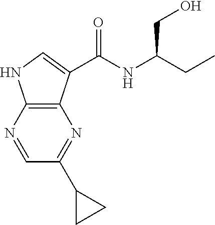 [SMILES: CC[C@H](CO)NC(=O)c1c[nH]c2ncc(C3CC3)nc12]

Prepared according to the procedure outlined in Example 54 substituting (R)-(−)-2-amino-1-butanol for isopropylamine. MS: (M+H)⁺=275; mp=250.0-253.0.

### Example 68

### 2-Cyclopropyl-5H-pyrrolo[2,3-b]pyrazine-7-carboxylic acid ((R)-1-cyclohexylethyl)-amide

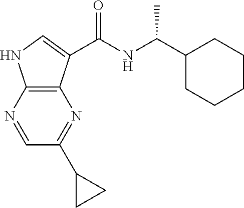 [SMILES: C[C@@H](NC(=O)c1c[nH]c2ncc(C3CC3)nc12)C1CCCCC1]

Prepared according to the procedure outlined in Example 54 substituting (R)-(−)-1-cyclohexylethylamine for isopropylamine. MS: (M+H)⁺=313; mp=253.0-255.0.

### Example 69

### 2-Cyclopropyl-5H-pyrrolo[2,3-b]pyrazine-7-carboxylic acid (2-cyano-1,2,2-trimethyl-ethyl)-amide

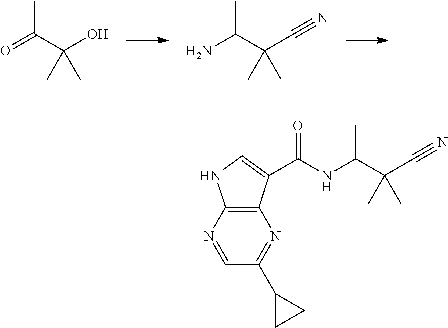

### Step 1

To a solution of 3-hydroxy-3-methyl-2-butanone (1.9 g, 18.6 mmol) and Et₃N (3.9 mL, 27.9 mmol) in CH₂Cl₂ (20 mL) at 0° C. was added a solution of methanesulfonyl chloride (1.6 mL, 20.5 mmol) in CH₂Cl₂ (10 mL). Stirred for 2 h at room temperature then poured into water and extracted with CH₂Cl₂. The organics were washed with 10% aqueous HCl and 5% aqueous NaHCO₃ then dried over MgSO₄ and concentrated to afford 1.8 g (54%) of methanesulfonic acid 1,1-dimethyl-2-oxo-propyl ester as a white solid.

### Step 2

To a solution of methanesulfonic acid 1,1-dimethyl-2-oxo-propyl ester (1.8 g, 10 mmol) in DMSO (10 mL) was added NaCN (1.47 g, 30 mmol). The reaction mixture was stirred at 45° C. overnight then quenched with water and extracted with diethyl ether (2×). The combined organics were washed with brine, dried over MgSO₄ and concentrated to provide 0.52 g (25%) of 2,2-dimethyl-3-oxo-butyronitrile as an oil which was used without further purification.

### Step 3

To a solution of 2,2-dimethyl-3-oxo-butyronitrile (0.52 g, 4.72 mmol) in MeOH (10 mL) was added ammonium acetate (3.64 g, 47.2 mmol) and NaCNBH₃ (0.296 g, 4.72 mmol). The reaction mixture was stirred at room temperature for 5 days then cooled to 0° C. and slowly treated with conc. HCl until pH=2 and stirred for 15 min at room temperature. The reaction mixture was concentrated and the residue was diluted with water and extracted with CH₂Cl₂. The aqueous layer was made basic (pH=10) with conc. NH₄OH then extracted with CH₂Cl₂. The organic layer was dried over MgSO₄ and concentrated to afford 0.031 g (6%) of 3-amino-2,2-dimethyl-butyronitrile as an oil.

### Step 4

2-Cyclopropyl-5H-pyrrolo[2,3-b]pyrazine-7-carboxylic acid (2-cyano-1,2,2-trimethyl-ethyl)-amide was prepared according to the procedure outlined in Example 54 substituting 3-amino-2,2-dimethyl-butyronitrile for isopropylamine. MS: (M+H)⁺=298; mp=295.0-297.0.

### Example 70

### 2-Cyclopropyl-5H-pyrrolo[2,3-b]pyrazine-7-carboxylic acid (3-hydroxy-1,2,2-trimethyl-propyl)-amide

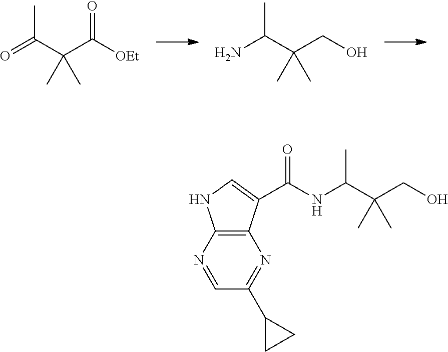

### Step 1

2,2-Dimethyl-3-oxo-butyric acid ethyl ester was prepared according to the procedure reported in J. Am. Chem. Soc. 1988, 110, 1539.

### Step 2

To a solution of 2,2-dimethyl-3-oxo-butyric acid ethyl ester (0.74 g, 4.67 mmol) in MeOH (10 mL) was added ammonium acetate (3.61 g, 46.7 mmol) and NaCNBH₃ (0.29 g, 4.67 mmol). The reaction mixture was stirred at room temperature overnight then cooled to 0° C. and slowly treated with conc. HCl until pH=2 and stirred for 15 min at room temperature. The reaction mixture was concentrated and the residue was diluted with water and extracted with CH₂Cl₂. The aqueous layer was made basic (pH=10) with conc. NH₄OH then extracted with CH₂Cl₂. The organic layer was dried over MgSO₄ and concentrated to afford 0.18 g (24%) of 3-amino-2,2-dimethyl-butyric acid ethyl ester as an oil which was used without further purification.

### Step 3

To a solution of 3-amino-2,2-dimethyl-butyric acid ethyl ester (0.18 g, 1.1 mmol) in dry THF (3 mL) at −78° C. was slowly added LiAlH₄ (1.0 M in THF, 1.2 mL, 1.2 mmol). The reaction mixture was allowed to warm to room temperature and stirred for 2 h then quenched with water and extracted with CH₂Cl₂. The organics were dried over MgSO₄ and concentrated to afford 0.85 g (66%) of 3-amino-2,2-dimethyl-butan-1-ol as an oil which was used without further purification.

### Step 4

2-Cyclopropyl-5H-pyrrolo[2,3-b]pyrazine-7-carboxylic acid (3-hydroxy-1,2,2-trimethyl-propyl)-amide was prepared according to the procedure outlined in Example 54 substituting 3-amino-2,2-dimethyl-butan-1-ol for isopropylamine. MS: (M+H)⁺=303; mp=228.0-270.0.

### Example 71

### 2-Cyclopropyl-5H-pyrrolo[2,3-b]pyrazine-7-carboxylic acid ((S)-sec-butyl)-amide

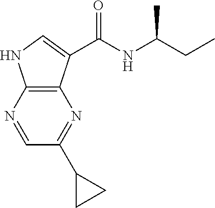 [SMILES: CC[C@H](C)NC(=O)c1c[nH]c2ncc(C3CC3)nc12]

Prepared according to the procedure outlined in Example 54 substituting (S)-(+)-2-aminobutane for isopropylamine. MS: (M+H)⁺=259; mp=280.0-282.0.

### Example 72

### 2-Cyclopropyl-5H-pyrrolo[2,3-b]pyrazine-7-carboxylic acid ((S)-2-hydroxy-1-isopropyl-2-methyl-propyl)-amide

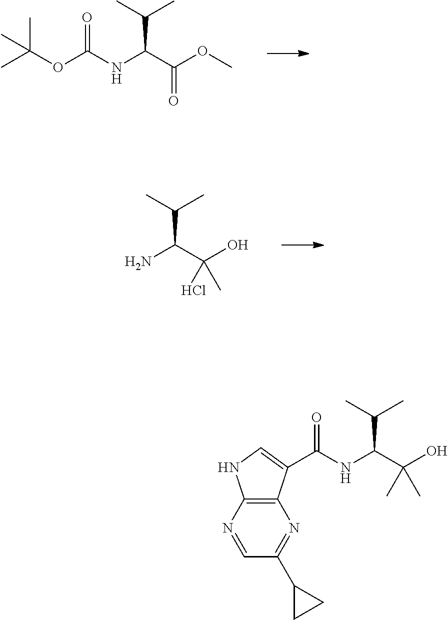

### Step 1

To a solution of N-Boc-L-valine methyl ester (1.5 g, 6.49 mmol) in THF (10 mL) at 0° C. was added methyl magnesium bromide (3.0 M in Et₂O, 9.3 mL, 27.9 mmol). The reaction mixture was stirred at room temperature overnight then quenched with water and extracted with CH₂Cl₂ (2×). The combined organics were dried over MgSO₄ and concentrated to provide 1.71 g of ((S)-2-hydroxy-1-isopropyl-2-methyl-propyl)-carbamic acid tert-butyl ester as a colorless oil which was used without further purification.

### Step 2

((S)-2-hydroxy-1-isopropyl-2-methyl-propyl)-carbamic acid tert-butyl ester (1.71 g, crude from step 1) was dissolved in hydrogen chloride (1.0 M in MeOH, 20 mL, 20 mmol). The solution was stirred at room temperature overnight then concentrated, chased with Et₂O, and dried under high vacuum to afford 1.42 g of (S)-3-amino-2,4-dimethyl-pentan-2-ol hydrochloride as a light brown oil which was used without further purification.

### Step 3

To a solution of 2-cyclopropyl-5-(2-trimethylsilanyl-ethoxymethyl)-5H-pyrrolo[2,3-b]pyrazine-7-carboxylic acid (100 mg, 0.30 mmol) in CH₂Cl₂ (3 mL) was added trifluoroacetic acid (1 mL). The reaction mixture was stirred at room temperature for 2 h then concentrated. The residue was dissolved in DMF (5 mL) and (S)-3-amino-2,4-dimethyl-pentan-2-ol hydrochloride (100 mg, 0.36 mmol), BOP (160 mg, 0.36 mmol), and Et₃N (0.21 mL, 1.5 mmol) were added. The reaction mixture was stirred at room temperature overnight then diluted with EtOAc and washed with aqueous NaHCO₃ (3×) and brine. The organic layer was dried and concentrated. The residue was purified by SiO₂ chromatography eluting with 0% to 100% EtOAc/hexanes to afford 35 mg (37%) of 2-cyclopropyl-5H-pyrrolo[2,3-b]pyrazine-7-carboxylic acid ((S)-2-hydroxy-1-isopropyl-2-methyl-propyl)-amide as a white solid. MS: (M+H)⁺=317; mp=232.0-234.0.

### Example 73

### 2-Cyclopropyl-5H-pyrrolo[2,3-b]pyrazine-7-carboxylic acid ((S)-1,2-dimethyl-propyl)-amide

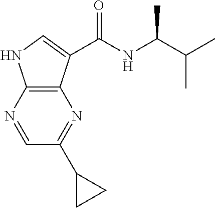 [SMILES: CC(C)[C@H](C)NC(=O)c1c[nH]c2ncc(C3CC3)nc12]

Prepared according to the procedure outlined in Example 72, step 3 substituting (S)-(+)-3-methyl-2-butylamine for (S)-3-amino-2,4-dimethyl-pentan-2-ol hydrochloride. MS: (M+H)⁺=273; mp=281.0-283.0.

### Example 74

### 2-Cyclopropyl-5H-pyrrolo[2,3-b]pyrazine-7-carboxylic acid ((R)-2-hydroxy-1,2-dimethyl-propyl)-amide

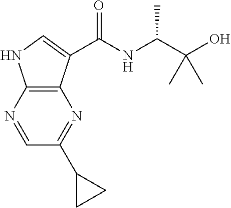 [SMILES: C[C@@H](NC(=O)c1c[nH]c2ncc(C3CC3)nc12)C(C)(C)O]

Prepared according to the procedure outlined in Example 72 substituting N-Boc-D-alanine methyl ester for N-Boc-L-valine methyl ester. MS: (M+H)⁺=289; mp=269.0-271.0.

### Example 75

### 2-Cyclopropyl-5H-pyrrolo[2,3-b]pyrazine-7-carboxylic acid ((R)-1-ethyl-2-hydroxy-2-methyl-propyl)-amide

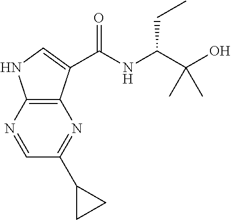 [SMILES: CC[C@@H](NC(=O)c1c[nH]c2ncc(C3CC3)nc12)C(C)(C)O]

Prepared according to the procedure outlined in Example 72 substituting (R)-2-tert-butoxycarbonylamino-butyric acid methyl ester for N-Boc-L-valine methyl ester. MS: (M+H)⁺=303; mp=218.0-222.0.

### Example 76

### 2-Cyclopropyl-5H-pyrrolo[2,3-b]pyrazine-7-carboxylic acid (2-hydroxy-1,1-dimethyl-ethyl)-amide

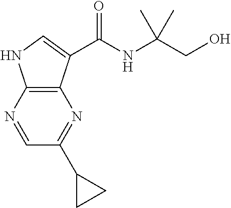 [SMILES: CC(C)(CO)NC(=O)c1c[nH]c2ncc(C3CC3)nc12]

Prepared according to the procedure outlined in Example 72, step 3 substituting 2-amino-2-methyl-1-propanol for (S)-3-amino-2,4-dimethyl-pentan-2-ol hydrochloride. MS: (M+H)⁺=275; mp=293.0-295.0.

### Example 77

### 2-Cyclopropyl-5H-pyrrolo[2,3-b]pyrazine-7-carboxylic acid [(S)-1-(1-hydroxy-1-methyl-ethyl)-pentyl]-amide

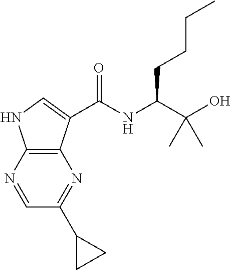 [SMILES: CCCC[C@H](NC(=O)c1c[nH]c2ncc(C3CC3)nc12)C(C)(C)O]

Prepared according to the procedure outlined in Example 72 substituting N-Boc-L-norleucine methyl ester for N-Boc-L-valine methyl ester. MS: (M+H)⁺=331; mp=170.0-172.0.

### Example 78

### 2-Cyclopropyl-5H-pyrrolo[2,3-b]pyrazine-7-carboxylic acid (1-cyclopropyl-ethyl)-amide

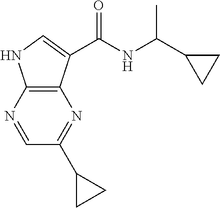 [SMILES: CC(NC(=O)c1c[nH]c2ncc(C3CC3)nc12)C1CC1]

Prepared according to the procedure outlined in Example 54 substituting 1-cyclopropyl-ethylamine for isopropylamine. MS: (M+H)⁺=271; mp=269.0-272.0.

### Example 79

### 2-Cyclopropyl-5H-pyrrolo[2,3-b]pyrazine-7-carboxylic acid (1-ethyl-propyl)-amide

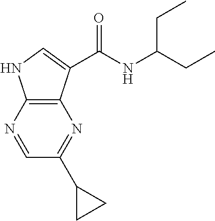 [SMILES: CCC(CC)NC(=O)c1c[nH]c2ncc(C3CC3)nc12]

Prepared according to the procedure outlined in Example 54 substituting 1-ethylpropylamine for isopropylamine. MS: (M+H)⁺=273; mp=245.0-246.0.

### Example 80

### 2-Cyclopropyl-5H-pyrrolo[2,3-b]pyrazine-7-carboxylic acid (2-dimethylamino-1-methyl-ethyl)-amide

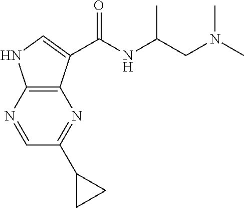 [SMILES: CC(CN(C)C)NC(=O)c1c[nH]c2ncc(C3CC3)nc12]

Prepared according to the procedure outlined in Example 54 substituting 1-dimethylamino-2-propylamine for isopropylamine. MS: (M+H)⁺=288; mp=225.0-229.0.

### Example 81

### 2-Cyclopropyl-5H-pyrrolo[2,3-b]pyrazine-7-carboxylic acid ((S)-1-cyclohexylethyl)-amide

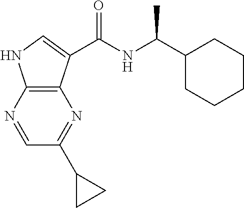 [SMILES: C[C@H](NC(=O)c1c[nH]c2ncc(C3CC3)nc12)C1CCCCC1]

Prepared according to the procedure outlined in Example 54 substituting (S)-(+)-1-cyclohexylethylamine for isopropylamine. MS: (M+H)⁺=313; mp=246.0-249.0.

### Example 82

### 2-Cyclopropyl-5H-pyrrolo[2,3-b]pyrazine-7-carboxylic acid ((R)-2-hydroxy-1-methyl-ethyl)-amide

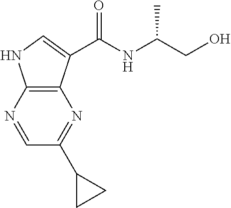 [SMILES: C[C@H](CO)NC(=O)c1c[nH]c2ncc(C3CC3)nc12]

Prepared according to the procedure outlined in Example 54 substituting D-alaninol for isopropylamine. MS: (M+H)⁺=261; mp=265.0-268.0.

### Example 83

### 2-Cyclopropyl-5H-pyrrolo[2,3-b]pyrazine-7-carboxylic acid ((S)-1-hydroxymethyl-propyl)-amide

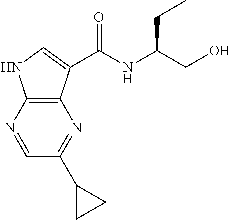 [SMILES: CC[C@@H](CO)NC(=O)c1c[nH]c2ncc(C3CC3)nc12]

Prepared according to the procedure outlined in Example 54 substituting (S)-(+)-2-amino-1-butanol for isopropylamine. MS: (M+H)⁺=275; mp=250.0-252.0.

### Example 84

### 2-Cyclopropyl-5H-pyrrolo[2,3-b]pyrazine-7-carboxylic acid methylamide

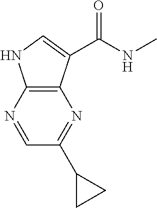 [SMILES: CNC(=O)c1c[nH]c2ncc(C3CC3)nc12]

Prepared according to the procedure outlined in Example 54 substituting methylamine hydrochloride for isopropylamine. MS: (M+H)⁺=217; mp=283.0-286.0.

### Example 85

### 2-Cyclopropyl-5H-pyrrolo[2,3-b]pyrazine-7-carboxylic acid (2,2-dimethyl-propyl)-amide

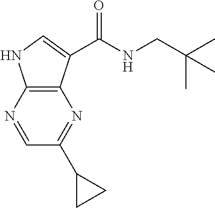 [SMILES: CC(C)(C)CNC(=O)c1c[nH]c2ncc(C3CC3)nc12]

Prepared according to the procedure outlined in Example 54 substituting 2,2-dimethyl-propylamine for isopropylamine. MS: (M+H)⁺=273.

### Example 86

### 2-Cyclopropyl-5H-pyrrolo[2,3-b]pyrazine-7-carboxylic acid [2-hydroxy-1-(2-hydroxy-ethyl)-2-methyl-propyl]-amide

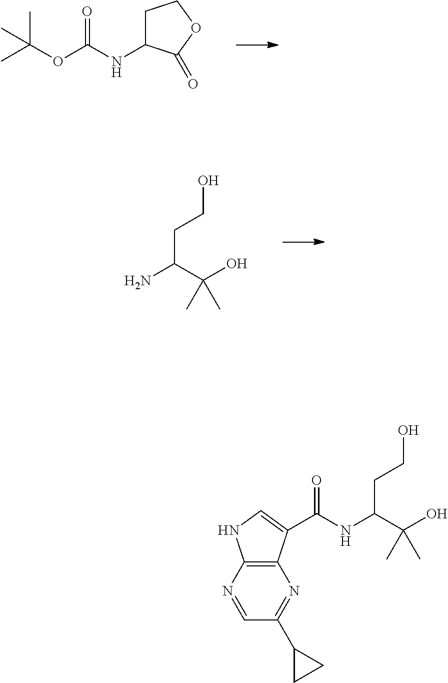

### Step 1

To a solution of tert-butyl-(tetrahydro-2-oxo-3-furanyl)-carbamate (2.1 g, 10.4 mmol) in THF (12 mL) at 0° C. was slowly added methylmagnesium bromide (3.0 M in Et₂O, 14.5 mL, 43.5 mmol). The reaction mixture was stirred at room temperature overnight then carefully quenched with water. The mixture was filtered through Celite, rinsing with CH₂Cl₂. The filtrate was washed with brine, dried over sodium sulfate and concentrated to afford 1.65 g (68%) of 2-hydroxy-1-(2-hydroxy-ethyl)-2-methyl-propyl]-carbamic acid tert-butyl ester as a white solid which was used without further purification.

### Step 2

In a microwave vial, 2-hydroxy-1-(2-hydroxy-ethyl)-2-methyl-propyl]-carbamic acid tert-butyl ester (100 mg, 0.43 mmol) was dissolved in hexafluoroisopropanol (5 mL). The vial was sealed and heated under microwave irradiation at 150° C. for 1 h. The solvent was removed in vacuo to provide 83 mg of 3-amino-4-methyl-pentane-1,4-diol as a light brown oil which was used without further purification.

### Step 3

2-Cyclopropyl-5H-pyrrolo[2,3-b]pyrazine-7-carboxylic acid [2-hydroxy-1-(2-hydroxy-ethyl)-2-methyl-propyl]-amide was prepared according to the procedure outlined in Example 54 substituting 3-amino-4-methyl-pentane-1,4-diol for isopropylamine. MS: (M+H)⁺=319; mp=195.0-198.0.

### Example 87

### 2-Cyclopropyl-5H-pyrrolo[2,3-b]pyrazine-7-carboxylic acid [(S)-1-(1H-pyrazol-3-yl)-ethyl]-amide

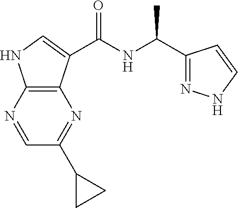 [SMILES: C[C@H](NC(=O)c1c[nH]c2ncc(C3CC3)nc12)c1cc[nH]n1]

### Step 1

2-Cyclopropyl-5-(2-trimethylsilanyl-ethoxymethyl)-5H-pyrrolo[2,3-b]pyrazine-7-carboxylic acid [(S)-1-(1H-pyrazol-3-yl)-ethyl]-amide was prepared according to the procedure outlined in Example 54, step 1 substituting (S)-1-(1H-pyrazol-3-yl)-ethylamine for isopropylamine.

### Step 2

To a solution of 2-cyclopropyl-5-(2-trimethylsilanyl-ethoxymethyl)-5H-pyrrolo[2,3-b]pyrazine-7-carboxylic acid [(S)-1-(1H-pyrazol-3-yl)-ethyl]-amide (230 mg, 0.54 mmol) in MeOH (9 mL) was added 6 M aqueous HCl (2 mL). The reaction mixture was stirred at room temperature for 4 h then heated at 80° C. overnight. The reaction was cooled to room temperature and K₂CO₃ (2 g) was added. The reaction was stirred at room temperature overnight then concentrated. The residue was diluted with water and extracted with EtOAc. Dried over MgSO4 and concentrated. The residue was triturated with EtOAc/hexanes to afford 130 mg (81%) of 2-cyclopropyl-5H-pyrrolo[2,3-b]pyrazine-7-carboxylic acid [(S)-1-(1H-pyrazol-3-yl)-ethyl]-amide. MS: (M+H)⁺=297.

### Example 88

### 2-Cyclopropyl-5H-pyrrolo[2,3-b]pyrazine-7-carboxylic acid ((R)-1-phenyl-ethyl)-amide

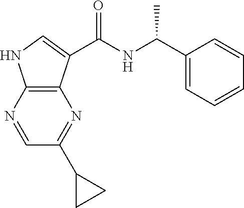 [SMILES: C[C@@H](NC(=O)c1c[nH]c2ncc(C3CC3)nc12)c1ccccc1]

Prepared according to the procedure outlined in Example 54 substituting (R)-(+)-1-phenylethylamine for isopropylamine. 1.0 M NaOH and THF were substituted for MeOH, H₂O, and Et₃N in step 2. MS: (M+H)⁺=307; mp=278.0-280.0.

### Example 89

### 2-Cyclopropyl-5H-pyrrolo[2,3-b]pyrazine-7-carboxylic acid ((S)-1-phenyl-ethyl)-amide

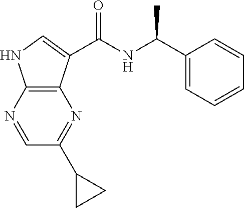 [SMILES: C[C@H](NC(=O)c1c[nH]c2ncc(C3CC3)nc12)c1ccccc1]

Prepared according to the procedure outlined in Example 54 substituting (S)-(−)-1-phenylethylamine for isopropylamine. 1.0 M NaOH and THF were substituted for MeOH, H₂O, and Et₃N in step 2. MS: (M+H)⁺=307; mp=272.0-274.0.

### Example 90

### 2-Cyclopropyl-5H-pyrrolo[2,3-b]pyrazine-7-carboxylic acid (3-hydroxy-butyl)-amide

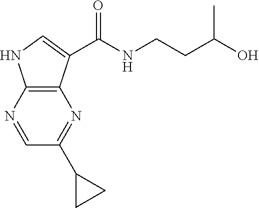 [SMILES: CC(O)CCNC(=O)c1c[nH]c2ncc(C3CC3)nc12]

Prepared according to the procedure outlined in Example 54 substituting 4-amino-2-butanol for isopropylamine. 1.0 M NaOH and THF were substituted for MeOH, H₂O, and Et₃N in step 2. MS: (M+H)⁺=275; mp=228.0-230.0.

### Example 91

### 2-Cyclopropyl-5H-pyrrolo[2,3-b]pyrazine-7-carboxylic acid (3-hydroxy-2-methyl-propyl)-amide

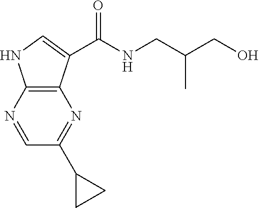 [SMILES: CC(CO)CNC(=O)c1c[nH]c2ncc(C3CC3)nc12]

Prepared according to the procedure outlined in Example 54 substituting 3-amino-2-methyl-1-propanol for isopropylamine. 1.0 M NaOH and THF were substituted for MeOH, H₂O, and Et₃N in step 2. MS: (M+H)⁺=275; mp=252.0-254.0.

### Example 92

### 2-Cyclopropyl-5H-pyrrolo[2,3-b]pyrazine-7-carboxylic acid (1-pyridin-2-yl-ethyl)-amide

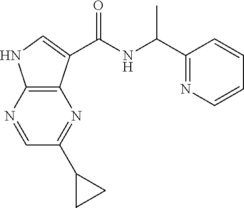 [SMILES: CC(NC(=O)c1c[nH]c2ncc(C3CC3)nc12)c1ccccn1]

Prepared according to the procedure outlined in Example 54 substituting 1-pyridin-2-yl-ethylamine for isopropylamine. 1.0 M NaOH and THF were substituted for MeOH, H₂O, and Et₃N in step 2. MS: (M+H)⁺=308; mp=217.0-219.0.

### Example 93

### 2-Pyridin-2-yl-5H-pyrrolo[2,3-b]pyrazine-7-carboxylic acid ((S)-1,2,2-trimethyl-propyl)-amide

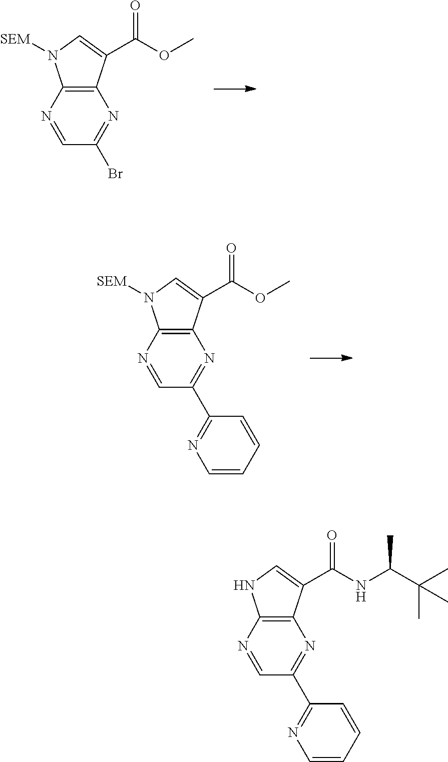 [SMILES: COC(=O)c1cn(COCC[Si](C)(C)C)c2ncc(Br)nc12 ; COC(=O)c1cn(COCC[Si](C)(C)C)c2ncc(-c3ccccn3)nc12 ; C[C@H](NC(=O)c1c[nH]c2ncc(-c3ccccn3)nc12)C(C)(C)C]

### Step 1

2-Bromo-5-(2-trimethylsilanyl-ethoxymethyl)-5H-pyrrolo[2,3-b]pyrazine-7-carboxylic acid methyl ester (105 mg, 0.27 mmol) in THF (1 mL) was added Pd(PPh₃)₄ (16 mg, 0.014 mmol). The reaction mixture was degassed with argon then 2-pyridylzinc bromide (0.5 M in THF, 1.35 mL, 0.675 mmol) was added. The reaction mixture was heated at 50° C. overnight. Cooled to room temperature, quenched with aqueous NaHCO₃ and extracted with EtOAc. The organic layer was dried over MgSO₄ and concentrated. The residue was purified by SiO₂ chromatography (1% to 10% MeOH/CH₂Cl₂) to afford 120 mg of 2-pyridin-2-yl-5-(2-trimethylsilanyl-ethoxymethyl)-5H-pyrrolo[2,3-b]pyrazine-7-carboxylic acid methyl ester as a yellow oil which contained some minor impurities.

### Step 2

To a solution of 2-pyridin-2-yl-5-(2-trimethylsilanyl-ethoxymethyl)-5H-pyrrolo[2,3-b]pyrazine-7-carboxylic acid methyl ester (120 mg, 0.27 mmol) in THF (2.5 mL) was added aqueous 1.0 M NaOH (1.0 mL). the reaction mixture was stirred at room temperature overnight. The reaction was neutralized to pH=7 with aqueous 1.0 M HCl. The resultant precipitate was collected by filtration then dissolved in 10% MeOH/CH₂Cl₂, dried and concentrated to afford 65 mg of 2-pyridin-2-yl-5-(2-trimethylsilanyl-ethoxymethyl)-5H-pyrrolo[2,3-b]pyrazine-7-carboxylic acid as a yellow oil.

### Step 3

2-Pyridin-2-yl-5H-pyrrolo[2,3-b]pyrazine-7-carboxylic acid ((S)-1,2,2-trimethyl-propyl)-amide was prepared according to the procedure outlined in Example 54 substituting 2-pyridin-2-yl-5-(2-trimethylsilanyl-ethoxymethyl)-5H-pyrrolo[2,3-b]pyrazine-7-carboxylic acid for 2-cyclopropyl-5-(2-trimethylsilanyl-ethoxymethyl)-5H-pyrrolo[2,3-b]pyrazine-7-carboxylic acid and (S)-1,2,2-trimethyl-propylamine for isopropylamine. 1.0 M NaOH and THF were substituted for MeOH, H₂O, and Et₃N in step 2. MS: (M+H)⁺=324; mp>300.0.

### Example 94

### 2-Cyclopropyl-5H-pyrrolo[2,3-b]pyrazine-7-carboxylic acid ((S)-1-cyclopropyl-2-hydroxy-2-methyl-propyl)-amide

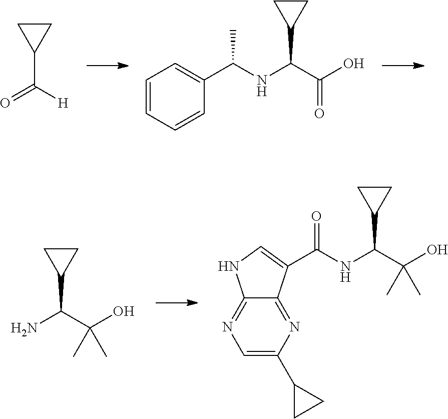

### Step 1

(S)-Cyclopropyl-((S)-1-phenyl-ethylamino)-acetic acid was prepared from cyclopropane carboxaldehyde following the procedure outlined in U.S. Pat. No. 6,191,306.

### Step 2

To a suspension of (S)-cyclopropyl-((S)-1-phenyl-ethylamino)-acetic acid (0.50 g, 2.28 mmol) in MeOH (20 mL) was slowly added (trimethylsilyl)diazomethane (2.0 M in Et₂O, 5.0 mL, 10 mmol) using an ice bath to periodically regulate the temperature. The homogeneous reaction mixture was stirred at room temperature for 1 h then poured into aqueous NaHCO₃ and extracted with CH₂Cl₂ (3×). The combined organics were dried over MgSO₄ and concentrated to provide 0.42 g (79%) of (S)-cyclopropyl-((S)-1-phenyl-ethylamino)-acetic acid methyl ester as an orange oil which was used without further purification.

### Step 3

To a solution of (S)-cyclopropyl-((S)-1-phenyl-ethylamino)-acetic acid methyl ester (0.42 g, 1.8 mmol) in THF (8 mL) at 0° C. was slowly added methylmagnesium bromide (3.0 M in Et₂₀, 1.5 mL, 4.5 mmol). The reaction mixture was stirred at 0° C. for 1 h then quenched with aqueous NH₄Cl, diluted with water and extracted with EtOAc (2×). The combined organics were dried over MgSO₄ and concentrated. The residue was purified by SiO₂ chromatography (20% to 50% EtOAc/hexanes) to afford 0.25 g (60%) of (S)-1-cyclopropyl-2-methyl-1-((S)-1-phenyl-ethylamino)-propan-2-ol as a light yellow oil.

### Step 4

To a solution of (S)-1-cyclopropyl-2-methyl-1-((S)-1-phenyl-ethylamino)-propan-2-ol (0.25 g, 1.07 mmol) in MeOH (8 mL) was added 20% Pd(OH)₂ on carbon (30 mg). The reaction mixture was stirred under H₂ (1 atm, balloon) for 18 h then filtered over Celite, rinsing with EtOAc. The filtrate was concentrated to provide 0.16 g of (S)-1-amino-1-cyclopropyl-2-methyl-propan-2-ol as a pale yellow oil which was used without further purification.

### Step 5

In a flask were combined 2-cyclopropyl-5H-pyrrolo[2,3-b]pyrazine-7-carboxylic acid (150 mg, 0.74 mmol), (S)-1-amino-1-cyclopropyl-2-methyl-propan-2-ol (115 mg, 0.89 mmol), EDC (155 mg, 0.81 mmol), and HOBt (109 mg, 0.81 mmol). Then added DMF (2 mL) followed by i-Pr₂NEt (0.19 mL, 1.11 mmol). The reaction mixture was stirred at room temperature overnight then quenched with H₂O and extracted with EtOAc (3×). The combined organics were washed with H₂O (3×) then dried over MgSO₄ and concentrated. The residue was purified by SiO₂ chromatography (50% to 100% EtOAc/hexanes) to afford 30 mg (13%) of 2-cyclopropyl-5H-pyrrolo[2,3-b]pyrazine-7-carboxylic acid ((S)-1-cyclopropyl-2-hydroxy-2-methyl-propyl)-amide as a white solid. MS: (M+H)⁺=315; mp=238.0-240.0.

### Example 95

### 2-Cyclopropyl-5H-pyrrolo[2,3-b]pyrazine-7-carboxylic acid ((R)-1-cyclopropyl-2-hydroxy-2-methyl-propyl)-amide

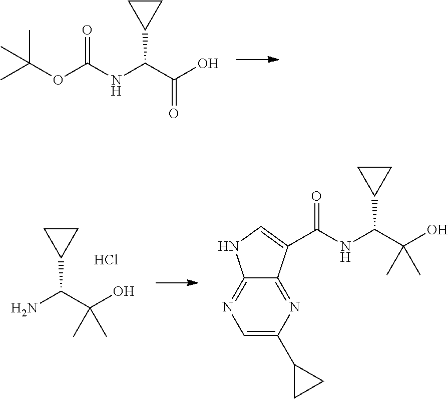

### Step 1

To a solution of N-Boc-D-cyclopropylglycine (0.50 g, 2.32 mmol) in MeOH (20 mL) at 0° C. was slowly added (trimethylsilyl)diazomethane (2.0 M in Et₂O, 5.0 mL, 10 mmol). The reaction mixture was stirred at room temperature for 1 h then quenched with a small portion of acetic acid and concentrated to afford 0.56 g of Boc-D-cyclopropylglycine methyl ester a colorless oil which was used without further purification.

### Step 2

To a solution of N-Boc-D-cyclopropylglycine acid methyl ester (0.56 g, 2.32 mmol) in THF (10 mL) at 0° C. was slowly added methylmagnesium bromide (3.0 M in diethyl ether, 2.7 mL, 8.1 mmol). The reaction mixture was stirred at 0° C. for 1 h then quenched with saturated aqueous NH₄Cl then extracted with EtOAc (2×). The combined organics were washed with water and brine then dried over MgSO₄ and concentrated. The residue was purified by chromatography over 24 g SiO₂ eluting with 10% to 30% EtOAc/hexanes to afford 0.44 g (82%) of ((R)-1-cyclopropyl-2-hydroxy-2-methyl-propyl)-carbamic acid tert-butyl ester as a colorless oil.

### Step 3

In a round-bottomed flask, ((R)-1-cyclopropyl-2-hydroxy-2-methyl-propyl)-carbamic acid tert-butyl ester (0.44 g, 1.92 mmol) was dissolved in 1.0 M HCl in MeOH (10.0 mL, 10.0 mmol). The reaction mixture was stirred at 50° C. for 4 h then cooled to room temperature and concentrated to afford 0.26 g (82%) of (R)-1-amino-1-cyclopropyl-2-methyl-propan-2-ol hydrochloride as a light pink solid.

### Step 4

In a flask were combined 2-cyclopropyl-5H-pyrrolo[2,3-b]pyrazine-7-carboxylic acid (150 mg, 0.74 mmol), (R)-1-amino-1-cyclopropyl-2-methyl-propan-2-ol hydrochloride (147 mg, 0.89 mmol), EDC (155 mg, 0.81 mmol), and HOBt (109 mg, 0.81 mmol). Then added DMF (2 mL) followed by i-Pr₂NEt (0.32 mL, 1.85 mmol). The reaction mixture was stirred at room temperature overnight then quenched with H₂O and extracted with EtOAc (3×). The combined organics were washed with H₂O (3×) then dried over MgSO₄ and concentrated. The residue was triturated with EtOAc/hexanes to afford 69 mg (30%) of 2-cyclopropyl-5H-pyrrolo[2,3-b]pyrazine-7-carboxylic acid ((R)-1-cyclopropyl-2-hydroxy-2-methyl-propyl)-amide as a white solid. MS: (M+H)⁺=315; mp=235.0-237.0.

### Example 96

### 2-Cyclopropyl-5H-pyrrolo[2,3-b]pyrazine-7-carboxylic acid (2-cyano-1-cyclopropyl-2,2-dimethyl-ethyl)-amide

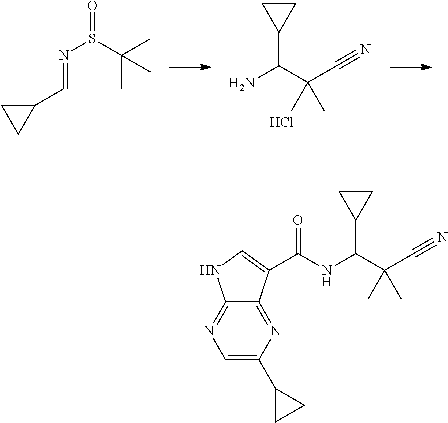

### Step 1

To a solution of isobutyronitrile (0.30 g, 4.35 mmol) in THF (8 mL) at −78° C. was added LiHMDS (1.0M in THF, 4.8 mL, 4.8 mmol). The pale yellow reaction mixture was stirred at −78° C. for 30 min then a solution of 2-methyl-propane-2-sulfinic acid 1-cyclopropyl-meth-(E)-ylideneamide (0.50 g, 2.90 mmol) [prepared according to WO2008/147800] in THF (2 mL) was slowly added. The reaction mixture was stirred at −78° C. for 2 h then quenched with saturated aqueous NH₄Cl and warmed to room temperature. The mixture was diluted with water and extracted with EtOAc (2×). The combined organics were dried over MgSO₄ and concentrated to afford 0.70 g of 2-methylpropane-2-sulfinic acid (2-cyano-1-cyclopropyl-2,2-dimethyl-ethyl)-amide as a viscous colorless oil.

### Step 2

To a solution of 2-methylpropane-2-sulfinic acid (2-cyano-1-cyclopropyl-2,2-dimethyl-ethyl)-amide (0.70 g, 2.90 mmol) in MeOH (5 mL) at room temperature was added 4.0 M HCl in dioxane (1.5 mL, 6.0 mmol). The reaction mixture was stirred at room temperature for 15 min then concentrated to afford 0.45 g (89%, 2 steps) of 3-amino-3-cyclopropyl-2,2-dimethyl-propionitrile hydrochloride as a white solid.

### Step 3

In a flask were combined 2-cyclopropyl-5-((2-(trimethylsilyl)ethoxy)methyl)-5H-pyrrolo[2,3-b]pyrazine-7-carboxylic acid (120 mg, 0.36 mmol), 3-amino-3-cyclopropyl-2,2-dimethyl-propionitrile hydrochloride (75 mg, 0.43 mmol), HOBt (54 mg, 0.40 mmol), and EDC (77 mg, 0.40 mmol). Then added DMF (2 mL) followed by diisopropylethylamine (0.16 mL, 0.90 mmol). The reaction mixture was stirred at room temperature overnight then quenched with water and extracted with EtOAc (3×). The combined organics were washed with water (3×) then dried over MgSO₄ and concentrated. The residue was purified by SiO₂ chromatography (30% to 50% EtOAc/hexanes) to afford 121 mg (74%) of 2-cyclopropyl-5-(2-trimethylsilanyl-ethoxymethyl)-5H-pyrrolo[2,3-b]pyrazine-7-carboxylic acid (2-cyano-1-cyclopropyl-2,2-dimethyl-ethyl)-amide as an off-white foam.

### Step 4

To a solution of 2-cyclopropyl-5-(2-trimethylsilanyl-ethoxymethyl)-5H-pyrrolo[2,3-b]pyrazine-7-carboxylic acid (2-cyano-1-cyclopropyl-2,2-dimethyl-ethyl)-amide (110 mg, 0.24 mmol) in CH₂Cl₂ (4 mL) was added TFA (1 mL). The yellow reaction mixture was stirred for 3 h then concentrated. The residue was redissolved in MeOH (8 ml) and water (1 mL) and triethylamine (1 mL) was added. The reaction mixture was stirred at room temperature overnight then concentrated. The residue was purified by SiO₂ chromatography (50% to 80% EtOAc/hexanes) followed by trituration with EtOAc to afford 45 mg (58%) of 2-cyclopropyl-5H-pyrrolo[2,3-b]pyrazine-7-carboxylic acid (2-cyano-1-cyclopropyl-2,2-dimethyl-ethyl)-amide as a white solid. MS: (M+H)⁺=324; mp=230.0-232.0.

### Example 97

### 3-Cyclopropyl-3-[(2-cyclopropyl-5H-pyrrolo[2,3-b]pyrazine-7-carbonyl)-amino]-2,2-dimethyl-propionic acid

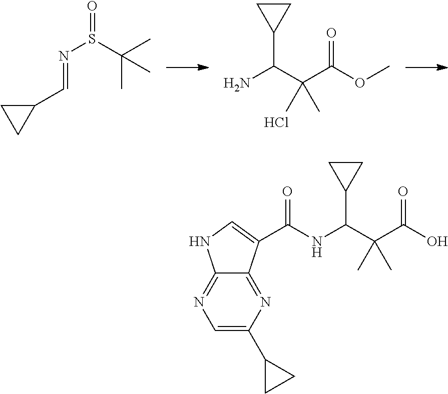 [SMILES: CC(C)(C)S(=O)/N=C/C1CC1 ; COC(=O)C(C)(C)C(N)C1CC1 ; Cl ; CC(C)(C(=O)O)C(NC(=O)c1c[nH]c2ncc(C3CC3)nc12)C1CC1]

### Step 1

To a solution of methyl isobutyrate (1.18 g, 11.5 mmol) in THF (15 mL) at −78° C. was added LiHMDS (1.0M in THF, 12.7 mL, 12.7 mmol). The pale yellow reaction mixture was stirred at −78° C. for 30 min then a solution of 2-methyl-propane-2-sulfinic acid 1-cyclopropyl-meth-(E)-ylideneamide (1.0 g, 5.8 mmol) [prepared according to WO2008/147800] in THF (5 mL) was slowly added. The reaction mixture was stirred at −78° C. for 2 h then warmed to room temperature over 1 h and quenched with saturated aqueous NH₄Cl. The mixture was diluted with water and extracted with EtOAc (2×). The combined organics were dried over MgSO₄ and concentrated. The residue was purified by SiO₂ chromatography (30% to 50% EtOAc/hexanes) to afford 1.15 g (72%) of 3-cyclopropyl-2,2-dimethyl-3-(2-methyl-propane-2-sulfinylamino)-propionic acid methyl ester as a colorless oil.

### Step 2

To a solution of 3-cyclopropyl-2,2-dimethyl-3-(2-methyl-propane-2-sulfinylamino)-propionic acid methyl ester (1.15 g, 4.17 mmol) in MeOH (10 mL) at room temperature was added 4.0 M HCl in dioxane (2.1 mL, 8.4 mmol). The reaction mixture was stirred at room temperature for 30 min then concentrated to afford 0.81 g (94%) of 3-amino-3-cyclopropyl-2,2-dimethyl-propionic acid methyl ester hydrochloride as a white solid.

### Step 3

In a flask were combined 2-cyclopropyl-5-((2-(trimethylsilyl)ethoxy)methyl)-5H-pyrrolo[2,3-b]pyrazine-7-carboxylic acid (200 mg, 0.60 mmol), 3-amino-3-cyclopropyl-2,2-dimethyl-propionic acid methyl ester hydrochloride (150 mg, 0.72 mmol), HOBt (90 mg, 0.66 mmol), and EDC (127 mg, 0.66 mmol). Then added DMF (3 mL) followed by diisopropylethylamine (0.26 mL, 1.50 mmol). The reaction mixture was stirred at room temperature overnight then quenched with water and extracted with EtOAc (3×). The combined organics were washed with water (3×) then dried over MgSO₄ and concentrated. The residue was purified by SiO₂ chromatography (30% EtOAc/hexanes) to afford 264 mg (90%) of 3-cyclopropyl-3-{[2-cyclopropyl-5-(2-trimethylsilanyl-ethoxymethyl)-5H-pyrrolo[2,3-b]pyrazine-7-carbonyl]-amino}-2,2-dimethyl-propionic acid methyl ester as a viscous colorless oil.

### Step 4

To a solution of 3-cyclopropyl-3-{[2-cyclopropyl-5-(2-trimethylsilanyl-ethoxymethyl)-5H-pyrrolo[2,3-b]pyrazine-7-carbonyl]-amino}-2,2-dimethyl-propionic acid methyl ester (110 mg, 0.23 mmol) in CH₂Cl₂ (4 mL) was added TFA (1 mL). The yellow reaction mixture was stirred for 3 h then concentrated. The residue was redissolved in CH₂Cl₂ (4 mL) and ethylenediamine (0.5 mL) was added. The reaction mixture was stirred at room temperature for 1 h then concentrated. The residue was purified by SiO₂ chromatography (50% to 80% EtOAc/hexanes) to isolate 56 mg (70%) of 3-cyclopropyl-3-[(2-cyclopropyl-5H-pyrrolo[2,3-b]pyrazine-7-carbonyl)-amino]-2,2-dimethyl-propionic acid methyl ester as a white solid.

### Step 5

A sample of 3-cyclopropyl-3-[(2-cyclopropyl-5H-pyrrolo[2,3-b]pyrazine-7-carbonyl)-amino]-2,2-dimethyl-propionic acid methyl ester (56 mg, 0.157 mmol) was dissolved in MeOH (1.5 mL), THF (1.5 mL) and H₂O (0.75 mL). Then added LiOH.H₂O (20 mg, 0.471 mmol) and stirred at 50° C. for 18 h. Cooled to room temperature and concentrated. The residue was diluted with water and acidified to pH=4 with 1.0 M HCl. The mixture was extracted with EtOAc (2×). The combined organics were dried over MgSO4 and concentrated to provide 54 mg (99%) of 3-cyclopropyl-3-[(2-cyclopropyl-5H-pyrrolo[2,3-b]pyrazine-7-carbonyl)-amino]-2,2-dimethyl-propionic acid as a white solid. MS: (M+H)⁺=343; mp=265.0-267.0.

### Example 98

### 2-Cyclopropyl-5H-pyrrolo[2,3-b]pyrazine-7-carboxylic acid (1-cyclopentyl-ethyl)-amide

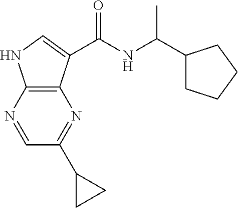 [SMILES: CC(NC(=O)c1c[nH]c2ncc(C3CC3)nc12)C1CCCC1]

Prepared according to the procedure outlined in Example 1, steps 4-5 substituting 1-cyclopentylethylamine for 1-((R)-1-amino-ethyl)-cyclopentanol hydrochloride. MS: (M+H)⁺=299.

### Example 99

### 2-Cyclopropyl-5H-pyrrolo[2,3-b]pyrazine-7-carboxylic acid ((1S,2R,3S)-1-cyclohexylmethyl-3-cyclopropyl-2,3-dihydroxy-propyl)-amide

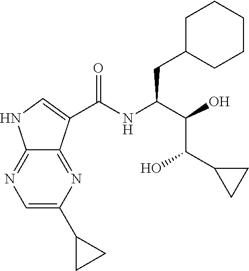 [SMILES: O=C(N[C@@H](CC1CCCCC1)[C@@H](O)[C@@H](O)C1CC1)c1c[nH]c2ncc(C3CC3)nc12]

Prepared according to the procedure outlined in Example 1, steps 4-5 substituting (1S,2R,3S)-3-amino-4-cyclohexyl-1-cyclopropyl-butane-1,2-diol [as prepared in Bioorg. Med. Chem. Lett. 2005, 15, 3292] for 1-((R)-1-amino-ethyl)-cyclopentanol hydrochloride. MS: (M+H)⁺=413.

### Example 100

### 2-Cyclopropyl-5H-pyrrolo[2,3-b]pyrazine-7-carboxylic acid (1-cyano-2-methyl-propyl)-amide

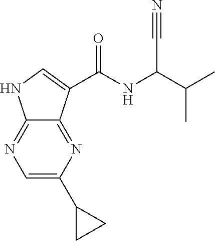 [SMILES: CC(C)C(C#N)NC(=O)c1c[nH]c2ncc(C3CC3)nc12]

Prepared according to the procedure outlined in Example 1, steps 4-5 substituting 2-amino-3-methyl-butyronitrile for 1-((R)-1-amino-ethyl)-cyclopentanol hydrochloride. MS: (M+H)⁺=284.

### Example 101

### 2-Cyclopropyl-5H-pyrrolo[2,3-b]pyrazine-7-carboxylic acid (cyano-cyclopropyl-methyl)-amide

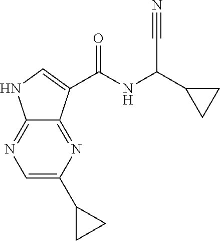 [SMILES: N#CC(NC(=O)c1c[nH]c2ncc(C3CC3)nc12)C1CC1]

Prepared according to the procedure outlined in Example 1, steps 3-5 substituting (cyano-cyclopropyl-methyl)-carbamic acid tert-butyl ester for [(R)-1-(1-hydroxycyclopentyl)-ethyl]-carbamic acid tert-butyl ester. MS: (M+H)⁺=282.

### Example 102

### 2-Cyclopropyl-5H-pyrrolo[2,3-b]pyrazine-7-carboxylic acid [(R)-cyclopropyl-(1-hydroxy-cyclopentyl)-methyl]-amide

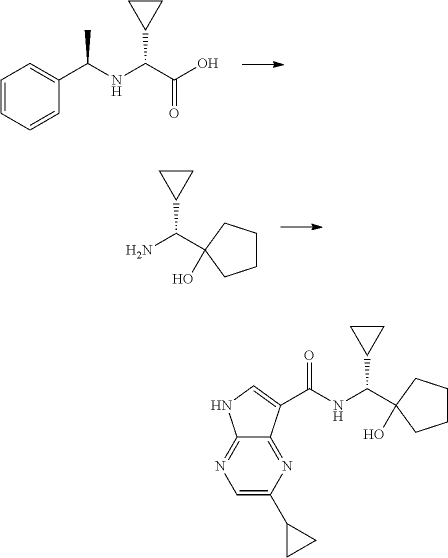 [SMILES: C[C@@H](N[C@@H](C(=O)O)C1CC1)c1ccccc1 ; N[C@H](C1CC1)C1(O)CCCC1 ; O=C(N[C@H](C1CC1)C1(O)CCCC1)c1c[nH]c2ncc(C3CC3)nc12]

### Step 1

(R)-Cyclopropyl-((R)-1-phenyl-ethylamino)-acetic acid was prepared from cyclopropane carboxaldehyde following the procedure outlined in U.S. Pat. No. 6,191,306.

### Step 2

To a suspension of (R)-cyclopropyl-((R)-1-phenyl-ethylamino)-acetic acid (0.50 g, 2.28 mmol) in MeOH (20 mL) at 0° C. was slowly added thionyl chloride (1.66 mL, 22.8 mmol). The homogeneous reaction mixture was stirred at room temperature for 4 h then heated to 60° C. overnight. The reaction was cooled to room temperature and concentrated. The residue was diluted with water and brought to pH=9 with 1.0 M NaOH. After extraction with Et₂O (2×), the organics were dried over MgSO₄ and concentrated to afford 0.37 g (70%) of (R)-cyclopropyl-((R)-1-phenyl-ethylamino)-acetic acid methyl ester as a light brown oil which was used without further purification.

### Step 3

To a solution of (R)-cyclopropyl-((R)-1-phenyl-ethylamino)-acetic acid methyl ester (0.37 g, 1.58 mmol) in THF (12 mL) at 0° C. was slowly added allyl magnesium bromide (1.0 M in Et₂O, 5.5 mL, 5.5 mmol). The resultant white slurry was stirred at 0° C. for 1 h then at room temperature for 3 h. The reaction mixture was cooled to 0° C. and quenched with saturated aqueous NH₄Cl then diluted with H₂O and extracted with EtOAc. The combined organics were washed with H₂O, dried over MgSO₄ and concentrated. The residue was purified by SiO₂ chromatography (10% to 25% EtOAc/hexanes) to afford 0.37 g (82%) of 4-[(R)-cyclopropyl-((R)-1-phenyl-ethylamino)-methyl]-hepta-1,6-dien-4-ol as a colorless oil.

### Step 4

To a solution of 4-[(R)-cyclopropyl-((R)-1-phenyl-ethylamino)-methyl]-hepta-1,6-dien-4-ol (0.37 g, 1.3 mmol) in toluene (40 mL) was added Grubbs 2^{nd} generation catalyst (0.044 g, 0.05 mmol). The maroon reaction mixture was heated at 100° C. overnight. The reaction mixture was concentrated and purified by SiO₂ chromatography (20% to 50% EtOAc/hexanes) to afford 134 mg (40%) of 1-[(R)-cyclopropyl-((R)-1-phenyl-ethylamino)-methyl]-cyclopent-3-enol as a brown oil.

### Step 5

To a solution of 1-[(R)-cyclopropyl-((R)-1-phenyl-ethylamino)-methyl]-cyclopent-3-enol (134 mg, 0.52 mmol) in MeOH (8 mL) was added 20% Pd(OH)₂ on carbon (20 mg). The reaction mixture was stirred under an atmosphere of H₂ (1 atm) overnight then filtered over Celite, rinsing with EtOAc. The filtrate was concentrated to afford 74 mg (90%) of 1-((R)-amino-cyclopropyl-methyl)-cyclopentanol as a light brown oil.

### Step 6

2-Cyclopropyl-5H-pyrrolo[2,3-b]pyrazine-7-carboxylic acid [(R)-cyclopropyl-(1-hydroxy-cyclopentyl)-methyl]-amide was prepared according to the procedure outlined in Example 1, steps 4-5 substituting 1-((R)-amino-cyclopropyl-methyl)-cyclopentanol for 1-((R)-1-amino-ethyl)-cyclopentanol hydrochloride. MS: (M+H)⁺=341; mp=195.0-197.0.

### Example 103

### 2-Cyclopropyl-5H-pyrrolo[2,3-b]pyrazine-7-carboxylic acid ((1S,2S)-2-hydroxy-1,2-dimethyl-butyl)-amide

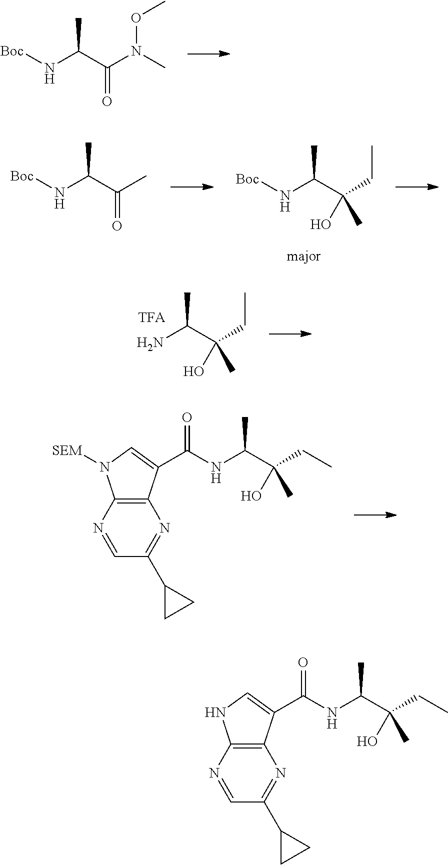

### Step 1

A solution of N-(tert-butoxycarbonyl)-L-alanine-N′-methoxy-N′-methyl amide (5.49 g, 23.64 mmol) in anhydrous THF (100 ml), under argon atmosphere was cooled to −25° C. To this was added a solution of methylmagnesium bromide (22 ml, 66 mmol, 3M in diethyl ether). The mixture was stirred for 1 hour at −25° C. and then warmed to ambient temperature overnight. The mixture was cooled in an ice bath and treated with a 1N hydrochloric acid solution (60 ml, aqueous) via drop-wise addition. Water (60 ml) and ethyl acetate (60 ml) were added and the material was shaken in a separatory funnel. The ethyl acetate phase was collected and washed consecutively with 2×120 ml of water. The aqueous phases were back extracted with ethyl acetate (2×80 mL). The organic phases were combined, dried (magnesium sulfate), filtered and concentrated on the rotovap. The crude material was purified via filtration through a short column of silica gel, eluting with 20% ethyl acetate/hexanes to provide 4.34 g of ((S)-1-methyl-2-oxo-propyl)-carbamic acid tert-butyl ester as a white solid. ¹H NMR (300 MHz, CHLOROFORM-d) δ ppm 1.35 (d, J=7.2 Hz, 3H) 1.44 (s, 9H) 1.61 (s, 3H) 4.28-4.37 (m, 1H) 5.27 (br s, 1H).

### Step 2

To a cold (ice bath, 0° C.) solution of ((S)-1-methyl-2-oxo-propyl)-carbamic acid tert-butyl ester (600 mg, 3.2 mmol) in tetrahydrofuran (20 mL) under argon was added a 1M solution of ethylmagnesium bromide (9.6 ml, 9.6 mmol) in diethyl ether, via slow drop-wise addition. The material was stirred at 0° C. for 20 minutes and then warmed to ambient temperature over night. A 0.5 N solution of hydrochloric acid (60 ml, aqueous) was added together with ethyl acetate (60 ml) and the material was shaken in a separatory funnel. The ethyl acetate phase was collected and washed with brine (60 ml). The aqueous phases were back extracted with ethyl acetate (2×40 ml). The organic phases were combined, dried over magnesium sulfate and filtered. The solvent was stripped and the remainder was filtered through a short plug of silica gel, eluting with 20% ethyl acetate/hexane to provide 630 mg of ((1S,2S)-2-hydroxy-1,2-dimethyl-butyl)-carbamic acid tert-butyl ester as a yellow-brown semi-viscous oil (major diastereomer: 3:1 mixture). (M+H)⁺=218.

### Step 3

To a solution of ((1S,2S)-2-hydroxy-1,2-dimethyl-butyl)-carbamic acid tert-butyl ester (620 mg, 3.2 mmol) in dry methylene chloride (4 ml) was added trifluoroacetic acid (4 ml), via drop-wise addition. The flask was capped and stirred for about 30 minutes. The volatiles were stripped and the resultant was taken up in toluene (25 ml) and the solvent was again stripped on the rotovap. This was repeated once more and the remainder was evacuated on a mechanical pump to provide the product (2S,3S)-2-amino-3-methyl-pentan-3-ol trifluoroacetate as a viscous brown-red oil, which was used without further purification in the next step.

### Step 4

The (2S,3S)-2-amino-3-methyl-pentan-3-ol trifluoroacetate from step 3 was reacted with 2-cyclopropyl-5-(2-trimethylsilanyl-ethoxymethyl)-5H-pyrrolo[2,3-b]pyrazine-7-carboxylic acid under the conditions shown for example 1, step 4 to provide 2-cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-butyl)-amide.

### Step 5

The 2-cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-butyl)-amide from step 4 was deprotected under the conditions described for Example 1, step 5 to provide 2-cyclopropyl-5H-pyrrolo[2,3-b]pyrazine-7-carboxylic acid ((1S,2S)-2-hydroxy-1,2-dimethyl-butyl)-amide as a white crystalline solid. MS: (M+H)⁺=303; mp=243.0-245.0.

### Example 104

### 2-Cyclopropyl-5H-pyrrolo[2,3-b]pyrazine-7-carboxylic acid ((1S,2R)-2-hydroxy-1,2-dimethyl-butyl)-amide

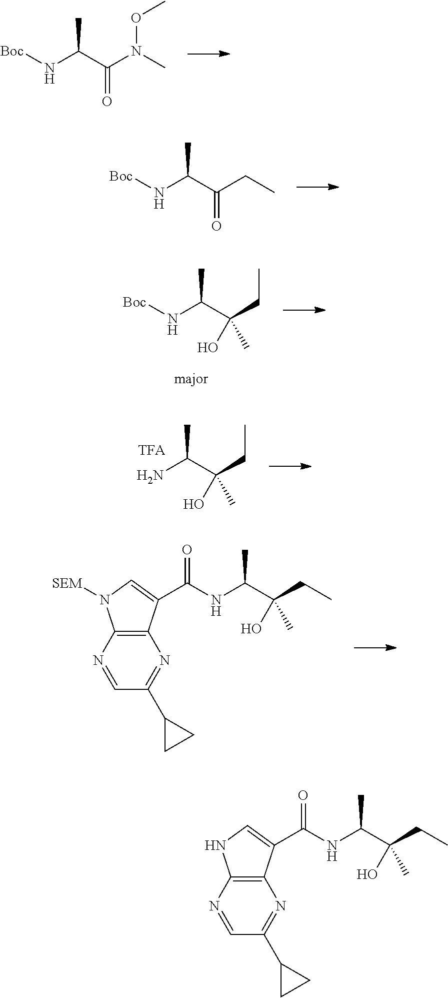

Prepared according to the procedure outlined in Example 103 substituting ethyl magnesium bromide for methyl magnesium bromide in step 1 and methyl magnesium bromide for ethyl magnesium bromide in step 2. The product of step 2 was a 3:2 mixture of diastereomers favoring the desired (1S,2R) configuration. The final product contained 18% of the (1S,2S) diasteromer. MS: (M+H)⁺=303; mp=262.0-264.0.

### Example 105

### 2-Cyclopropyl-5H-pyrrolo[2,3-b]pyrazine-7-carboxylic acid ((1S,2S)-3-cyclopropyl-2-hydroxy-1,2-dimethyl-propyl)-amide

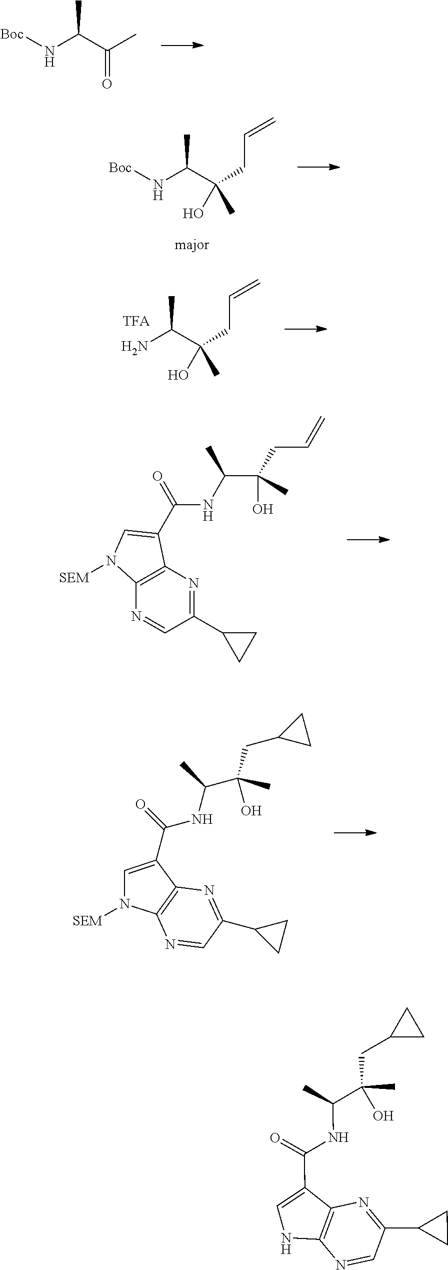

### Step 1

((1S,2S)-2-hydroxy-1,2-dimethyl-pent-4-enyl)-carbamic acid tert-butyl ester was prepared according to the procedure outlined in Example 103, step 2 substituting allyl magnesium bromide for ethyl magnesium bromide.

### Step 2

(2S,3S)-2-amino-3-methylhex-5-en-3-ol trifluoroacetate was prepared according to the procedure outlined in Example 103, step 3 substituting ((1S,2S)-2-hydroxy-1,2-dimethyl-pent-4-enyl)-carbamic acid tert-butyl ester for ((1S,2S)-2-hydroxy-1,2-dimethyl-butyl)-carbamic acid tert-butyl ester.

### Step 3

2-Cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-pent-4-enyl)-amide was prepared according to the procedure outlined in Example 103, step 4 substituting (2S,3S)-2-amino-3-methylhex-5-en-3-ol trifluoroacetate for (2S,3S)-2-amino-3-methyl-pentan-3-ol trifluoroacetate.

### Step 4

To a solution of 2-cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-pent-4-enyl)-amide (98 mg, 0.23 mmol) in tetrahydrofuran (2 mL) and diethyl ether (0.5 mL) was added palladium acetate (5 mg. catalytic) and the mixture was cooled in an ice bath. Diazomethane solution (6-8 mL, 0.5 M in ether) was added drop-wise and the material was left for 30 minutes, with cooling and occasional agitation. Additional diazomethane solution (4 mL) was added, with occasional agitation. After 10 minutes the material was filtered through a plug of celite, rinsing well with ethyl acetate. The volatiles were evaporated to provide 132 mg of crude 2-cyclopropyl-5-(2-trimethylsilanyl-ethoxymethyl)-5H-pyrrolo[2,3-b]pyrazine-7-carboxylic acid ((1S,2S)-3-cyclopropyl-2-hydroxy-1,2-dimethyl-propyl)-amide, which was used in the next step without further purification.

### Step 5

2-Cyclopropyl-5H-pyrrolo[2,3-b]pyrazine-7-carboxylic acid ((1S,2S)-3-cyclopropyl-2-hydroxy-1,2-dimethyl-propyl)-amide was prepared according to the procedure outlined in Example 103, step 5 substituting 2-cyclopropyl-5-(2-trimethylsilanyl-ethoxymethyl)-5H-pyrrolo[2,3-b]pyrazine-7-carboxylic acid ((1S,2S)-3-cyclopropyl-2-hydroxy-1,2-dimethyl-propyl)-amide for 2-cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-butyl)-amide. The product was isolated as a light yellow solid. MS: (M+H)⁺=329.

### Example 106

### 2-Cyclopropyl-5H-pyrrolo[2,3-b]pyrazine-7-carboxylic acid ((1S,2R)-3-cyclopropyl-2-hydroxy-1,2-dimethyl-propyl)-amide

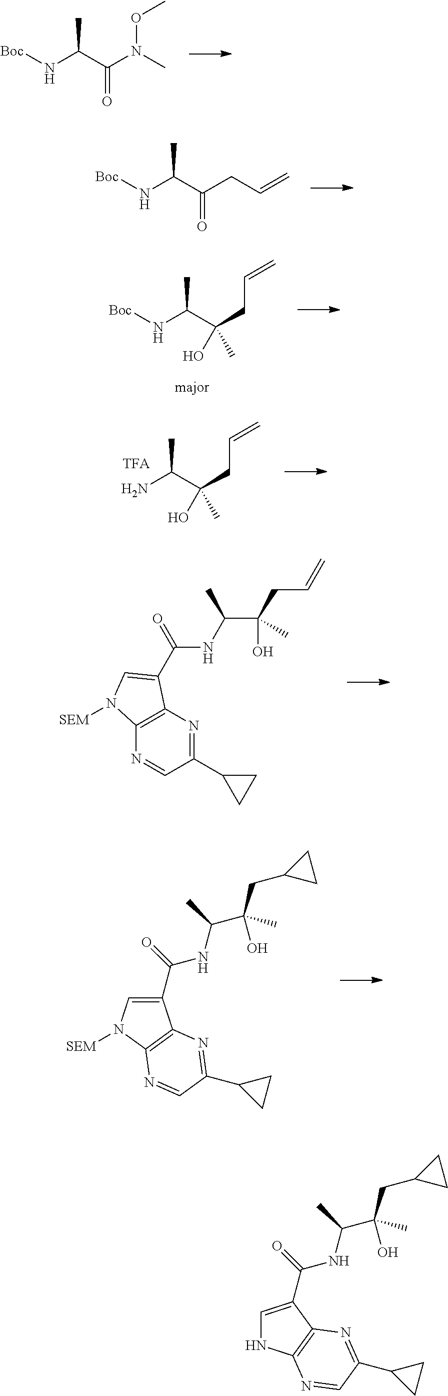

### Step 1

((S)-1-methyl-2-oxo-pent-4-enyl)-carbamic acid tert-butyl ester was prepared according to the procedure outlined in Example 103, step 1 substituting allyl magnesium bromide for methyl magnesium bromide.

### Step 2

((1S,2R)-2-hydroxy-1,2-dimethyl-pent-4-enyl)-carbamic acid tert-butyl ester was prepared according to the procedure outlined in Example 103, step 2 substituting methyl magnesium bromide for ethyl magnesium bromide.

### Step 3

(2S,3R)-2-amino-3-methylhex-5-en-3-ol trifluoroacetate was prepared according to the procedure outlined in Example 103, step 3 substituting ((1S,2R)-2-hydroxy-1,2-dimethyl-pent-4-enyl)-carbamic acid tert-butyl ester for ((1S,2S)-2-hydroxy-1,2-dimethyl-butyl)-carbamic acid tert-butyl ester.

### Step 4

2-Cyclopropyl-5-(2-trimethylsilanyl-ethoxymethyl)-5H-pyrrolo[2,3-b]pyrazine-7-carboxylic acid ((1S,2R)-2-hydroxy-1,2-dimethyl-pent-4-enyl)-amide was prepared according to the procedure outlined in Example 103, step 4 substituting (2S,3R)-2-amino-3-methylhex-5-en-3-ol trifluoroacetate for (2S,3S)-2-amino-3-methyl-pentan-3-ol trifluoroacetate.

### Step 5

2-Cyclopropyl-5-(2-trimethylsilanyl-ethoxymethyl)-5H-pyrrolo[2,3-b]pyrazine-7-carboxylic acid ((1S,2R)-3-cyclopropyl-2-hydroxy-1,2-dimethyl-propyl)-amide was prepared according to the procedure outlined in Example 105, step 4 substituting 2-cyclopropyl-5-(2-trimethylsilanyl-ethoxymethyl)-5H-pyrrolo[2,3-b]pyrazine-7-carboxylic acid ((1S,2R)-2-hydroxy-1,2-dimethyl-pent-4-enyl)-amide for 2-cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-pent-4-enyl)-amide.

### Step 6

2-Cyclopropyl-5H-pyrrolo[2,3-b]pyrazine-7-carboxylic acid ((1S,2R)-3-cyclopropyl-2-hydroxy-1,2-dimethyl-propyl)-amide was prepared according to the procedure outlined in Example 103, step 5 substituting 2-cyclopropyl-5-(2-trimethylsilanyl-ethoxymethyl)-5H-pyrrolo[2,3-b]pyrazine-7-carboxylic acid ((1S,2R)-3-cyclopropyl-2-hydroxy-1,2-dimethyl-propyl)-amide for 2-cyclopropyl-5-(2-trimethylsilanyl-ethoxymethyl)-5H-pyrrolo[2,3-b]pyrazine-7-carboxylic acid ((1S,2S)-2-hydroxy-1,2-dimethyl-butyl)-amide. MS: (M+H)⁺=329.

### Example 107

### 2-Cyclopropyl-5H-pyrrolo[2,3-b]pyrazine-7-carboxylic acid ((1S,2R)-3,3,3-trifluoro-2-hydroxy-1,2-dimethyl-propyl)-amide

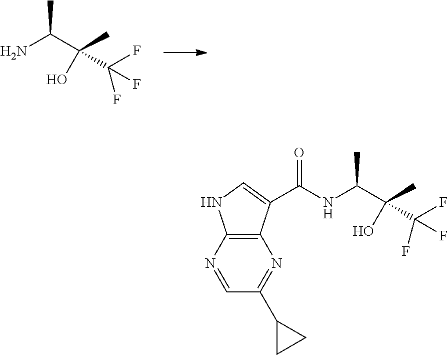

(2R,3S)-3-amino-1,1,1-trifluoro-2-methyl-butan-2-ol was prepared from (S)-2-dibenzylamino-propionaldehyde according to the procedure of Andres, J. M.; Pedrosa, R.; Perez-Encabo, A. Eur. J. Org. Chem. 2004, 1558-1566 and references therein. 2-Cyclopropyl-5H-pyrrolo[2,3-b]pyrazine-7-carboxylic acid ((1S,2R)-3,3,3-trifluoro-2-hydroxy-1,2-dimethyl-propyl)-amide was prepared according to the procedure outlined in Example 1, steps 4-5 substituting (2R,3S)-3-amino-1,1,1-trifluoro-2-methyl-butan-2-ol for 1-((R)-1-amino-ethyl)-cyclopentanol hydrochloride. MS: (M+H)⁺=343; mp=280.0-283.0.

### Example 108

### 2-Cyclopropyl-5H-pyrrolo[2,3-b]pyrazine-7-carboxylic acid ((1S,2S)-3,3,3-trifluoro-2-hydroxy-1,2-dimethyl-propyl)-amide

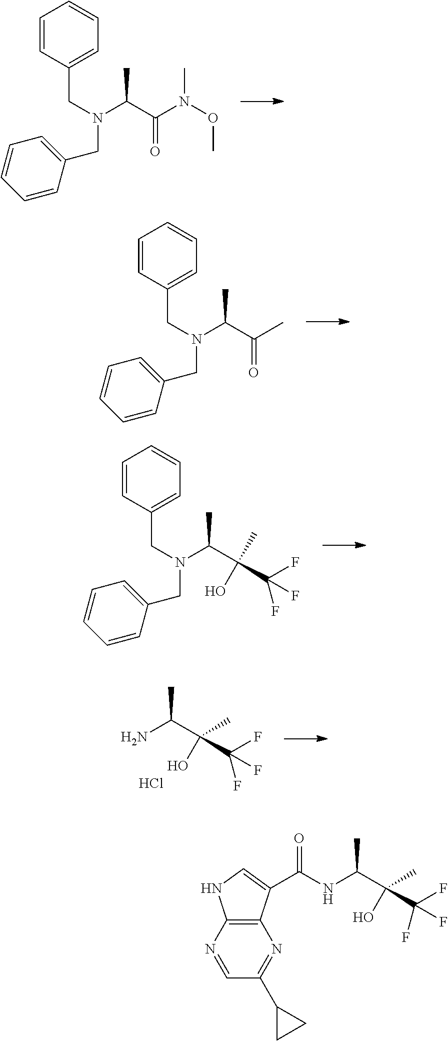

### Step 1

(S)-2-dibenzylamino-N-methoxy-N-methyl-propionamide (492 mg, 1.57 mmol) [synthesis described in: Josop Bonjoch et al., Tetrahedron 2006, 62, 9166-9173] was dissolved in dry tetrahydrofuran (10 mL) at 0° C. (ice bath) under argon atmosphere. A 3 M solution of methylmagnesium bromide (2.1 mL, 6.3 mmol) was added via drop-wise addition and the reaction mixture was stirred for 3 hours at 0° C. A solution of saturated ammonium chloride (20 mL, aqueous) was added followed by the addition of water (40 mL) and ethyl acetate (60 mL). The mixture was transferred to a separatory funnel and shaken. The ethyl acetate phase was collected and washed with brine (60 mL). The aqueous phases were back extracted with ethyl acetate (2×40 mL), dried from magnesium sulfate, filtered and stripped to provide a crude product. The residue was taken up in methylene chloride and filtered through a short column of silica gel, providing 401 mg of (S)-3-dibenzylamino-butan-2-one as a yellow-brown mobile oil. (M+H)⁺=268.

### Step 2

To a solution of (S)-3-dibenzylamino-butan-2-one (400 mg, 1.5 mmol) in dry tetrahydrofuran (7 mL) was added a solution of tetra-N-butylammonium fluoride (0.08 mL, 1.0 M in THF) and the reaction mixture was cooled to 0° C. (ice bath) under argon atmosphere. Added trimethyl(trifluoromethyl)silane (0.35 mL, 2.25 mmol), via drop-wise addition and stirred for 30 minutes at 0° C. A solution of saturated ammonium chloride (20 mL, aqueous) was added and most of the solvent was stripped on the rotary evaporator. The remainder was taken up ether (40 mL) and water (40 mL) and transferred to a separatory funnel. The mixture was shaken and the ether phase collected and washed with brine. The aqueous phases were back extracted with ether (2×30 mL), combined, dried from magnesium sulfate, filtered and stripped to provide a crude silyl ether intermediate. The material was purified by preparative TLC (use 2 plates, elute with 30% ethyl acetate/hexanes), to provide a semi-mobile oil (462 mg). This material was taken up in dry tetrahydrofuran (5 mL) and a solution of tetra-N-butylammonium fluoride (0.4 mL, 1.0 M in THF) was added. The material was stirred for 1 hour and then worked up as described above. The crude product was taken up in dichloromethane and filtered through a short plug of silica. The solvent was stripped providing 402 mg of the desired (2S,3S)-3-dibenzylamino-1,1,1-trifluoro-2-methyl-butan-2-ol. as a clear semi-viscous oil. (M+H)⁺=338.

### Step 3

(2S,3S)-3-dibenzylamino-1,1,1-trifluoro-2-methyl-butan-2-ol (130 mg, 0.42 mmol) was dissolved in methanol (4 ml) and Pearlmann's catalyst (40 mg) was added. The flask was evacuated and place under a hydrogen balloon. The mixture was stirred over night and then filtered through a plug of celite, rinsing well with methanol. To this material was added a solution of hydrochloric acid (1.5 ml, approximately 50% in ethanol). The solvent was stripped on the rotary evaporator providing (2S,3S)-3-amino-1,1,1-trifluoro-2-methyl-butan-2-ol hydrochloride as an off-white semi-solid (82 mg), which was used without further purification.

### Step 4

2-Cyclopropyl-5H-pyrrolo[2,3-b]pyrazine-7-carboxylic acid ((1S,2S)-3,3,3-trifluoro-2-hydroxy-1,2-dimethyl-propyl)-amide was prepared according to the procedure outlined in Example 1, steps 4-5 substituting (2S,3S)-3-amino-1,1,1-trifluoro-2-methyl-butan-2-ol hydrochloride for 1-((R)-1-amino-ethyl)-cyclopentanol hydrochloride. MS: (M+H)⁺=343; mp=290.0-292.0.

### Example 109

### 2-Cyclopropyl-5H-pyrrolo[2,3-b]pyrazine-7-carboxylic acid ((1R,2R)-2-hydroxy-1,2-dimethyl-butyl)-amide

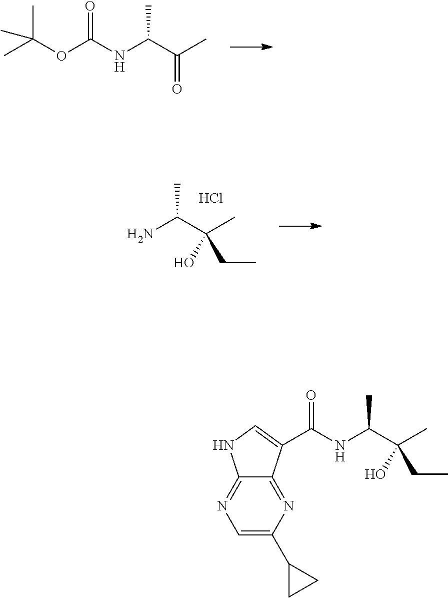

Prepared according to the procedure outlined in Example 103 substituting N-(tert-butoxycarbonyl)-D-alanine-N′-methoxy-N′-methyl amide for N-(tert-butoxycarbonyl)-L-alanine-N′-methoxy-N′-methyl amide in step 1. The product of step 2 was a 4:1 mixture of diastereomers favoring the desired (1R,2R) configuration. In step 3, HCl/MeOH was used instead of TFA for Boc group deprotection. After step 4 the diastereomers were separated using preparative HPLC. MS: (M+H)⁺=303; mp=245.0-247.0.

### Example 110

### 2-Cyclopropyl-5H-pyrrolo[2,3-b]pyrazine-7-carboxylic acid ((1R,2R)-2-hydroxy-1,2-dimethyl-pentyl)-amide

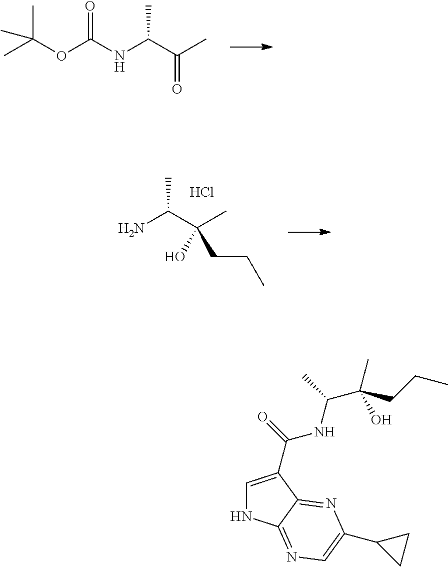

Prepared according to the procedure outlined in Example 103 substituting N-(tert-butoxycarbonyl)-D-alanine-N′-methoxy-N′-methyl amide for N-(tert-butoxycarbonyl)-L-alanine-N′-methoxy-N′-methyl amide in step 1 and propyl magnesium chloride for ethyl magnesium bromide in step 2. The product of step 2 was a 4:1 mixture of diastereomers favoring the desired (1R,2R) configuration. In step 3, HCl/MeOH was used instead of TFA for Boc group deprotection. After step 4 the diastereomers were separated using preparative HPLC. MS: (M+H)⁺=317; mp=222.0-224.0.

### Example 111

### 2-Cyclopropyl-5H-pyrrolo[2,3-b]pyrazine-7-carboxylic acid ((1R,2R)-3-cyano-2-hydroxy-1,2-dimethyl-propyl)-amide

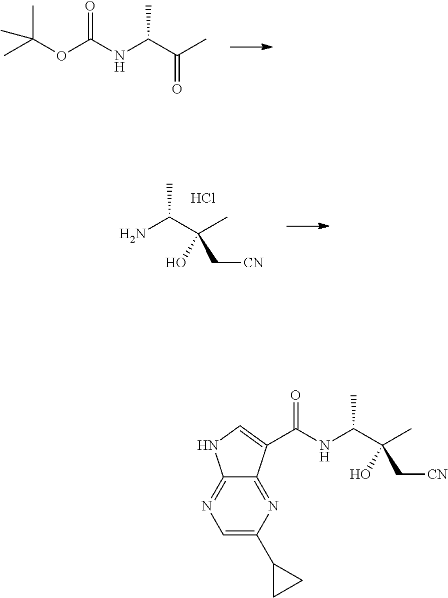

### Step 1

((R)-1-Methyl-2-oxo-propyl)-carbamic acid tert-butyl ester was prepared according to the procedure outlined in Example 21, step 1 substituting N-(tert-butoxycarbonyl)-D-alanine-N′-methoxy-N′-methyl amide for N-(tert-butoxycarbonyl)-L-alanine-N′-methoxy-N′-methyl amide.

### Step 2

To a solution of acetonitrile (0.50 mL, 9.5 mmol) in THF (25 mL) at −78° C. was added lithium bis(trimethylsilyl)amide (1.0 M in THF, 9.5 mL, 9.5 mmol). The reaction mixture was stirred at −78° C. for 30 min then a solution of ((R)-1-methyl-2-oxo-propyl)-carbamic acid tert-butyl ester (400 mg, 2.1 mmol) in THF (5 mL) was added dropwise. The reaction mixture was stirred at −78° C. for 2 h then quenched with sat'd aqueous NH₄Cl and warmed to room temperature. The mixture was diluted with water and extracted with EtOAc (2×). The combined organic layers were washed with water and brine then dried over sodium sulfate and concentrated. The residue was purified by chromatography over 24 g of SiO₂ eluting with 0% to 40% EtOAc/hexanes to afford 453 mg (93%) of ((1R,2R)-3-cyano-2-hydroxy-1,2-dimethyl-propyl)-carbamic acid tert-butyl ester as a light yellow oil and having a 95:5 dr as judged by

NMR analysis.

### Step 3

((1R,2R)-3-Cyano-2-hydroxy-1,2-dimethyl-propyl)-carbamic acid tert-butyl ester (180 mg, 0.78 mmol) was dissolved in hydrogen chloride (1.0 in MeOH, 5 mL, 5 mmol). The solution was stirred at room temperature overnight then concentrated to provide 87 mg of (3R,4R)-4-amino-3-hydroxy-3-methyl-pentanenitrile hydrochloride as a brown solid which was used without further purification.

### Step 4

2-Cyclopropyl-5H-pyrrolo[2,3-b]pyrazine-7-carboxylic acid ((1R,2R)-3-cyano-2-hydroxy-1,2-dimethyl-propyl)-amide was prepared according to the procedure outlined in Example 1, steps 4-5 substituting (3R,4R)-4-amino-3-hydroxy-3-methyl-pentanenitrile hydrochloride for 1-((R)-1-amino-ethyl)-cyclopentanol hydrochloride. MS: (M+H)⁺=314; mp=234.0-236.0.

### Example 112

### 2-Cyclopropyl-5H-pyrrolo[2,3-b]pyrazine-7-carboxylic acid cyclohexylmethyl-amide

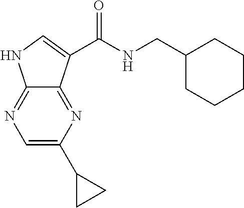 [SMILES: O=C(NCC1CCCCC1)c1c[nH]c2ncc(C3CC3)nc12]

### Step 1

To a solution of 2-cyclopropyl-5-(2-trimethylsilanyl-ethoxymethyl)-5H-pyrrolo[2,3-b]pyrazine-7-carboxylic acid (80 mg, 0.24 mmol) in THF (2 mL) was added 1,1′-carbonyldiimidazole (47 mg, 0.29 mmol). The reaction mixture was stirred at 60° C. for 45 min then cooled to room temperature and cyclohexanemethylamine (0.31 mL, 2.4 mmol) was added. The reaction mixture was stirred at room temperature for 3 h then quenched with water and extracted with EtOAc (2×). The combined organic layers were washed with water and brine then dried over sodium sulfate and concentrated. The residue was purified by chromatography over 8 g of SiO₂ eluting with 0% to 40% EtOAc/hexanes to afford 102 mg (99%) of 2-cyclopropyl-5-(2-trimethylsilanyl-ethoxymethyl)-5H-pyrrolo[2,3-b]pyrazine-7-carboxylic acid cyclohexylmethyl-amide as an off-white solid.

### Step 2

2-Cyclopropyl-5H-pyrrolo[2,3-b]pyrazine-7-carboxylic acid cyclohexylmethyl-amide was prepared according to the procedure outlined in Example 1, step 5 substituting 2-cyclopropyl-5-(2-trimethylsilanyl-ethoxymethyl)-5H-pyrrolo[2,3-b]pyrazine-7-carboxylic acid cyclohexylmethyl-amide for 2-cyclopropyl-5-(2-trimethylsilanylethoxymethyl)-5H-pyrrolo[2,3-b]pyrazine-7-carboxylic acid [(R)-1-(1-hydroxy-cyclopentyl)-ethyl]-amide. MS: (M+H)⁺=299; mp=284.2.0-284.7.

### Example 113

### 2-Cyclopropyl-5H-pyrrolo[2,3-b]pyrazine-7-carboxylic acid (1-methanesulfonyl-piperidin-3-ylmethyl)-amide

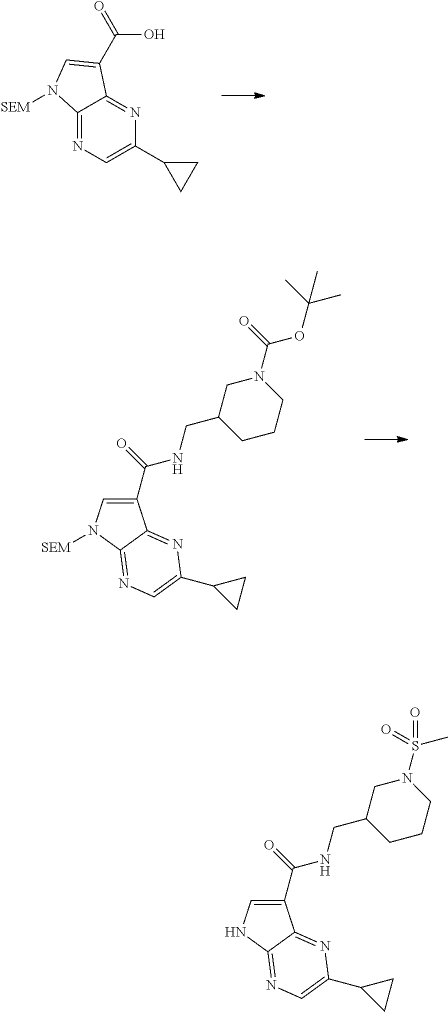 [SMILES: C[Si](C)(C)CCOCn1cc(C(=O)O)c2nc(C3CC3)cnc21 ; CC(C)(C)OC(=O)N1CCCC(CNC(=O)c2cn(COCC[Si](C)(C)C)c3ncc(C4CC4)nc23)C1 ; CS(=O)(=O)N1CCCC(CNC(=O)c2c[nH]c3ncc(C4CC4)nc23)C1]

### Step 1

A 10 mL round-bottomed flask was charged with 2-cyclopropyl-5-((2-(trimethylsilyl)ethoxy)methyl)-5H-pyrrolo[2,3-b]pyrazine-7-carboxylic acid (250 mg, 0.75 mmol), 3-(aminomethyl)-1-N-Boc-piperidine (241 mg, 1.12 mmol), HOBT (111 mg, 0.82 mmol) and EDC (158 mg, 0.82 mmol). Then added DMF (3.3 mL) followed by N,N-diisopropylethylamine (0.20 mL, 1.12 mmol). The yellow reaction mixture was stirred at room temperature overnight then quenched with H₂O (5 mL) and extracted with Et₂O (2×50 mL). The combined organic layers were washed twice with H₂O and once with brine then dried over Na₂SO₄, filtered and concentrated. The residue was purified by chromatography over 24 g SiO₂ using EtOAc/Hexanes (gradient: 0-40% EtOAc) to afford 393 mg (99%) of 3-({[2-cyclopropyl-5-(2-trimethylsilanyl-ethoxymethyl)-5H-pyrrolo[2,3-b]pyrazine-7-carbonyl]-amino}-methyl)-piperidine-1-carboxylic acid tert-butyl ester as a pale yellow oil.

### Step 2

In a 25 mL round-bottomed flask, 3-({[2-cyclopropyl-5-(2-trimethylsilanyl-ethoxymethyl)-5H-pyrrolo[2,3-b]pyrazine-7-carbonyl]-amino}-methyl)-piperidine-1-carboxylic acid tert-butyl ester (0.39 g, 0.74 mmol) was dissolved in MeOH (6.0 mL). The solution was cooled to 0° C. and acetyl chloride (1.05 mL, 14.8 mmol) was added dropwise over 10 min. The ice bath was removed and the reaction mixture was stirred at room temperature for 1.5 h. The solvent was evaporated at room temperature and the residue was dried under high vacuum to afford 339 mg (98%) of 2-cyclopropyl-5-(2-trimethylsilanyl-ethoxymethyl)-5H-pyrrolo[2,3-b]pyrazine-7-carboxylic acid (piperidin-3-ylmethyl)-amide hydrochloride as a light yellow foam.

### Step 3

In a 15 mL round-bottomed flask, 2-cyclopropyl-5-(2-trimethylsilanyl-ethoxymethyl)-5H-pyrrolo[2,3-b]pyrazine-7-carboxylic acid (piperidin-3-ylmethyl)-amide hydrochloride (160 mg, 0.34 mmol) was dissolved in CH₂Cl₂ (3 mL) and cooled to 0° C. Added triethylamine (0.11 mL, 0.75 mmol) followed by methanesulfonyl chloride (0.032 mL, 0.41 mmol). The reaction mixture was stirred at room temperature for 7 h then diluted with 25 mL of CH₂Cl₂ and washed with water (5 mL). The aqueous layer was extracted with CH₂Cl₂ (25 mL) and the combined organic layers were dried over Na₂SO₄, filtered and concentrated. The residue was purified by chromatography over 8 g SiO₂ with EtOAc/Hexanes (gradient: 0-100% EtOAc) to provide 171 mg (98%) of 2-cyclopropyl-5-(2-trimethylsilanyl-ethoxymethyl)-5H-pyrrolo[2,3-b]pyrazine-7-carboxylic acid (1-methanesulfonyl-piperidin-3-ylmethyl)-amide as an off-white foam.

### Step 4

In a 10 mL round-bottomed flask, 2-cyclopropyl-5-(2-trimethylsilanyl-ethoxymethyl)-5H-pyrrolo[2,3-b]pyrazine-7-carboxylic acid (1-methanesulfonyl-piperidin-3-ylmethyl)-amide (159 mg, 0.32 mmol) was dissolved in CH₂Cl₂ (1.3 mL). Trifluoroacetic acid (1.0 mL, 13.0 mmol) was added and the light yellow reaction mixture was stirred at room temperature for 2 h then concentrated. The residue was taken up in toluene (3 mL), concentrated and then dried under high vacuum. The residue was dissolved in CH₂Cl₂ (1.3 mL) and ethylenediamine (1.3 mL, 19.3 mmol) was added. The reaction mixture was stirred at room temperature for 2.5 h then H₂O and EtOAc were added. The resultant suspension was filtered, rinsing with H₂O and EtOAc and dried under high vacuum to afford 59 mg (50%) of 2-cyclopropyl-5H-pyrrolo[2,3-b]pyrazine-7-carboxylic acid (1-methanesulfonyl-piperidin-3-ylmethyl)-amide as a light yellow solid. MS: (M+H)⁺=378; mp=247.6-248.4.

### Example 114

### 2-Cyclopropyl-5H-pyrrolo[2,3-b]pyrazine-7-carboxylic acid (1-acetyl-piperidin-3-ylmethyl)-amide

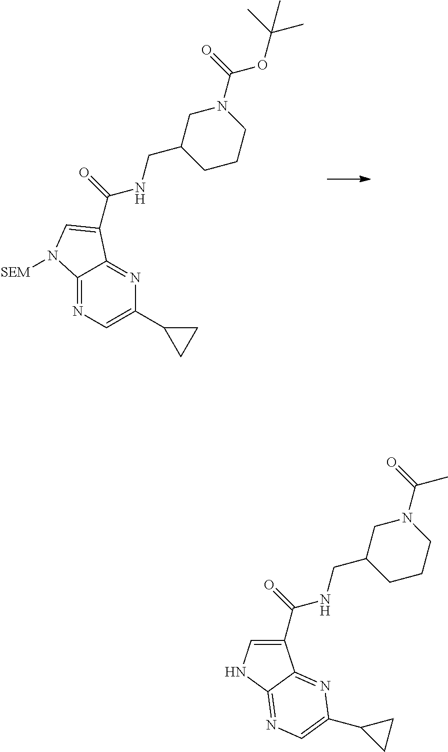 [SMILES: CC(C)(C)OC(=O)N1CCCC(CNC(=O)c2cn(COCC[Si](C)(C)C)c3ncc(C4CC4)nc23)C1 ; CC(=O)N1CCCC(CNC(=O)c2c[nH]c3ncc(C4CC4)nc23)C1]

### Step 1

In a 25 mL round-bottomed flask, 3-({[2-cyclopropyl-5-(2-trimethylsilanyl-ethoxymethyl)-5H-pyrrolo[2,3-b]pyrazine-7-carbonyl]-amino}-methyl)-piperidine-1-carboxylic acid tert-butyl ester (0.39 g, 0.74 mmol) was dissolved in MeOH (6.0 mL). The solution was cooled to 0° C. and acetyl chloride (1.05 mL, 14.8 mmol) was added dropwise over 10 min. The ice bath was removed and the reaction mixture was stirred at room temperature for 1.5 h. The solvent was evaporated at room temperature and the residue was dried under high vacuum to afford 339 mg (98%) of 2-cyclopropyl-5-(2-trimethylsilanyl-ethoxymethyl)-5H-pyrrolo[2,3-b]pyrazine-7-carboxylic acid (piperidin-3-ylmethyl)-amide hydrochloride as a light yellow foam.

### Step 2

In a 15 mL round-bottomed flask, 2-cyclopropyl-5-(2-trimethylsilanyl-ethoxymethyl)-5H-pyrrolo[2,3-b]pyrazine-7-carboxylic acid (piperidin-3-ylmethyl)-amide hydrochloride (175 mg, 0.38 mmol, from Example 31, step 2) was dissolved in CH₂Cl₂ (3 mL) and cooled to 0° C. Added triethylamine (0.12 mL, 0.83 mmol) followed by acetyl chloride (0.032 mL, 0.45 mmol). The reaction mixture was stirred at room temperature for 7.5 h then diluted with 30 mL of CH₂Cl₂ and washed with water (5 mL). The aqueous layer was extracted with CH₂Cl₂ (30 mL) and the combined organic layers were dried over Na₂SO₄, filtered and concentrated. The residue was purified by chromatography over 8 g SiO₂ with EtOAc/Hexanes (gradient: 50-100% EtOAc) then MeOH/EtOAc (gradient: 0-10% MeOH) to provide 159 mg (90%) of 2-cyclopropyl-5-(2-trimethylsilanyl-ethoxymethyl)-5H-pyrrolo[2,3-b]pyrazine-7-carboxylic acid (1-acetyl-piperidin-3-ylmethyl)-amide as a light yellow oil.

### Step 3

2-Cyclopropyl-5H-pyrrolo[2,3-b]pyrazine-7-carboxylic acid (1-acetyl-piperidin-3-ylmethyl)-amide was prepared according to the procedure outlined in Example 113, step 4 substituting 2-cyclopropyl-5-(2-trimethylsilanyl-ethoxymethyl)-5H-pyrrolo[2,3-b]pyrazine-7-carboxylic acid (1-acetyl-piperidin-3-ylmethyl)-amide for 2-cyclopropyl-5-(2-trimethylsilanyl-ethoxymethyl)-5H-pyrrolo[2,3-b]pyrazine-7-carboxylic acid (1-methanesulfonyl-piperidin-3-ylmethyl)-amide. MS: (M+H)⁺=342; mp=198.4-199.1.

### Example 115

### 2-Cyclopropyl-5H-pyrrolo[2,3-b]pyrazine-7-carboxylic acid (1-methanesulfonyl-pyrrolidin-3-ylmethyl)-amide

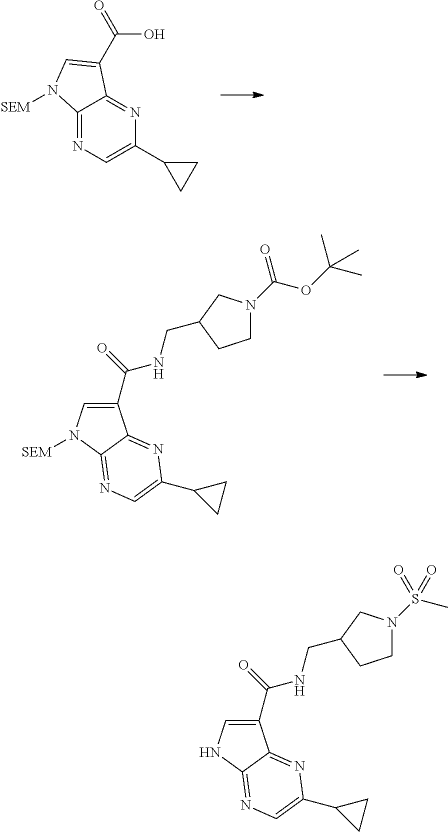 [SMILES: C[Si](C)(C)CCOCn1cc(C(=O)O)c2nc(C3CC3)cnc21 ; CC(C)(C)OC(=O)N1CCC(CNC(=O)c2cn(COCC[Si](C)(C)C)c3ncc(C4CC4)nc23)C1 ; CS(=O)(=O)N1CCC(CNC(=O)c2c[nH]c3ncc(C4CC4)nc23)C1]

### Step 1

A 10 mL round-bottomed flask was charged with 2-cyclopropyl-5-((2-(trimethylsilyl)ethoxy)methyl)-5H-pyrrolo[2,3-b]pyrazine-7-carboxylic acid (260 mg, 0.78 mmol), 3-(aminomethyl)-1-N-Boc-pyrrolidine (234 mg, 1.17 mmol), HOBT (116 mg, 0.86 mmol) and EDC (164 mg, 0.86 mmol). Then added DMF (3.4 mL) followed by N,N-diisopropylethylamine (0.20 mL, 1.12 mmol). The yellow reaction mixture was stirred at room temperature overnight then quenched with H₂O (5 mL) and extracted with Et₂O (2×50 mL). The combined organic layers were washed twice with H₂O and once with brine then dried over Na₂SO₄, filtered and concentrated. The residue was purified by chromatography over 24 g SiO₂ using EtOAc/Hexanes (gradient: 0-50% EtOAc) to afford 339 mg (84%) of 3-({[2-cyclopropyl-5-(2-trimethylsilanyl-ethoxymethyl)-5H-pyrrolo[2,3-b]pyrazine-7-carbonyl]-amino}-methyl)-pyrrolidine-1-carboxylic acid tert-butyl ester as a pale yellow oil.

### Step 2

2-Cyclopropyl-5H-pyrrolo[2,3-b]pyrazine-7-carboxylic acid (1-methanesulfonyl-pyrrolidin-3-ylmethyl)-amide was prepared according to the procedure outlined in Example 113, steps 2-4 substituting 3-({[2-cyclopropyl-5-(2-trimethylsilanyl-ethoxymethyl)-5H-pyrrolo[2,3-b]pyrazine-7-carbonyl]-amino}-methyl)-pyrrolidine-1-carboxylic acid tert-butyl ester for 3-({[2-cyclopropyl-5-(2-trimethylsilanyl-ethoxymethyl)-5H-pyrrolo[2,3-b]pyrazine-7-carbonyl]-amino}-methyl)-piperidine-1-carboxylic acid tert-butyl ester. MS: (M+H)⁺=364; mp=248.0-249.0.

### Example 116

### 2-Cyclopropyl-5H-pyrrolo[2,3-b]pyrazine-7-carboxylic acid (1-acetyl-pyrrolidin-3-ylmethyl)-amide

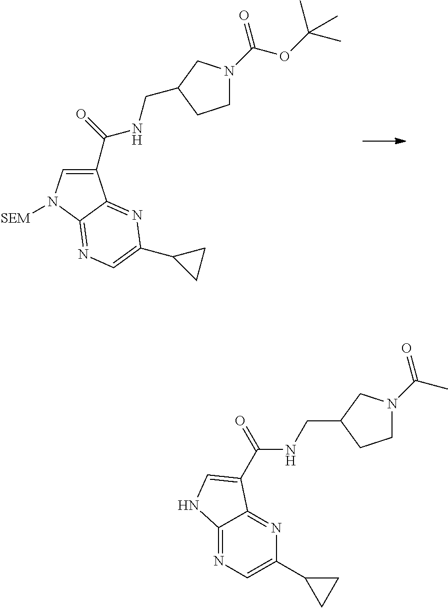 [SMILES: CC(C)(C)OC(=O)N1CCC(CNC(=O)c2cn(COCC[Si](C)(C)C)c3ncc(C4CC4)nc23)C1 ; CC(=O)N1CCC(CNC(=O)c2c[nH]c3ncc(C4CC4)nc23)C1]

Prepared according to the procedure outlined in Example 114, substituting 3-({[2-cyclopropyl-5-(2-trimethylsilanyl-ethoxymethyl)-5H-pyrrolo[2,3-b]pyrazine-7-carbonyl]-amino}-methyl)-pyrrolidine-1-carboxylic acid tert-butyl ester for 3-({[2-cyclopropyl-5-(2-trimethylsilanyl-ethoxymethyl)-5H-pyrrolo[2,3-b]pyrazine-7-carbonyl]-amino}-methyl)-piperidine-1-carboxylic acid tert-butyl ester. MS: (M+H)⁺=328; mp=233.8-235.0.

### Example 117

### 2-Phenoxy-5H-pyrrolo[2,3-b]pyrazine-7-carboxylic acid (1-cyclopropyl-ethyl)-amide

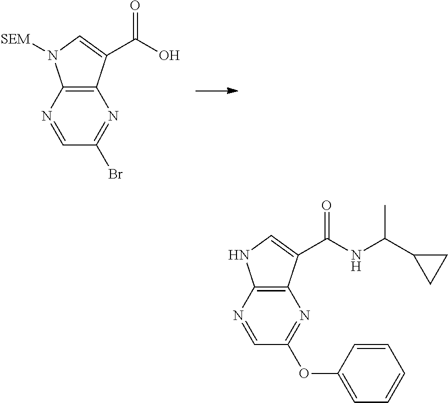 [SMILES: C[Si](C)(C)CCOCn1cc(C(=O)O)c2nc(Br)cnc21 ; CC(NC(=O)c1c[nH]c2ncc(Oc3ccccc3)nc12)C1CC1]

### Step 1

A solution of 2-bromo-5-(2-trimethylsilanyl-ethoxymethyl)-5H-pyrrolo[2,3-b]pyrazine-7-carboxylic acid (0.276 g, 0.741 mmol), 7 mL of dichloromethane, 4-dimethylaminopyridine (0.0850 g, 0.696 mmol), 1-cyclopropylethylamine (0.151 g, 1.77 mmol) and (3-dimethylamino-propyl)-ethyl-carbodiimide (0.285 g, 1.49 mmol) was stirred for 20 h, then concentrated to a yellow oil. The oil was partitioned between 10 mL of ethyl acetate and 10 mL of a 10% citric acid solution, and the organic layer was sequentially washed with 10 mL of water and 10 mL of a sat. aq. NaCl solution; dried over MgSO₄, filtered and concentrated to a yellow oil. Column chromatography (0->33% EtOAc/hexanes) afforded 0.190 g (58%) of 2-bromo-5-(2-trimethylsilanyl-ethoxymethyl)-5H-pyrrolo[2,3-b]pyrazine-7-carboxylic acid (1-cyclopropyl-ethyl)-amide as a white solid.

### Step 2

A mixture of 2-bromo-5-(2-trimethylsilanyl-ethoxymethyl)-5H-pyrrolo[2,3-b]pyrazine-7-carboxylic acid (1-cyclopropyl-ethyl)-amide (0.092 g, 0.210 mmol), phenol (0.0246 g, 0.261 mmol), K₃PO₄ (0.106 g, 0.498 mmol), [2′-(di-tert-butyl-phosphanyl)-biphenyl-2-yl]-dimethyl-amine (0.0036 g, 0.011 mmol), Pd(OAc)₂ (0.0018 g, 0.0080 mmol) and 2 mL of toluene was stirred under nitrogen in a sealed tube at 150° C. for 38 h, then allowed to cool and partitioned between 10 mL of ethyl acetate and 10 mL of water. The aqueous layer was extracted with 10 mL of ethyl acetate, and the combined organic layers were dried over MgSO₄, filtered and concentrated to an orange residue. Column chromatography (0->33% EtOAc/hexanes) afforded 0.047 g (46%) of 2-phenoxy-5-(2-trimethylsilanyl-ethoxymethyl)-5H-pyrrolo[2,3-b]pyrazine-7-carboxylic acid (1-cyclopropyl-ethyl)-amide as a pale yellow oil.

### Step 3

A solution of 2-phenoxy-5-(2-trimethylsilanyl-ethoxymethyl)-5H-pyrrolo[2,3-b]pyrazine-7-carboxylic acid (1-cyclopropyl-ethyl)-amide (0.047 g, 0.105 mmol) in 1 mL of dichloromethane and 1 mL of trifluoroacetic acid was stirred for 2 h, then concentrated, chasing with toluene, to a yellow residue. The residue was treated with 0.6 mL of dichloromethane and 0.6 mL of ethylenediamine. The resulting solution was stirred for 1 h, then partitioned between 10 mL of ethyl acetate and 5 mL of water. The aqueous layer was extracted with 10 mL of ethyl acetate, and the combined organic layers were concentrated to a yellow solid. Column chromatography (20->100% EtOAc/hexanes) afforded 0.024 g (70%) of 2-phenoxy-5H-pyrrolo[2,3-b]pyrazine-7-carboxylic acid (1-cyclopropyl-ethyl)-amide as a light yellow solid. MS: (M+H)⁺=323; mp=242.0-245.0.

### Example 118

### 2-(2,4-Difluoro-phenoxy)-5H-pyrrolo[2,3-b]pyrazine-7-carboxylic acid (1-cyclopropyl-ethyl)-amide

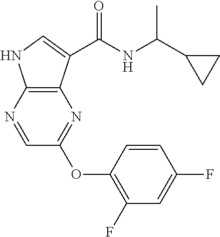 [SMILES: CC(NC(=O)c1c[nH]c2ncc(Oc3ccc(F)cc3F)nc12)C1CC1]

Prepared according to the procedure outlined in Example 117 substituting 2,4-difluorophenol for phenol in step 2. MS: (M+H)⁺=359.

### Example 119

### 2-(4-Fluoro-phenoxy)-5H-pyrrolo[2,3-b]pyrazine-7-carboxylic acid (1-cyclopropyl-ethyl)-amide

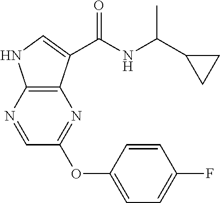 [SMILES: CC(NC(=O)c1c[nH]c2ncc(Oc3ccc(F)cc3)nc12)C1CC1]

Prepared according to the procedure outlined in Example 117 substituting 4-fluorophenol for phenol in step 2. MS: (M+H)⁺=341.

### Example 120

### 2-(2-Fluoro-phenoxy)-5H-pyrrolo[2,3-b]pyrazine-7-carboxylic acid (1-cyclopropyl-ethyl)-amide

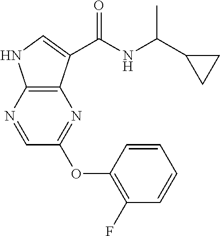 [SMILES: CC(NC(=O)c1c[nH]c2ncc(Oc3ccccc3F)nc12)C1CC1]

Prepared according to the procedure outlined in Example 117 substituting 2-fluorophenol for phenol in step 2. MS: (M+H)⁺=341.

### Example 121

### 2-Phenoxy-5H-pyrrolo[2,3-b]pyrazine-7-carboxylic acid ((R)-2-hydroxy-1,2-dimethyl-propyl)-amide

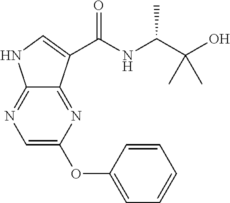 [SMILES: C[C@@H](NC(=O)c1c[nH]c2ncc(Oc3ccccc3)nc12)C(C)(C)O]

Prepared according to the procedure outlined in Example 117, steps 2-3 substituting 2-bromo-5-(2-trimethylsilanyl-ethoxymethyl)-5H-pyrrolo[2,3-b]pyrazine-7-carboxylic acid ((R)-2-hydroxy-1,2-dimethyl-propyl)-amide [from Example 32, step 3] for 2-bromo-5-(2-trimethylsilanyl-ethoxymethyl)-5H-pyrrolo[2,3-b]pyrazine-7-carboxylic acid (1-cyclopropyl-ethyl)-amide. MS: (M+H)⁺=341.

### Example 122

### 2-Phenoxy-5H-pyrrolo[2,3-b]pyrazine-7-carboxylic acid isopropylamide

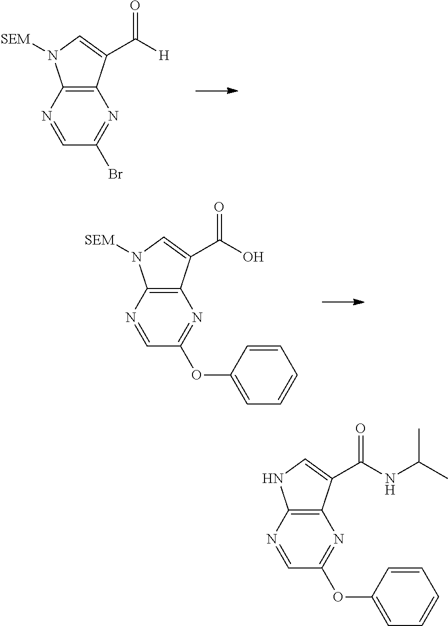 [SMILES: C[Si](C)(C)CCOCn1cc(C=O)c2nc(Br)cnc21 ; C[Si](C)(C)CCOCn1cc(C(=O)O)c2nc(Oc3ccccc3)cnc21 ; CC(C)NC(=O)c1c[nH]c2ncc(Oc3ccccc3)nc12]

### Step 1

A mixture of 2-bromo-5-(2-trimethylsilanyl-ethoxymethyl)-5H-pyrrolo[2,3-b]pyrazine-7-carbaldehyde (3.29 g, 9.23 mmol), phenol (1.04 g, 11.08 mmol), K₃PO₄ (3.92 g, 18.46 mmol), [2′-(di-tert-butyl-phosphanyl)-biphenyl-2-yl]-dimethyl-amine (0.157 g, 0.46 mmol), Pd(OAc)₂ (0.103 g, 0.46 mmol) and degassed toluene (50 mL) was stirred under nitrogen in a sealed tube at 150° C. overnight. The reaction mixture was cooled to room temperature and partitioned between ethyl acetate and water. The aqueous layer was extracted with ethyl acetate, and the combined organic layers were dried over MgSO₄, filtered and concentrated. The residue was purified by SiO₂ column chromatography (0-30% EtOAc/hexanes) to afford 2.09 g (61%) of 2-phenoxy-5-(2-trimethylsilanyl-ethoxymethyl)-5H-pyrrolo[2,3-b]pyrazine-7-carbaldehyde as a beige solid.

### Step 2

A stock solution of Jones reagent (2.67 M) was prepared by carefully adding concentrated H₂SO₄ (2.3 mL) to CrO₃ (2.67 g) then diluting to 10 mL with H₂O. To a solution of 2-phenoxy-5-(2-trimethylsilanyl-ethoxymethyl)-5H-pyrrolo[2,3-b]pyrazine-7-carbaldehyde (2.35 g, 6.37 mmol) in acetone (75 mL) at 0° C. was added Jones reagent (5 mL, 13.4 mmol) dropwise. The reaction mixture was stirred at room temperature for 2 h then quenched with i-PrOH (2 mL), diluted with EtOAc, and filtered over Celite, rinsing with EtOAc. The filtrate was washed with cold water (3×) and brine then dried over MgSO₄ and concentrated. The residue was purified by SiO₂ column chromatography (30-70% EtOAc/hexanes) to provide 1.59 g (65%) of 2-phenoxy-5-(2-trimethylsilanyl-ethoxymethyl)-5H-pyrrolo[2,3-b]pyrazine-7-carboxylic acid as a light yellow solid.

### Step 3

To a solution of 2-phenoxy-5-(2-trimethylsilanyl-ethoxymethyl)-5H-pyrrolo[2,3-b]pyrazine-7-carboxylic acid (0.115 g, 0.30 mmol), 4-dimethylaminopyridine (0.048 g, 0.39 mmol) and (3-dimethylamino-propyl)-ethyl-carbodiimide (0.075 g, 0.39 mmol) in CH₂Cl₂ (2 mL) was added a solution of isopropylamine (0.023 g, 0.39 mmol) in CH₂Cl₂ (0.5 mL). The reaction mixture was stirred at room temperature overnight, then quenched with water and extracted with ethyl acetate (3×). The organic layer was washed with water and saturated aqueous NaCl solution and dried over MgSO₄, filtered and concentrated to afford 2-phenoxy-5-(2-trimethylsilanyl-ethoxymethyl)-5H-pyrrolo[2,3-b]pyrazine-7-carboxylic acid isopropylamide which was used without further purification.

### Step 4

To a solution of 2-phenoxy-5-(2-trimethylsilanyl-ethoxymethyl)-5H-pyrrolo[2,3-b]pyrazine-7-carboxylic acid isopropylamide from Step 3 in dichloromethane (0.7 mL) was added trifluoroacetic acid (0.7 mL). The reaction mixture was stirred at room temperature overnight then concentrated. The residue was stirred with THF (1 mL), water (0.5 mL), and Et₃N (0.5 mL) for 2 h then concentrated. The residue was partitioned between ethyl acetate and water and the aqueous layer was extracted with ethyl acetate. The combined organic layers were dried over MgSO₄ and concentrated. The residue was purified by SiO₂ column chromatography (5% MeOH/CH₂Cl₂) to provide 0.070 g (78%, 2 steps) of 2-phenoxy-5H-pyrrolo[2,3-b]pyrazine-7-carboxylic acid isopropylamide as a yellow solid. MS: (M+H)⁺=297; mp=263.0-265.0.

### Example 123

### 2-Phenoxy-5H-pyrrolo[2,3-b]pyrazine-7-carboxylic acid ((S)-1,2,2-trimethyl-propyl)-amide

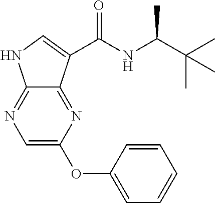 [SMILES: C[C@H](NC(=O)c1c[nH]c2ncc(Oc3ccccc3)nc12)C(C)(C)C]

Prepared according to the procedure outlined in Example 122 substituting (S)-1,2,2-trimethyl-propylamine for isopropylamine in step 3. MS: (M+H)⁺=339; mp=270.0-273.0.

### Example 124

### 2-Phenoxy-5H-pyrrolo[2,3-b]pyrazine-7-carboxylic acid ((S)-sec-butyl)-amide

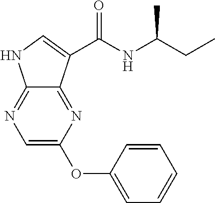 [SMILES: CC[C@H](C)NC(=O)c1c[nH]c2ncc(Oc3ccccc3)nc12]

Prepared according to the procedure outlined in Example 122 substituting (S)-sec-butylamine for isopropylamine in step 3. MS: (M+H)⁺=311; mp=227.0-229.0.

### Example 125

### 2-Phenoxy-5H-pyrrolo[2,3-b]pyrazine-7-carboxylic acid ((S)-1,2-dimethyl-propyl)-amide

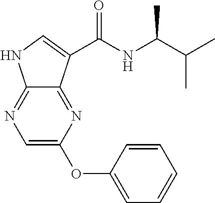 [SMILES: CC(C)[C@H](C)NC(=O)c1c[nH]c2ncc(Oc3ccccc3)nc12]

Prepared according to the procedure outlined in Example 122 substituting (S)-1,2-dimethyl-propylamine for isopropylamine in step 3. MS: (M+H)⁺=325; mp=234.0-235.0.

### Example 126

### 2-Phenoxy-5H-pyrrolo[2,3-b]pyrazine-7-carboxylic acid ((S)-1-cyclohexyl-ethyl)-amide

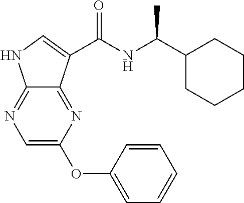 [SMILES: C[C@H](NC(=O)c1c[nH]c2ncc(Oc3ccccc3)nc12)C1CCCCC1]

Prepared according to the procedure outlined in Example 122 substituting (S)-(+)-1-cyclohexylethylamine for isopropylamine in step 3. MS: (M+H)⁺=365; mp=227.0-230.0.

### Example 127

### 2-Phenoxy-5H-pyrrolo[2,3-b]pyrazine-7-carboxylic acid ((S)-2-hydroxy-1,2-dimethyl-propyl)-amide

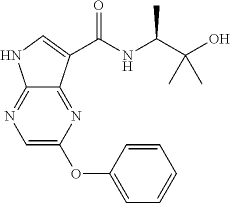 [SMILES: C[C@H](NC(=O)c1c[nH]c2ncc(Oc3ccccc3)nc12)C(C)(C)O]

Prepared according to the procedure outlined in Example 122 substituting (S)-3-amino-2-methyl-butan-2-ol for isopropylamine in step 3. MS: (M+H)⁺=341; mp=232.0-232.0.

### Example 128

### 2-Phenoxy-5H-pyrrolo[2,3-b]pyrazine-7-carboxylic acid ((R)-1-cyclohexyl-ethyl)-amide

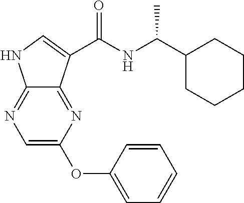 [SMILES: C[C@@H](NC(=O)c1c[nH]c2ncc(Oc3ccccc3)nc12)C1CCCCC1]

Prepared according to the procedure outlined in Example 122 substituting (R)-(−)-1-cyclohexylethylamine for isopropylamine in step 3. MS: (M+H)⁺=365; mp=231.0-232.0.

### Example 129

### 2-Phenoxy-5H-pyrrolo[2,3-b]pyrazine-7-carboxylic acid ((R)-1,2,2-trimethyl-propyl)-amide

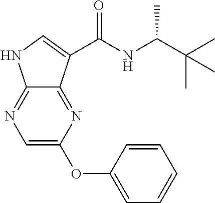 [SMILES: C[C@@H](NC(=O)c1c[nH]c2ncc(Oc3ccccc3)nc12)C(C)(C)C]

Prepared according to the procedure outlined in Example 122 substituting (R)-1,2,2-trimethyl-propylamine for isopropylamine in step 3. MS: (M+H)⁺=339; mp=273.0-274.0.

### Example 130

### 2-Phenoxy-5H-pyrrolo[2,3-b]pyrazine-7-carboxylic acid ethylamide

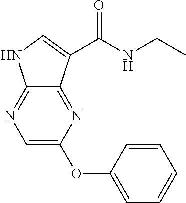 [SMILES: CCNC(=O)c1c[nH]c2ncc(Oc3ccccc3)nc12]

Prepared according to the procedure outlined in Example 122 substituting 70% aqueous ethylamine for isopropylamine in step 3. MS: (M+H)⁺=283; mp=230.0-232.0.

### Example 131

### 2-(2,4-Difluoro-phenoxy)-5H-pyrrolo[2,3-b]pyrazine-7-carboxylic acid isopropylamide

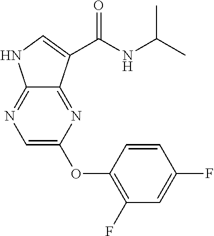 [SMILES: CC(C)NC(=O)c1c[nH]c2ncc(Oc3ccc(F)cc3F)nc12]

Prepared according to the procedure outlined in Example 122 substituting 2,4-difluorophenol for phenol in step 1. MS: (M+H)⁺=333.

### Example 132

### 2-(2,4-Difluoro-phenoxy)-5H-pyrrolo[2,3-b]pyrazine-7-carboxylic acid ((S)-1,2,2-trimethyl-propyl)-amide

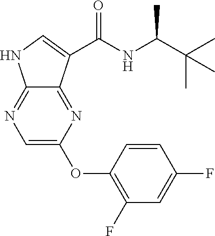 [SMILES: C[C@H](NC(=O)c1c[nH]c2ncc(Oc3ccc(F)cc3F)nc12)C(C)(C)C]

Prepared according to the procedure outlined in Example 122 substituting 2,4-difluorophenol for phenol in step 1 and (S)-1,2,2-trimethyl-propylamine for isopropylamine in step 3. MS: (M+H)⁺=375.

### Example 133

### 2-(2,4-Difluoro-phenoxy)-5H-pyrrolo[2,3-b]pyrazine-7-carboxylic acid ((R)-1,2,2-trimethyl-propyl)-amide

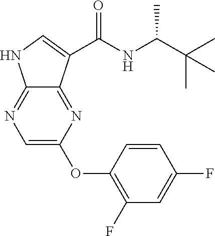 [SMILES: C[C@@H](NC(=O)c1c[nH]c2ncc(Oc3ccc(F)cc3F)nc12)C(C)(C)C]

Prepared according to the procedure outlined in Example 122 substituting 2,4-difluorophenol for phenol in step 1 and (R)-1,2,2-trimethyl-propylamine for isopropylamine in step 3. MS: (M+H)⁺=375.

### Example 134

### 2-(2,4-Difluoro-phenoxy)-5H-pyrrolo[2,3-b]pyrazine-7-carboxylic acid ethylamide

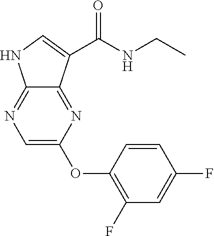 [SMILES: CCNC(=O)c1c[nH]c2ncc(Oc3ccc(F)cc3F)nc12]

Prepared according to the procedure outlined in Example 122 substituting 2,4-difluorophenol for phenol in step 1 and ethylamine for isopropylamine in step 3. MS: (M+H)⁺=319.

### Example 135

### 2-(2,4-Difluoro-phenoxy)-5H-pyrrolo[2,3-b]pyrazine-7-carboxylic acid ((S)-1-cyclohexyl-ethyl)-amide

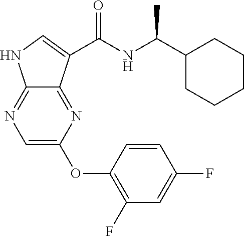 [SMILES: C[C@H](NC(=O)c1c[nH]c2ncc(Oc3ccc(F)cc3F)nc12)C1CCCCC1]

Prepared according to the procedure outlined in Example 122 substituting 2,4-difluorophenol for phenol in step 1 and (S)-(+)-1-cyclohexylethylamine for isopropylamine in step 3. MS: (M+H)⁺=401; mp=233.0-235.0.

### Example 136

### 2-(2,4-Difluoro-phenoxy)-5H-pyrrolo[2,3-b]pyrazine-7-carboxylic acid ((R)-1-cyclohexyl-ethyl)-amide

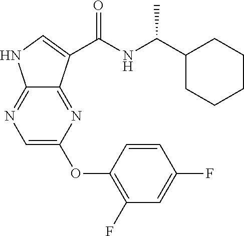 [SMILES: C[C@@H](NC(=O)c1c[nH]c2ncc(Oc3ccc(F)cc3F)nc12)C1CCCCC1]

Prepared according to the procedure outlined in Example 122 substituting 2,4-difluorophenol for phenol in step 1 and (R)-(+)-1-cyclohexylethylamine for isopropylamine in step 3. MS: (M+H)⁺=401; mp=233.0-235.0.

### Example 137

### 2-(2,4-Difluoro-phenoxy)-5H-pyrrolo[2,3-b]pyrazine-7-carboxylic acid ((R)-sec-butyl)-amide

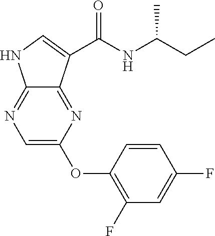 [SMILES: CC[C@@H](C)NC(=O)c1c[nH]c2ncc(Oc3ccc(F)cc3F)nc12]

Prepared according to the procedure outlined in Example 122 substituting 2,4-difluorophenol for phenol in step 1 and (R)-sec-butylamine for isopropylamine in step 3. MS: (M+H)⁺=347; mp=246.0-248.0.

### Example 138

### 2-(2,4-Difluoro-phenoxy)-5H-pyrrolo[2,3-b]pyrazine-7-carboxylic acid ((S)-2-hydroxy-1,2-dimethyl-propyl)-amide

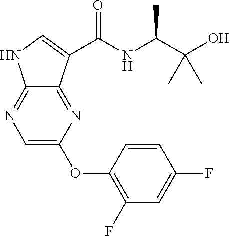 [SMILES: C[C@H](NC(=O)c1c[nH]c2ncc(Oc3ccc(F)cc3F)nc12)C(C)(C)O]

Prepared according to the procedure outlined in Example 122 substituting 2,4-difluorophenol for phenol in step 1 and (S)-3-amino-2-methyl-butan-2-ol for isopropylamine in step 3. MS: (M+H)⁺=377; mp=224.0-226.0.

### Example 139

### 2-(2,4-Difluoro-phenoxy)-5H-pyrrolo[2,3-b]pyrazine-7-carboxylic acid ((R)-1,2-dimethyl-propyl)-amide

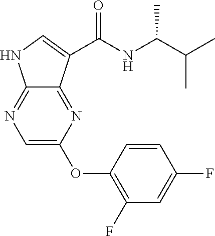 [SMILES: CC(C)[C@@H](C)NC(=O)c1c[nH]c2ncc(Oc3ccc(F)cc3F)nc12]

Prepared according to the procedure outlined in Example 122 substituting 2,4-difluorophenol for phenol in step 1 and (R)-1,2-dimethyl-propylamine for isopropylamine in step 3. MS: (M+H)⁺=361; mp=235.0-237.0.

### Example 140

### 2-(1-Ethyl-1H-pyrazol-4-yl)-5H-pyrrolo[2,3-b]pyrazine-7-carboxylic acid [(S)-1-(1-hydroxy-cyclopentyl)-ethyl]-amide

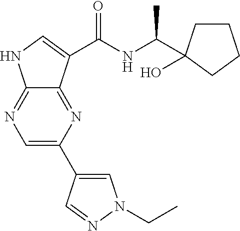 [SMILES: CCn1cc(-c2cnc3[nH]cc(C(=O)N[C@@H](C)C4(O)CCCC4)c3n2)cn1]

Prepared according to the procedure outlined in Example 1, substituting Boc-L-alanine methyl ester for Boc-D-alanine methyl ester in step 1 and 2-(1-ethyl-1H-pyrazol-4-yl)-5-(2-trimethylsilanyl-ethoxymethyl)-5H-pyrrolo[2,3-b]pyrazine-7-carboxylic acid for 2-cyclopropyl-5-(2-trimethylsilanyl-ethoxymethyl)-5H-pyrrolo[2,3-b]pyrazine-7-carboxylic acid in step 4. MS: (M+H)⁺=369.

### Example 141

### 2-Cyclopropyl-5H-pyrrolo[2,3-b]pyrazine-7-carboxylic acid ((S)-2-cyano-1,2,2-trimethyl-ethyl)-amide

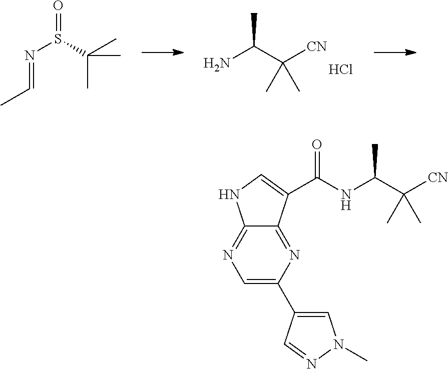

### Step 1

In a flask (R)-2-methyl-propane-2-sulfinic acid amide (4.00 g, 33.0 mmol) was dissolved in CH₂Cl₂ (14.0 mL). Acetaldehyde (16.7 mL, 297 mmol), MgSO₄ (11.9 g, 99.0 mmol) and pyridinium tosylate (415 mg, 1.65 mmol) were added. The reaction mixture was stirred overnight at room temperature, filtered and concentrated to give 5.21 g of (R)-2-methyl-propane-2-sulfinic acid (E)-ethylideneamide as a yellow oil which was used without further purification.

### Step 2

In a flask, isobutyronitrile (6.39 g, 92.4 mmol) was dissolved in diethyl ether (190 mL) and cooled at −78° C. NaHMDS (1.0 M in THF, 99.0 mL, 99.0 mmol) was added and the mixture stirred for 30 min at −78° C. A solution of (R)-2-methyl-propane-2-sulfinic acid (E)-ethylideneamide (crude from step 1, 5.21 g, 33.0 mmol) in THF (50.0 mL) was slowly added. The mixture was stirred at −78° C. for 2 h then allowed to warm to room temperature overnight. The reaction mixture was quenched with saturated aqueous ammonium chloride and extracted with EtOAc. The combined organics were washed with brine, dried over MgSO₄ and concentrated. The residue was purified by SiO₂ chromatography (20-100% EtOAc/hexane) to afford 2.93 g (41%) (R)-2-methyl-propane-2-sulfinic acid ((S)-2-cyano-1,2,2-trimethyl-ethyl)-amide as a light yellow oil.

### Step 3

(R)-2-Methyl-propane-2-sulfinic acid (2-cyano-1,2,2-trimethyl-ethyl)-amide (2.93 g, 13.6 mmol) was dissolved in MeOH and HCl (4.0 M in 1,4-dioxane, 6.8 mL, 27.2 mmol) was added. The reaction mixture was stirred at room temperature for 1 h then concentrated to give 1.90 g (94%) of (S)-3-amino-2,2-dimethyl-butyronitrile hydrochloride as a white solid which was used without further purification.

### Step 4

2-(1-Methyl-1H-pyrazol-4-yl)-5-(2-trimethylsilanyl-ethoxymethyl)-5H-pyrrolo[2,3-b]pyrazine-7-carboxylic acid was prepared according to the Procedure 4 substituting 1-methyl-4-(4,4,5,5-tetramethyl-1,3,2-dioxaborolan-2-yl)-1H-pyrazole for 1-ethyl-4-(4,4,5,5-tetramethyl-1,3,2-dioxaborolan-2-yl)-1H-pyrazole in step 1.

### Step 5

In a flask were combined 2-(1-methyl-1H-pyrazol-4-yl)-5-(2-trimethylsilanyl-ethoxymethyl)-5H-pyrrolo[2,3-b]pyrazine-7-carboxylic acid (120 mg, 0.32 mmol), (S)-3-amino-2,2-dimethyl-butyronitrile hydrochloride (72 mg, 0.48 mmol), EDC (142 mg, 0.74 mmol) and HOBt (125 mg, 0.74 mmol). DMF (4.0 mL) was added followed by i-Pr₂NEt (0.39 mL, 2.25 mmol). The reaction mixture was stirred at room temperature for 18 h and then quenched with water and extracted with EtOAc. The organics were washed with 10% citric acid, sat'd NaHCO₃, sat'd LiCl, and sat'd NaCl then dried over MgSO₄ and concentrated. The residue was purified by SiO₂ chromatography (50-100% EtOAc/hexane) to give 150 mg (99%) 2-(1-methyl-1H-pyrazol-4-yl)-5-(2-trimethylsilanyl-ethoxymethyl)-5H-pyrrolo[2,3-b]pyrazine-7-carboxylic acid ((S)-2-cyano-1,2,2-trimethyl-ethyl)-amide as a pale yellow viscous oil.

### Step 6

In a flask 2-(1-methyl-1H-pyrazol-4-yl)-5-(2-trimethylsilanyl-ethoxymethyl)-5H-pyrrolo[2,3-b]pyrazine-7-carboxylic acid ((S)-2-cyano-1,2,2-trimethyl-ethyl)-amide (150 mg, 0.32 mmol) was dissolved in CH₂Cl₂ (2.25 mL) and TFA (0.75 mL) added. The reaction mixture was stirred for 2 h and concentrated. The residue was dissolved in CH₂Cl₂/MeOH/NH₄OH (60:10:1) (3 mL) and stirred at room temperature overnight. The reaction mixture was then concentrated and the residue purified by SiO₂ chromatography (0-10% MeOH/CH₂Cl₂) to afford 72 mg (67%) of 2-(1-methyl-1H-pyrazol-4-yl)-5H-pyrrolo[2,3-b]pyrazine-7-carboxylic acid ((S)-2-cyano-1,2,2-trimethyl-ethyl)-amide as a white powder. MS: (M+H)⁺=338.

### Example 142

### 2-(1-Methyl-1H-pyrazol-4-yl)-5H-pyrrolo[2,3-b]pyrazine-7-carboxylic acid ((S)-1-cyclohexyl-ethyl)-amide

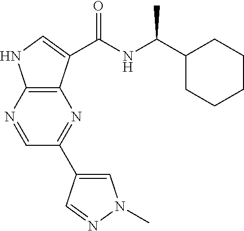 [SMILES: C[C@H](NC(=O)c1c[nH]c2ncc(-c3cnn(C)c3)nc12)C1CCCCC1]

Prepared according to the procedure outlined in Example 1, steps 4-5 substituting (S)-(+)-1-cyclohexylethylamine for 1-((R)-1-amino-ethyl)-cyclopentanol hydrochloride and 2-(1-methyl-1H-pyrazol-4-yl)-5-(2-trimethylsilanyl-ethoxymethyl)-5H-pyrrolo[2,3-b]pyrazine-7-carboxylic acid for 2-cyclopropyl-5-(2-trimethylsilanyl-ethoxymethyl)-5H-pyrrolo[2,3-b]pyrazine-7-carboxylic acid in step 4. MS: (M+H)⁺=353.

### Example 143

### 2-(1-Methyl-1H-pyrazol-4-yl)-5H-pyrrolo[2,3-b]pyrazine-7-carboxylic acid ((S)-1,2,2-trimethyl-propyl)-amide

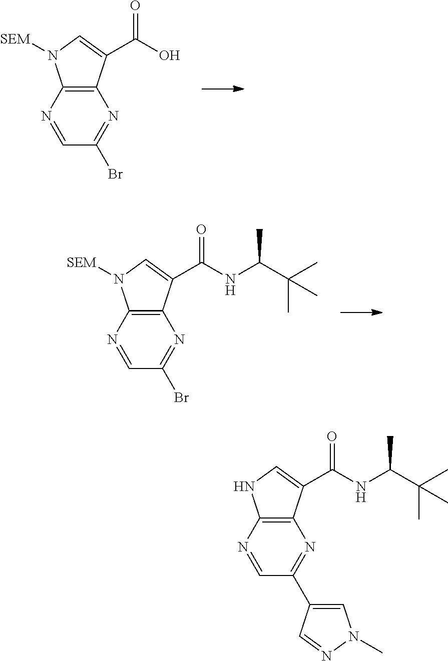 [SMILES: C[Si](C)(C)CCOCn1cc(C(=O)O)c2nc(Br)cnc21 ; C[C@H](NC(=O)c1cn(COCC[Si](C)(C)C)c2ncc(Br)nc12)C(C)(C)C ; C[C@H](NC(=O)c1c[nH]c2ncc(-c3cnn(C)c3)nc12)C(C)(C)C]

### Step 1

2-Bromo-5-(2-trimethylsilanyl-ethoxymethyl)-5H-pyrrolo[2,3-b]pyrazine-7-carboxylic acid (1.5 g, 4.8 mmol) was partly dissolved in dichloromethane (40 mL). 1-Ethyl-3-(3-(dimethylamino)propyl)carbodiimide (1.54 g, 8.06 mmol), 4-dimethylaminopyridine (0.49 g, 4 mmol), N,N-diisopropylethylamine (1.4 mL, 8.06 mmol), and then (S)-3,3-dimethylbutan-2-amine (0.49 g, 4.8 mmol) were added and the reaction was stirred for 16 h. The reaction mixture was diluted with HCl solution and the aqueous layer was extracted twice with dichloromethane. The combined organic layers were washed with sodium bicarbonate solution, dried over sodium sulfate and concentrated. The residue was purified by silica gel chromatography (ethyl acetate/hexanes) to give 1.23 g (67%) of 2-bromo-5-(2-trimethylsilanyl-ethoxymethyl)-5H-pyrrolo[2,3-b]pyrazine-7-carboxylic acid ((S)-1,2,2-trimethyl-propyl)-amide.

### Step 2

In microwave vial, a mixture of 1,4-dioxane (1.8 mL) and water (0.4 mL) was purged with argon gas. 2-Bromo-5-(2-trimethylsilanyl-ethoxymethyl)-5H-pyrrolo[2,3-b]pyrazine-7-carboxylic acid ((S)-1,2,2-trimethyl-propyl)-amide (100 mg, 0.22 mmol), 1-methyl-4-(4,4,5,5-tetramethyl-[1,3,2]dioxaborolan-2-yl)-1H-pyrazole (50 mg, 0.24 mmol), palladium tetrakis(triphenylphosphine) (12.7 mg, 0.011 mmol), and then potassium carbonate (91 mg, 0.66 mmol) were added. The vial was sealed and heated in a microwave reactor at 140° C. for 1 h. The reaction was cooled and water, sodium bicarbonate solution and ethyl acetate were added. The aqueous layer was extracted twice more with ethyl acetate then the combined organic layers were washed with brine, dried over sodium sulfate and concentrated. The residue was purified by silica gel chromatography (ethyl acetate/hexanes) to yield 89 mg (88%) of 2-(1-methyl-1H-pyrazol-4-yl)-5-(2-trimethylsilanyl-ethoxymethyl)-5H-pyrrolo[2,3-b]pyrazine-7-carboxylic acid ((S)-1,2,2-trimethyl-propyl)-amide.

### Step 3

2-(1-Methyl-1H-pyrazol-4-yl)-5-(2-trimethylsilanyl-ethoxymethyl)-5H-pyrrolo[2,3-b]pyrazine-7-carboxylic acid ((S)-1,2,2-trimethyl-propyl)-amide (87 mg, 0.19 mmol) was dissolved in dichloromethane (1.3 mL) and then stirred in an ice bath. Trifluoroacetic acid (0.6 mL) was added slowly and the ice bath was removed. The reaction was stirred for 3 h then recooled in an ice bath. Sodium bicarbonate solution was added and the mixture was extracted three times with ethyl acetate. The combined organic layers were washed with brine, dried over sodium sulfate and concentrated. The residue was dissolved in absolute ethanol (8 mL) and sodium acetate (313 mg, 3.8 mmol) was added. The reaction mixture was stirred for 20 h at 60° C. then cooled and water and ethyl acetate were added. The aqueous layer was extracted twice more with ethyl acetate then the combined organic layers were washed with brine, dried over sodium sulfate and concentrated. The residue was purified by silica gel chromatography to afford 36 mg (57%) of 2-(1-methyl-1H-pyrazol-4-yl)-5H-pyrrolo[2,3-b]pyrazine-7-carboxylic acid ((S)-1,2,2-trimethyl-propyl)-amide. MS: (M+H)⁺=327; mp=296-297° C.

### Example 144

### 2-Thiophen-2-yl-5H-pyrrolo[2,3-b]pyrazine-7-carboxylic acid ((S)-1,2,2-trimethyl-propyl)-amide

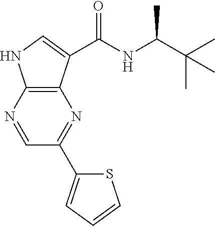 [SMILES: C[C@H](NC(=O)c1c[nH]c2ncc(-c3cccs3)nc12)C(C)(C)C]

Prepared according to the procedure outlined in Example 143, steps 2-3 substituting thiophen-2-ylboronic acid for 1-methyl-4-(4,4,5,5-tetramethyl-[1,3,2]dioxaborolan-2-yl)-1H-pyrazole in Step 2. MS: (M+H)⁺=329; mp=311-312° C.

### Example 145

### 2-(4-Trifluoromethyl-phenyl)-5H-pyrrolo[2,3-b]pyrazine-7-carboxylic acid ((S)-1,2,2-trimethyl-propyl)-amide

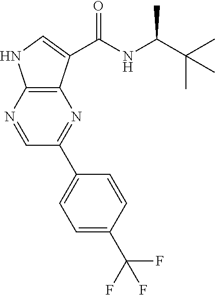 [SMILES: C[C@H](NC(=O)c1c[nH]c2ncc(-c3ccc(C(F)(F)F)cc3)nc12)C(C)(C)C]

Prepared according to the procedure outlined in Example 143, steps 2-3 substituting 4-(trifluoromethyl)phenylboronic acid for 1-methyl-4-(4,4,5,5-tetramethyl-[1,3,2]dioxaborolan-2-yl)-1H-pyrazole in Step 2. MS: (M+H)⁺=391; mp>300° C.

### Example 146

### 2-Cyclopropyl-5H-pyrrolo[2,3-b]pyrazine-7-carboxylic acid ((S)-3-methanesulfonyl-1,2,2-trimethyl-propyl)amide

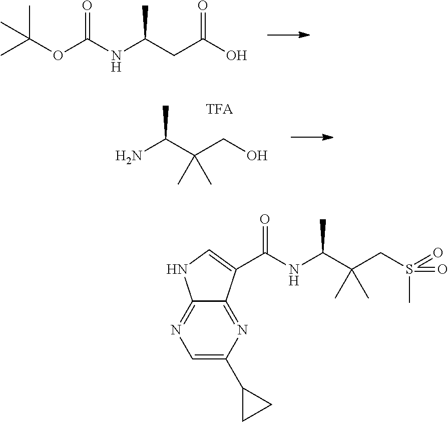

### Step 1

(S)-3-(tert-Butoxycarbonylamino)butanoic acid (1.0 g, 4.9 mmol) was dissolved in toluene (38 mL) and methanol (11 mL). The solution was cooled in an ice/water bath and trimethylsilyl diazomethane (2 M solution in hexanes, 12.3 mL, 24.6 mmol) was added slowly. The reaction was stirred at 20° C. for 18 h then concentrated. The residue was absorbed on to silica gel and purified by silica gel chromatography (ethyl acetate/hexanes) to give 1.06 g (99%) of (S)-3-tert-butoxycarbonylamino-butyric acid methyl ester.

### Step 2

(S)-3-tert-Butoxycarbonylamino-butyric acid methyl ester (1.06 g, 2.9 mmol) was dissolved in THF (29 mL) and stirred in a dry ice/acetone bath. Lithium diisopropylamide was prepared in a separate flask by addition of butyl lithium solution (2.6 M in hexanes, 4.2 mL, 10.8 mmol) to a dry ice/acetone bath cooled solution of diisopropylamine (1.54 mL, 10.8 mmol) in THF (4 mL), and then stirred for 45 min. The lithium diisopropylamide solution was added via cannula to the ester solution over 20 min and the reaction was stirred for another 30 min at dry ice/acetone temperature. Iodomethane (0.7 mL, 10.8 mmol) was added to the reaction and the mixture stirred for 2 h. Additional iodomethane (0.7 mL, 10.8 mmol) was added over 20 min and the reaction was then allowed to warm to 0° C. with stirring over 16 h. Ammonium chloride solution was added and the mixture was extracted twice with ethyl acetate. The combined organic layers were washed with brine, dried over sodium sulfate and evaporated. The residue was purified by silica gel chromatography (diethyl ether/hexanes) to give 0.49 g (39%) of (S)-3-tert-butoxycarbonylamino-2,2-dimethyl-butyric acid methyl ester.

### Step 3

(S)-3-tert-Butoxycarbonylamino-2,2-dimethyl-butyric acid methyl ester (0.47 g, 1.92 mmol) was dissolved in THF (11 mL) and cooled to −35° C. Lithium aluminum hydride (1.0 M in THF, 1.9 mL, 1.9 mmol) was added dropwise. The reaction was stirred as the temperature gradually reached 5° C. after 2 h. Approximately 75 uL water was then added carefully, followed by 120 uL of 10% NaOH solution and then 190 uL water. The resulting solid was filtered, rinsed with ether, and evaporated to give 0.37 g (88%) of ((S)-3-hydroxy-1,2,2-trimethyl-propyl)-carbamic acid tert-butyl ester as a white solid.

### Step 4

((S)-3-Hydroxy-1,2,2-trimethyl-propyl)-carbamic acid tert-butyl ester (244 mg, 1.12 mmol) was dissolved in dichlormethane (7.5 mL) and stirred in an ice bath. Trifluoroacetic acid (3.5 mL) was slowly added and the reaction was warmed to room temperature and stirred for 1 h then evaporated to dryness to afford (S)-3-amino-2,2-dimethyl-butan-1-ol trifluoroacetate which was used without further purification.

### Step 5

(S)-3-Amino-2,2-dimethyl-butan-1-ol trifluoroacetate (crude from step 4) was dissolved in acetonitrile (3.75 mL). 2-Cyclopropyl-5-(2-trimethylsilanyl-ethoxymethyl)₅H-pyrrolo[2,3-b]pyrazine-7-carboxylic acid (250 mg, 0.75 mmol) and O-benzotriazol-1-yl-N,N,N′,N′-tetramethyluronium tetrafluoroborate (361 mg, 1.12 mmol) and N,N-diisopropylethylamine (0.46 mL, 2.62 mmol) were added and the mixture was stirred at room temperature for 18 h. Water and ethyl acetate were added, the layers were separated and the aqueous layer was extracted twice more with ethyl acetate. The combined organic layers were washed with brine, dried over sodium sulfate and concentrated. The residue was purified by silica gel chromatography (ethyl acetate/dichloromethane) to give 130 mg (40%) of 2-cyclopropyl-5-(2-trimethylsilanyl-ethoxymethyl)-5H-pyrrolo[2,3-b]pyrazine-7-carboxylic acid ((S)-3-hydroxy-1,2,2-trimethyl-propyl)-amide.

### Step 6

2-Cyclopropyl-5-(2-trimethylsilanyl-ethoxymethyl)-5H-pyrrolo[2,3-b]pyrazine-7-carboxylic acid ((S)-3-hydroxy-1,2,2-trimethyl-propyl)-amide (0.13 g, 0.3 mmol) was dissolved in 1.5 mL of dichloromethane and cooled in an ice bath. N,N-diisopropylethylamine (0.08 mL, 0.45 mmol) was added, followed by slow addition of methanesulfonyl chloride (0.041 mL, 0.36 mmol). The reaction was warmed to room temperature over 5 h. Ammonium chloride solution was added to the reaction and then extracted three times with ethyl acetate. The combined organic layers were washed with brine, dried over sodium sulfate and evaporated to give methanesulfonic acid (S)-3-{[2-cyclopropyl-5-(2-trimethylsilanyl-ethoxymethyl)-5H-pyrrolo[2,3-b]pyrazine-7-carbonyl]-amino}-2,2-dimethyl-butyl ester which was used without further purification.

### Step 7

In a microwave vial methanesulfonic acid (S)-3-{[2-cyclopropyl-5-(2-trimethylsilanyl-ethoxymethyl)-5H-pyrrolo[2,3-b]pyrazine-7-carbonyl]-amino}-2,2-dimethyl-butyl ester (crude from step 6) was dissolved in DMF (3 mL). Sodium thiomethoxide (0.2 g, 2.8 mmol) was added followed by 0.3 mL of water. The vial was sealed and heated in a microwave reactor at 110° C. for 1 h. The reaction was cooled and poured into ethyl acetate and sodium bicarbonate solution. The aqueous layer was extracted once more with ethyl acetate. The combined organic layers were washed with water and brine, dried over sodium sulfate and concentrated. The residue was purified by silica gel chromatography (methanol/ethyl acetate) to give 35 mg (32%) of 2-cyclopropyl-5-(2-trimethylsilanyl-ethoxymethyl)-5H-pyrrolo[2,3-b]pyrazine-7-carboxylic acid ((S)-1,2,2-trimethyl-3-methylsulfanyl-propyl)-amide.

### Step 8

2-Cyclopropyl-5-(2-trimethylsilanyl-ethoxymethyl)-5H-pyrrolo[2,3-b]pyrazine-7-carboxylic acid ((S)-1,2,2-trimethyl-3-methylsulfanyl-propyl)-amide (45 mg, 0.097 mmol) was dissolved in THF (0.35 mL). Oxone (0.18 g, 0.29 mmol) suspended in THF (1.3 mL) was added and the reaction was stirred for 5 h, then stored in a freezer overnight. Water and ethyl acetate were added. The aqueous layer was extracted twice more with ethyl acetate. The combined organic layers were washed with sodium bicarbonate solution, dried over sodium sulfate and evaporated to give 45 mg of 2-cyclopropyl-5-(2-trimethylsilanyl-ethoxymethyl)-5H-pyrrolo[2,3-b]pyrazine-7-carboxylic acid ((S)-3-methanesulfonyl-1,2,2-trimethyl-propyl)-amide which was used without further purification.

### Step 9

2-Cyclopropyl-5-(2-trimethylsilanyl-ethoxymethyl)-5H-pyrrolo[2,3-b]pyrazine-7-carboxylic acid ((S)-3-methanesulfonyl-1,2,2-trimethyl-propyl)-amide (45 mg, 0.097 mmol) was dissolved in dichloromethane (0.7 mL) and then stirred in an ice bath. Trifluoroacetic acid (0.3 mL) was added slowly and the ice bath was removed. The reaction was stirred for 3 h then recooled in an ice bath. Sodium bicarbonate solution was added and the mixture was extracted three times with ethyl acetate. The combined organic layers were washed with brine, dried over sodium sulfate and concentrated. The residue was dissolved in absolute ethanol (4 mL) and sodium acetate (159 mg, 1.94 mmol) was added. The reaction mixture was stirred for 16 h at 60° C. then cooled and water and ethyl acetate were added. The aqueous layer was extracted twice more with ethyl acetate then the combined organic layers were washed with brine, dried over sodium sulfate and concentrated. The residue was purified by silica gel chromatography (MeOH/dichloromethane) to afford 17 mg (47%) of 2-cyclopropyl-5H-pyrrolo[2,3-b]pyrazine-7-carboxylic acid ((S)-3-methanesulfonyl-1,2,2-trimethyl-propyl)amide. MS: (M+H)⁺=365; mp=232-234° C.

### Example 147

### 2-[1-(3-Chlorophenyl)-1H-imidazol-4-yl]-5H-pyrrolo[2,3-b]pyrazine-7-carboxylic acid ((5)-1,2,2-trimethyl-propyl)-amide

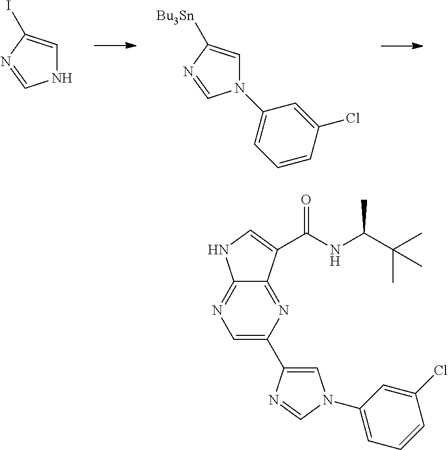

### Step 1

4-Iodo-1H-imidazole (1.0 g, 5.16 mmol) was dissolved in THF (32 mL). Copper TMEDA catalyst (480 mg, 1.03 mmol, Aldrich) and then 3-chlorophenylboronic acid (0.56 g, 3.6 mmol) were added. Oxygen gas was bubbled into the reaction mixture for 20 min, then the mixture was stirred for 90 min. An additional 0.28 g of 3-chlorophenylboronic acid was added followed by an additional 20 min of oxygen gas bubbling and 75 min of stirring at room temperature. An additional 0.28 g of 3-chlorophenylboronic acid was added followed by an additional 20 min of oxygen gas bubbling and then stirring at room temperature for 20 h. The reaction mixture was filtered through a bed of neutral alumina and the filtrate was concentrated. The residue was purified by silica gel chromatography (ethyl acetate/hexanes) to give 0.76 g (48%) of 4-iodo-1-(3-chlorophenyl)-1H-imidazole.

### Step 2

4-Iodo-1-(3-chlorophenyl)-1H-imidazole (0.76 g, 2.5 mmol) was dissolved in anhydrous THF (13 mL). Isopropylmagnesium chloride (2.0 M in THF, 1.56 mL, 3.12 mmol) was added dropwise. The reaction was stirred for 1 h at room temperature. Tributylstannyl chloride (0.71 mL, 2.6 mmol) was added slowly. After the reaction was judged to be complete by TLC, ammonium chloride solution and ethyl acetate were added. The aqueous layer was extracted twice more with ethyl acetate and the combined organic layers were washed with brine, dried over sodium sulfate, and concentrated. The residue was purified by silica gel chromatography (triethylamine/ethyl acetate/hexanes) to give 0.45 g (38%) of 1-(3-chlorophenyl)-4-tributylstannanyl-1H-imidazole.

### Step 3

2-Bromo-5-(2-trimethylsilanyl-ethoxymethyl)-5H-pyrrolo[2,3-b]pyrazine-7-carboxylic acid ((S)-1,2,2-trimethyl-propyl)-amide (100 mg, 0.19 mmol) and 1-(3-chlorophenyl)-4-tributylstannanyl-1H-imidazole (107 mg, 0.229 mmol) were dissolved in DMF (1.9 mL) and the reaction mixture was purged with Ar gas. Tetrakis(triphenylphosphine)palladium (11 mg, 0.010 mmol) and then copper (I) iodide (7 mg, 0.038 mmol) were added and the reaction was sealed and stirred in a 100° C. oil bath for 2 h. The reaction was cooled and water, ethyl acetate, and sodium bicarbonate solution were added. The aqueous layer was extracted twice more with ethyl acetate. The combined organic layers were washed with water and brine, then dried over sodium sulfate and concentrated. The residue was purified by silica gel chromatography (ethyl acetate/hexanes) to give 70 mg (68%) of 2-[1-(3-chloro-phenyl)-1H-imidazol-4-yl]-5-(2-trimethylsilanyl-ethoxymethyl)-5H-pyrrolo[2,3-b]pyrazine-7-carboxylic acid ((S)-1,2,2-trimethyl-propyl)-amide. (M+H)⁺=553.

### Step 4

2-[1-(3-Chloro-phenyl)-1H-imidazol-4-yl]-5-(2-trimethylsilanyl-ethoxymethyl)-5H-pyrrolo[2,3-b]pyrazine-7-carboxylic acid ((S)-1,2,2-trimethyl-propyl)-amide (145 mg, 0.26 mmol) was dissolved in dichloromethane (1.6 mL) and then stirred in an ice bath. Trifluoroacetic acid (0.8 mL) was added slowly and the ice bath was removed. The reaction was stirred for 2.5 h then recooled in an ice bath. Sodium bicarbonate solution was added and the mixture was extracted three times with ethyl acetate. The combined organic layers were washed with brine, dried over sodium sulfate and concentrated. The residue was dissolved in absolute ethanol (10 mL) and sodium acetate (430 mg, 5.24 mmol) was added. The reaction mixture was stirred for 16 h at 60° C. then cooled and water and ethyl acetate were added. The aqueous layer was extracted twice more with ethyl acetate then the combined organic layers were washed with brine, dried over sodium sulfate and concentrated. The residue was purified by silica gel chromatography (MeOH/dichloromethane) to afford 75 mg (68%) of 2-[1-(3-chlorophenyl)-1H-imidazol-4-yl]-5H-pyrrolo[2,3-b]pyrazine-7-carboxylic acid ((S)-1,2,2-trimethyl-propyl)-amide. MS: (M+H)⁺=423; mp=337-339° C.

### Example 148

### 2-[1-(3-Trifluoromethylphenyl)-1H-imidazol-4-yl]-5H-pyrrolo[2,3-b]pyrazine-7-carboxylic acid

((S)-1,2,2-trimethyl-propyl)-amide

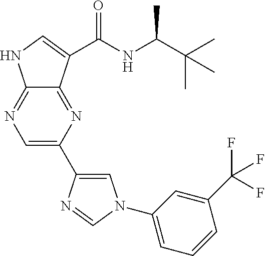 [SMILES: C[C@H](NC(=O)c1c[nH]c2ncc(-c3cn(-c4cccc(C(F)(F)F)c4)cn3)nc12)C(C)(C)C]

Prepared according to the procedure outlined in Example 147 substituting 3-(trifluoromethyl)phenylboronic acid for 3-chlorophenylboronic acid in Step 1. MS: (M+Na)⁺=479; mp=332-333° C.

### Example 149

### 2-[1-(5-Chloro-2-fluorophenyl)-1H-imidazol-4-yl]-5H-pyrrolo[2,3-b]pyrazine-7-carboxylic acid

((S)-1,2,2-trimethyl-propyl)-amide

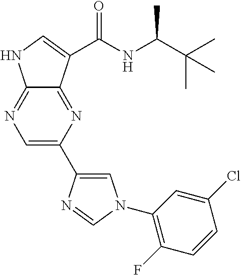 [SMILES: C[C@H](NC(=O)c1c[nH]c2ncc(-c3cn(-c4cc(Cl)ccc4F)cn3)nc12)C(C)(C)C]

Prepared according to the procedure outlined in Example 147 substituting 2-fluoro-5-chlorophenylboronic acid for 3-chlorophenylboronic acid in Step 1. MS: (M+Na)⁺=463; mp=337-339° C.

### Example 150

### 2-[1-(2-Fluoro-5-methylphenyl)-1H-imidazol-4-yl]-5H-pyrrolo[2,3-b]pyrazine-7-carboxylic acid

((S)-1,2,2-trimethyl-propyl)-amide

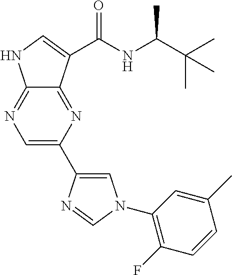 [SMILES: Cc1ccc(F)c(-n2cnc(-c3cnc4[nH]cc(C(=O)N[C@@H](C)C(C)(C)C)c4n3)c2)c1]

Prepared according to the procedure outlined in Example 147 substituting 2-fluoro-5-methylphenylboronic acid for 3-chlorophenylboronic acid in Step 1. MS: (M+Na)⁺=443; mp=331-332° C.

### Example 151

### 2-[1-(2-Fluoro-5-trifluoromethylphenyl)-1H-imidazol-4-yl]-5H-pyrrolo[2,3-b]pyrazine-7-carboxylic acid ((S)-1,2,2-trimethyl-propyl)-amide

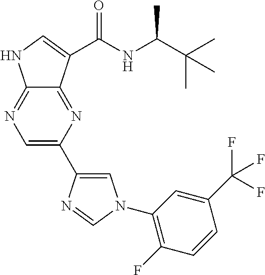 [SMILES: C[C@H](NC(=O)c1c[nH]c2ncc(-c3cn(-c4cc(C(F)(F)F)ccc4F)cn3)nc12)C(C)(C)C]

Prepared according to the procedure outlined in Example 147 substituting 2-fluoro-5-(trifluoromethyl)phenylboronic acid for 3-chlorophenylboronic acid in Step 1. MS: (M+Na)⁺=497; mp>300° C.

### Example 152

### 2-(1-(3-Methylphenyl)-1H-imidazol-4-yl)-5H-pyrrolo[2,3-b]pyrazine-7-carboxylic acid ((5)-1,2,2-trimethyl-propyl)-amide

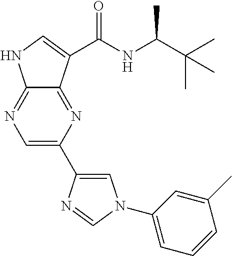 [SMILES: Cc1cccc(-n2cnc(-c3cnc4[nH]cc(C(=O)N[C@@H](C)C(C)(C)C)c4n3)c2)c1]

Prepared according to the procedure outlined in Example 147 substituting 3-methylphenylboronic acid for 3-chlorophenylboronic acid in Step 1. MS: (M+Na)⁺=425; mp=314-316° C.

### Example 153

### 2-(1-(3-Ethylphenyl)-1H-imidazol-4-yl)-5H-pyrrolo[2,3-b]pyrazine-7-carboxylic acid ((S)-1,2,2-trimethyl-propyl)-amide

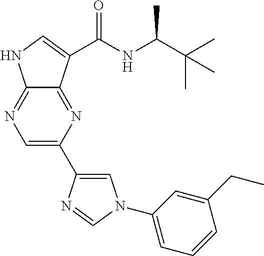 [SMILES: CCc1cccc(-n2cnc(-c3cnc4[nH]cc(C(=O)N[C@@H](C)C(C)(C)C)c4n3)c2)c1]

Prepared according to the procedure outlined in Example 147 substituting 3-ethylphenylboronic acid for 3-chlorophenylboronic acid in Step 1. MS: (M+Na)⁺=439; mp=284-287° C.

### Example 154

### 2-[1-(3-Isopropylphenyl)-1H-imidazol-4-yl]-5H-pyrrolo[2,3-b]pyrazine-7-carboxylic acid ((5)-1,2,2-trimethyl-propyl)-amide

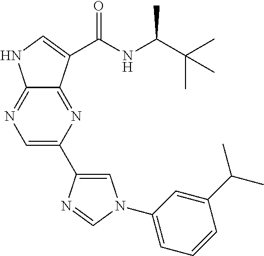 [SMILES: CC(C)c1cccc(-n2cnc(-c3cnc4[nH]cc(C(=O)N[C@@H](C)C(C)(C)C)c4n3)c2)c1]

Prepared according to the procedure outlined in Example 147 substituting 3-isopropylphenylboronic acid for 3-chlorophenylboronic acid in Step 1. MS: (M+Na)⁺=453; mp=242-245° C.

### Example 155

### 2-[1-(3-tert-Butylphenyl)-1H-imidazol-4-yl]-5H-pyrrolo[2,3-b]pyrazine-7-carboxylic acid ((5)-1,2,2-trimethyl-propyl)-amide

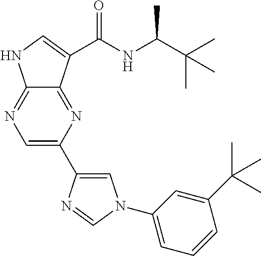 [SMILES: C[C@H](NC(=O)c1c[nH]c2ncc(-c3cn(-c4cccc(C(C)(C)C)c4)cn3)nc12)C(C)(C)C]

Prepared according to the procedure outlined in Example 147 substituting 3-tert-butylphenylboronic acid for 3-chlorophenylboronic acid in Step 1. MS: (M+H)⁺=445; mp=226-228° C.

### Example 156

### 2-[1-(3-Vinylphenyl)-1H-imidazol-4-yl]-5H-pyrrolo[2,3-b]pyrazine-7-carboxylic acid ((S)-1,2,2-trimethyl-propyl)-amide

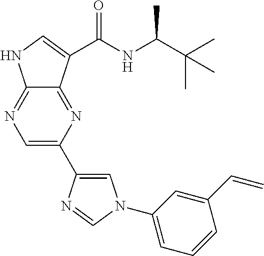 [SMILES: C=Cc1cccc(-n2cnc(-c3cnc4[nH]cc(C(=O)N[C@@H](C)C(C)(C)C)c4n3)c2)c1]

Prepared according to the procedure outlined in Example 147 substituting 3-vinylphenylboronic acid for 3-chlorophenylboronic acid in Step 1. MS: (M+H)⁺=415; mp=253-257° C.

### Example 157

### 2-(1,3-Dimethyl-1H-pyrazol-4-yl)-5H-pyrrolo[2,3-b]pyrazine-7-carboxylic acid ((S)-2-methoxy-1-methyl-ethyl)-amide

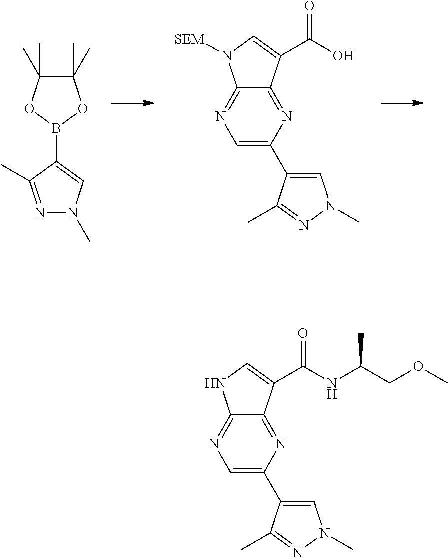

### Step 1

In a 25 mL pressure vessel, 1,3-dimethyl-4-(4,4,5,5-tetramethyl-1,3,2-dioxaborolan-2-yl)-1H-pyrazole (439 mg, 1.98 mmol), lithium chloride (52 mg, 1.23 mmol) and 2-bromo-5-(2-trimethylsilanyl-ethoxymethyl)-5H-pyrrolo[2,3-b]pyrazine-7-carbaldehyde (440 mg, 1.23 mmol) were combined with ethanol (7 mL) and toluene (7 mL) and the mixture was purged with N₂. Potassium phosphate tribasic (917 mg, 4.32 mmol) was dissolved in 4 mL water and added to the mixture. After more purging with N₂ bis(triphenylphosphine)palladium (II) dichloride (87 mg, 0.12 mmol) was added, the vessel capped and stirred at 60-65° C. for 20 h. The reaction was cooled, then diluted with ethyl acetate and water. The organic layer was washed with brine, dried and evaporated. The crude material was purified by flash chromatography (silica gel, 80 g, 100% EtOAc to 20% THF/EtOAc) to give 360 mg (71% yield; 90% purity) of 2-(1,3-dimethyl-1H-pyrazol-4-yl)-5-(2-trimethylsilanyl-ethoxymethyl)-5H-pyrrolo[2,3-b]pyrazine-7-carbaldehyde.

### Step 2

To a solution of 2-(1,3-dimethyl-1H-pyrazol-4-yl)-5-(2-trimethylsilanyl-ethoxymethyl)-5 H-pyrrolo[2,3-b]pyrazine-7-carbaldehyde (440 mg, 1.18 mmol) in 1,4-dioxane (20 mL) at 5° C. was added a solution of sulfamic acid (690 mg, 7.11 mmol) in water (7 mL). Then a solution of NaClO₂ (139 mg, 1.54 mmol) and KH₂PO₄ (161 mg, 1.18 mmol) in water (4 mL) was slowly added over 5 min. The ice bath was removed and the yellow cloudy reaction mixture was stirred at r.t. for 2 h. Half of the solvent was evaporated and the remainder was poured into brine and extracted with 80% EtOAc/hexanes (2×). The combined organics were washed with brine and concentrated. The residue was purified by silica gel chromatography (MeOH/dichloromethane) followed by trituration with cold diethyl ether/hexanes to afford 320 mg (66%) of 2-(1,3-dimethyl-1H-pyrazol-4-yl)-5-(2-trimethylsilanyl-ethoxymethyl)-5H-pyrrolo[2,3-b]pyrazine-7-carboxylic acid as a white solid.

### Step 3

In a round-bottomed flask, (S)-(+)-1-methoxy-2-propylamine (23.2 μL, 0.22 mmol), N,N-diisopropylethylamine (38 μL, 0.22 mmol) and HATU (83 mg, 0.22 mmol) and 2-(1,3-dimethyl-1H-pyrazol-4-yl)-5-(2-trimethylsilanyl-ethoxymethyl)-5H-pyrrolo[2,3-b]pyrazine-7-carboxylic acid (85 mg, 0.22 mmol) were combined with DMF (10 mL) and stirred at room temperature for 20 h. The reaction mixture was diluted with EtOAc (50 mL) and hexane (10 mL), poured into 30% brine/water, and extracted with EtOAc (2×). The combined organics were washed with brine and concentrated. The residue was purified by silica gel chromatography (MeOH/dichloromethane) to afford 2-(1,3-dimethyl-1H-pyrazol-4-yl)-5-(2-trimethylsilanyl-ethoxymethyl)-5H-pyrrolo[2,3-b]pyrazine-7-carboxylic acid ((S)-2-methoxy-1-methyl-ethyl)-amide.

### Step 4

2-(1,3-Dimethyl-1H-pyrazol-4-yl)-5H-pyrrolo[2,3-b]pyrazine-7-carboxylic acid ((S)-2-methoxy-1-methyl-ethyl)-amide. Prepared according to the procedure outlined in Example 1, step 5 substituting 2-(1,3-dimethyl-1H-pyrazol-4-yl)-5-(2-trimethylsilanyl-ethoxymethyl)-5H-pyrrolo[2,3-b]pyrazine-7-carboxylic acid ((S)-2-methoxy-1-methyl-ethyl)-amide for 2-cyclopropyl-5-(2-trimethylsilanylethoxymethyl)-5H-pyrrolo[2,3-b]pyrazine-7-carboxylic acid [(R)-1-(1-hydroxy-cyclopentyl)-ethyl]-amide. MS: (M+H)⁺=329.

### Example 158

### 2-(5-Ethylcarbamoyl-thiophen-2-yl)-5H-pyrrolo[2,3-b]pyrazine-7-carboxylic acid ((S)-1,2,2-trimethyl-propyl)-amide

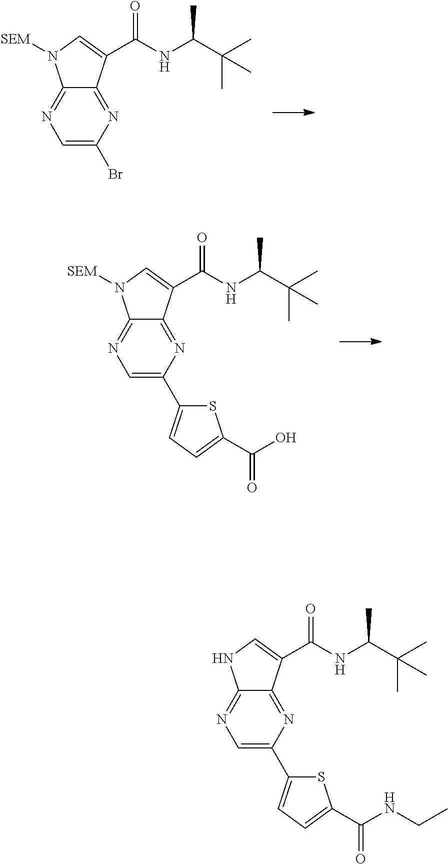 [SMILES: C[C@H](NC(=O)c1cn(COCC[Si](C)(C)C)c2ncc(Br)nc12)C(C)(C)C ; C[C@H](NC(=O)c1cn(COCC[Si](C)(C)C)c2ncc(-c3ccc(C(=O)O)s3)nc12)C(C)(C)C ; CCNC(=O)c1ccc(-c2cnc3[nH]cc(C(=O)N[C@@H](C)C(C)(C)C)c3n2)s1]

### Step 1

To a stirred solution of 2-bromo-5-(2-trimethylsilanyl-ethoxymethyl)-5H-pyrrolo[2,3-b]pyrazine-7-carboxylic acid ((S)-1,2,2-trimethyl-propyl)-amide (500 mg, 1.10 mmol) in 4:1 dioxane/water (15 mL) in a pressure tube, were added 2-formylthiophene-5-boronic acid (274 mg, 1.76 mmol) and K₂CO₃ (455 mg, 3.29 mmol). The reaction mixture was purged with argon for 15 min, followed by the addition of PdCl₂dppf CH₂Cl₂ (90 mg, 0.11 mmol). The tube was sealed and heated at 120° C. for 18 h then cooled to room temperature and partitioned between water and EtOAc. The organic layer was dried over Na₂SO₄ and evaporated under reduced pressure. The crude residue was purified by silica gel column chromatography, using EtOAc/hexane=1:5 as eluent, to obtain 0.32 g (60%) of 2-(5-formyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide as a light yellow solid. LC-MS: 487 (M+H)⁺.

### Step 2

To a stirred solution of 2-(5-formyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide (1.5 g, 3.09 mmol) in 1:1 dioxane/water (50 mL), were added sulfamic acid (1.8 g, 18.51 mmol), sodium chlorite (0.36 g, 4.01 mmol) and KH₂PO₄ (5.04 g, 37.03 mmol). The reaction mixture was stirred at 25° C. for 30 h then partitioned between water and EtOAc. The organic layer was dried over Na₂SO₄ and evaporated under reduced pressure to afford 1.3 g (84%) of 5-[7-((S)-1,2,2-trimethyl-propylcarbamoyl)-5-(2-trimethylsilanyl-ethoxymethyl)-5H-pyrrolo[2,3-b]pyrazin-2-yl]-thiophene-2-carboxylic acid as light yellow solid which was used without further purification. LC-MS: 503 [M+H]⁺.

### Step 3

To a stirred solution of 5-[7-((S)-1,2,2-trimethyl-propylcarbamoyl)-5-(2-trimethylsilanyl-ethoxymethyl)-5H-pyrrolo[2,3-b]pyrazin-2-yl]-thiophene-2-carboxylic acid (200 mg, 0.40 mmol) in THF, were added triethylamine (0.22 mL, 1.6 mmol), PyBOP (416 mg, 0.80 mmol) and ethylamine (2.0M in THF, 0.90 mL, 1.80 mmol). The reaction mixture was stirred at 25° C. for 18 h then partitioned between water and EtOAc. The organic layer was dried over Na₂SO₄ and evaporated under reduced pressure. The crude residue was purified by silica gel column chromatography (EtOAc/hexane) to afford 160 mg (76%) of 2-(5-ethylcarbamoyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide as a light yellow solid. LC-MS: 530 [M+H]⁺.

### Step 4

A stirred solution of 2-(5-ethylcarbamoyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide (150 mg, 0.28 mmol) in 1.0 M HCl in AcOH was heated at 60° C. for 3 h. The reaction mixture was concentrated under reduced pressure and the residue was dissolved in 1:1 MeOH/CH₂Cl₂ (3 mL) and ethylenediamine (0.3 mL) was added. Reaction mixture was stirred at 25° C. for 16 h then concentrated under reduced pressure. The crude residue was purified by silica gel column chromatography (MeOH/CH₂Cl₂) to afford 100 mg (89%) of 2-(5-ethylcarbamoyl-thiophen-2-yl)-5H-pyrrolo[2,3-b]pyrazine-7-carboxylic acid ((S)-1,2,2-trimethyl-propyl)-amide as an off-white solid. MS: (M+H)⁺=400.

### Example 159

### 2-(5-Isopropylcarbamoyl-thiophen-2-yl)-5H-pyrrolo[2,3-b]pyrazine-7-carboxylic acid ((S)-1,2,2-trimethyl-propyl)-amide

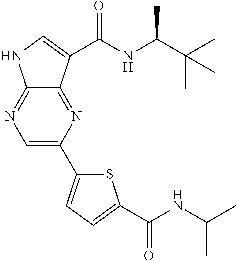 [SMILES: CC(C)NC(=O)c1ccc(-c2cnc3[nH]cc(C(=O)N[C@@H](C)C(C)(C)C)c3n2)s1]

Prepared according to the procedure outlined in Example 158 substituting isopropylamine for ethylamine in Step 3. MS: (M+H)⁺=414.

### Example 160

### 2-(5-tert-Butylcarbamoyl-thiophen-2-yl)-5H-pyrrolo[2,3-b]pyrazine-7-carboxylic acid ((5)-1,2,2-trimethyl-propyl)-amide

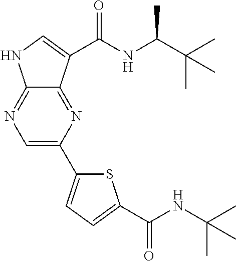 [SMILES: C[C@H](NC(=O)c1c[nH]c2ncc(-c3ccc(C(=O)NC(C)(C)C)s3)nc12)C(C)(C)C]

Prepared according to the procedure outlined in Example 158 substituting tert-butylamine for ethylamine in Step 3. MS: (M+H)⁺=428.

### Example 161

### 2-[5-(1-Methyl-2-pyrazol-1-yl-ethylcarbamoyl)-thiophen-2-yl]-5H-pyrrolo[2,3-b]pyrazine-7-carboxylic acid ((S)-1,2,2-trimethyl-propyl)-amide

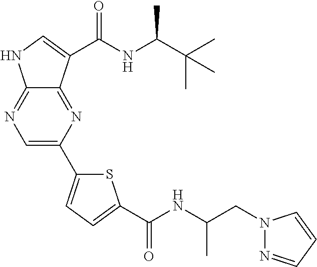 [SMILES: CC(Cn1cccn1)NC(=O)c1ccc(-c2cnc3[nH]cc(C(=O)N[C@@H](C)C(C)(C)C)c3n2)s1]

Prepared according to the procedure outlined in Example 158 substituting 1-methyl-2-pyrazol-1-yl-ethylamine for ethylamine in Step 3. MS: (M+H)⁺=480.

### Example 162

### 2-{5-[2-(4-Fluoro-phenyl)-1-methyl-ethylcarbamoyl]-thiophen-2-yl}-5H-pyrrolo[2,3-b]pyrazine-7-carboxylic acid ((S)-1,2,2-trimethyl-propyl)-amide

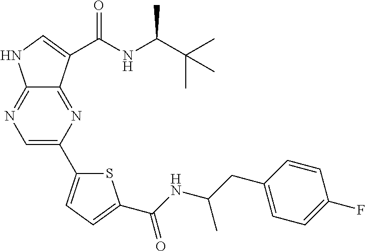 [SMILES: CC(Cc1ccc(F)cc1)NC(=O)c1ccc(-c2cnc3[nH]cc(C(=O)N[C@@H](C)C(C)(C)C)c3n2)s1]

Prepared according to the procedure outlined in Example 158 substituting 2-(4-fluoro-phenyl)-1-methyl-ethylamine for ethylamine in Step 3. MS: (M+H)⁺=508.

### Example 163

### 2-(5-Diethylcarbamoyl-thiophen-2-yl)-5H-pyrrolo[2,3-b]pyrazine-7-carboxylic acid ((S)-1,2,2-trimethyl-propyl)-amide

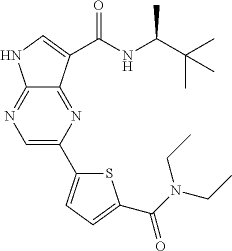 [SMILES: CCN(CC)C(=O)c1ccc(-c2cnc3[nH]cc(C(=O)N[C@@H](C)C(C)(C)C)c3n2)s1]

Prepared according to the procedure outlined in Example 158 substituting diethylamine for ethylamine in Step 3. MS: (M+H)⁺=428.

### Example 164

### 2-[5-(4-Methyl-piperazine-1-carbonyl)-thiophen-2-yl]-5H-pyrrolo[2,3-b]pyrazine-7-carboxylic acid ((S)-1,2,2-trimethyl-propyl)-amide

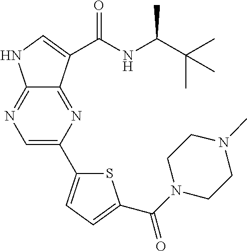 [SMILES: C[C@H](NC(=O)c1c[nH]c2ncc(-c3ccc(C(=O)N4CCN(C)CC4)s3)nc12)C(C)(C)C]

Prepared according to the procedure outlined in Example 158 substituting 1-methylpiperazine for ethylamine in Step 3. MS: (M+H)⁺=455.

### Example 165

### 2-[5-((R)-1-Cyclopropylethylcarbamoyl)-thiophen-2-yl]-5H-pyrrolo[2,3-b]pyrazine-7-carboxylic acid ((S)-1,2,2-trimethyl-propyl)-amide

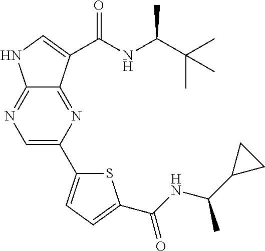 [SMILES: C[C@H](NC(=O)c1c[nH]c2ncc(-c3ccc(C(=O)N[C@H](C)C4CC4)s3)nc12)C(C)(C)C]

Prepared according to the procedure outlined in Example 158 substituting (R)-1-cyclopropylethylamine for ethylamine in Step 3. MS: (M+H)⁺=440.

### Example 166

### 2-{5-[(Pyridin-3-ylmethyl)-carbamoyl]-thiophen-2-yl}-5H-pyrrolo[2,3-b]pyrazine-7-carboxylic acid ((S)-1,2,2-trimethyl-propyl)-amide

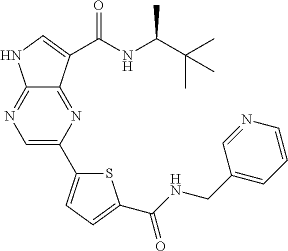 [SMILES: C[C@H](NC(=O)c1c[nH]c2ncc(-c3ccc(C(=O)NCc4cccnc4)s3)nc12)C(C)(C)C]

Prepared according to the procedure outlined in Example 158 substituting 3-(aminomethyl)pyridine for ethylamine in Step 3. MS: (M+H)⁺=463.

### Example 167

### 2-{5-[(Pyridin-4-ylmethyl)-carbamoyl]-thiophen-2-yl}-5H-pyrrolo[2,3-b]pyrazine-7-carboxylic acid ((S)-1,2,2-trimethyl-propyl)-amide

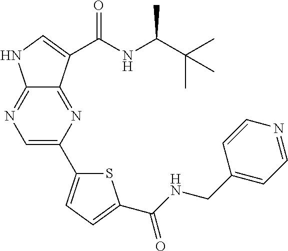 [SMILES: C[C@H](NC(=O)c1c[nH]c2ncc(-c3ccc(C(=O)NCc4ccncc4)s3)nc12)C(C)(C)C]

Prepared according to the procedure outlined in Example 158 substituting 4-(aminomethyl)pyridine for ethylamine in Step 3. MS: (M+H)⁺=463.

### Example 168

### 2-{5-[(Pyridin-2-ylmethyl)-carbamoyl]-thiophen-2-yl}-5H-pyrrolo[2,3-b]pyrazine-7-carboxylic acid ((S)-1,2,2-trimethyl-propyl)-amide

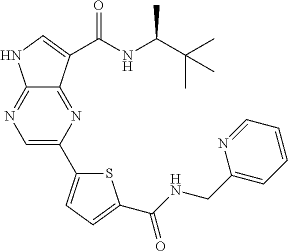 [SMILES: C[C@H](NC(=O)c1c[nH]c2ncc(-c3ccc(C(=O)NCc4ccccn4)s3)nc12)C(C)(C)C]

Prepared according to the procedure outlined in Example 158 substituting 2-(aminomethyl)pyridine for ethylamine in Step 3. MS: (M+H)⁺=463.

### Example 169

2-[5-(4-Cyano-piperidine-1-carbonyl)-thiophen-2-yl]-5H-pyrrolo[2,3-b]pyrazine-7-carboxylic acid ((S)-1,2,2-trimethyl-propyl)-amide

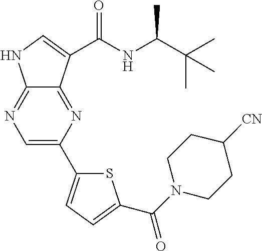 [SMILES: C[C@H](NC(=O)c1c[nH]c2ncc(-c3ccc(C(=O)N4CCC(C#N)CC4)s3)nc12)C(C)(C)C]

Prepared according to the procedure outlined in Example 158 substituting piperidine-4-carbonitrile for ethylamine in Step 3. MS: (M+H)⁺=465.

### Example 170

### 2-[5-(Cyclopentylmethyl-carbamoyl)-thiophen-2-yl]-5H-pyrrolo[2,3-b]pyrazine-7-carboxylic acid ((S)-1,2,2-trimethyl-propyl)-amide

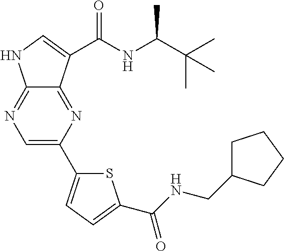 [SMILES: C[C@H](NC(=O)c1c[nH]c2ncc(-c3ccc(C(=O)NCC4CCCC4)s3)nc12)C(C)(C)C]

Prepared according to the procedure outlined in Example 158 substituting cyclopentylmethyl amine for ethylamine in Step 3. MS: (M+H)⁺=454.

### Example 171

### 2-[5-((R)-2-Hydroxy-1-methyl-ethylcarbamoyl)-thiophen-2-yl]-5H-pyrrolo[2,3-b]pyrazine-7-carboxylic acid ((S)-1,2,2-trimethyl-propyl)-amide

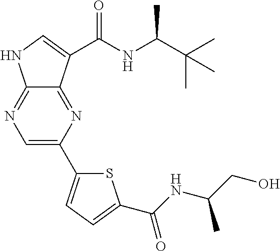 [SMILES: C[C@H](CO)NC(=O)c1ccc(-c2cnc3[nH]cc(C(=O)N[C@@H](C)C(C)(C)C)c3n2)s1]

Prepared according to the procedure outlined in Example 158 substituting (R)-2-aminopropan-1-ol for ethylamine in Step 3. MS: (M+H)⁺=430.

### Example 172

### 2-[5-((R)-1-Methyl-2-phenyl-ethylcarbamoyl)-thiophen-2-yl]-5H-pyrrolo[2,3-b]pyrazine-7-carboxylic acid ((S)-1,2,2-trimethyl-propyl)-amide

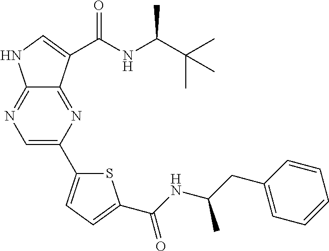 [SMILES: C[C@H](Cc1ccccc1)NC(=O)c1ccc(-c2cnc3[nH]cc(C(=O)N[C@@H](C)C(C)(C)C)c3n2)s1]

Prepared according to the procedure outlined in Example 158 substituting (R)-1-methyl-2-phenylethylamine hydrochloride for ethylamine in Step 3. MS: (M+H)⁺=490.

### Example 173

### 2-[5-(1-Pyridin-3-yl-ethylcarbamoyl)-thiophen-2-yl]-5H-pyrrolo[2,3-b]pyrazine-7-carboxylic acid ((S)-1,2,2-trimethyl-propyl)-amide

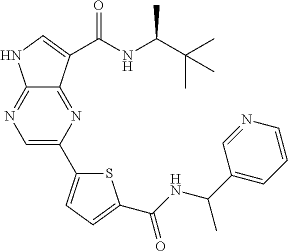 [SMILES: CC(NC(=O)c1ccc(-c2cnc3[nH]cc(C(=O)N[C@@H](C)C(C)(C)C)c3n2)s1)c1cccnc1]

Prepared according to the procedure outlined in Example 158 substituting 1-pyridin-3-yl-ethylamine for ethylamine in Step 3. MS: (M+H)⁺=477.

### Example 174

### 2-[5-(Cyanomethyl-carbamoyl)-thiophen-2-yl]-5H-pyrrolo[2,3-b]pyrazine-7-carboxylic acid

((S)-1,2,2-trimethyl-propyl)-amide

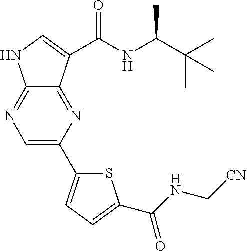 [SMILES: C[C@H](NC(=O)c1c[nH]c2ncc(-c3ccc(C(=O)NCC#N)s3)nc12)C(C)(C)C]

Prepared according to the procedure outlined in Example 158 substituting aminoacetonitrile for ethylamine in Step 3. The SEM deprotection in Step 4 was accomplished using TBAF (1.0 M in THF) in THF at reflux for 16 h followed by treatment with ethylenediamine. MS: (M+H)⁺=411.

### Example 175

### 2-[5-(2-Sulfamoyl-ethylcarbamoyl)-thiophen-2-yl]-5H-pyrrolo[2,3-b]pyrazine-7-carboxylic acid

((S)-1,2,2-trimethyl-propyl)-amide

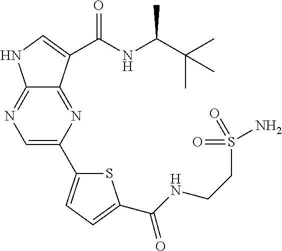 [SMILES: C[C@H](NC(=O)c1c[nH]c2ncc(-c3ccc(C(=O)NCCS(N)(=O)=O)s3)nc12)C(C)(C)C]

Prepared according to the procedure outlined in Example 158 substituting 2-amino-ethanesulfonic acid amide for ethylamine in Step 3. The SEM deprotection in Step 4 was accomplished using TBAF (1.0 M in THF) in THF at reflux for 16 h followed by treatment with ethylenediamine. MS: (M+H)⁺=479.

### Example 176

### 2-[5-(2-Imidazol-1-yl-1-methyl-ethylcarbamoyl)-thiophen-2-yl]-5H-pyrrolo[2,3-b]pyrazine-7-carboxylic acid ((S)-1,2,2-trimethyl-propyl)-amide

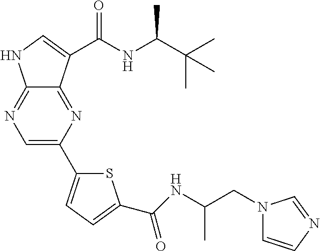 [SMILES: CC(Cn1ccnc1)NC(=O)c1ccc(-c2cnc3[nH]cc(C(=O)N[C@@H](C)C(C)(C)C)c3n2)s1]

Prepared according to the procedure outlined in Example 158 substituting 2-imidazol-1-yl-1-methylethylamine for ethylamine in Step 3. MS: (M+H)⁺=480.

### Example 177

2-[5-(4-Hydroxy-4-methyl-piperidine-1-carbonyl)-thiophen-2-yl]-5H-pyrrolo[2,3-b]pyrazine-7-carboxylic acid ((S)-1,2,2-trimethyl-propyl)-amide

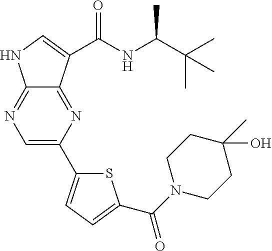 [SMILES: C[C@H](NC(=O)c1c[nH]c2ncc(-c3ccc(C(=O)N4CCC(C)(O)CC4)s3)nc12)C(C)(C)C]

Prepared according to the procedure outlined in Example 158 substituting 4-methyl-piperidin-4-ol hydrochloride for ethylamine in Step 3. MS: (M+H)⁺=470.

### Example 178

### 2-[5-(1-Methyl-2-pyridin-2-yl-ethylcarbamoyl)-thiophen-2-yl]-5H-pyrrolo[2,3-b]pyrazine-7-carboxylic acid ((S)-1,2,2-trimethyl-propyl)-amide

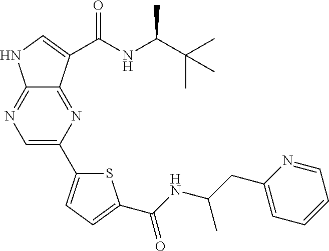 [SMILES: CC(Cc1ccccn1)NC(=O)c1ccc(-c2cnc3[nH]cc(C(=O)N[C@@H](C)C(C)(C)C)c3n2)s1]

Prepared according to the procedure outlined in Example 158 substituting 1-methyl-2-pyridin-2-yl-ethylamine for ethylamine in Step 3. MS: (M+H)⁺=491.

### Example 179

2-[5-(7-Aza-bicyclo[2.2.1]heptane-7-carbonyl)-thiophen-2-yl]-5H-pyrrolo[2,3-b]pyrazine-7-carboxylic acid ((S)-1,2,2-trimethyl-propyl)-amide

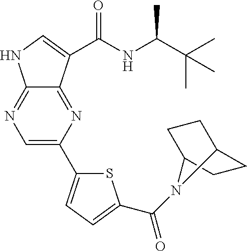 [SMILES: C[C@H](NC(=O)c1c[nH]c2ncc(-c3ccc(C(=O)N4C5CCC4CC5)s3)nc12)C(C)(C)C]

Prepared according to the procedure outlined in Example 158 substituting 7-aza-bicyclo[2.2.1]heptane hydrochloride for ethylamine in Step 3. MS: (M+H)⁺=452.

### Example 180

### 2-[5-(3-Cyano-azetidine-1-carbonyl)-thiophen-2-yl]-5H-pyrrolo[2,3-b]pyrazine-7-carboxylic acid ((S)-1,2,2-trimethyl-propyl)-amide

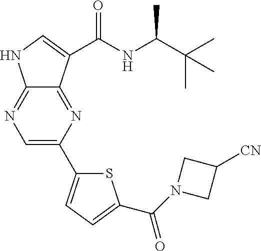 [SMILES: C[C@H](NC(=O)c1c[nH]c2ncc(-c3ccc(C(=O)N4CC(C#N)C4)s3)nc12)C(C)(C)C]

Prepared according to the procedure outlined in Example 158 substituting azetidine-3-carbonitrile for ethylamine in Step 3. The SEM deprotection in Step 4 was accomplished using TBAF (1.0 M in THF) in THF at reflux for 16 h followed by treatment with ethylenediamine. MS: (M+H)⁺=437.

### Example 181

### 2-[5-(3-Carbamoyl-azetidine-1-carbonyl)-thiophen-2-yl]-5H-pyrrolo[2,3-b]pyrazine-7-carboxylic acid ((S)-1,2,2-trimethyl-propyl)-amide

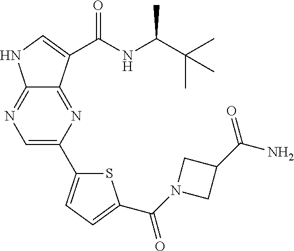 [SMILES: C[C@H](NC(=O)c1c[nH]c2ncc(-c3ccc(C(=O)N4CC(C(N)=O)C4)s3)nc12)C(C)(C)C]

Isolated as an additional product from Example 180, Step 4. MS: (M+H)⁺=455.

### Example 182

### 2-[5-(Azetidine-1-carbonyl)-thiophen-2-yl]-5H-pyrrolo[2,3-b]pyrazine-7-carboxylic acid ((5)-1,2,2-trimethyl-propyl)-amide

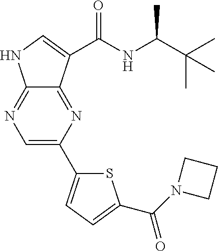 [SMILES: C[C@H](NC(=O)c1c[nH]c2ncc(-c3ccc(C(=O)N4CCC4)s3)nc12)C(C)(C)C]

Prepared according to the procedure outlined in Example 158 substituting azetidine hydrochloride for ethylamine in Step 3. The SEM deprotection in Step 4 was accomplished using TBAF (1.0 M in THF) in THF at reflux for 16 h followed by treatment with ethylenediamine. MS: (M+H)⁺=412.

### Example 183

### 2-[5-(2,6-Dimethylpiperidine-1-carbonyl)-thiophen-2-yl]-5H-pyrrolo[2,3-b]pyrazine-7-carboxylic acid ((S)-1,2,2-trimethyl-propyl)-amide

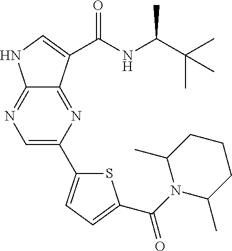 [SMILES: CC1CCCC(C)N1C(=O)c1ccc(-c2cnc3[nH]cc(C(=O)N[C@@H](C)C(C)(C)C)c3n2)s1]

Prepared according to the procedure outlined in Example 158 substituting 2,6-dimethylpiperidine for ethylamine in Step 3. MS: (M+H)⁺=468.

### Example 184

### 1-{5-[7-((S)-1,2,2-Trimethyl-propylcarbamoyl)-5H-pyrrolo[2,3-b]pyrazin-2-yl]-thiophene-2-carbonyl}-piperidine-4-carboxylic acid

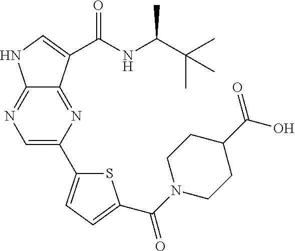 [SMILES: C[C@H](NC(=O)c1c[nH]c2ncc(-c3ccc(C(=O)N4CCC(C(=O)O)CC4)s3)nc12)C(C)(C)C]

Prepared according to the procedure outlined in Example 158 substituting piperidine-4-carboxylic acid methyl ester for ethylamine in Step 3. The methyl ester was hydrolyzed to the acid after coupling. MS: (M+H)⁺=484.

### Example 185

### 2-[5-(4-Acetylamino-piperidine-1-carbonyl)-thiophen-2-yl]-5H-pyrrolo[2,3-b]pyrazine-7-carboxylic acid ((S)-1,2,2-trimethyl-propyl)-amide

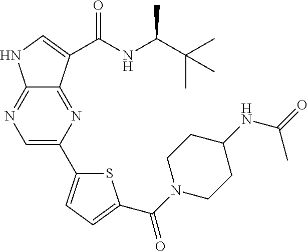 [SMILES: CC(=O)NC1CCN(C(=O)c2ccc(-c3cnc4[nH]cc(C(=O)N[C@@H](C)C(C)(C)C)c4n3)s2)CC1]

Prepared according to the procedure outlined in Example 158 substituting N-piperidin-4-yl-acetamide for ethylamine in Step 3. MS: (M+H)⁺=497.

### Example 186

### 2-[5-(4-Methylbenzylcarbamoyl)-thiophen-2-yl]-5H-pyrrolo[2,3-b]pyrazine-7-carboxylic acid ((S)-1,2,2-trimethyl-propyl)-amide

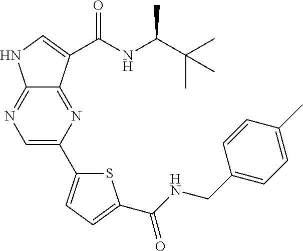 [SMILES: Cc1ccc(CNC(=O)c2ccc(-c3cnc4[nH]cc(C(=O)N[C@@H](C)C(C)(C)C)c4n3)s2)cc1]

Prepared according to the procedure outlined in Example 158 substituting 4-methylbenzylamine for ethylamine in Step 3. MS: (M+H)⁺=476.

### Example 187

### 2-[5-(4-Fluorobenzylcarbamoyl)-thiophen-2-yl]-5H-pyrrolo[2,3-b]pyrazine-7-carboxylic acid ((S)-1,2,2-trimethyl-propyl)-amide

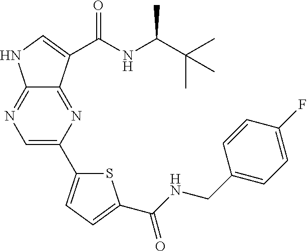 [SMILES: C[C@H](NC(=O)c1c[nH]c2ncc(-c3ccc(C(=O)NCc4ccc(F)cc4)s3)nc12)C(C)(C)C]

Prepared according to the procedure outlined in Example 158 substituting 4-fluorobenzylamine for ethylamine in Step 3. MS: (M+H)⁺=480.

### Example 188

### 2-[5-(2,3-Dichlorobenzylcarbamoyl)-thiophen-2-yl]-5H-pyrrolo[2,3-b]pyrazine-7-carboxylic acid ((S)-1,2,2-trimethyl-propyl)-amide

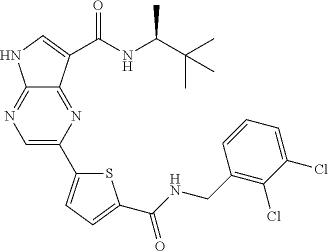 [SMILES: C[C@H](NC(=O)c1c[nH]c2ncc(-c3ccc(C(=O)NCc4cccc(Cl)c4Cl)s3)nc12)C(C)(C)C]

Prepared according to the procedure outlined in Example 158 substituting 2,3-dichlorobenzylamine for ethylamine in Step 3. MS: (M+H)⁺=531.

### Example 189

### 2-[5-(2-Methylbenzylcarbamoyl)-thiophen-2-yl]-5H-pyrrolo[2,3-b]pyrazine-7-carboxylic acid ((S)-1,2,2-trimethyl-propyl)-amide

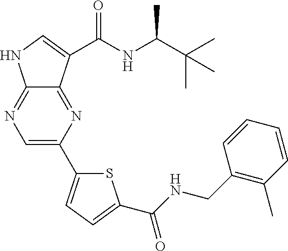 [SMILES: Cc1ccccc1CNC(=O)c1ccc(-c2cnc3[nH]cc(C(=O)N[C@@H](C)C(C)(C)C)c3n2)s1]

Prepared according to the procedure outlined in Example 158 substituting 2-methylbenzylamine for ethylamine in Step 3. MS: (M+H)⁺=476.

### Example 190

### 2-[5-(2,6-Difluorobenzylcarbamoyl)-thiophen-2-yl]-5H-pyrrolo[2,3-b]pyrazine-7-carboxylic acid ((S)-1,2,2-trimethyl-propyl)-amide

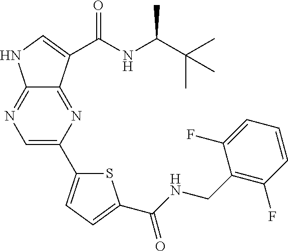 [SMILES: C[C@H](NC(=O)c1c[nH]c2ncc(-c3ccc(C(=O)NCc4c(F)cccc4F)s3)nc12)C(C)(C)C]

Prepared according to the procedure outlined in Example 158 substituting 2,6-difluorobenzylamine for ethylamine in Step 3. MS: (M+H)⁺=498.

### Example 191

### 2-[5-(2-Chloro-6-fluorobenzylcarbamoyl)-thiophen-2-yl]-5H-pyrrolo[2,3-b]pyrazine-7-carboxylic acid ((S)-1,2,2-trimethyl-propyl)-amide

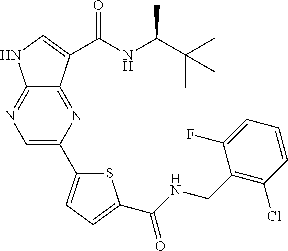 [SMILES: C[C@H](NC(=O)c1c[nH]c2ncc(-c3ccc(C(=O)NCc4c(F)cccc4Cl)s3)nc12)C(C)(C)C]

Prepared according to the procedure outlined in Example 158 substituting 2-chloro-6-fluorobenzylamine for ethylamine in Step 3. MS: (M+H)⁺=515.

### Example 192

### 2-[5-(2-Methylcyclohexylcarbamoyl)-thiophen-2-yl]-5H-pyrrolo[2,3-b]pyrazine-7-carboxylic acid ((S)-1,2,2-trimethyl-propyl)-amide

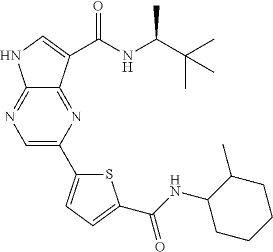 [SMILES: CC1CCCCC1NC(=O)c1ccc(-c2cnc3[nH]cc(C(=O)N[C@@H](C)C(C)(C)C)c3n2)s1]

Prepared according to the procedure outlined in Example 158 substituting 2-methylcyclohexylamine for ethylamine in Step 3. MS: (M+H)⁺=468.

### Example 193

### 2-[5-((1S,2R)-2-Phenylcyclopropylcarbamoyl)-thiophen-2-yl]-5H-pyrrolo[2,3-b]pyrazine-7-carboxylic acid ((S)-1,2,2-trimethyl-propyl)-amide

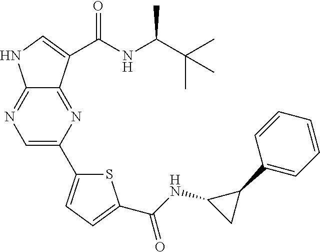 [SMILES: C[C@H](NC(=O)c1c[nH]c2ncc(-c3ccc(C(=O)N[C@H]4C[C@@H]4c4ccccc4)s3)nc12)C(C)(C)C]

Prepared according to the procedure outlined in Example 158 substituting (1S,2R)-2-phenylcyclopropylamine hydrochloride for ethylamine in Step 3. MS: (M+H)⁺=488.

### Example 194

### 2-{5-[(4-Methylthiophen-2-ylmethyl)-carbamoyl]-thiophen-2-yl}-5H-pyrrolo[2,3-b]pyrazine-7-carboxylic acid ((S)-1,2,2-trimethyl-propyl)-amide

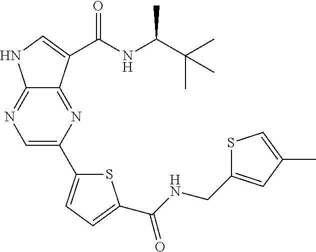 [SMILES: Cc1csc(CNC(=O)c2ccc(-c3cnc4[nH]cc(C(=O)N[C@@H](C)C(C)(C)C)c4n3)s2)c1]

Prepared according to the procedure outlined in Example 158 substituting (4-methylthiophen-2-yl)-methylamine for ethylamine in Step 3. The SEM deprotection in Step 4 was accomplished using TBAF (1.0 M in THF) in THF at reflux for 16 h followed by treatment with ethylenediamine. MS: (M+H)⁺=482.

### Example 195

### 2-{5-[(5-Methylfuran-2-ylmethyl)-carbamoyl]-thiophen-2-yl}-5H-pyrrolo[2,3-b]pyrazine-7-carboxylic acid ((S)-1,2,2-trimethyl-propyl)-amide

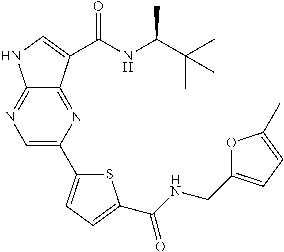 [SMILES: Cc1ccc(CNC(=O)c2ccc(-c3cnc4[nH]cc(C(=O)N[C@@H](C)C(C)(C)C)c4n3)s2)o1]

Prepared according to the procedure outlined in Example 158 substituting (5-methylfuran-2-yl)-methylamine for ethylamine in Step 3. The SEM deprotection in Step 4 was accomplished using TBAF (1.0 M in THF) in THF at reflux for 16 h followed by treatment with ethylenediamine. MS: (M+H)⁺=466.

### Example 196

### 2-[5-(Adamantan-1-ylcarbamoyl)-thiophen-2-yl]-5H-pyrrolo[2,3-b]pyrazine-7-carboxylic acid ((S)-1,2,2-trimethyl-propyl)-amide

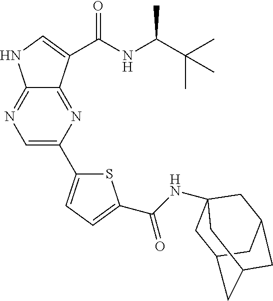 [SMILES: C[C@H](NC(=O)c1c[nH]c2ncc(-c3ccc(C(=O)NC45CC6CC(CC(C6)C4)C5)s3)nc12)C(C)(C)C]

Prepared according to the procedure outlined in Example 158 substituting adamantan-1-ylamine hydrochloride for ethylamine in Step 3. MS: (M+H)⁺=504.

### Example 197

### 2-{5-[1-(4-Fluoro-phenyl)-ethylcarbamoyl]-thiophen-2-yl}-5H-pyrrolo[2,3-b]pyrazine-7-carboxylic acid ((S)-1,2,2-trimethyl-propyl)-amide

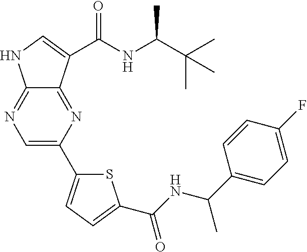 [SMILES: CC(NC(=O)c1ccc(-c2cnc3[nH]cc(C(=O)N[C@@H](C)C(C)(C)C)c3n2)s1)c1ccc(F)cc1]

Prepared according to the procedure outlined in Example 158 substituting 1-(4-fluorophenyl)-ethylamine for ethylamine in Step 3. MS: (M+H)⁺=494.

### Example 198

### 2-[5-(Methoxymethylcarbamoyl)-thiophen-2-yl]-5H-pyrrolo[2,3-b]pyrazine-7-carboxylic acid ((S)-1,2,2-trimethyl-propyl)-amide

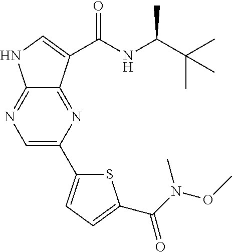 [SMILES: CON(C)C(=O)c1ccc(-c2cnc3[nH]cc(C(=O)N[C@@H](C)C(C)(C)C)c3n2)s1]

Prepared according to the procedure outlined in Example 158 substituting N,O-dimethylhydroxylamine hydrochloride for ethylamine in Step 3. MS: (M+H)⁺=416.

### Example 199

### 2-(5-Methoxycarbamoylthiophen-2-yl)-5H-pyrrolo[2,3-b]pyrazine-7-carboxylic acid ((S)-1,2,2-trimethyl-propyl)-amide

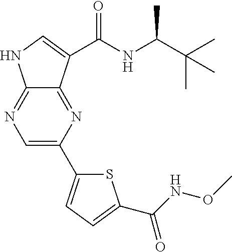 [SMILES: CONC(=O)c1ccc(-c2cnc3[nH]cc(C(=O)N[C@@H](C)C(C)(C)C)c3n2)s1]

Prepared according to the procedure outlined in Example 158 substituting O-methylhydroxylamine hydrochloride for ethylamine in Step 3. MS: (M+H)⁺=402.

### Example 200

### 2-(5-Prop-2-ynylcarbamoyl-thiophen-2-yl)-5H-pyrrolo[2,3-b]pyrazine-7-carboxylic acid ((5)-1,2,2-trimethyl-propyl)-amide

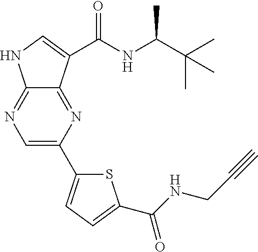 [SMILES: C#CCNC(=O)c1ccc(-c2cnc3[nH]cc(C(=O)N[C@@H](C)C(C)(C)C)c3n2)s1]

Prepared according to the procedure outlined in Example 158 substituting propargylamine for ethylamine in Step 3. MS: (M+H)⁺=410.

### Example 201

### 2-{5-[(R)-2-(3H-Imidazol-4-yl)-1-methyl-ethylcarbamoyl]-thiophen-2-yl}-5H-pyrrolo[2,3-b]pyrazine-7-carboxylic acid ((S)-1,2,2-trimethyl-propyl)-amide

Prepared according to the procedure outlined in Example 158 substituting (R)-2-(3H-imidazol-4-yl)-1-methylethylamine dihydrochloride for ethylamine in Step 3. MS: (M+H)⁺=480.

### Example 202

### 2-[5-(5,6,7,8-Tetrahydronaphthalen-2-ylcarbamoyl)-thiophen-2-yl]-5H-pyrrolo[2,3-b]pyrazine-7-carboxylic acid ((S)-1,2,2-trimethyl-propyl)-amide

Prepared according to the procedure outlined in Example 158 substituting 5,6,7,8-tetrahydronaphthalen-2-ylamine for ethylamine in Step 3. MS: (M+H)⁺=502.

### Example 203

### 2-(5-Phenylcarbamoyl-thiophen-2-yl)-5H-pyrrolo[2,3-b]pyrazine-7-carboxylic acid ((S)-1,2,2-trimethyl-propyl)-amide

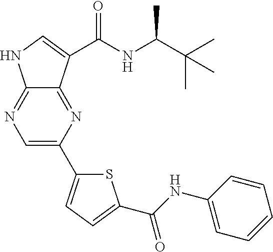 [SMILES: C[C@H](NC(=O)c1c[nH]c2ncc(-c3ccc(C(=O)Nc4ccccc4)s3)nc12)C(C)(C)C]

Prepared according to the procedure outlined in Example 158 substituting aniline for ethylamine in Step 3. MS: (M+H)⁺=448.

### Example 204

### 2-[5-((R)-1-p-Tolylethylcarbamoyl)-thiophen-2-yl]-5H-pyrrolo[2,3-b]pyrazine-7-carboxylic acid ((S)-1,2,2-trimethyl-propyl)-amide

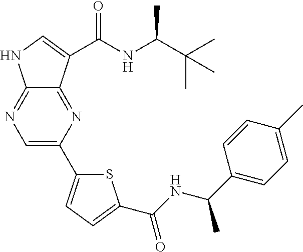 [SMILES: Cc1ccc([C@@H](C)NC(=O)c2ccc(-c3cnc4[nH]cc(C(=O)N[C@@H](C)C(C)(C)C)c4n3)s2)cc1]

Prepared according to the procedure outlined in Example 158 substituting (R)-1-(4-methylphenyl)-ethylamine for ethylamine in Step 3. MS: (M+H)⁺=490.

### Example 205

### 2-[5-(2-Methoxybenzylcarbamoyl)-thiophen-2-yl]-5H-pyrrolo[2,3-b]pyrazine-7-carboxylic acid ((S)-1,2,2-trimethyl-propyl)-amide

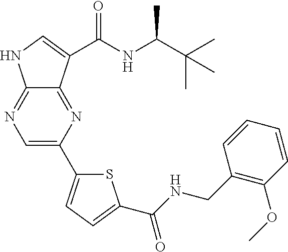 [SMILES: COc1ccccc1CNC(=O)c1ccc(-c2cnc3[nH]cc(C(=O)N[C@@H](C)C(C)(C)C)c3n2)s1]

Prepared according to the procedure outlined in Example 158 substituting 2-methoxybenzylamine for ethylamine in Step 3. MS: (M+H)⁺=492.

### Example 206

### 2-[5-(2,5-Dimethoxybenzylcarbamoyl)-thiophen-2-yl]-5H-pyrrolo[2,3-b]pyrazine-7-carboxylic acid ((S)-1,2,2-trimethyl-propyl)-amide

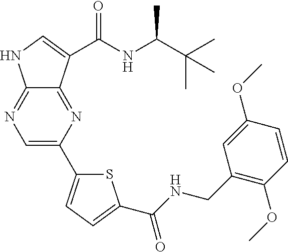 [SMILES: COc1ccc(OC)c(CNC(=O)c2ccc(-c3cnc4[nH]cc(C(=O)N[C@@H](C)C(C)(C)C)c4n3)s2)c1]

Prepared according to the procedure outlined in Example 158 substituting 2,5-dimethoxybenzylamine for ethylamine in Step 3. MS: (M+H)⁺=522.

### Example 207

### 2-{5-[(4-Fluorobenzyl)-methyl-carbamoyl]-thiophen-2-yl}-5H-pyrrolo[2,3-b]pyrazine-7-carboxylic acid ((S)-1,2,2-trimethyl-propyl)-amide

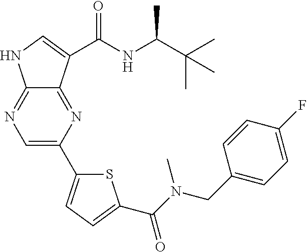 [SMILES: C[C@H](NC(=O)c1c[nH]c2ncc(-c3ccc(C(=O)N(C)Cc4ccc(F)cc4)s3)nc12)C(C)(C)C]

Prepared according to the procedure outlined in Example 158 substituting (4-fluorobenzyl)-methylamine for ethylamine in Step 3. MS: (M+H)⁺=494.

### Example 208

### 2-[5-(3-Methoxybenzylcarbamoyl)-thiophen-2-yl]-5H-pyrrolo[2,3-b]pyrazine-7-carboxylic acid ((S)-1,2,2-trimethyl-propyl)-amide

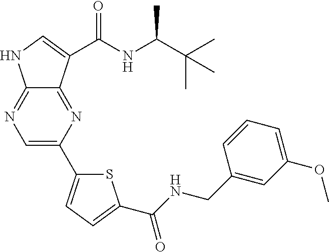 [SMILES: COc1cccc(CNC(=O)c2ccc(-c3cnc4[nH]cc(C(=O)N[C@@H](C)C(C)(C)C)c4n3)s2)c1]

Prepared according to the procedure outlined in Example 158 substituting 3-methoxybenzylamine for ethylamine in Step 3. MS: (M+H)⁺=492.

### Example 209

### 2-[5-(3-Trifluoromethylbenzylcarbamoyl)-thiophen-2-yl]-5H-pyrrolo[2,3-b]pyrazine-7-carboxylic acid ((S)-1,2,2-trimethyl-propyl)-amide

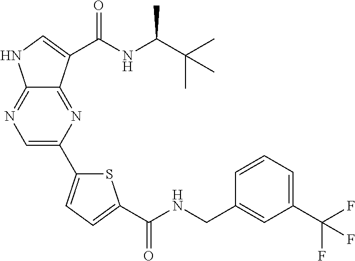 [SMILES: C[C@H](NC(=O)c1c[nH]c2ncc(-c3ccc(C(=O)NCc4cccc(C(F)(F)F)c4)s3)nc12)C(C)(C)C]

Prepared according to the procedure outlined in Example 158 substituting 3-trifluoromethylbenzylamine for ethylamine in Step 3. MS: (M+H)⁺=530.

### Example 210

### 2-[5-(2-Chloro-4-iodophenylcarbamoyl)-thiophen-2-yl]-5H-pyrrolo[2,3-b]pyrazine-7-carboxylic acid ((S)-1,2,2-trimethyl-propyl)-amide

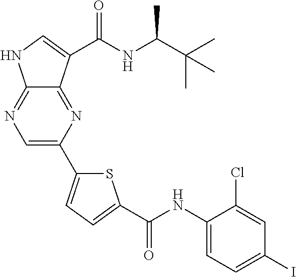 [SMILES: C[C@H](NC(=O)c1c[nH]c2ncc(-c3ccc(C(=O)Nc4ccc(I)cc4Cl)s3)nc12)C(C)(C)C]

### Step 1

To a stirred solution of 5-[7-((S)-1,2,2-trimethyl-propylcarbamoyl)-5-(2-trimethylsilanyl-ethoxymethyl)-5H-pyrrolo[2,3-b]pyrazin-2-yl]-thiophene-2-carboxylic acid (150 mg, 0.30 mmol) in dry pyridine was added HATU (228 mg, 0.60 mmol) and 2-chloro-4-iodoaniline (380 mg, 1.50 mmol). The reaction mixture was stirred at room temperature for 72 h then evaporated under reduced pressure and portioned between water and EtOAc. The organic layer was dried over Na₂SO₄ and concentrated. The crude residue was purified by silica gel column chromatography (EtOAc/hexanes) to give 80 mg (36%) of 2-[5-(2-chloro-4-iodo-phenylcarbamoyl)-thiophen-2-yl]-5-(2-trimethylsilanyl-ethoxymethyl)-5H-pyrrolo[2,3-b]pyrazine-7-carboxylic acid ((S)-1,2,2-trimethyl-propyl)-amide as a yellow solid.

### Step 2

2-[5-(2-Chloro-4-iodophenylcarbamoyl)-thiophen-2-yl]-5H-pyrrolo[2,3-b]pyrazine-7-carboxylic acid ((S)-1,2,2-trimethyl-propyl)-amide. Prepared according to the procedure outlined in Example 170, step 4 substituting 2-[5-(2-chloro-4-iodo-phenylcarbamoyl)-thiophen-2-yl]-5-(2-trimethylsilanyl-ethoxymethyl)-5H-pyrrolo[2,3-b]pyrazine-7-carboxylic acid ((S)-1,2,2-trimethyl-propyl)-amide for 2-(5-ethylcarbamoyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide. MS: (M+H)⁺=608.

### Example 211

### 2-[5-((R)-1,2,2-Trimethyl-propylcarbamoyl)-thiophen-2-yl]-5H-pyrrolo[2,3-b]pyrazine-7-carboxylic acid ((S)-1,2,2-trimethyl-propyl)-amide

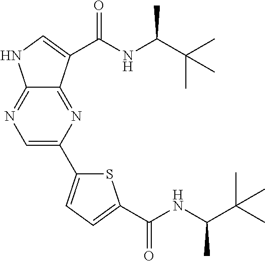 [SMILES: C[C@H](NC(=O)c1c[nH]c2ncc(-c3ccc(C(=O)N[C@H](C)C(C)(C)C)s3)nc12)C(C)(C)C]

### Step 1

A solution of 5-[7-((S)-1,2,2-trimethyl-propylcarbamoyl)-5-(2-trimethylsilanyl-ethoxymethyl)-5H-pyrrolo[2,3-b]pyrazin-2-yl]-thiophene-2-carboxylic acid (0.042 g, 0.084 mmol), 2 mL of anhydrous dichloromethane, (R)-3,3-dimethylbutan-2-amine (0.025 mL, 0.19 mmol), 4-dimethylaminopyridine (0.012 g, 0.101 mmol)) and N-(3-dimethylaminopropyl)-N′-ethylcarbodiimide hydrochloride (0.037 g, 0.190 mmol) was stirred at room temperature for 4 h. Dichloromethane (10 mL) was added, and the solution was sequentially washed with 10 mL of a 1 M citric acid solution, 10 mL of water, 10 mL of a 10% NaOH solution, and 10 mL of water then dried over Na₂SO₄, filtered and concentrated to afford 0.074 g (>100%) of 2-[5-(R)-1,2,2-trimethyl-propylcarbamoyl)-thiophen-2-yl]-5-(2-trimethylsilanyl-ethoxymethyl)-5H-pyrrolo[2,3-b]pyrazine-7-carboxylic acid ((S)-1,2,2-trimethyl-propyl)-amide as a yellow film which was used without further purification. MS: (M+Na)⁺=608.

### Step 2

A solution of the above-prepared crude 2-[5-((R)-1,2,2-trimethyl-propylcarbamoyl)-thiophen-2-yl]-5-(2-trimethylsilanyl-ethoxymethyl)-5H-pyrrolo[2,3-b]pyrazine-7-carboxylic acid ((S)-1,2,2-trimethyl-propyl)-amide, 1 mL of CH₂Cl₂ and 1 mL of trilfluoroacetic acid was stirred at room temperature for 2 h, then concentrated to a yellow residue. To the residue was added 0.5 mL of dichloromethane and 0.5 mL of ethylenediamine. The yellow solution was stirred for 90 min then partitioned between 10 mL of ethyl acetate and 5 mL of water. The aqueous layer was extracted with 10 mL of ethyl acetate. The combined organic layers were dried over Na₂SO₄, filtered, and concentrated to a yellow oily residue. Column chromatography (80-100% EtOAc/hexanes) afforded 0.018 g (46%, two steps) of 2-[5-((R)-1,2,2-trimethyl-propylcarbamoyl)-thiophen-2-yl]-5H-pyrrolo[2,3-b]pyrazine-7-carboxylic acid ((S)-1,2,2-trimethyl-propyl)-amide as a pale yellow solid. MS: (M+Na)⁺=478.

### Example 212

### 2-[5-(2,2-Dimethyl-propylcarbamoyl)-thiophen-2-yl]-5H-pyrrolo[2,3-b]pyrazine-7-carboxylic acid ((S)-1,2,2-trimethyl-propyl)-amide

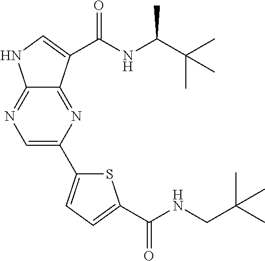 [SMILES: C[C@H](NC(=O)c1c[nH]c2ncc(-c3ccc(C(=O)NCC(C)(C)C)s3)nc12)C(C)(C)C]

Prepared according to the procedure outlined in Example 211 substituting 2,2-dimethyl-propylamine for (R)-3,3-dimethylbutan-2-amine in Step 1. MS: (M+Na)⁺=464.

### Example 213

### 2-[5-((R)-2-Methanesulfonyl-1-methyl-ethylcarbamoyl)-thiophen-2-yl]-5H-pyrrolo[2,3-b]pyrazine-7-carboxylic acid ((S)-1,2,2-trimethyl-propyl)-amide

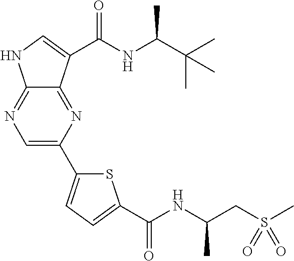 [SMILES: C[C@H](CS(C)(=O)=O)NC(=O)c1ccc(-c2cnc3[nH]cc(C(=O)N[C@@H](C)C(C)(C)C)c3n2)s1]

Prepared according to the procedure outlined in Example 158 substituting (R)-1-(methylsulfonyl)propan-2-amine hydrochloride for ethylamine in Step 3. SEM deprotection in Step 4 was conducted with TFA followed by CH₂Cl₂/MeOH/NH₄OH (80:19:1). MS: (M+H)⁺=492.

### Example 214

### 2-[5-(1,1-Dioxo-hexahydro-1-thiopyran-4-ylcarbamoyl)-thiophen-2-yl]-5H-pyrrolo[2,3-b]pyrazine-7-carboxylic acid ((S)-1,2,2-trimethyl-propyl)-amide

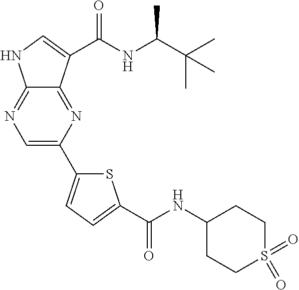 [SMILES: C[C@H](NC(=O)c1c[nH]c2ncc(-c3ccc(C(=O)NC4CCS(=O)(=O)CC4)s3)nc12)C(C)(C)C]

Prepared according to the procedure outlined in Example 158 substituting (1,1-dioxidotetrahydro-2H-thiopiran-4-yl)amine hydrochloride for ethylamine in Step 3. SEM deprotection in Step 4 was conducted with TFA followed by CH₂Cl₂/MeOH/NH₄OH (80:19:1). MS: (M+H)⁺=504.

### Example 215

### 2-[5-(1,1-Dioxo-1-thiomorpholine-4-carbonyl)-thiophen-2-yl]-5H-pyrrolo[2,3-b]pyrazine-7-carboxylic acid ((S)-1,2,2-trimethyl-propyl)-amide

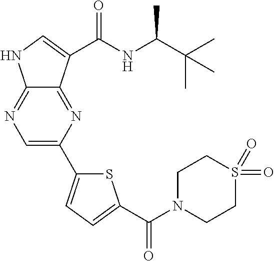 [SMILES: C[C@H](NC(=O)c1c[nH]c2ncc(-c3ccc(C(=O)N4CCS(=O)(=O)CC4)s3)nc12)C(C)(C)C]

Prepared according to the procedure outlined in Example 158 substituting thiomorpholine 1,1-dioxide for ethylamine in Step 3. SEM deprotection in Step 4 was conducted with TFA followed by CH₂Cl₂/MeOH/NH₄OH (80:19:1). MS: (M+H)⁺=490.

### Example 216

### 2-[5-(2-Methoxy-1-methylethylcarbamoyl)-thiophen-2-yl]-5H-pyrrolo[2,3-b]pyrazine-7-carboxylic acid ((S)-1,2,2-trimethyl-propyl)-amide

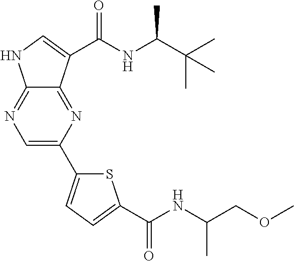 [SMILES: COCC(C)NC(=O)c1ccc(-c2cnc3[nH]cc(C(=O)N[C@@H](C)C(C)(C)C)c3n2)s1]

Prepared according to the procedure outlined in Example 158 substituting 1-methoxypropan-2-amine for ethylamine in Step 3. SEM deprotection in Step 4 was conducted with TFA followed by CH₂Cl₂/MeOH/NH₄OH (80:19:1). MS: (M+H)⁺=444.

### Example 217

### 2-(5-Carbamoyl-thiophen-2-yl)-5H-pyrrolo[2,3-b]pyrazine-7-carboxylic acid ((S)-1,2,2-trimethyl-propyl)-amide

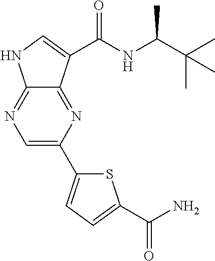 [SMILES: C[C@H](NC(=O)c1c[nH]c2ncc(-c3ccc(C(N)=O)s3)nc12)C(C)(C)C]

Prepared according to the procedure outlined in Example 158 substituting 1,1,1-trifluoropropan-2-amine for ethylamine in Step 3. The title compound was presumed to be a result of either hydrolysis of the initially formed 1,1,1-trifluoro-2-propylamide or impure 1,1,1-trifluoropropan-2-amine starting material. SEM deprotection in Step 4 was conducted with TFA followed by CH₂Cl₂/MeOH/NH₄OH (80:19:1). MS: (M+H)⁺=372.

### Example 218

### 2-[5-(3,3,3-Trifluoropropylcarbamoyl)-thiophen-2-yl]-5H-pyrrolo[2,3-b]pyrazine-7-carboxylic acid ((S)-1,2,2-trimethyl-propyl)-amide

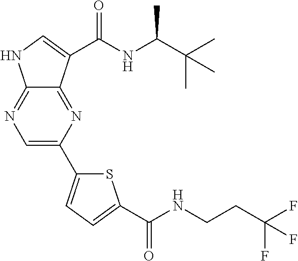 [SMILES: C[C@H](NC(=O)c1c[nH]c2ncc(-c3ccc(C(=O)NCCC(F)(F)F)s3)nc12)C(C)(C)C]

Prepared according to the procedure outlined in Example 158 substituting 3,3,3-trifluoropropan-1-amine hydrochloride for ethylamine in Step 3. SEM deprotection in Step 4 was conducted with TFA followed by CH₂Cl₂/MeOH/NH₄OH (80:19:1). MS: (M+H)⁺=468.

### Example 219

### 2-[5-(2-Oxa-6-azaspiro[3.3]heptane-6-carbonyl)-thiophen-2-yl]-5H-pyrrolo[2,3-b]pyrazine-7-carboxylic acid ((S)-1,2,2-trimethyl-propyl)-amide

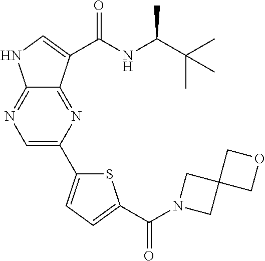 [SMILES: C[C@H](NC(=O)c1c[nH]c2ncc(-c3ccc(C(=O)N4CC5(COC5)C4)s3)nc12)C(C)(C)C]

Prepared according to the procedure outlined in Example 158 substituting 2-oxa-6-azaspiro[3.3]heptane hemioxalate for ethylamine in Step 3. SEM deprotection in Step 4 was conducted with TFA followed by CH₂Cl₂/MeOH/NH₄OH (80:19:1). MS: (M+H)⁺=454.

### Example 220

### 2-[5-(3,3-Bishydroxymethyl-azetidine-1-carbonyl)-thiophen-2-yl]-5H-pyrrolo[2,3-b]pyrazine-7-carboxylic acid ((S)-1,2,2-trimethyl-propyl)-amide

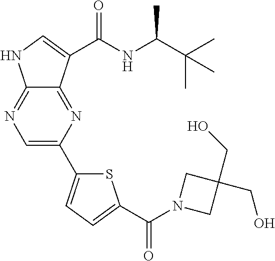 [SMILES: C[C@H](NC(=O)c1c[nH]c2ncc(-c3ccc(C(=O)N4CC(CO)(CO)C4)s3)nc12)C(C)(C)C]

Isolated as a byproduct from Example 219, step 4. MS: (M+H)⁺=472.

### Example 221

### 2-[4-Methyl-5-(tetrahydropyran-4-ylcarbamoyl)-thiophen-2-yl]-5H-pyrrolo[2,3-b]pyrazine-7-carboxylic acid ((S)-1,2,2-trimethyl-propyl)-amide

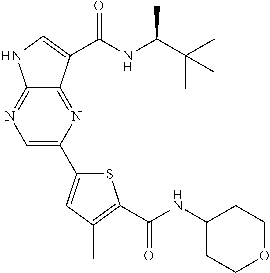 [SMILES: Cc1cc(-c2cnc3[nH]cc(C(=O)N[C@@H](C)C(C)(C)C)c3n2)sc1C(=O)NC1CCOCC1]

Prepared according to the procedure outlined in Example 158 substituting 5-formyl-4-methylthiophen-2-ylboronic acid for 2-formylthiophene-5-boronic acid in Step 1 and tetrahydro-2H-pyran-4-amine hydrochloride for ethylamine in Step 3. SEM deprotection in Step 4 was conducted with TFA followed by CH₂Cl₂/MeOH/NH₄OH (80:19:1). MS: (M+H)⁺=470.

### Example 222

### 2-[5-(1,1-Dioxo-1-thiomorpholine-4-carbonyl)-4-methyl-thiophen-2-yl]-5H-pyrrolo[2,3-b]pyrazine-7-carboxylic acid ((S)-1,2,2-trimethyl-propyl)-amide

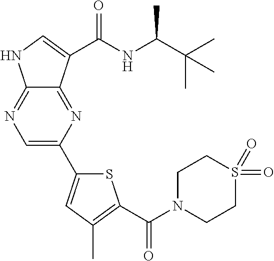 [SMILES: Cc1cc(-c2cnc3[nH]cc(C(=O)N[C@@H](C)C(C)(C)C)c3n2)sc1C(=O)N1CCS(=O)(=O)CC1]

Prepared according to the procedure outlined in Example 158 substituting 5-formyl-4-methylthiophen-2-ylboronic acid for 2-formylthiophene-5-boronic acid in Step 1 and thiomorpholine 1,1-dioxide for ethylamine in Step 3. SEM deprotection in Step 4 was conducted with TFA followed by CH₂Cl₂/MeOH/NH₄OH (80:19:1). MS: (M+H)⁺=504.

### Example 223

### 2-[4-Methyl-5-(2-oxa-6-aza-spiro[3.3]heptane-6-carbonyl)-thiophen-2-yl]-5H-pyrrolo[2,3-b]pyrazine-7-carboxylic acid ((S)-1,2,2-trimethyl-propyl)-amide

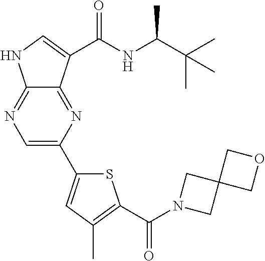 [SMILES: Cc1cc(-c2cnc3[nH]cc(C(=O)N[C@@H](C)C(C)(C)C)c3n2)sc1C(=O)N1CC2(COC2)C1]

Prepared according to the procedure outlined in Example 158 substituting 5-formyl-4-methylthiophen-2-ylboronic acid for 2-formylthiophene-5-boronic acid in Step 1 and 2-oxa-6-azaspiro[3.3]heptane hemioxalate for ethylamine in Step 3. SEM deprotection in Step 4 was conducted with TFA followed by CH₂Cl₂/MeOH/NH₄OH (80:19:1). MS: (M+H)⁺=468.

### Example 224

### 2-[5-(3,3-Bishydroxymethyl-azetidine-1-carbonyl)-4-methyl-thiophen-2-yl]-5H-pyrrolo[2,3-b]pyrazine-7-carboxylic acid ((S)-1,2,2-trimethyl-propyl)-amide

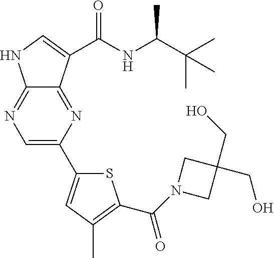 [SMILES: Cc1cc(-c2cnc3[nH]cc(C(=O)N[C@@H](C)C(C)(C)C)c3n2)sc1C(=O)N1CC(CO)(CO)C1]

Isolated as a byproduct from Example 223, step 4. MS: (M−H)⁻=484.

### Example 225

### 2-[5-(Tetrahydropyran-4-ylcarbamoyl)-thiophen-2-yl]-5H-pyrrolo[2,3-b]pyrazine-7-carboxylic acid ((S)-2-cyano-1,2,2-trimethyl-ethyl)-amide

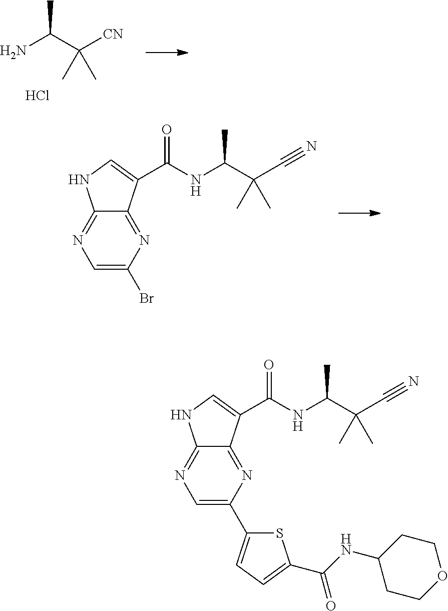

### Step 1

(S)-3-Amino-2,2-dimethyl-butyronitrile hydrochloride was prepared according to Example 141, Steps 1-3.

### Step 2

In a flask were combined 2-bromo-5-(2-trimethylsilanylethoxymethyl)-5H-pyrrolo[2,3-b]pyrazine-7-carboxylic acid (1.10 g, 2.95 mmol), (S)-3-amino-2,2-dimethyl-butyronitrile hydrochloride (439 mg, 2.95 mmol), EDC (1.30 g, 6.80 mmol) and HOBt (1.15 g, 6.80 mmol). DMF (27 mL) was added followed by i-Pr₂NEt (3.6 mL, 20.7 mmol). The reaction mixture was stirred at room temperature for 1.5 h and then quenched with water and extracted with EtOAc. The organics were washed with 10% citric acid, sat'd NaHCO₃, sat'd LiCl, and sat'd NaCl then dried over MgSO₄ and concentrated. The residue was purified by SiO₂ chromatography (20-100% EtOAc/hexane) to give 1.32 g (96%) 2-bromo-5-(2-trimethylsilanyl-ethoxymethyl)-5H-pyrrolo[2,3-b]pyrazine-7-carboxylic acid ((S)-2-cyano-1,2,2-trimethyl-ethyl)-amide as an off-white solid.

### Step 3

2-[5-(Tetrahydropyran-4-ylcarbamoyl)-thiophen-2-yl]-5H-pyrrolo[2,3-b]pyrazine-7-carboxylic acid ((S)-2-cyano-1,2,2-trimethyl-ethyl)-amide was prepared according to the procedure outlined in Example 158 substituting 2-bromo-5-(2-trimethylsilanyl-ethoxymethyl)-5H-pyrrolo[2,3-b]pyrazine-7-carboxylic acid ((S)-2-cyano-1,2,2-trimethyl-ethyl)-amide for 2-bromo-5-(2-trimethylsilanyl-ethoxymethyl)-5H-pyrrolo[2,3-b]pyrazine-7-carboxylic acid ((5)-1,2,2-trimethyl-propyl)-amide in Step 1 and tetrahydro-2H-pyran-4-amine hydrochloride for ethylamine in Step 3. SEM deprotection in Step 4 was conducted with TFA followed by ethylenediamine. MS: (M+H)⁺=467.

### Example 226

### 2-[5-(Piperidine-1-carbonyl)-thiophen-2-yl]-5H-pyrrolo[2,3-b]pyrazine-7-carboxylic acid ((S)-2-cyano-1,2,2-trimethyl-ethyl)-amide

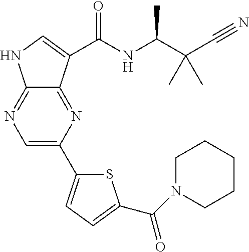 [SMILES: C[C@H](NC(=O)c1c[nH]c2ncc(-c3ccc(C(=O)N4CCCCC4)s3)nc12)C(C)(C)C#N]

Prepared according to the procedure outlined in Example 158 substituting 2-bromo-5-(2-trimethylsilanyl-ethoxymethyl)-5H-pyrrolo[2,3-b]pyrazine-7-carboxylic acid ((S)-2-cyano-1,2,2-trimethyl-ethyl)-amide for 2-bromo-5-(2-trimethylsilanyl-ethoxymethyl)-5H-pyrrolo[2,3-b]pyrazine-7-carboxylic acid ((S)-1,2,2-trimethyl-propyl)-amide in Step 1 and piperidine for ethylamine in Step 3. SEM deprotection in Step 4 was conducted with TFA followed by CH₂Cl₂/MeOH/NH₄OH (90:9:1). MS: (M+H)⁺=467; mp=253-257.

### Example 227

### 2-[5-(Tetrahydro-pyran-4-ylcarbamoyl)-thiophen-2-yl]-5H-pyrrolo[2,3-b]pyrazine-7-carboxylic acid ((S)-1,2,2-trimethyl-propyl)-amide

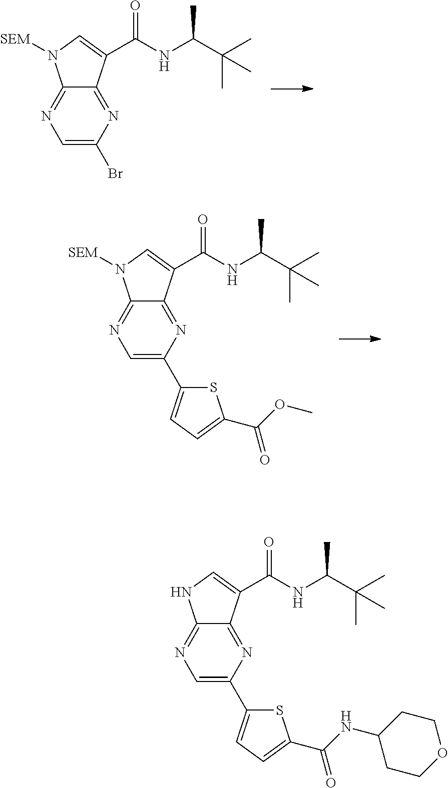 [SMILES: C[C@H](NC(=O)c1cn(COCC[Si](C)(C)C)c2ncc(Br)nc12)C(C)(C)C ; COC(=O)c1ccc(-c2cnc3c(n2)c(C(=O)N[C@@H](C)C(C)(C)C)cn3COCC[Si](C)(C)C)s1 ; C[C@H](NC(=O)c1c[nH]c2ncc(-c3ccc(C(=O)NC4CCOCC4)s3)nc12)C(C)(C)C]

### Step 1

In a microwave vial 2-bromo-5-(2-trimethylsilanyl-ethoxymethyl)-5H-pyrrolo[2,3-b]pyrazine-7-carboxylic acid ((S)-1,2,2-trimethyl-propyl)-amide (100 mg, 0.22 mmol) was dissolved in 5:1 dioxane/water (6 mL). The vial was purged with argon then 5-(methoxycarbonyl)thiophen-2-yl boronic acid (45 mg, 0.24 mmol), Na₂CO₃ (70 mg, 0.66 mmol) and Pd(PPh₃)₄ (13 mg, 0.011 mmol) were added. The vial was sealed and heated in a microwave reactor at 140° C. for 1 h. An additional amount of 5-(methoxycarbonyl)thiophen-2-yl boronic acid (23 mg, 0.12 mmol) and Pd(PPh₃)₄ (6 mg, 0.005 mmol) were added and the reaction was again heated in a microwave reactor at 140° C. for 1 h. The reaction was repeated on the same scale and the crude reaction mixtures from the two runs were combined and partitioned between water and EtOAc. Saturated NaHCO₃ was added and the aqueous layer was extracted with EtOAc (2×). The combined organic layers were washed with brine then dried over Na₂SO₄ and evaporated under reduced pressure. The crude residue was purified by silica gel column chromatography (0-50% EtOAc/hexanes) to afford 90 mg (40%, combined, 2 runs) of 5-[7-((S)-1,2,2-trimethyl-propylcarbamoyl)-5-(2-trimethylsilanyl-ethoxymethyl)-5H-pyrrolo[2,3-b]pyrazin-2-yl]-thiophene-2-carboxylic acid methyl ester as an off-white solid.

### Step 2

5-[7-((S)-1,2,2-Trimethyl-propylcarbamoyl)-5-trimethylsilanyl-ethoxymethyl)-5H-pyrrolo[2,3-b]pyrazin-2-yl]-thiophene-2-carboxylic acid methyl ester (90 mg, 0.17 mmol) was dissolved in THF (1 mL) and methanol (0.5 mL). Lithium hydroxide (29 mg, 0.70 mmol) in water (1 mL) was added slowly. The solution was stirred for 2 h and then water and ethyl acetate were added. The pH was adjusted to 3, the layers were separated and the aqueous layer was extracted twice more with ethyl acetate. The combined organic layers were washed with brine, dried over sodium sulfate and concentrated to afford 5-[7-(S)-1,2,2-trimethyl-propylcarbamoyl)-5-(2-trimethylsilanyl-ethoxymethyl)-5H-pyrrolo[2,3-b]pyrazin-2-yl]-thiophene-2-carboxylic acid which was used without further purification.

### Step 3

5-[7-((S)-1,2,2-Trimethyl-propylcarbamoyl)-5-(2-trimethylsilanyl-ethoxymethyl)-5H-pyrrolo[2,3-b]pyrazin-2-yl]-thiophene-2-carboxylic acid (87 mg, 0.17 mmol), O-benzotriazol-1-yl-N,N,N′,N′-tetramethyluronium tetrafluoroborate (61 mg, 0.19 mmol) and N,N-diisopropylethylamine (0.10 mL, 0.52 mmol) were dissolved in acetonitrile (1.7 mL). Tetrahydro-2H-pyran-4-amine hydrochloride (26 mg, 0.19 mmol) was added and the mixture was stirred at room temperature for 18 h. Water, dilute HCl solution and ethyl acetate were added, the layers were separated and the aqueous layer was extracted twice more with ethyl acetate. The combined organic layers were washed with sodium bicarbonate solution, dried over sodium sulfate and concentrated. The residue was purified by silica gel chromatography (ethyl acetate/hexanes) to give 80 mg (76%) of 2-[5-(tetrahydro-pyran-4-ylcarbamoyl)-thiophen-2-yl]-5-(2-trimethylsilanyl-ethoxymethyl)-5H-pyrrolo[2,3-b]pyrazine-7-carboxylic acid ((S)-1,2,2-trimethyl-propyl)-amide.

### Step 4

2-[5-(Tetrahydro-pyran-4-ylcarbamoyl)-thiophen-2-yl]-5-(2-trimethylsilanyl-ethoxymethyl)-5H-pyrrolo[2,3-b]pyrazine-7-carboxylic acid ((S)-1,2,2-trimethyl-propyl)-amide (80 mg, 0.137 mmol) was dissolved in dichloromethane (1 mL) and then stirred in an ice bath. Trifluoroacetic acid (0.4 mL) was added slowly and the ice bath was removed. The reaction was stirred for 3 h and then cooled in ice bath. Sodium bicarbonate solution was added and the mixture was extracted three times with ethyl acetate. The combined organic layers were washed with brine and dried over sodium sulfate. After evaporation, the residue was dissolved in absolute ethanol (6 mL) and sodium acetate (224 mg, 2.7 mmol) was added. The mixture was stirred at 60° C. for 20 h. The reaction was cooled, and water and ethyl acetate were added. The aqueous layer was extracted twice more with ethyl acetate. The combined organic layers were washed with brine, dried over sodium sulfate and evaporated. The residue was purified by silica gel chromatography (MeOH/dichloromethane) to afford 49 mg (79%) of 2-[5-(tetrahydro-pyran-4-ylcarbamoyl)-thiophen-2-yl]-5H-pyrrolo[2,3-b]pyrazine-7-carboxylic acid ((S)-1,2,2-trimethyl-propyl)-amide as an off-white solid. MS: (M+H)⁺=456; mp=333-334° C.

### Example 228

### 2-(5-Benzylcarbamoyl-thiophen-2-yl)-5H-pyrrolo[2,3-b]pyrazine-7-carboxylic acid ((S)-1,2,2-trimethyl-propyl)-amide

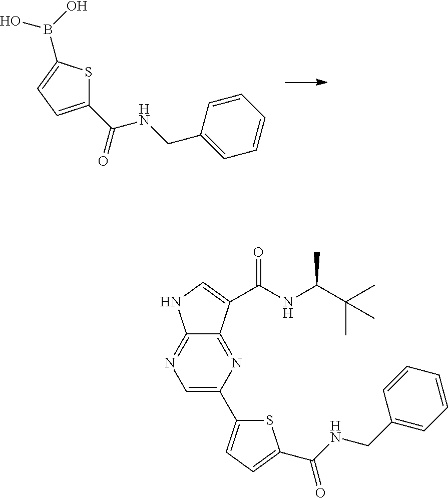 [SMILES: O=C(NCc1ccccc1)c1ccc(B(O)O)s1 ; C[C@H](NC(=O)c1c[nH]c2ncc(-c3ccc(C(=O)NCc4ccccc4)s3)nc12)C(C)(C)C]

### Step 1

Thiophenecarboxylate boronic acid (0.5 g, 2.9 mmol) was dissolved in THF (12 mL). 1,1′-carbonyldiimidazole (0.47 g, 2.9 mmol) was added and reaction was stirred at room temperature for 1 h. Benzylamine (0.32 mL, 2.9 mmol) was slowly added, and the reaction was stirred for 18 h. The solvent was evaporated and the residue was partitioned between ethyl acetate and water. The organic layer was washed with ammonium chloride solution, dried over sodium sulfate and evaporated to give 0.55 g of 5-(benzylcarbamoyl)thiophen-2-yl boronic acid which was used without further purification. LCMS: (M+Na)⁺=284.

### Step 2

In a microwave vial 2-bromo-5-(2-trimethylsilanyl-ethoxymethyl)-5H-pyrrolo[2,3-b]pyrazine-7-carboxylic acid ((S)-1,2,2-trimethyl-propyl)-amide (100 mg, 0.22 mmol) was dissolved in 5:1 dioxane/water (6 mL). The vial was purged with argon then 5-(benzylcarbamoyl)thiophen-2-yl boronic acid (86 mg, 0.33 mmol), Na₂CO₃ (70 mg, 0.66 mmol) and Pd(PPh₃)₄ (13 mg, 0.011 mmol) were added. The vial was sealed and heated in a microwave reactor at 150° C. for 0.5 h. An additional amount of 5-(benzylcarbamoyl)thiophen-2-yl boronic acid (40 mg, 0.15 mmol) and Pd(PPh₃)₄ (6 mg, 0.005 mmol) were added and the reaction was again heated in a microwave reactor at 140° C. for 1 h. The reaction mixture was cooled and partitioned between water and EtOAc. Saturated NaHCO₃ was added and the aqueous layer was extracted with EtOAc (2×). The combined organic layers were washed with brine then dried over Na₂SO₄ and evaporated under reduced pressure. The crude residue was purified by silica gel column chromatography (EtOAc/hexanes) to afford 68 mg (52%) of 2-(5-benzylcarbamoyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide as an off-white solid.

### Step 3

2-(5-Benzylcarbamoyl-thiophen-2-yl)-5-(2-trimethylsilanyl-ethoxymethyl)-5H-pyrrolo[2,3-b]pyrazine-7-carboxylic acid ((S)-1,2,2-trimethyl-propyl)-amide (65 mg, 0.11 mmol) was dissolved in dichloromethane (0.8 mL) and then stirred in an ice bath. Trifluoroacetic acid (0.4 mL) was added slowly and the ice bath was removed. The reaction was stirred for 3 h and then cooled in ice bath. Sodium bicarbonate solution was added and the mixture was extracted three times with ethyl acetate. The combined organic layers were washed with brine and dried over sodium sulfate. After evaporation, the residue was dissolved in absolute ethanol (7 mL) and sodium acetate (180 mg, 2.2 mmol) was added. The mixture was stirred at 60° C. for 20 h. The reaction was cooled, and water and ethyl acetate were added. The aqueous layer was extracted twice more with ethyl acetate. The combined organic layers were washed with brine, dried over sodium sulfate and evaporated. The residue was purified by silica gel chromatography (MeOH/dichloromethane) to afford 40 mg (79%) of 2-(5-benzylcarbamoyl-thiophen-2-yl)-5H-pyrrolo[2,3-b]pyrazine-7-carboxylic acid ((S)-1,2,2-trimethyl-propyl)-amide as an off-white solid. MS: (M+H)⁺=462; mp=225-226° C.

### Example 229

### 2-[5-(3-Cyanobenzylcarbamoyl)-thiophen-2-yl]-5H-pyrrolo[2,3-b]pyrazine-7-carboxylic acid ((S)-1,2,2-trimethyl-propyl)-amide

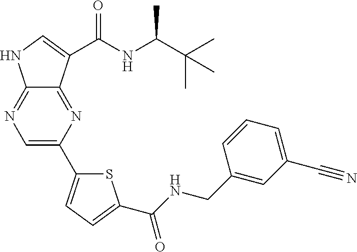 [SMILES: C[C@H](NC(=O)c1c[nH]c2ncc(-c3ccc(C(=O)NCc4cccc(C#N)c4)s3)nc12)C(C)(C)C]

Prepared according to the procedure outlined in Example 228 substituting 3-cyanobenzylamine for benzylamine in Step 1. MS: (M+H)⁺=487; mp=171-174° C.

### Example 230

### 2-(3-Cyanophenoxy)-5H-pyrrolo[2,3-b]pyrazine-7-carboxylic acid isopropylamide

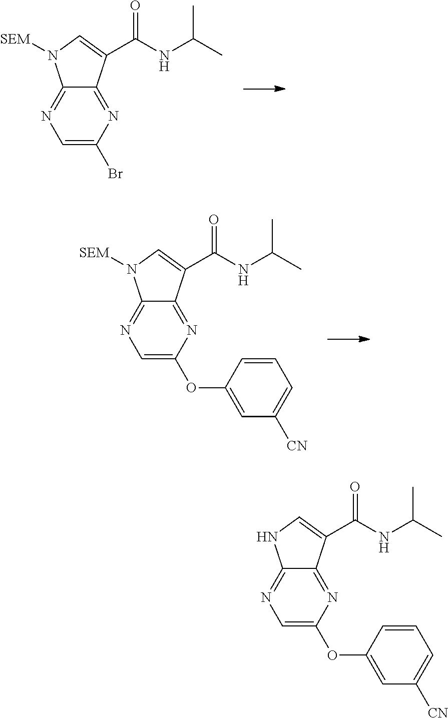 [SMILES: CC(C)NC(=O)c1cn(COCC[Si](C)(C)C)c2ncc(Br)nc12 ; CC(C)NC(=O)c1cn(COCC[Si](C)(C)C)c2ncc(Oc3cccc(C#N)c3)nc12 ; CC(C)NC(=O)c1c[nH]c2ncc(Oc3cccc(C#N)c3)nc12]

### Step 1

To a stirred solution of 2-bromo-5-(2-trimethylsilanyl-ethoxymethyl)-5H-pyrrolo[2,3-b]pyrazine-7-carboxylic acid isopropylamide (200 mg, 0.48 mmol) in toluene (5 mL) in a pressure tube, were added 3-cyanophenol (87 mg, 0.73 mmol), K₃PO₄ (204 mg, 0.96 mmol), and 2-di-tert-butylphosphino-2′-(N,N-dimethylamino)biphenyl (24 mg, 0.07 mmol). The reaction mixture was purged thoroughly with argon gas for 20 min, then Pd(OAc)₂ (11 mg, 0.05 mmol) was added. The tube was sealed and the reaction mixture was heated at 140° C. for 18 h then cooled to room temperature, quenched with water (20 mL) and extracted with EtOAc (3×15 mL). The combined organic layers were washed with brine, dried over anhydrous Na₂SO₄ and concentrated under reduced pressure. The residue was purified by column chromatography over silica gel (100-200 mesh) using 20-60% EtOAc/hexane as the eluting solvent to afford 160 mg (73%) of 2-(3-cyano-phenoxy)-5-(2-trimethylsilanyl-ethoxymethyl)-5H-pyrrolo[2,3-b]pyrazine-7-carboxylic acid isopropylamide as a yellow-brown oil.

### Step 2

A stirred solution of 2-(3-cyano-phenoxy)-5-(2-trimethylsilanyl-ethoxymethyl)-5H-pyrrolo[2,3-b]pyrazine-7-carboxylic acid isopropylamide (160 mg, 0.35 mmol) in 1.0 M HCl in AcOH (5 mL) was heated at 65° C. for 3 h. The reaction mixture was concentrated under reduced pressure, the residue was dissolved in MeOH/Et₃N/H₂O (8:1:1, 3 mL) and ethylenediamine (0.1 mL) was added at 0° C. The reaction mixture was stirred at 25° C. for 18 h then concentrated under reduced pressure. The crude residue was purified by silica gel column chromatography (MeOH/CH₂Cl₂) to afford 50 mg (44%) of 2-(3-cyanophenoxy)-5H-pyrrolo[2,3-b]pyrazine-7-carboxylic acid isopropylamide as an off-white solid. MS: (M+H)⁺=322.

### Example 231

### 2-(3-Methoxyphenoxy)-5H-pyrrolo[2,3-b]pyrazine-7-carboxylic acid isopropylamide

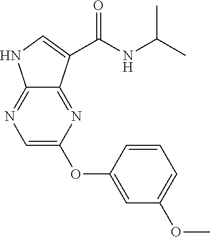 [SMILES: COc1cccc(Oc2cnc3[nH]cc(C(=O)NC(C)C)c3n2)c1]

Prepared according to the procedure outlined in Example 230 substituting 3-methoxyphenol for 3-cyanophenol in Step 1. MS: (M+H)⁺=327.

### Example 232

### 2-(3-Trifluoromethoxyphenoxy)-5H-pyrrolo[2,3-b]pyrazine-7-carboxylic acid isopropylamide

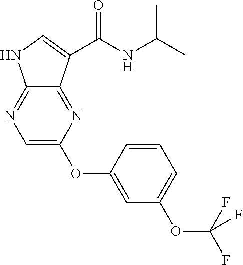 [SMILES: CC(C)NC(=O)c1c[nH]c2ncc(Oc3cccc(OC(F)(F)F)c3)nc12]

Prepared according to the procedure outlined in Example 230 substituting 3-(trifluoromethoxy)phenol for 3-cyanophenol in Step 1. MS: (M+H)⁺=381.

### Example 233

### 2-(3-tert-Butylphenoxy)-5H-pyrrolo[2,3-b]pyrazine-7-carboxylic acid isopropylamide

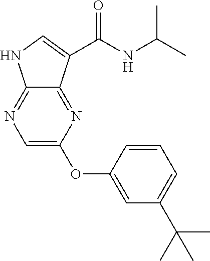 [SMILES: CC(C)NC(=O)c1c[nH]c2ncc(Oc3cccc(C(C)(C)C)c3)nc12]

Prepared according to the procedure outlined in Example 230 substituting 3-tert-butylphenol for 3-cyanophenol in Step 1. MS: (M+H)⁺=353.

### Example 234

### 2-(3-Methylphenoxy)-5H-pyrrolo[2,3-b]pyrazine-7-carboxylic acid isopropylamide

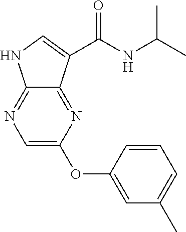 [SMILES: Cc1cccc(Oc2cnc3[nH]cc(C(=O)NC(C)C)c3n2)c1]

Prepared according to the procedure outlined in Example 230 substituting 3-methylphenol for 3-cyanophenol in Step 1. MS: (M+H)⁺=311.

### Example 235

### 2-(3-Ethylphenoxy)-5H-pyrrolo[2,3-b]pyrazine-7-carboxylic acid isopropylamide

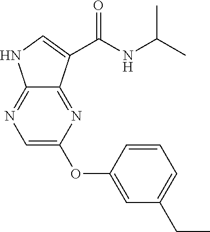 [SMILES: CCc1cccc(Oc2cnc3[nH]cc(C(=O)NC(C)C)c3n2)c1]

Prepared according to the procedure outlined in Example 230 substituting 3-ethylphenol for 3-cyanophenol in Step 1. MS: (M+H)⁺=325.

### Example 236

### 2-(3-Isopropylphenoxy)-5H-pyrrolo[2,3-b]pyrazine-7-carboxylic acid isopropylamide

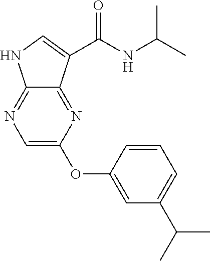 [SMILES: CC(C)NC(=O)c1c[nH]c2ncc(Oc3cccc(C(C)C)c3)nc12]

Prepared according to the procedure outlined in Example 230 substituting 3-isopropylphenol for 3-cyanophenol in Step 1. MS: (M+H)⁺=339.

### Example 237

### 2-(3-Trifluoromethylphenoxy)-5H-pyrrolo[2,3-b]pyrazine-7-carboxylic acid isopropylamide

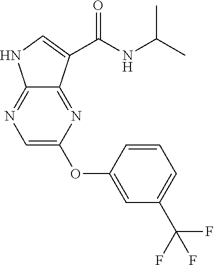 [SMILES: CC(C)NC(=O)c1c[nH]c2ncc(Oc3cccc(C(F)(F)F)c3)nc12]

Prepared according to the procedure outlined in Example 230 substituting 3-(trifluoromethyl)phenol for 3-cyanophenol in Step 1. MS: (M+H)⁺=365.

### Example 238

### 2-(2-Trifluoromethylphenoxy)-5H-pyrrolo[2,3-b]pyrazine-7-carboxylic acid isopropylamide

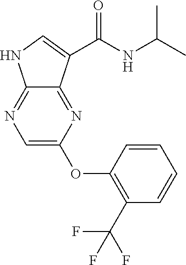 [SMILES: CC(C)NC(=O)c1c[nH]c2ncc(Oc3ccccc3C(F)(F)F)nc12]

Prepared according to the procedure outlined in Example 230 substituting 2-(trifluoromethyl)phenol for 3-cyanophenol in Step 1. MS: (M+H)⁺=365.

### Example 239

### 2-(2-Benzylphenoxy)-5H-pyrrolo[2,3-b]pyrazine-7-carboxylic acid isopropylamide

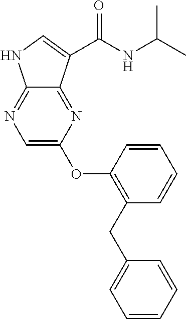 [SMILES: CC(C)NC(=O)c1c[nH]c2ncc(Oc3ccccc3Cc3ccccc3)nc12]

Prepared according to the procedure outlined in Example 230 substituting 2-benzylphenol for 3-cyanophenol in Step 1. MS: (M+H)⁺=387.

### Example 240

### 2-(2-Ethylphenoxy)-5H-pyrrolo[2,3-b]pyrazine-7-carboxylic acid isopropylamide

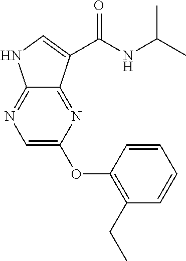 [SMILES: CCc1ccccc1Oc1cnc2[nH]cc(C(=O)NC(C)C)c2n1]

Prepared according to the procedure outlined in Example 230 substituting 2-ethylphenol for 3-cyanophenol in Step 1. MS: (M+H)⁺=325.

### Example 241

### 2-(5,6,7,8-Tetrahydronaphthalen-1-yloxy)-5H-pyrrolo[2,3-b]pyrazine-7-carboxylic acid isopropylamide

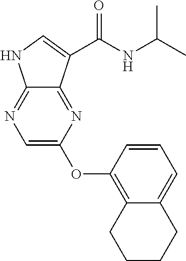 [SMILES: CC(C)NC(=O)c1c[nH]c2ncc(Oc3cccc4c3CCCC4)nc12]

Prepared according to the procedure outlined in Example 230 substituting 5,6,7,8-tetrahydronaphthalen-1-ol for 3-cyanophenol in Step 1. MS: (M+H)⁺=351.

### Example 242

### 2-(5,6,7,8-Tetrahydronaphthalen-2-yloxy)-5H-pyrrolo[2,3-b]pyrazine-7-carboxylic acid isopropylamide

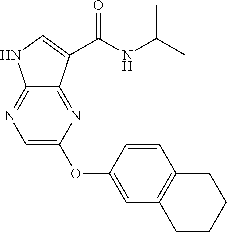 [SMILES: CC(C)NC(=O)c1c[nH]c2ncc(Oc3ccc4c(c3)CCCC4)nc12]

Prepared according to the procedure outlined in Example 230 substituting 5,6,7,8-tetrahydronaphthalen-2-ol for 3-cyanophenol in Step 1. MS: (M+H)⁺=351.

### Example 243

### 2-(Naphthalen-1-yloxy)-5H-pyrrolo[2,3-b]pyrazine-7-carboxylic acid isopropylamide

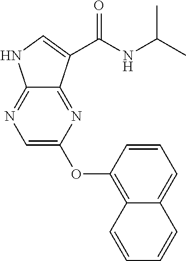 [SMILES: CC(C)NC(=O)c1c[nH]c2ncc(Oc3cccc4ccccc34)nc12]

Prepared according to the procedure outlined in Example 230 substituting naphthalen-1-ol for 3-cyanophenol in Step 1. MS: (M+H)⁺=347.

### Example 244

### 2-(Naphthalen-2-yloxy)-5H-pyrrolo[2,3-b]pyrazine-7-carboxylic acid isopropylamide

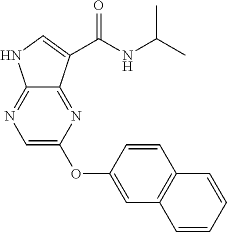 [SMILES: CC(C)NC(=O)c1c[nH]c2ncc(Oc3ccc4ccccc4c3)nc12]

Prepared according to the procedure outlined in Example 230 substituting naphthalen-2-ol for 3-cyanophenol in Step 1. MS: (M+H)⁺=347.

### Example 245

### 2-(3-Chlorophenoxy)-5H-pyrrolo[2,3-b]pyrazine-7-carboxylic acid isopropylamide

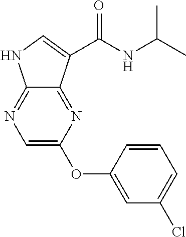 [SMILES: CC(C)NC(=O)c1c[nH]c2ncc(Oc3cccc(Cl)c3)nc12]

Prepared according to the procedure outlined in Example 230 substituting 3-chlorophenol for 3-cyanophenol in Step 1. MS: (M+H)⁺=332.

### Example 246

### 2-(3-Chlorophenoxy)-5H-pyrrolo[2,3-b]pyrazine-7-carboxylic acid ethylami

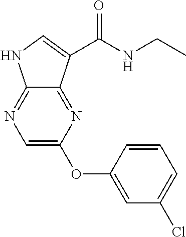 [SMILES: CCNC(=O)c1c[nH]c2ncc(Oc3cccc(Cl)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-chlorophenol for 3-cyanophenol in Step 1. MS: (M+H)⁺=318.

### Example 247

### 2-(3-Cyanophenoxy)-5H-pyrrolo[2,3-b]pyrazine-7-carboxylic acid ethylami

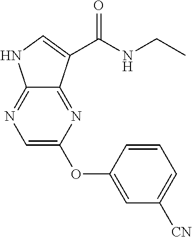 [SMILES: CCNC(=O)c1c[nH]c2ncc(Oc3cccc(C#N)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide in Step 1. MS: (M+H)⁺=308.

### Example 248

### 2-(3-Trifluoromethoxyphenoxy)-5H-pyrrolo[2,3-b]pyrazine-7-carboxylic acid ethylamide

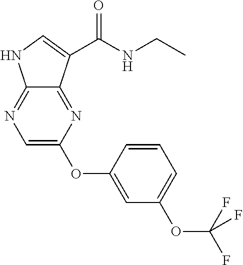 [SMILES: CCNC(=O)c1c[nH]c2ncc(Oc3cccc(OC(F)(F)F)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-(trifluoromethoxy)phenol for 3-cyanophenol in Step 1. MS: (M+H)⁺=367.

### Example 249

### 2-(3-tert-Butylphenoxy)-5H-pyrrolo[2,3-b]pyrazine-7-carboxylic acid ethylamide

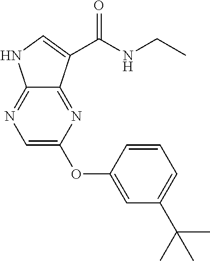 [SMILES: CCNC(=O)c1c[nH]c2ncc(Oc3cccc(C(C)(C)C)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-tert-butylphenol for 3-cyanophenol in Step 1. MS: (M+H)⁺=339.

### Example 250

### 2-(3-Methylphenoxy)-5H-pyrrolo[2,3-b]pyrazine-7-carboxylic acid ethylamide

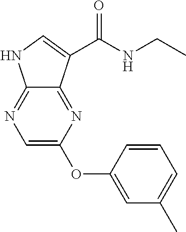 [SMILES: CCNC(=O)c1c[nH]c2ncc(Oc3cccc(C)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-methylphenol for 3-cyanophenol in Step 1. MS: (M+H)⁺=297.

### Example 251

### 2-(3-Ethylphenoxy)-5H-pyrrolo[2,3-b]pyrazine-7-carboxylic acid ethylamide

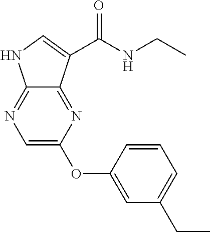 [SMILES: CCNC(=O)c1c[nH]c2ncc(Oc3cccc(CC)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-ethylphenol for 3-cyanophenol in Step 1. MS: (M+H)⁺=311.

### Example 252

### 2-(3-Isopropylphenoxy)-5H-pyrrolo[2,3-b]pyrazine-7-carboxylic acid ethylamide

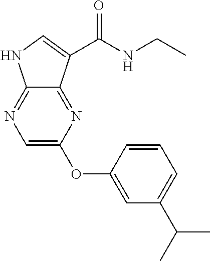 [SMILES: CCNC(=O)c1c[nH]c2ncc(Oc3cccc(C(C)C)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-isopropylphenol for 3-cyanophenol in Step 1. MS: (M+H)⁺=325.

### Example 253

### 2-(3-Trifluoromethylphenoxy)-5H-pyrrolo[2,3-b]pyrazine-7-carboxylic acid ethylamide

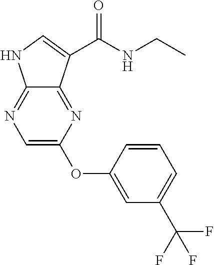 [SMILES: CCNC(=O)c1c[nH]c2ncc(Oc3cccc(C(F)(F)F)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-(trifluoromethyl)phenol for 3-cyanophenol in Step 1. MS: (M+H)⁺=351.

### Example 254

### 2-(2-Methylphenoxy)-5H-pyrrolo[2,3-b]pyrazine-7-carboxylic acid isopropylamide

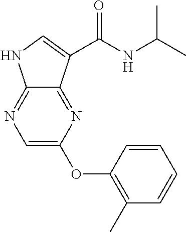 [SMILES: Cc1ccccc1Oc1cnc2[nH]cc(C(=O)NC(C)C)c2n1]

Prepared according to the procedure outlined in Example 230 substituting 2-methylphenol for 3-cyanophenol in Step 1. MS: (M+H)⁺=311.

### Example 255

### 2-(2-Trifluoromethoxyphenoxy)-5H-pyrrolo[2,3-b]pyrazine-7-carboxylic acid isopropylamide

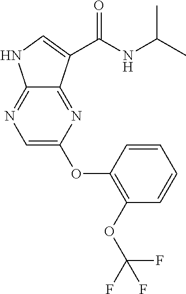 [SMILES: CC(C)NC(=O)c1c[nH]c2ncc(Oc3ccccc3OC(F)(F)F)nc12]

Prepared according to the procedure outlined in Example 230 substituting 2-(trifluoromethoxy)phenol for 3-cyanophenol in Step 1. MS: (M+H)⁺=381.

### Example 256

### 2-(2,2-Dimethyl-2,3-dihydrobenzofuran-7-yloxy)-5H-pyrrolo[2,3-b]pyrazine-7-carboxylic acid isopropylamide

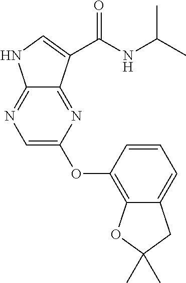 [SMILES: CC(C)NC(=O)c1c[nH]c2ncc(Oc3cccc4c3OC(C)(C)C4)nc12]

Prepared according to the procedure outlined in Example 230 substituting 2,2-dimethyl-2,3-dihydro-benzofuran-7-ol for 3-cyanophenol in Step 1. MS: (M+H)⁺=367.

### Example 257

### 2-(2-Chlorophenoxy)-5H-pyrrolo[2,3-b]pyrazine-7-carboxylic acid isopropylamide

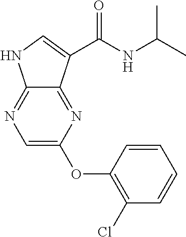 [SMILES: CC(C)NC(=O)c1c[nH]c2ncc(Oc3ccccc3Cl)nc12]

Prepared according to the procedure outlined in Example 230 substituting 2-chlorophenol for 3-cyanophenol in Step 1. MS: (M+H)⁺=332.

### Example 258

### 2-(2-Methoxyphenoxy)-5H-pyrrolo[2,3-b]pyrazine-7-carboxylic acid isopropylamide

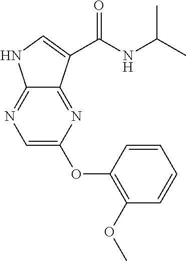 [SMILES: COc1ccccc1Oc1cnc2[nH]cc(C(=O)NC(C)C)c2n1]

Prepared according to the procedure outlined in Example 230 substituting 2-methoxyphenol for 3-cyanophenol in Step 1. MS: (M+H)⁺=327.

### Example 259

### 2-(2-Methylphenoxy)-5H-pyrrolo[2,3-b]pyrazine-7-carboxylic acid ethylamide

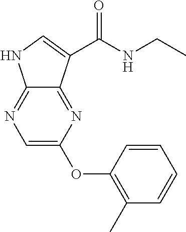 [SMILES: CCNC(=O)c1c[nH]c2ncc(Oc3ccccc3C)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 2-methylphenol for 3-cyanophenol in Step 1. MS: (M+H)⁺=297.

### Example 260

### 2-(3,5-Dimethoxyphenoxy)-5H-pyrrolo[2,3-b]pyrazine-7-carboxylic acid isopropylamide

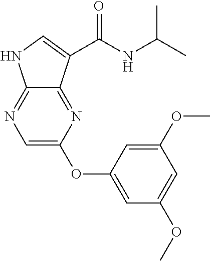 [SMILES: COc1cc(OC)cc(Oc2cnc3[nH]cc(C(=O)NC(C)C)c3n2)c1]

Prepared according to the procedure outlined in Example 230 substituting 3,5-dimethoxyphenol for 3-cyanophenol in Step 1. MS: (M+H)⁺=357.

### Example 261

### 2-(5,6,7,8-Tetrahydronaphthalen-1-yloxy)-5H-pyrrolo[2,3-b]pyrazine-7-carboxylic acid ethylamide

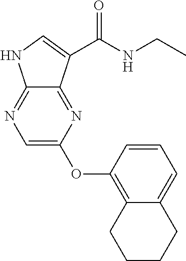 [SMILES: CCNC(=O)c1c[nH]c2ncc(Oc3cccc4c3CCCC4)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 5,6,7,8-tetrahydronaphthalen-1-ol for 3-cyanophenol in Step 1. MS: (M+H)⁺=337.

### Example 262

### 2-(5,6,7,8-Tetrahydronaphthalen-2-yloxy)-5H-pyrrolo[2,3-b]pyrazine-7-carboxylic acid ethylamide

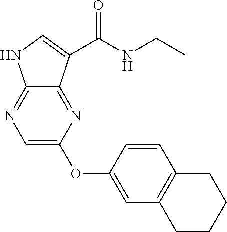 [SMILES: CCNC(=O)c1c[nH]c2ncc(Oc3ccc4c(c3)CCCC4)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 5,6,7,8-tetrahydronaphthalen-2-ol for 3-cyanophenol in Step 1. MS: (M+H)⁺=337.

### Example 263

### 2-(Naphthalen-1-yloxy)-5H-pyrrolo[2,3-b]pyrazine-7-carboxylic acid ethylamide

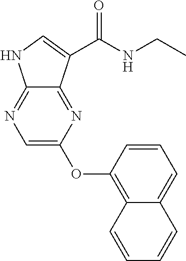 [SMILES: CCNC(=O)c1c[nH]c2ncc(Oc3cccc4ccccc34)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and naphthalen-1-ol for 3-cyanophenol in Step 1. MS: (M+H)⁺=333.

### Example 264

### 2-(Naphthalen-2-yloxy)-5H-pyrrolo[2,3-b]pyrazine-7-carboxylic acid ethylamide

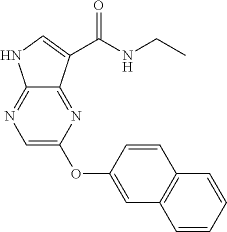 [SMILES: CCNC(=O)c1c[nH]c2ncc(Oc3ccc4ccccc4c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and naphthalen-2-ol for 3-cyanophenol in Step 1. MS: (M+H)⁺=333.

### Example 265

### 2-(3,5-Dimethoxyphenoxy)-5H-pyrrolo[2,3-b]pyrazine-7-carboxylic acid ethylamide

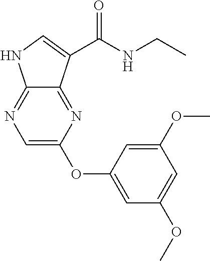 [SMILES: CCNC(=O)c1c[nH]c2ncc(Oc3cc(OC)cc(OC)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3,5-dimethoxyphenol for 3-cyanophenol in Step 1. MS: (M+H)⁺=343.

### Example 266

### 2-(3-Methoxyphenoxy)-5H-pyrrolo[2,3-b]pyrazine-7-carboxylic acid ethylamide

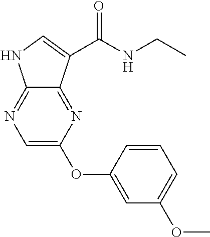 [SMILES: CCNC(=O)c1c[nH]c2ncc(Oc3cccc(OC)c3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 3-methoxyphenol for 3-cyanophenol in Step 1. MS: (M+H)⁺=313.

### Example 267

### 2-(2-Chlorophenoxy)-5H-pyrrolo[2,3-b]pyrazine-7-carboxylic acid ethylamide

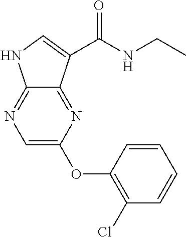 [SMILES: CCNC(=O)c1c[nH]c2ncc(Oc3ccccc3Cl)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 2-chlorophenol for 3-cyanophenol in Step 1. MS: (M+H)⁺=318.

### Example 268

### 2-(4-Cyanophenoxy)-5H-pyrrolo[2,3-b]pyrazine-7-carboxylic acid isopropylamide

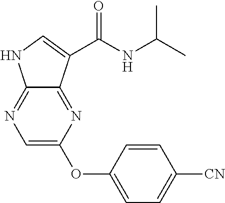 [SMILES: CC(C)NC(=O)c1c[nH]c2ncc(Oc3ccc(C#N)cc3)nc12]

Prepared according to the procedure outlined in Example 230 substituting 4-cyanophenol for 3-cyanophenol in Step 1. MS: (M+H)⁺=322.

### Example 269

### 2-(4-Cyanophenoxy)-5H-pyrrolo[2,3-b]pyrazine-7-carboxylic acid ethylamide

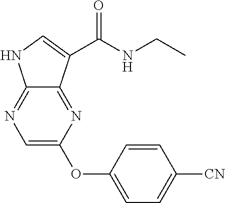 [SMILES: CCNC(=O)c1c[nH]c2ncc(Oc3ccc(C#N)cc3)nc12]

Prepared according to the procedure outlined in Example 230 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide and 4-cyanophenol for 3-cyanophenol in Step 1. MS: (M+H)⁺=308.

### Example 270

### 2-((R)-3-Methanesulfonylaminoindan-5-yloxy)-5H-pyrrolo[2,3-b]pyrazine-7-carboxylic acid isopropylamide

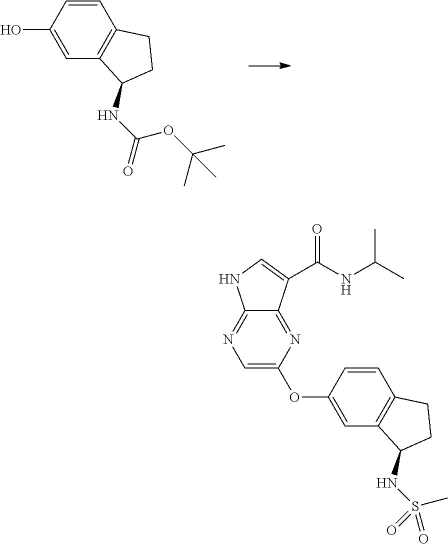 [SMILES: CC(C)(C)OC(=O)N[C@@H]1CCc2ccc(O)cc21 ; CC(C)NC(=O)c1c[nH]c2ncc(Oc3ccc4c(c3)[C@H](NS(C)(=O)=O)CC4)nc12]

### Step 1

To a solution of (R)-3-aminoindan-5-ol (100 mg, 0.67 mmol) in THF (4 mL) were added di-tert-butyl dicarbonate (0.13 mL, 0.60 mmol) and triethylamine (0.11 mL, 0.80 mmol). The reaction mixture was stirred at room temperature for 18 h then the solvent was evaporated. The residue was partitioned between water and EtOAc. The aqueous layer was extracted with EtOAc and the combined organic layers were dried over Na₂SO₄ and concentrated under reduced pressure. The residue was purified by silica gel chromatography using EtOAc/hexanes as the eluting solvent to afford 100 mg (60%) of ((R)-6-hydroxyindan-1-yl)-carbamic acid tert-butyl ester as a white solid.

### Step 2

To a stirred solution of 2-bromo-5-(2-trimethylsilanyl-ethoxymethyl)-5H-pyrrolo[2,3-b]pyrazine-7-carboxylic acid isopropylamide (250 mg, 0.60 mmol) in toluene (5 mL) were added ((R)-6-hydroxyindan-1-yl)-carbamic acid tert-butyl ester (190 mg, 0.78 mmol), K₃PO₄ (250 mg, 1.20 mmol), and 2-di-tert-butylphosphino-2′-(N,N-dimethylamino)biphenyl (41 mg, 0.12 mmol). The reaction mixture was purged thoroughly with argon gas for 20 min, then Pd(OAc)₂ (13 mg, 0.06 mmol) was added. The reaction mixture was heated at 140° C. for 18 h then cooled to room temperature, quenched with water (20 mL) and extracted with EtOAc (3×15 mL). The combined organic layers were washed with brine, dried over anhydrous Na₂SO₄ and concentrated under reduced pressure. The residue was purified by column chromatography over silica gel (100-200 mesh) using 20-60% EtOAc/hexane as the eluting solvent to afford 220 mg (65%) of {(R)-6-[7-isopropylcarbamoyl-5-(2-trimethylsilanyl-ethoxymethyl)-5H-pyrrolo[2,3-b]pyrazin-2-yloxy]-indan-1-yl}-carbamic acid tert-butyl ester as a brown solid.

### Step 3

To a stirred solution of {(R)-6-[7-isopropylcarbamoyl-5-(2-trimethylsilanyl-ethoxymethyl)-5H-pyrrolo[2,3-b]pyrazin-2-yloxy]-indan-1-yl}-carbamic acid tert-butyl ester (450 mg, 0.77 mol) in dry MeOH (10 mL) at 0° C. was added dropwise acetyl chloride (1.09 mL, 15.46 mmol). After the addition, the reaction was allowed to warm to room temperature and stirred for 2 h. The reaction mixture was concentrated under reduced pressure and at room temperature to afford 2-((R)-3-amino-indan-5-yloxy)-5-(2-trimethylsilanyl-ethoxymethyl)-5H-pyrrolo[2,3-b]pyrazine-7-carboxylic acid isopropylamide hydrochloride as a brown solid which was used without further purification.

### Step 4

To a stirred solution of 2-((R)-3-amino-indan-5-yloxy)-5-(2-trimethylsilanyl-ethoxymethyl)-5H-pyrrolo[2,3-b]pyrazine-7-carboxylic acid isopropylamide hydrochloride (200 mg, 0.38 mmol) in dichloromethane (8 mL) at 0° C. was added diisopropylethylamine (0.29 mL, 1.66 mmol) followed by methanesulfonyl chloride (0.038 mL, 0.49 mmol). The reaction mixture was stirred at 0° C. for 10 min, at room temperature for 16 h, then quenched with water and extracted with EtOAc (3×). The combined organics were dried over Na₂SO₄ and concentrated. The residue was purified by column chromatography over silica gel (100-200 mesh) using EtOAc/hexane as the eluting solvent to afford 170 mg (73%) of 2-((R)-3-methanesulfonylamino-indan-5-yloxy)-5-(2-trimethylsilanyl-ethoxymethyl)-5H-pyrrolo[2,3-b]pyrazine-7-carboxylic acid isopropylamide as a grey solid.

### Step 5

To a stirred solution of 2-((R)-3-methanesulfonylamino-indan-5-yloxy)-5-(2-trimethylsilanyl-ethoxymethyl)-5H-pyrrolo[2,3-b]pyrazine-7-carboxylic acid isopropylamide (170 mg, 0.30 mmol) in anhydrous THF (5 mL) were added tetrabutylammonium fluoride (1.0 M in THF, 6.0 mL, 6 mmol) and ethylenediamine (0.40 mL, 6.0 mmol). The reaction mixture was heated at reflux for 18 h then cooled to room temperature, quenched with water and extracted with ethyl acetate (3×). The combined organics were dried over Na₂SO₄ and concentrated. The residue was purified by column chromatography over silica gel (100-200 mesh) using 2-5% MeOH/CH₂Cl₂ as the eluting solvent to afford 43 mg (34%) of 2-((R)-3-methanesulfonylaminoindan-5-yloxy)-5H-pyrrolo[2,3-b]pyrazine-7-carboxylic acid isopropylamide as an off-white solid. MS: (M+H)⁺=430.

### Example 271

### 2-((R)-3-Acetylaminoindan-5-yloxy)-5H-pyrrolo[2,3-b]pyrazine-7-carboxylic acid isopropylamide

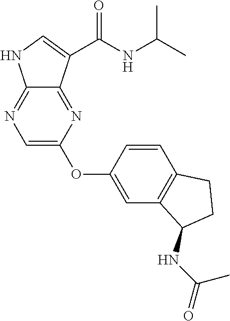 [SMILES: CC(=O)N[C@@H]1CCc2ccc(Oc3cnc4[nH]cc(C(=O)NC(C)C)c4n3)cc21]

Prepared according to the procedure outlined in Example 270 substituting acetic anhydride and pyridine for methanesulfonyl chloride and diisopropylethylamine in Step 4. MS: (M+H)⁺=394.

### Example 272

### 2-((R)-3-Methanesulfonylaminoindan-5-yloxy)-5H-pyrrolo[2,3-b]pyrazine-7-carboxylic acid ethylamide

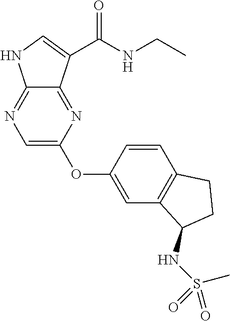 [SMILES: CCNC(=O)c1c[nH]c2ncc(Oc3ccc4c(c3)[C@H](NS(C)(=O)=O)CC4)nc12]

Prepared according to the procedure outlined in Example 270 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide in Step 2. MS: (M+H)⁺=416.

### Example 273

### 2-((R)-3-Acetylaminoindan-5-yloxy)-5H-pyrrolo[2,3-b]pyrazine-7-carboxylic acid ethylamide

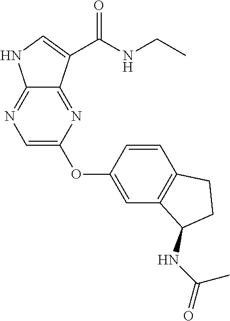 [SMILES: CCNC(=O)c1c[nH]c2ncc(Oc3ccc4c(c3)[C@H](NC(C)=O)CC4)nc12]

Prepared according to the procedure outlined in Example 270 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide in Step 2 and acetic anhydride and pyridine for methanesulfonyl chloride and diisopropylethylamine in Step 4. MS: (M+H)⁺=380.

### Example 274

### 2-(1H-Indol-6-yloxy)-5H-pyrrolo[2,3-b]pyrazine-7-carboxylic acid isopropylamide

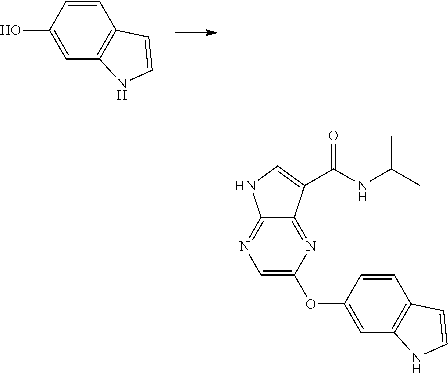 [SMILES: Oc1ccc2cc[nH]c2c1 ; CC(C)NC(=O)c1c[nH]c2ncc(Oc3ccc4cc[nH]c4c3)nc12]

### Step 1

To a stirred solution of 6-hydroxyindole (0.40 g, 3.00 mmol) in MeCN (15 mL), were added di-tert-butyl dicarbonate (1.9 mL, 9.00 mmol), DMAP (0.184 g, 1.5 mmol) and triethylamine (1.2 mL, 9.00 mmol). The reaction mixture was stirred at 25° C. for 16 h then the solvent was completely distilled off. The residue was purified by column chromatography over silica gel (100-200 mesh) using 10-20% EtOAc/hexane as the eluting solvent to afford 0.87 g (87%) of 6-tert-butoxycarbonyloxy-indole-1-carboxylic acid tert-butyl ester as a colorless oil.

### Step 2

To a stirred solution of 6-tert-butoxycarbonyloxy-indole-1-carboxylic acid tert-butyl ester (1.0 g, 3.00 mmol) in dichloromethane (20 mL) was added morpholine (7.8 mL, 90.1 mmol). The reaction mixture was stirred at 25° C. for 20 h then the solvent was completely distilled off. The residue was purified by column chromatography over silica gel (100-200 mesh) using 10-20% EtOAc/hexane as eluting solvent to afford 0.35 g (50%) of 6-hydroxyindole-1-carboxylic acid tert-butyl ester as a colorless oil.

### Step 3

To a stirred solution of 2-bromo-5-(2-trimethylsilanyl-ethoxymethyl)-5H-pyrrolo[2,3-b]pyrazine-7-carboxylic acid isopropylamide (550 mg, 1.33 mmol) in toluene (10 mL) were added 6-hydroxyindole-1-carboxylic acid tert-butyl ester (466 mg, 2.00 mmol), K₃PO₄ (564 mg, 2.66 mmol), and 2-di-tert-butylphosphino-2′-(N,N-dimethylamino)biphenyl (136 mg, 0.40 mmol). The reaction mixture was purged thoroughly with argon gas for 20 min, then Pd(OAc)₂ (58 mg, 0.26 mmol) was added. The reaction mixture was heated at 140° C. for 18 h then cooled to room temperature, quenched with water (20 mL) and extracted with EtOAc (3×15 mL). The combined organic layers were washed with brine, dried over anhydrous Na₂SO₄ and concentrated under reduced pressure. The residue was purified by column chromatography over silica gel (100-200 mesh) using 20-60% EtOAc/hexane as the eluting solvent to afford 320 mg (52%) of 2-(1H-indol-6-yloxy)-5-(2-trimethylsilanyl-ethoxymethyl)-5H-pyrrolo[2,3-b]pyrazine-7-carboxylic acid isopropylamide as a yellow-brown oil.

### Step 4

To a stirred solution of 2-(1H-indol-6-yloxy)-5-(2-trimethylsilanyl-ethoxymethyl)-5H-pyrrolo[2,3-b]pyrazine-7-carboxylic acid isopropylamide (63 mg, 0.135 mmol) in dichloromethane (5 mL) at 0° C. was added trifluoroacetic acid (1 mL). The reaction mixture was stirred at room temperature for 4 h then the solvent was removed under reduced pressure. The residue was dissolved in 1:1 MeOH/dichloromethane (5 mL) and ethylenediamine (0.2 mL) was added at 0° C. The reaction mixture was stirred at room temperature for 18 h then concentrated under reduced pressure. The crude residue was purified by preparative HPLC to afford 3.8 mg (8%) of 2-(1H-indol-6-yloxy)-5H-pyrrolo[2,3-b]pyrazine-7-carboxylic acid isopropylamide. MS: (M+H)⁺=336.

### Example 275

### 2-(1H-Indol-6-yloxy)-5H-pyrrolo[2,3-b]pyrazine-7-carboxylic acid ethylamide

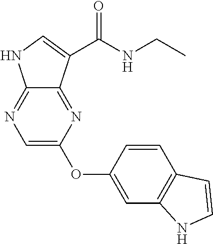 [SMILES: CCNC(=O)c1c[nH]c2ncc(Oc3ccc4cc[nH]c4c3)nc12]

Prepared according to the procedure outlined in Example 274 substituting 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide in Step 3. MS: (M+H)⁺=322.

### Example 276

### 2-(1H-Indol-4-yloxy)-5H-pyrrolo[2,3-b]pyrazine-7-carboxylic acid isopropylamide

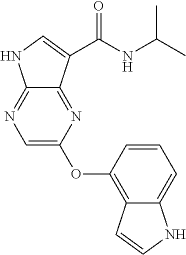 [SMILES: CC(C)NC(=O)c1c[nH]c2ncc(Oc3cccc4[nH]ccc34)nc12]

Prepared according to the procedure outlined in Example 274 substituting 4-hydroxyindole for 6-hydroxyindole in Step 1. MS: (M+H)⁺=336.

### Example 277

### 2-(1H-Indol-4-yloxy)-5H-pyrrolo[2,3-b]pyrazine-7-carboxylic acid ethylamide

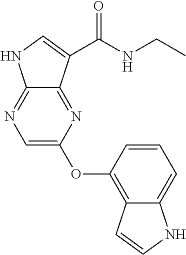 [SMILES: CCNC(=O)c1c[nH]c2ncc(Oc3cccc4[nH]ccc34)nc12]

Prepared according to the procedure outlined in Example 274 substituting 4-hydroxyindole for 6-hydroxyindole in Step 1 and 2-bromo-5-(2-trimethylsilanyl-ethoxymethyl)-5H-pyrrolo[2,3-b]pyrazine-7-carboxylic acid ethylamide for 2-bromo-5-(2-trimethylsilanyl-ethoxymethyl)-5H-pyrrolo[2,3-b]pyrazine-7-carboxylic acid isopropylamide in Step 3. MS: (M+H)⁺=322.

### Example 278

### 2-(1-Methyl-1H-indol-6-yloxy)-5H-pyrrolo[2,3-b]pyrazine-7-carboxylic acid isopropylamide

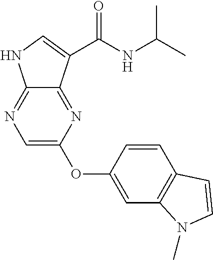 [SMILES: CC(C)NC(=O)c1c[nH]c2ncc(Oc3ccc4ccn(C)c4c3)nc12]

### Step 1

To a stirred suspension of NaH (60% in mineral oil, 36 mg, 0.90 mmol) in anhydrous DMF (10 mL) at 0° C. was added a solution of 2-(1H-indol-6-yloxy)-5-(2-trimethylsilanyl-ethoxymethyl)-5H-pyrrolo[2,3-b]pyrazine-7-carboxylic acid isopropylamide (275 mg, 0.59 mmol) in anhydrous DMF (5 mL). The reaction mixture was allowed to stir at 25° C. for 30 min then cooled 0° C. and iodomethane (44 uL, 0.70 mmol) was slowly added. The reaction mixture was stirred at 25° C. for 3 h then the DMF was distilled off. The crude residue was purified by column chromatography over silica gel with 7% ethyl acetate in hexane to afford 160 mg (56%) of 2-(1-methyl-1H-indol-6-yloxy)-5-(2-trimethylsilanyl-ethoxymethyl)-5H-pyrrolo[2,3-b]pyrazine-7-carboxylic acid isopropylamide as a yellow oil.

### Step 2

To a stirred solution of 2-(1-methyl-1H-indol-6-yloxy)-5-(2-trimethylsilanyl-ethoxymethyl)-5H-pyrrolo[2,3-b]pyrazine-7-carboxylic acid isopropylamide (160 mg, 0.33 mmol) in anhydrous THF (5 mL) were added tetrabutylammonium fluoride (1.0 M in THF, 6.6 mL, 6.6 mmol) and ethylenediamine (0.44 mL, 6.6 mmol). The reaction mixture was heated at reflux for 16 h then cooled to room temperature, quenched with water and extracted with ethyl acetate (4×). The combined organics were dried over Na₂SO₄ and concentrated. The residue was purified by column chromatography over silica gel (100-200 mesh) using 2-6% MeOH/CH₂Cl₂ as the eluting solvent to afford 59 mg (52%) of 2-(1-methyl-1H-indol-6-yloxy)-5H-pyrrolo[2,3-b]pyrazine-7-carboxylic acid isopropylamide as a light yellow solid. MS: (M+H)⁺=350.

### Example 279

### 2-(1H-Indol-5-yloxy)-5H-pyrrolo[2,3-b]pyrazine-7-carboxylic acid isopropylamide

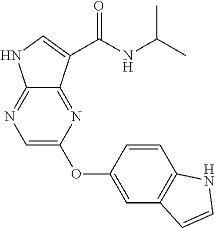 [SMILES: CC(C)NC(=O)c1c[nH]c2ncc(Oc3ccc4[nH]ccc4c3)nc12]

### Step 1

To a stirred solution of 5-hydroxyindole (1.0 g, 7.50 mmol) in MeCN (35 mL), were added di-tert-butyl dicarbonate (4.9 g, 22.5 mmol), DMAP (0.46 g, 3.75 mmol) and triethylamine (3.2 mL, 22.5 mmol). The reaction mixture was stirred at 25° C. for 16 h then the solvent was completely distilled off. The residue was purified by column chromatography over silica gel (100-200 mesh) using 10-20% EtOAc/hexane as the eluting solvent to afford 2.5 g (100%) of 5-tert-butoxycarbonyloxy-indole-1-carboxylic acid tert-butyl ester as a colorless oil.

### Step 2

To a stirred solution of 5-tert-butoxycarbonyloxy-indole-1-carboxylic acid tert-butyl ester (1.5 g, 4.50 mmol) in dichloromethane (30 mL) was added morpholine (11.8 mL, 135 mmol). The reaction mixture was stirred at 25° C. for 16 h then the solvent was completely distilled off. The residue was purified by column chromatography over silica gel (100-200 mesh) using 10-20% EtOAc/hexane as eluting solvent to afford 0.8 g (77%) of 5-hydroxyindole-1-carboxylic acid tert-butyl ester as a colorless oil.

### Step 3

To a stirred solution of 2-bromo-5-(2-trimethylsilanyl-ethoxymethyl)-5H-pyrrolo[2,3-b]pyrazine-7-carboxylic acid isopropylamide (100 mg, 0.24 mmol) in DMF (4 mL) in a microwave vial were added 5-hydroxyindole-1-carboxylic acid tert-butyl ester (68 mg, 0.29 mmol) and Cs₂CO₃ (235 mg, 0.72 mmol). The vial was sealed and heated in a microwave reactor at 120° C. for 1 h. The reaction was quenched with water (20 mL) and extracted with EtOAc (3×). The combined organic layer was washed with brine, dried over Na₂SO₄ and concentrated under reduced pressure. The residue was purified by column chromatography over silica gel (100-200 mesh) using 20-60% EtOAc/hexane as eluting solvent to afford 60 mg (36%) of 2-(1H-indol-5-yloxy)-5-(2-trimethylsilanyl-ethoxymethyl)-5H-pyrrolo[2,3-b]pyrazine-7-carboxylic acid isopropylamide as a brown oil.

### Step 4

To a stirred solution of 2-(1H-indol-5-yloxy)-5-(2-trimethylsilanyl-ethoxymethyl)-5H-pyrrolo[2,3-b]pyrazine-7-carboxylic acid isopropylamide (60 mg, 0.13 mmol) in anhydrous THF (4 mL) was added tetrabutylammonium fluoride (1.0 M in THF, 2.6 mL, 2.6 mmol). The reaction mixture was heated at reflux for 16 h then cooled to room temperature, quenched with water and extracted with ethyl acetate (4×). The combined organics were dried over Na₂SO₄ and concentrated. The residue was purified by column chromatography using MeOH/CH₂Cl₂ as the eluting solvent to afford 3.4 mg (8%) of 2-(1H-indol-5-yloxy)-5H-pyrrolo[2,3-b]pyrazine-7-carboxylic acid isopropylamide as an off-white solid. MS: (M+H)⁺=336.

### Example 280

### 2-(6-Methylpyridin-2-yloxy)-5H-pyrrolo[2,3-b]pyrazine-7-carboxylic acid isopropylamide

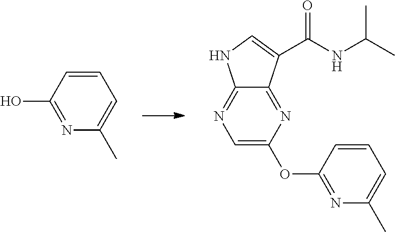

### Step 1

To a stirred solution of 2-bromo-5-(2-trimethylsilanyl-ethoxymethyl)-5H-pyrrolo[2,3-b]pyrazine-7-carboxylic acid isopropylamide (145 mg, 0.35 mmol) in DMF (4 mL) in a pressure tube were added 6-methylpyridin-2-ol (115 mg, 1.05 mmol) and Cs₂CO₃ (342 mg, 1.05 mmol). The tube was sealed and heated at 140° C. for 18 h. The reaction was cooled then quenched with water (20 mL) and extracted with EtOAc (4×). The combined organic layer was washed with brine, dried over Na₂SO₄ and concentrated under reduced pressure. The residue was purified by column chromatography over silica gel (100-200 mesh) using 20-60% EtOAc/hexane as eluting solvent to afford 95 mg (61%) of 2-(6-methylpyridin-2-yloxy)-5-(2-trimethylsilanyl-ethoxymethyl)-5H-pyrrolo[2,3-b]pyrazine-7-carboxylic acid isopropylamide as a colorless oil.

### Step 4

To a stirred solution of 2-(6-methylpyridin-2-yloxy)-5-(2-trimethylsilanyl-ethoxymethyl)-5H-pyrrolo[2,3-b]pyrazine-7-carboxylic acid isopropylamide (160 mg, 0.36 mmol) in anhydrous THF (4 mL) were added tetrabutylammonium fluoride (1.0 M in THF, 7.2 mL, 7.2 mmol) and ethylenediamine (0.48 mL, 7.2 mmol). The reaction mixture was heated at reflux for 16 h then cooled to room temperature, quenched with water and extracted with ethyl acetate (4×). The combined organics were dried over Na₂SO₄ and concentrated. The residue was purified by column chromatography using 2-10% MeOH/CH₂Cl₂ as the eluting solvent to afford 65 mg (58%) of 2-(6-methylpyridin-2-yloxy)-5H-pyrrolo[2,3-b]pyrazine-7-carboxylic acid isopropylamide as an off-white solid. MS: (M+H)⁺=312.

### Example 281

### 2-(4,6-Dimethylpyridin-2-yloxy)-5H-pyrrolo[2,3-b]pyrazine-7-carboxylic acid isopropylamide

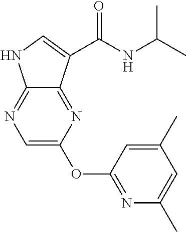 [SMILES: Cc1cc(C)nc(Oc2cnc3[nH]cc(C(=O)NC(C)C)c3n2)c1]

Prepared according to the procedure outlined in Example 280 substituting 4,6-dimethylpyridin-2-ol for 6-methylpyridin-2-ol in Step 1. MS: (M+H)⁺=326.

### Example 282

### 2-(2-Methylpyridin-3-yloxy)-5H-pyrrolo[2,3-b]pyrazine-7-carboxylic acid isopropylamide

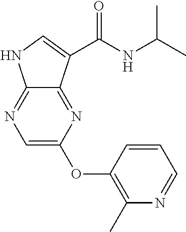 [SMILES: Cc1ncccc1Oc1cnc2[nH]cc(C(=O)NC(C)C)c2n1]

Prepared according to the procedure outlined in Example 280 substituting 2-methylpyridin-3-ol for 6-methylpyridin-2-ol in Step 1. MS: (M+H)⁺=312.

### Example 283

### 2-((R)-3-Aminoindan-5-yloxy)-5H-pyrrolo[2,3-b]pyrazine-7-carboxylic acid isopropylamide

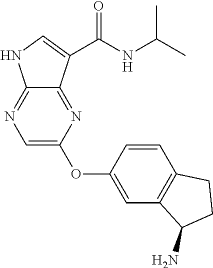 [SMILES: CC(C)NC(=O)c1c[nH]c2ncc(Oc3ccc4c(c3)[C@H](N)CC4)nc12]

Prepared according to the procedure outlined in Example 270, but omitting Step 4. MS: (M+H)⁺=352.

### Example 284

### 2-((R)-3-Propionylaminoindan-5-yloxy)-5H-pyrrolo[2,3-b]pyrazine-7-carboxylic acid isopropylamide

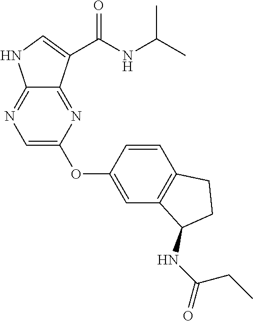 [SMILES: CCC(=O)N[C@@H]1CCc2ccc(Oc3cnc4[nH]cc(C(=O)NC(C)C)c4n3)cc21]

Prepared according to the procedure outlined in Example 270 substituting propionyl chloride and pyridine for methanesulfonyl chloride and diisopropylethylamine in Step 4. MS: (M+H)⁺=408.

### Example 285

### 2-{(R)-3-[(Tetrahydropyran-4-carbonyl)-amino]-indan-5-yloxy}-5H-pyrrolo[2,3-b]pyrazine-7-carboxylic acid isopropylamide

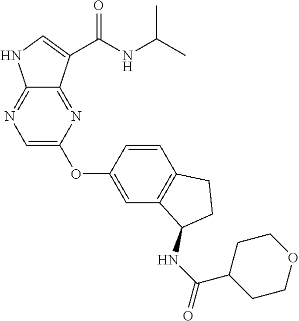 [SMILES: CC(C)NC(=O)c1c[nH]c2ncc(Oc3ccc4c(c3)[C@H](NC(=O)C3CCOCC3)CC4)nc12]

Prepared according to the procedure outlined in Example 270 substituting tetrahydropyran-4-carbonyl chloride and pyridine for methanesulfonyl chloride and diisopropylethylamine in Step 4. MS: (M+H)⁺=464.

### Example 286

### 2-[(R)-3-(Cyclopropanecarbonyl-amino)-indan-5-yloxy]-5H-pyrrolo[2,3-b]pyrazine-7-carboxylic acid isopropylamide

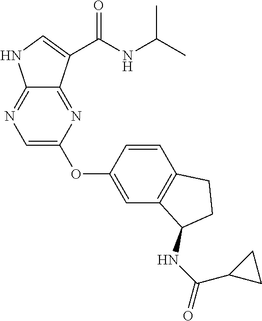 [SMILES: CC(C)NC(=O)c1c[nH]c2ncc(Oc3ccc4c(c3)[C@H](NC(=O)C3CC3)CC4)nc12]

Prepared according to the procedure outlined in Example 270 substituting cyclopropanecarbonyl chloride and pyridine for methanesulfonyl chloride and diisopropylethylamine in Step 4. MS: (M+H)⁺=420.

### Example 287

### 2-[(R)-3-(2,2-Dimethyl-propionylamino)-indan-5-yloxy]-5H-pyrrolo[2,3-b]pyrazine-7-carboxylic acid isopropylamide

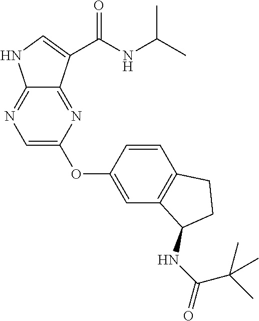 [SMILES: CC(C)NC(=O)c1c[nH]c2ncc(Oc3ccc4c(c3)[C@H](NC(=O)C(C)(C)C)CC4)nc12]

Prepared according to the procedure outlined in Example 270 substituting trimethylacetyl chloride and pyridine for methanesulfonyl chloride and diisopropylethylamine in Step 4. MS: (M+H)⁺=436.

### Example 288

### 2-((R)-3-Benzoylaminoindan-5-yloxy)-5H-pyrrolo[2,3-b]pyrazine-7-carboxylic acid isopropylamide

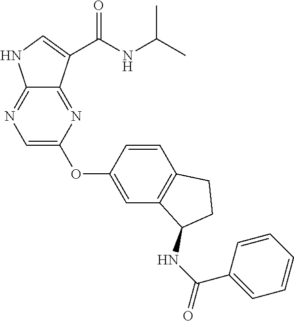 [SMILES: CC(C)NC(=O)c1c[nH]c2ncc(Oc3ccc4c(c3)[C@H](NC(=O)c3ccccc3)CC4)nc12]

Prepared according to the procedure outlined in Example 270 substituting benzoyl chloride and pyridine for methanesulfonyl chloride and diisopropylethylamine in Step 4. MS: (M+H)⁺=456.

### Example 289

### 2-((R)-3-Acetylaminoindan-5-yloxy)-5H-pyrrolo[2,3-b]pyrazine-7-carboxylic acid ((S)-1,2,2-trimethyl-propyl)-amide

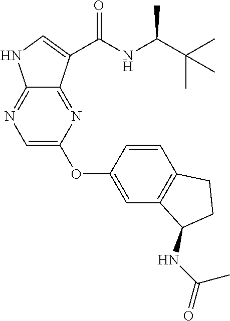 [SMILES: CC(=O)N[C@@H]1CCc2ccc(Oc3cnc4[nH]cc(C(=O)N[C@@H](C)C(C)(C)C)c4n3)cc21]

Prepared according to the procedure outlined in Example 270 substituting 2-bromo-5-(2-trimethylsilanyl-ethoxymethyl)-5H-pyrrolo[2,3-b]pyrazine-7-carboxylic acid ((S)-1,2,2-trimethyl-propyl)-amide for 2-bromo-5-(2-trimethylsilanyl-ethoxymethyl)-5H-pyrrolo[2,3-b]pyrazine-7-carboxylic acid isopropylamide in Step 2 and acetyl chloride and pyridine for methanesulfonyl chloride and diisopropylethylamine in Step 4. MS: (M+H)⁺=436.

### Example 290

### 2-((S)-3-Acetylaminoindan-5-yloxy)-5H-pyrrolo[2,3-b]pyrazine-7-carboxylic acid isopropylamide

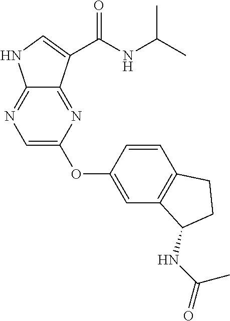 [SMILES: CC(=O)N[C@H]1CCc2ccc(Oc3cnc4[nH]cc(C(=O)NC(C)C)c4n3)cc21]

Prepared according to the procedure outlined in Example 270 substituting (S)-3-aminoindan-5-ol for (R)-3-aminoindan-5-ol in Step 1 and acetyl chloride and pyridine for methanesulfonyl chloride and diisopropylethylamine in Step 4. MS: (M+H)⁺=394.

### Example 291

### 2-((S)-3-Aminoindan-5-yloxy)-5H-pyrrolo[2,3-b]pyrazine-7-carboxylic acid isopropylamide

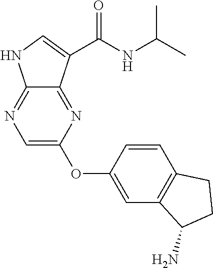 [SMILES: CC(C)NC(=O)c1c[nH]c2ncc(Oc3ccc4c(c3)[C@@H](N)CC4)nc12]

Prepared according to the procedure outlined in Example 270 substituting (S)-3-aminoindan-5-ol for (R)-3-aminoindan-5-ol in Step 1 and omitting Step 4. MS: (M+H)⁺=352.

### Example 292

### 2-(Indan-5-yloxy)-5H-pyrrolo[2,3-b]pyrazine-7-carboxylic acid isopropylamide

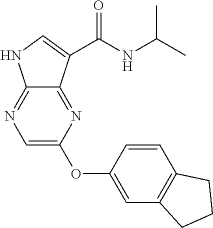 [SMILES: CC(C)NC(=O)c1c[nH]c2ncc(Oc3ccc4c(c3)CCC4)nc12]

Prepared according to the procedure outlined in Example 270, Steps 2 and 5 substituting indan-5-ol for ((R)-6-hydroxyindan-1-yl)-carbamic acid tert-butyl ester in Step 2. MS: (M+H)⁺=337.

### Example 293

### 2-((R)-1-Acetylaminoindan-5-yloxy)-5H-pyrrolo[2,3-b]pyrazine-7-carboxylic acid ((S)-1,2,2-trimethyl-propyl)-amide

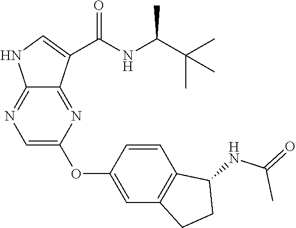 [SMILES: CC(=O)N[C@@H]1CCc2cc(Oc3cnc4[nH]cc(C(=O)N[C@@H](C)C(C)(C)C)c4n3)ccc21]

Prepared according to the procedure outlined in Example 270 substituting (R)-1-amino-indan-5-ol hydrochloride for (R)-3-aminoindan-5-ol in Step 1,2-bromo-5-(2-trimethylsilanyl-ethoxymethyl)-5H-pyrrolo[2,3-b]pyrazine-7-carboxylic acid ((S)-1,2,2-trimethyl-propyl)-amide for 2-bromo-5-(2-trimethylsilanyl-ethoxymethyl)-5H-pyrrolo[2,3-b]pyrazine-7-carboxylic acid isopropylamide in Step 2, and acetyl chloride and pyridine for methanesulfonyl chloride and diisopropylethylamine in Step 4. MS: (M+H)⁺=436.

### Example 294

### 2-((R)-1-Acetylaminoindan-5-yloxy)-5H-pyrrolo[2,3-b]pyrazine-7-carboxylic acid ((S)-sec-butyl)-amide

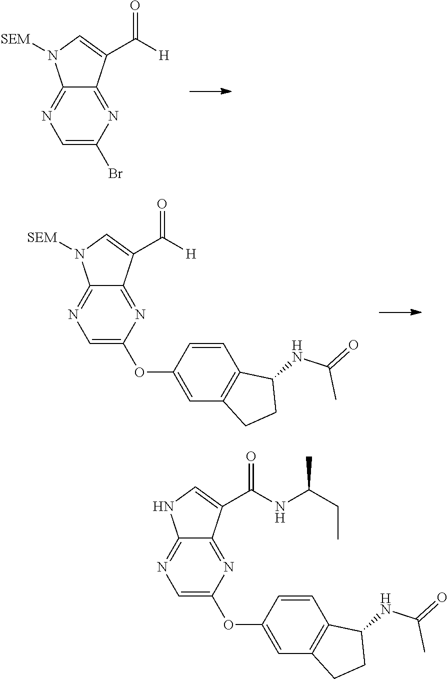

### Step 1

To a stirred solution of 2-bromo-5-(2-trimethylsilanyl-ethoxymethyl)-5H-pyrrolo[2,3-b]pyrazine-7-carbaldehyde (0.25 g, 0.70 mmol) in toluene in a pressure tube were added ((R)-6-hydroxyindan-1-yl)-carbamic acid tert-butyl ester (0.209 g, 0.84 mmol), 2-di-tert-butylphosphino-2′-(N,N-dimethylamino)biphenyl (0.072 g, 0.210 mmol), and K₃PO₄ (0.298 g, 1.404 mmol). The reaction was purged with nitrogen gas for 20 minutes and Pd(OAc)₂ (0.032 g, 0.140 mmol) was added. The tube was sealed and stirred at 90° C. for 16 h. The reaction was cooled and the solvent was evaporated under vacuum. The crude residue was purified by column chromatography over silica gel (100-200 mesh) by using EtOAc/hexane (1015%) as eluting solvent to afford 0.10 g (27%) of {(R)-6-[7-Formyl-5-(2-trimethylsilanyl-ethoxymethyl)-5H-pyrrolo[2,3-b]pyrazin-2-yloxy]-indan-1-yl}-carbamic acid tert-butyl ester as a brown solid. LC-MS: (M+H)⁺=525.

### Step 2

To a stirred solution of {(R)-6-[7-formyl-5-(2-trimethylsilanyl-ethoxymethyl)-5H-pyrrolo[2,3-b]pyrazin-2-yloxy]-indan-1-yl}-carbamic acid tert-butyl ester (1.0 g, 1.91 mmol) in methanol at 0° C., was slowly added acetyl chloride (2.71 mL, 38.1 mmol). The reaction mixture was stirred for 10 min at 0° C. and then for 2 h at 25° C. The solvent was evaporated under reduced pressure at room temperature to afford 0.90 g of 2-((R)-3-aminoindan-5-yloxy)-5-(2-trimethylsilanyl-ethoxymethyl)-5H-pyrrolo[2,3-b]pyrazine-7-carbaldehyde hydrochloride salt as a brown solid which was used without further purification.

### Step 3

A stirred solution of 2-((R)-3-Aminoindan-5-yloxy)-5-(2-trimethylsilanyl-ethoxymethyl)-5H-pyrrolo[2,3-b]pyrazine-7-carbaldehyde) hydrochloride salt (0.90 g, 2.12 mmol) in dichloromethane at 0° C. was neutralized to pH=7 with pyridine (0.671 g, 8.492 mmol) and stirred at 0° C. for 20 minutes. Ac₂O (0.325 g, 3.18 mmol) was added and the reaction mixture was stirred at 25° C. for 16 h. The solvent was evaporated under reduced pressure and the crude residue was purified by column chromatography over silica gel (100-200 mesh) by using EtOAc/hexane (1015%) as eluent to afford 0.45 g (45%) of N-{(R)-6-[7-formyl-5-(2-trimethylsilanyl-ethoxymethyl)-5H-pyrrolo[2,3-b]pyrazin-2-yloxy]-indan-1-yl}-acetamide as a pale yellow solid. LC-MS: (M+H)⁺=467.

### Step 4

To a stirred solution of N-{(R)-6-[7-formyl-5-(2-trimethylsilanyl-ethoxymethyl)-5H-pyrrolo[2,3-b]pyrazin-2-yloxy]-indan-1-yl}-acetamide (0.45 g, 0.97 mmol) in dioxane (10 mL), was added NH₂SO₃H (0.56 g, 5.80 mmol) followed by a solution of NaClO₂ (0.114 g, 1.25 mmol) and KH₂PO₄(1.57 g, 11.592 mmol) in water (5 mL). The reaction mixture was stirred at 25° C. for 16 h then diluted with water and extracted with EtOAc (3×50 mL). The combined organic layer was dried over Na₂SO₄ and concentrated under reduced pressure to afford 0.35 g (75%) of 2-((R)-3-acetylaminoindan-5-yloxy)-5-(2-trimethylsilanyl-ethoxymethyl)-5H-pyrrolo[2,3-b]pyrazine-7-carboxylic acid as white solid which was used without further purification.

### Step 5

To a stirred solution of 2-((R)-3-acetylaminoindan-5-yloxy)-5-(2-trimethylsilanyl-ethoxymethyl)-5H-pyrrolo[2,3-b]pyrazine-7-carboxylic acid (0.20 g, 0.41 mmol) in dichloromethane (10 mL), were added (S)-1-sec-butyl amine (0.033 g, 0.46 mmol) and HATU (175 mg, 0.46 mmol). Diisopropylethylamine (0.21 mL, 1.23 mmol) was added at 0° C. The reaction mixture was stirred at 25° C. for 12 h then quenched with water and extracted with dichloromethane. The combined organic layer was dried over Na₂SO₄ and concentrated under reduced pressure. The crude residue was purified over silica gel (100-200 mesh) by column chromatography to afford 0.12 g (54%) of 2-((R)-3-acetylaminoindan-5-yloxy)-5-(2-trimethylsilanyl-ethoxymethyl)-5H-pyrrolo[2,3-b]pyrazine-7-carboxylic acid ((S)-sec-butyl)-amide as yellow sticky solid.

### Step 6

A stirred solution of 2-((R)-3-acetylaminoindan-5-yloxy)-5-(2-trimethylsilanyl-ethoxymethyl)-5H-pyrrolo[2,3-b]pyrazine-7-carboxylic acid ((S)-sec-butyl)-amide (0.12 g, 0.22 mmol)) in 1.0 M HCl in acetic acid (5 mL) was heated at 65° C. for 3 h. The solvent was completely distilled off under reduced pressure. The residue was dissolved in MeOH:dichloromethane (1:1) and ethylenediamine (20.0 eqv) was added at 0° C. The reaction mixture was stirred at 25° C. for 18 h then the solvent was completely evaporated under reduced pressure and the crude residue was purified by column chromatography over silica gel (100-200 mesh) using MeOH/DCM (2-6%) as eluent to afford 17 mg (19%) of 2-((R)-3-acetylaminoindan-5-yloxy)-5H-pyrrolo[2,3-b]pyrazine-7-carboxylic acid ((S)-sec-butyl)-amide as an off-white solid. MS: (M+H)⁺=408.

### Example 295

### 2-((R)-1-Acetylaminoindan-5-yloxy)-5H-pyrrolo[2,3-b]pyrazine-7-carboxylic acid ((R)-1-cyclopropyl-ethyl)-amide

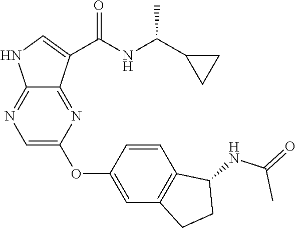 [SMILES: CC(=O)N[C@@H]1CCc2cc(Oc3cnc4[nH]cc(C(=O)N[C@H](C)C5CC5)c4n3)ccc21]

Prepared according to the procedure outlined in Example 294, substituting (R)-1-cyclopropyl-ethylamine for (S)-1-sec-butyl amine in Step 5. MS: (M+H)⁺=420.

### Example 296

### 2-((R)-1-Acetylaminoindan-5-yloxy)-5H-pyrrolo[2,3-b]pyrazine-7-carboxylic acid ((S)-1-cyclopropyl-ethyl)-amide

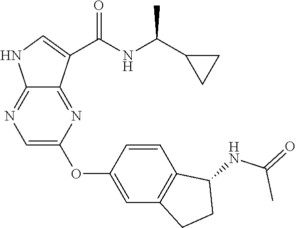 [SMILES: CC(=O)N[C@@H]1CCc2cc(Oc3cnc4[nH]cc(C(=O)N[C@@H](C)C5CC5)c4n3)ccc21]

Prepared according to the procedure outlined in Example 294, substituting (S)-1-cyclopropyl-ethylamine for (S)-1-sec-butyl amine in Step 5. MS: (M+H)⁺=420.

### JAK Assay Information

### Determination of IC₅₀ of Janus Kinase (JAK) Inhibition:

Enzymes and peptide substrate used are described below:
JAK1: Recombinant human kinase domain from Invitrogen (Cat # PV4774)
JAK3: Recombinant human kinase domain from Millipore (Cat #14-629) or prepared.
JAK2: Recombinant human kinase domain from Millipore (Cat #14-640)
Substrate: N-terminally biotinylated 14-mer peptide derived from activation loop of JAK1 with sequence of the peptide substrate: Biotin-KAIETDKEYYTVKD

Assay Conditions Used are Described Below:
Assay Buffer: JAK Kinase Buffer: 50 mM Hepes [pH 7.2], 10 mM MgCl₂, 1 mM DTT,
1 mg/ml BSA. The assay is carried out in this buffer.
Assay Format The kinase activity of all three JAK kinases is measured using a radioactive, end-point assay and with trace amounts of ³³P-ATP. The assays are carried out in 96-well polypropylene plates.

### Experimental Method:

All concentrations are final in the reaction mixture and all incubations are carried at room temperature. Assay steps are described below:
1) Compounds are serially diluted in 100% DMSO typically at a 10× starting concentration of 1 mM. Final concentration of DMSO in the reaction is 10%.
2) Compounds are preincubated with enzyme (0.5 nM JAK3 (commercially available), 0.2 nM JAK3 (prepared), 1 nM JAK2, 5 nM JAK1) for 10 minutes.
3) Reactions are initiated by the addition of a cocktail of the two substrates (ATP and peptide premixed in the JAK Kinase Buffer). In the JAK2/JAK3 assays, ATP and the peptide are used at concentrations of 1.5 uM and 50 uM, respectively. JAK1 assay is carried out at an ATP concentration of 10 uM and a peptide concentration of 50 uM.
4) The duration of the assay for JAK2 and JAK3 is 20 minutes. JAK1 assay is carried out for 40 minutes. With all three enzymes, reactions are terminated by the addition of 0.5M EDTA to a final concentration of 100 mM.
5) 25 ul of terminated reactions are transferred to 150 ul of a 7.5% (v/v) slurry of streptavidin-coated sepharose beads in MgCl₂- and CaCl₂-free 1× Phosphate Buffered Saline containing 50 mM of EDTA in 96-well, 1.2 um MultiScreen-BV filter plates.
6) After a 30-minute incubation, the beads are washed under vacuum with the following buffers:
   a. 3 to 4 washes with 200 ul of 2M NaCl.
   b. 3 to 4 washes with 200 ul of 2M NaCl plus 1% (v/v) phosphoric acid.
   c. 1 wash with water.
7) Washed plates are dried in a 60° C. oven for between 1 to 2 hours.
8) 70 ul of Microscint 20 scintillation fluid is added to each well of filter plates and after at least 30 minutes of incubation, radioactive counts are measured in a Perkin-Elmer microplate scintillation counter.

Representative IC₅₀ results are in Table II below:

TABLE II

| | Ic50 h-jak3(810- |
|---|---|
| | 1124)-sf9-c:no |
| Compound | additive (μM) |
| I-1 | 0.22486 |
| I-2 | 0.00523 |
| I-3 | 0.01219 |
| I-4 | 0.03485 |
| I-5 | 0.30171 |
| I-6 | 0.12621 |
| I-7 | 0.00451 |
| I-8 | 0.04879 |
| I-9 | 0.01960 |
| I-10 | 0.03433 |
| I-11 | 0.15173 |
| I-12 | 0.19236 |
| I-13 | 0.43629 |
| I-14 | 0.40877 |
| I-15 | 0.00749 |
| I-16 | 0.45888 |
| I-17 | 0.08448 |
| I-18 | 0.00305 |
| I-19 | 0.00614 |
| I-20 | 0.02977 |
| I-21 | 0.04500 |
| I-22 | 0.00590 |
| I-24 | 0.17964 |
| I-25 | 0.00673 |
| I-26 | 0.00408 |
| I-27 | 0.93386 |
| I-28 | 0.68064 |
| I-29 | 0.05427 |
| I-30 | 0.01905 |
| I-31 | 0.03948 |
| I-32 | 0.02008 |
| I-33 | 0.49272 |
| I-34 | 0.03643 |
| I-35 | 1.03872 |
| I-36 | 0.03361 |
| I-37 | 0.02204 |
| I-38 | 0.11500 |
| I-39 | 0.30185 |
| I-40 | 0.12828 |
| I-41 | 0.11153 |
| I-42 | 0.13101 |
| I-43 | 0.12077 |
| I-44 | 0.04604 |
| I-45 | 0.20773 |
| I-46 | 0.22824 |
| I-47 | 0.34309 |
| I-48 | 0.11565 |
| I-49 | 0.45253 |
| I-50 | 0.00903 |
| I-51 | 0.41420 |
| I-52 | 0.01107 |
| I-53 | 0.00838 |
| I-54 | 0.00546 |
| I-55 | 0.02940 |
| I-56 | 0.00984 |
| I-57 | 0.21673 |
| I-58 | 0.03768 |
| I-59 | 0.05887 |
| I-60 | 0.01505 |
| I-61 | 0.00257 |
| I-62 | 0.00746 |
| I-63 | 0.03047 |
| I-64 | 0.03513 |
| I-65 | 0.26197 |
| I-66 | 0.04997 |
| I-67 | 0.42702 |
| I-68 | 0.35826 |
| I-69 | 0.14348 |
| I-70 | 0.08700 |
| I-71 | 0.00380 |
| I-72 | 0.01000 |
| I-73 | 0.09242 |
| I-74 | 0.02706 |
| I-75 | 0.06867 |
| I-76 | 0.13209 |
| I-77 | 0.01367 |
| I-78 | 0.07861 |
| I-79 | 0.04069 |
| I-80 | 0.00390 |
| I-81 | 0.01599 |
| I-82 | 0.01516 |
| I-83 | 0.04725 |
| I-84 | 0.06514 |
| I-85 | 0.07251 |
| I-86 | 0.03919 |
| I-87 | 0.00182 |
| I-88 | 1.41346 |
| I-89 | 0.01339 |
| I-90 | 0.01454 |
| I-91 | 0.03868 |
| I-92 | 0.09507 |
| I-93 | 0.05784 |
| I-94 | 0.87751 |
| I-95 | 0.00999 |
| I-96 | 0.02595 |
| I-97 | 0.01804 |
| I-98 | 0.01113 |
| I-99 | 0.00661 |
| I-100 | 0.03846 |
| I-101 | 0.01203 |
| I-102 | 0.01032 |
| I-103 | 0.00725 |
| I-104 | 0.01632 |
| I-105 | 0.10280 |
| I-106 | 0.01475 |
| I-107 | 0.06129 |
| I-108 | 0.16295 |
| I-109 | 0.23090 |
| I-110 | 0.04545 |
| I-111 | 0.27573 |
| I-112 | 0.08615 |
| I-113 | 0.03568 |
| I-114 | 0.06343 |
| I-115 | 3.30096 |
| I-116 | 0.68209 |
| I-117 | 0.02677 |
| I-118 | 0.14998 |
| I-119 | 0.15058 |
| I-120 | 0.02436 |
| I-121 | 0.05950 |
| I-122 | 0.06382 |
| I-123 | 1.33371 |
| I-124 | 0.44223 |
| I-125 | 0.50236 |
| I-126 | 0.19021 |
| I-127 | 0.14283 |
| I-128 | 0.21127 |
| I-129 | 0.22225 |
| I-130 | 0.65874 |
| I-131 | 1.02865 |
| I-132 | 0.41477 |
| I-133 | 0.15380 |
| I-134 | 0.89130 |
| I-135 | 0.02064 |
| I-136 | 0.28345 |
| I-137 | 0.34633 |
| I-138 | 0.00501 |
| I-139 | 0.00055 |
| I-140 | 0.00088 |
| I-141 | 0.00019 |
| I-142 | 0.00018 |
| I-143 | 0.00628 |
| I-144 | 0.00340 |
| I-145 | 0.00461 |
| I-146 | 0.01312 |
| I-147 | 0.00246 |
| I-148 | 0.00325 |
| I-149 | 0.00299 |
| I-150 | 0.00512 |
| I-151 | 0.01244 |
| I-152 | 0.02528 |
| I-153 | 0.07538 |
| I-155 | 0.00032 |
| I-156 | 0.00035 |
| I-157 | 0.00043 |
| I-158 | 0.00032 |
| I-159 | 0.00053 |
| I-160 | 0.00032 |
| I-161 | 0.00032 |
| I-162 | 0.00032 |
| I-163 | 0.00300 |
| I-164 | 0.00032 |
| I-165 | 0.00017 |
| I-166 | 0.00011 |
| I-167 | 0.00032 |
| I-168 | 0.00022 |
| I-169 | 0.00029 |
| I-170 | 0.00058 |
| I-171 | 0.00023 |
| I-172 | 0.00014 |
| I-173 | 0.00020 |
| I-174 | 0.00022 |
| I-175 | 0.00010 |
| I-176 | 0.00046 |
| I-177 | 0.00032 |
| I-178 | 0.00032 |
| I-179 | 0.00032 |
| I-180 | 0.00032 |
| I-181 | 0.00062 |
| I-182 | 0.00032 |
| I-183 | 0.00032 |
| I-184 | 0.00032 |
| I-185 | 0.00032 |
| I-186 | 0.00046 |
| I-187 | 0.00046 |
| I-188 | 0.00041 |
| I-189 | 0.00060 |
| I-190 | 0.00069 |
| I-191 | 0.00055 |
| I-192 | 0.00040 |
| I-193 | 0.00042 |
| I-194 | 0.00134 |
| I-195 | 0.00046 |
| I-196 | 0.00047 |
| I-197 | 0.00032 |
| I-198 | 0.00054 |
| I-199 | 0.00065 |
| I-200 | 0.00073 |
| I-201 | 0.00057 |
| I-202 | 0.00052 |
| I-203 | 0.00040 |
| I-204 | 0.00056 |
| I-205 | 0.00076 |
| I-206 | 0.00032 |
| I-207 | 0.00073 |
| I-209 | 0.00047 |
| I-210 | 0.00071 |
| I-211 | 0.00022 |
| I-212 | 0.00016 |
| I-213 | 0.00011 |
| I-214 | 0.00028 |
| I-215 | 0.00021 |
| I-216 | 0.00018 |
| I-217 | 0.00011 |
| I-218 | 0.00013 |
| I-219 | 0.00024 |
| I-220 | 0.00023 |
| I-221 | 0.00008 |
| I-222 | 0.00011 |
| I-223 | 0.00091 |
| I-224 | 0.00036 |
| I-225 | 0.00019 |
| I-226 | 0.00050 |
| I-227 | 0.01056 |
| I-228 | 0.61869 |
| I-229 | 0.18371 |
| I-230 | 2.61358 |
| I-231 | 4.22410 |
| I-232 | 0.17718 |
| I-233 | 0.40678 |
| I-234 | 0.97958 |
| I-235 | 0.78443 |
| I-236 | 2.96483 |
| I-237 | 5.35592 |
| I-238 | 3.84823 |
| I-239 | 5.19661 |
| I-240 | 0.31533 |
| I-241 | 0.67717 |
| I-242 | 1.17718 |
| I-243 | 0.30967 |
| I-244 | 1.07179 |
| I-245 | 3.30868 |
| I-246 | 3.45523 |
| I-247 | 6.45564 |
| I-248 | 1.03367 |
| I-249 | 1.59120 |
| I-250 | 4.58553 |
| I-251 | 4.03349 |
| I-252 | 0.91369 |
| I-253 | 2.17078 |
| I-254 | 2.98880 |
| I-255 | 0.38180 |
| I-256 | 0.93054 |
| I-257 | 2.74335 |
| I-258 | 0.24613 |
| I-259 | 6.70858 |
| I-260 | 1.52760 |
| I-261 | 2.98802 |
| I-262 | 1.87094 |
| I-263 | 2.34964 |
| I-264 | 0.81002 |
| I-265 | 1.10243 |
| I-266 | 0.38114 |
| I-267 | 2.35818 |
| I-268 | 0.15540 |
| I-269 | 0.08561 |
| I-270 | 0.73976 |
| I-271 | 1.27124 |
| I-272 | 0.02931 |
| I-273 | 0.46951 |
| I-274 | 0.35325 |
| I-275 | 0.62606 |
| I-276 | 0.57423 |
| I-277 | 0.22900 |
| I-278 | 0.49109 |
| I-279 | 1.10872 |
| I-280 | 3.97752 |
| I-281 | 0.13848 |
| I-282 | 0.12540 |
| I-283 | 0.05667 |
| I-284 | 0.04997 |
| I-285 | 0.23431 |
| I-286 | 0.15244 |
| I-287 | 0.02015 |
| I-288 | 0.29715 |
| I-289 | 0.53285 |
| I-290 | 0.26103 |
| I-291 | 0.03697 |
| I-292 | 0.09174 |
| I-293 | 0.16189 |
| I-294 | 0.01234 |
| I-295 | 0.00964 |
| I-296 | 0.02288 |
| I-297 | 0.57324 |
| I-298 | 0.38682 |
| I-299 | 0.14336 |
| I-300 | 0.19924 |
| I-301 | 0.11839 |
| I-302 | 0.15723 |
| I-303 | 0.13997 |
| I-304 | 0.15495 |

### SYK Assay Information

### Determination of IC₅₀ of Spleen Tyrosine Kinase (SYK) Inhibition:

SYK kinase assay is a standard kinase assay adapted to a 96 well plate format. This assay is performed in 96-well format for IC₅₀ determination with 8 samples which represented 10 half log dilutions and a 40 μL reaction volume. The assay measures the incorporation of radiolabeled ³³PγATP into an N-terminally biotinylated peptide substrate, derived from naturally occurring phosphoacceptor consensus sequence (Biotin-11aa DY*E). Phosphorylated products were detected upon termination of reactions with EDTA and the addition of Streptavidin coated beads. Representative results are in Table II above.
Assay plates: 96-well MultiScreen 0.65 um filter plates (Millipore Cat. No.: MADVNOB10)
Streptavidin coated beads: Streptavidin Sepharose™, suspension 5.0 mL, in 50 mM EDTA/PBS diluted (1:100), (Amersham, Cat. No.: 17-5113-01)
Compounds: 10 mM in 100% dimethylsulfoxide (DMSO), final conc.: compound 0.003-100 uM in 10% DMSO
Enzyme: SYK RPA purified, truncated construct of Spleen Tyrosine Kinase aa 360-635, stock solution 1 mg/mL, MW: 31.2 KDa, final conc.:0.0005 μM.
Peptide 1: biotinylated peptide is derived from a naturally occurring phosphor-acceptor consensus sequence (Biotin-EPEGDYEEVLE), special order from QCB, stock solution 20 mM, final conc.: 5.0 μM.
ATP: Adenosine-5′-triphosphate 20 mM, (ROCHE Cat. No.: 93202720), final concentration: 20 μM
Buffer: HEPES: 2-Hydroxyethyl piperazine-2-ethanesulfonic acid (Sigma, Cat. No.: H-3375) final concentration: 50 mM HEPES pH7.5
BSA: Bovine Serum Albumin Fraction V, fatty acid free (Roche Diagnostics GmbH, Cat. No. 9100221) diluted to a final concentration of 0.1%
EDTA: EDTA stock solution 500 mM, (GIBCO, Cat. No.: 15575-038) final concentration: 0.1 mM
DTT: 1,4-Dithiothreitol (Roche Diagnostics GmbH, Cat. No.: 197777), final conc.: 1 mM
MgCl₂×6H₂O: MERCK, Cat. No.: 105833.1000, final concentration: 10 mM
Assay Dilution Buffer (ADB): 50 mM HEPES, 0.1 mM EGTA, 0.1 mM Na Vanadate, 0.1 mM β-glycerophosphate, 10 mM MgCl₂, 1 mM DTT, 0.1% BSA, pH 7.5
Bead wash buffer: 10 g/L PBS (Phosphate buffered saline) with 2M NaCl+1% phosphoric acid.

### Experimental Method:

In 40 μL volume, 26 μL of ADB diluted, purified recombinant human SYK360-635 [0.5 nM] was mixed with 4 μL of 10× concentrations of the test compounds, [usually 100 μM-0.003 μM] in [10%] DMSO and the mixture was incubated for 10 min at RT.

The kinase reaction was initiated by the addition of 10 μL 4× substrate cocktail containing the DYE peptide substrate [0 or 5 μM], ATP [20 μM] and ³³PγATP [2 μCi/r×n]. After incubation at 30° C. for 15 min, the reaction was terminated by the transfer of 25 μL of the reaction sample to a 96 well 0.65 μm Millipore MADVNOB membrane/plate containing 200 μL 5 mM EDTA and 20% Streptavidine coated beads in PBS.

The unbound radionucleotides were washed under vacuum with 3×250 μL 2M NaCl; 2×250 μL 2M NaCl+1% phosphoric acid; 1×250 μL H₂O. After the last wash membrane/plates were transferred to an adaptor plate, heat dried for 15 min at 60° C., and 50 μL scintillation cocktail was added to each well and 4 h later the amount of radioactivity was counted in a top counter.

The percent inhibition was calculated based on the uninhibited enzyme rate:

% Inhibition=100/(1+(IC₅₀/Inhibitor conc)ⁿ)
The IC₅₀ was calculated using a non-linear curve fit with XLfit software (ID Business Solution Ltd., Guilford, Surrey, UK).

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

What is claimed is:

1. A compound of Formula I 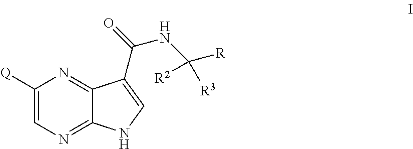
wherein:
R is H, cyano, lower alkyl, R′ or 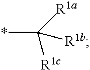
R′ is cycloalkyl, heterocycloalkyl, heteroaryl, or phenyl, wherein each is optionally substituted with one or more R″;
each R″ is independently halo, hydroxy, cyano, lower alkyl, lower haloalkyl, lower alkoxy, lower hydroxyalkyl, cycloalkyl, C(═O)R′″, or S(═O)₂R′″;
each R′″ is independently OH or lower alkyl;
R^{1a} and R^{1b} are each independently H, hydroxy, halo, lower alkyl, lower alkenyl, lower alkynyl, lower haloalkyl, lower alkoxy, lower haloalkoxy, lower hydroxyalkyl, amino, lower alkylamino, lower dialkylamino, cyano, C(═O)R′″, S(═O)₂R′″ or CH₂S(═O)₂R′″;
R^{1c} is phenyl, cycloalkyl, heterocycloalkyl, or heteroaryl, optionally substituted with one or more R^{1d};
each R^{1d} is independently hydroxy, halo, lower alkyl, lower hydroxyalkyl, lower halo alkyl, or lower alkoxy;
R² is H, hydroxy lower alkyl, lower haloalkyl, or lower alkyl;
R³ is H, hydroxy, cyano, cyano lower alkyl, or R^{3′};
each R^{3′} is independently lower alkyl, hydroxy lower alkyl, lower alkoxy, lower haloalkyl, lower haloalkoxy, phenyl lower alkyl, cycloalkyl or cycloalkyl lower alkyl, each optionally substituted with one or more R^{3″};
each R^{3″} is independently lower alkyl, halo, hydroxy, lower alkoxy, lower haloalkyl, lower hydroxy alkyl, oxo, amino, cyano, cyano lower alkyl, S(═O)₂R^{3′″}, C(═O)R^{3′″}, cycloalkyl, heterocycloalkyl, heteroaryl, or heterocycloalkenyl;
each R^{3′″} is independently H, hydroxy or lower alkyl;
Q is Q²;
Q² is optionally substituted with one or more Q^{2a};
Q^{2a} is Q^{2b} or Q^{2c};
each Q^{2b} is independently halogen, oxo, hydroxy, —CN, —SCH₃, —S(O)₂CH₃, or —S(═O)CH₃;
each Q^{2a} is independently Q^{2d} or Q^{2e};
or two Q^{2a} come together to form a bicyclic ring system, optionally substituted with one or more Q^{2b} or Q^{2c};
each Q^{2d} is independently —O(Q^{2e}), —S(═O)₂(Q^{2e}), —C(═O)N(Q^{2e})₂, —S(O)₂(Q^{2e}), —C(═O)(Q^{2e}), —C(═O)O(Q^{2e}), —N(Q^{2e})C(═O)(Q^{2e}), —N(Q^{2e})C(═O)O(Q^{2e}), or —N(Q^{2e})C(═O)N(Q^{2e})₂;
each Q^{2e} is independently H or Q^{2e′};
each Q^{2e′} is independently lower alkyl, phenyl, benzyl, 5,6,7,8-Tetrahydro-naphthalene, lower haloalkyl, lower alkoxy, cycloalkyl, cycloalkenyl, heterocycloalkyl, spirocyclic heterocycloalkyl, or heteroaryl, optionally substituted with one or more Q^{2f};
each Q^{2f} is independently Q^{2g} or Q^{2h};
each Q^{2g} is independently halogen, hydroxy, cyano, oxo, —S(═O)₂(Q^{2i′}), S(═O)₂N(Q^{2i′})₂, —C(═O)OH, C(═O)N(Q^{2i′})₂, or —C(═O)(Q^{2i′});
each Q^{2h} is independently lower alkyl, lower alkenyl, lower haloalkyl, lower alkoxy, amino, phenyl, benzyl, cycloalkyl, heterocycloalkyl, or heteroaryl, optionally substituted with one or more Q²ⁱ; and
each Q²ⁱ is independently halogen, hydroxy, cyano, lower alkyl, lower haloalkyl, or lower alkoxy;
each Q^{2i′} is independently H or lower alkyl;
with the proviso that the compound of Formula I is not 2-Thiophen-2-yl-5H-pyrrolo[2,3-b]pyrazine-7-carboxylic acid isopropylamide; 2-Thiophen-2-yl-5H-pyrrolo[2,3-b]pyrazine-7-carboxylic acid (3-hydroxy-2,2-dimethyl-propyl)-amide; or compound with trifluoro-acetic acid;
or a pharmaceutically acceptable salt thereof.

2. The compound of claim 1, wherein either R² or R³ is lower alkyl and the other is H.

3. The compound of claim 1, wherein both R² and R³ are methyl.

4. The compound of claim 3 wherein R^{1a} is lower alkyl, hydroxy, lower haloalkyl, lower alkoxy, cyano, or lower hydroxyalkyl.

5. The compound of claim 4, wherein R^{1a} is methyl.

6. The compound of claim 5, wherein R^{1b} is methyl.

7. The compound of claim 5, wherein R^{1d} is lower alkyl, hydroxy, lower hydroxyalkyl, lower alkoxy, lower haloalkyl, cyano, or methanesulfonylmethylenyl.

8. The compound of claim 7, wherein R^{1d} is H, hydroxy, or lower alkyl.

9. The compound of claim 8, wherein R^{1d} is methyl or hydroxy.

10. The compound of claim 5, wherein R^{1b} is lower alkyl or lower haloalkyl.

11. The compound of claim 6 wherein Q is thienyl, or pyrazolyl, each optionally substituted with one or more Q^{2a}.

12. A pharmaceutical composition comprising the compound of claim 1, admixed with at least one pharmaceutically acceptable carrier, excipient or diluent.

13. A compound of claim 1, selected from the group consisting of:
2-Thiophen-2-yl-5H-pyrrolo[2,3-b]pyrazine-7-carboxylic acid ((S)-2-hydroxy-1,2-dimethyl-propyl)-amide;
2-(2-Methyl-pyridin-4-yl)-5H-pyrrolo[2,3-b]pyrazine-7-carboxylic acid ((S)-2-hydroxy-1,2-dimethyl-propyl)-amide;
2-(6-Methyl-pyridin-3-yl)-5H-pyrrolo[2,3-b]pyrazine-7-carboxylic acid ((S)-2-hydroxy-1,2-dimethyl-propyl)-amide;
2-Pyridin-2-yl-5H-pyrrolo[2,3-b]pyrazine-7-carboxylic acid ((S)-1,2,2-trimethyl-propyl)-amide;
2-(1-Ethyl-1H-pyrazol-4-yl)-5H-pyrrolo[2,3-b]pyrazine-7-carboxylic acid ((R)-2-hydroxy-1,2-dimethyl-propyl)-amide;
2-(1-Methyl-1H-pyrazol-4-yl)-5H-pyrrolo[2,3-b]pyrazine-7-carboxylic acid ((R)-2-hydroxy-1,2-dimethyl-propyl)-amide;
2-Thiophen-2-yl-5H-pyrrolo[2,3-b]pyrazine-7-carboxylic acid ((R)-2-hydroxy-1,2-dimethyl-propyl)-amide;
2-Thiazol-2-yl-5H-pyrrolo[2,3-b]pyrazine-7-carboxylic acid ((R)-2-hydroxy-1,2-dimethyl-propyl)-amide;
2-Pyridin-2-yl-5H-pyrrolo[2,3-b]pyrazine-7-carboxylic acid ((R)-2-hydroxy-1,2-dimethyl-propyl)-amide;
2-(1-Ethyl-1H-pyrazol-4-yl)-5H-pyrrolo[2,3-b]pyrazine-7-carboxylic acid [(S)-1-(1-hydroxy-cyclopentyl)-ethyl]-amide;
2-(1-Methyl-1H-pyrazol-4-yl)-5H-pyrrolo[2,3-b]pyrazine-7-carboxylic acid ((S)-2-cyano-1,2,2-trimethyl-ethyl)-amide;
2-(1-Methyl-1H-pyrazol-4-yl)-5H-pyrrolo[2,3-b]pyrazine-7-carboxylic acid ((S)-1-cyclohexyl-ethyl)-amide;
2-(1-Methyl-1H-pyrazol-4-yl)-5H-pyrrolo[2,3-b]pyrazine-7-carboxylic acid ((S)-1,2,2-trimethyl-propyl)-amide;
2-Thiophen-2-yl-5H-pyrrolo[2,3-b]pyrazine-7-carboxylic acid ((S)-1,2,2-trimethyl-propyl)-amide;
2-[1-(3-Chloro-phenyl)-1H-imidazol-4-yl]-5H-pyrrolo[2,3-b]pyrazine-7-carboxylic acid ((S)-1,2,2-trimethyl-propyl)-amide;
2-[1-(3-Trifluoromethyl-phenyl)-1H-imidazol-4-yl]-5H-pyrrolo[2,3-b]pyrazine-7-carboxylic acid ((S)-1,2,2-trimethyl-propyl)-amide;
2-[1-(5-Chloro-2-fluoro-phenyl)-1H-imidazol-4-yl]-5H-pyrrolo[2,3-b]pyrazine-7-carboxylic acid ((S)-1,2,2-trimethyl-propyl)-amide;
2-[1-(2-Fluoro-5-methyl-phenyl)-1H-imidazol-4-yl]-5H-pyrrolo[2,3-b]pyrazine-7-carboxylic acid ((S)-1,2,2-trimethyl-propyl)-amide;
2-[1-(2-Fluoro-5-trifluoromethyl-phenyl)-1H-imidazol-4-yl]-5H-pyrrolo[2,3-b]pyrazine-7-carboxylic acid ((S)-1,2,2-trimethyl-propyl)-amide;
2-(1-m-Tolyl-1H-imidazol-4-yl)-5H-pyrrolo[2,3-b]pyrazine-7-carboxylic acid ((S)-1,2,2-trimethyl-propyl)-amide;
2-[1-(3-Ethyl-phenyl)-1H-imidazol-4-yl]-5H-pyrrolo[2,3-b]pyrazine-7-carboxylic acid ((S)-1,2,2-trimethyl-propyl)-amide;
2-[1-(3-Isopropyl-phenyl)-1H-imidazol-4-yl]-5H-pyrrolo[2,3-b]pyrazine-7-carboxylic acid ((S)-1,2,2-trimethyl-propyl)-amide;
2-[1-(3-tert-Butyl-phenyl)-1H-imidazol-4-yl]-5H-pyrrolo[2,3-b]pyrazine-7-carboxylic acid ((S)-1,2,2-trimethyl-propyl)-amide;
2-(1,3-Dimethyl-1H-pyrazol-4-yl)-5H-pyrrolo[2,3-b]pyrazine-7-carboxylic acid ((S)-2-methoxy-1-methyl-ethyl)-amide;
2-(5-Ethylcarbamoyl-thiophen-2-yl)-5H-pyrrolo[2,3-b]pyrazine-7-carboxylic acid ((S)-1,2,2-trimethyl-propyl)-amide;
2-(5-Isopropylcarbamoyl-thiophen-2-yl)-5H-pyrrolo[2,3-b]pyrazine-7-carboxylic acid ((S)-1,2,2-trimethyl-propyl)-amide;
2-(5-tert-Butylcarbamoyl-thiophen-2-yl)-5H-pyrrolo[2,3-b]pyrazine-7-carboxylic acid ((S)-1,2,2-trimethyl-propyl)-amide;
2-[5-(1-Methyl-2-pyrazol-1-yl-ethylcarbamoyl)-thiophen-2-yl]-5H-pyrrolo[2,3-b]pyrazine-7-carboxylic acid ((S)-1,2,2-trimethyl-propyl)-amide;
2-{5-[2-(4-Fluoro-phenyl)-1-methyl-ethylcarbamoyl]-thiophen-2-yl}-5H-pyrrolo[2,3-b]pyrazine-7-carboxylic acid ((S)-1,2,2-trimethyl-propyl)-amide;
2-(5-Diethylcarbamoyl-thiophen-2-yl)-5H-pyrrolo[2,3-b]pyrazine-7-carboxylic acid ((S)-1,2,2-trimethyl-propyl)-amide;
2-[5-(4-Methyl-piperazine-1-carbonyl)-thiophen-2-yl]-5H-pyrrolo[2,3-b]pyrazine-7-carboxylic acid ((S)-1,2,2-trimethyl-propyl)-amide;
2-[5-((R)-1-Cyclopropyl-ethylcarbamoyl)-thiophen-2-yl]-5H-pyrrolo[2,3-b]pyrazine-7-carboxylic acid ((S)-1,2,2-trimethyl-propyl)-amide;
2-[1-(3-Vinyl-phenyl)-1H-imidazol-4-yl]-5H-pyrrolo[2,3-b]pyrazine-7-carboxylic acid ((S)-1,2,2-trimethyl-propyl)-amide;
2-{5-[(Pyridin-3-ylmethyl)-carbamoyl]-thiophen-2-yl}-5H-pyrrolo[2,3-b]pyrazine-7-carboxylic acid ((S)-1,2,2-trimethyl-propyl)-amide;
2-{5-[(Pyridin-4-ylmethyl)-carbamoyl]-thiophen-2-yl}-5H-pyrrolo[2,3-b]pyrazine-7-carboxylic acid ((S)-1,2,2-trimethyl-propyl)-amide;
2-{5-[(Pyridin-2-ylmethyl)-carbamoyl]-thiophen-2-yl}-5H-pyrrolo[2,3-b]pyrazine-7-carboxylic acid ((S)-1,2,2-trimethyl-propyl)-amide;
2-[5-(4-Cyano-piperidine-1-carbonyl)-thiophen-2-yl]-5H-pyrrolo[2,3-b]pyrazine-7-carboxylic acid ((S)-1,2,2-trimethyl-propyl)-amide;
2-[5-(Cyclopentylmethyl-carbamoyl)-thiophen-2-yl]-5H-pyrrolo[2,3-b]pyrazine-7-carboxylic acid ((S)-1,2,2-trimethyl-propyl)-amide;
2-[5-((R)-2-Hydroxy-1-methyl-ethylcarbamoyl)-thiophen-2-yl]-5H-pyrrolo[2,3-b]pyrazine-7-carboxylic acid ((S)-1,2,2-trimethyl-propyl)-amide;
2-[5-((R)-1-Methyl-2-phenyl-ethylcarbamoyl)-thiophen-2-yl]-5H-pyrrolo[2,3-b]pyrazine-7-carboxylic acid ((S)-1,2,2-trimethyl-propyl)-amide;
2-[5-(1-Pyridin-3-yl-ethylcarbamoyl)-thiophen-2-yl]-5H-pyrrolo[2,3-b]pyrazine-7-carboxylic acid ((S)-1,2,2-trimethyl-propyl)-amide;
2-[5-(Cyanomethyl-carbamoyl)-thiophen-2-yl]-5H-pyrrolo[2,3-b]pyrazine-7-carboxylic acid ((S)-1,2,2-trimethyl-propyl)-amide;
2-[5-(2-Sulfamoyl-ethylcarbamoyl)-thiophen-2-yl]-5H-pyrrolo[2,3-b]pyrazine-7-carboxylic acid ((S)-1,2,2-trimethyl-propyl)-amide;
2-[5-(2-Imidazol-1-yl-1-methyl-ethylcarbamoyl)-thiophen-2-yl]-5H-pyrrolo[2,3-b]pyrazine-7-carboxylic acid ((S)-1,2,2-trimethyl-propyl)-amide;
2-[5-(4-Hydroxy-4-methyl-piperidine-1-carbonyl)-thiophen-2-yl]-5H-pyrrolo[2,3-b]pyrazine-7-carboxylic acid ((S)-1,2,2-trimethyl-propyl)-amide;
2-[5-(1-Methyl-2-pyridin-2-yl-ethylcarbamoyl)-thiophen-2-yl]-5H-pyrrolo[2,3-b]pyrazine-7-carboxylic acid ((S)-1,2,2-trimethyl-propyl)-amide;
2-[5-(7-Aza-bicyclo[2.2.1]heptane-7-carbonyl)-thiophen-2-yl]-5H-pyrrolo[2,3-b]pyrazine-7-carboxylic acid ((S)-1,2,2-trimethyl-propyl)-amide;
2-[5-(3-Cyano-azetidine-1-carbonyl)-thiophen-2-yl]-5H-pyrrolo[2,3-b]pyrazine-7-carboxylic acid ((S)-1,2,2-trimethyl-propyl)-amide;
2-[5-(3-Carbamoyl-azetidine-1-carbonyl)-thiophen-2-yl]-5H-pyrrolo[2,3-b]pyrazine-7-carboxylic acid ((S)-1,2,2-trimethyl-propyl)-amide;
2-[5-(Azetidine-1-carbonyl)-thiophen-2-yl]-5H-pyrrolo[2,3-b]pyrazine-7-carboxylic acid ((S)-1,2,2-trimethyl-propyl)-amide;
2-[5-(2,6-Dimethyl-piperidine-1-carbonyl)-thiophen-2-yl]-5H-pyrrolo[2,3-b]pyrazine-7-carboxylic acid ((S)-1,2,2-trimethyl-propyl)-amide;
1-{5-[7-((S)-1,2,2-Trimethyl-propylcarbamoyl)-5H-pyrrolo[2,3-b]pyrazin-2-yl]-thiophene-2-carbonyl}-piperidine-4-carboxylic acid;
2-[5-(4-Acetylamino-piperidine-1-carbonyl)-thiophen-2-yl]-5H-pyrrolo[2,3-b]pyrazine-7-carboxylic acid ((S)-1,2,2-trimethyl-propyl)-amide;
2-[5-(4-Methyl-benzylcarbamoyl)-thiophen-2-yl]-5H-pyrrolo[2,3-b]pyrazine-7-carboxylic acid ((S)-1,2,2-trimethyl-propyl)-amide;
2-[5-(4-Fluoro-benzylcarbamoyl)-thiophen-2-yl]-5H-pyrrolo[2,3-b]pyrazine-7-carboxylic acid ((S)-1,2,2-trimethyl-propyl)-amide;
2-[5-(2,3-Dichloro-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Methyl-benzylcarbamoyl)-thiophen-2-yl]-5H-pyrrolo[2,3-b]pyrazine-7-carboxylic acid ((S)-1,2,2-trimethyl-propyl)-amide;
2-[5-(2,6-Difluoro-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Chloro-6-fluoro-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Methyl-cyclohexylcarbamoyl)-thiophen-2-yl]-5H-pyrrolo[2,3-b]pyrazine-7-carboxylic acid ((S)-1,2,2-trimethyl-propyl)-amide;
2-[5-((1S,2R)-2-Phenyl-cyclopropylcarbamoyl)-thiophen-2-yl]-5H-pyrrolo[2,3-b]pyrazine-7-carboxylic acid ((S)-1,2,2-trimethyl-propyl)-amide;
2-{5-[(4-Methyl-thiophen-2-ylmethyl)-carbamoyl]-thiophen-2-yl}-5H-pyrrolo[2,3-b]pyrazine-7-carboxylic acid ((S)-1,2,2-trimethyl-propyl)-amide;
2-{5-[(5-Methyl-furan-2-ylmethyl)-carbamoyl]-thiophen-2-yl}-5H-pyrrolo[2,3-b]pyrazine-7-carboxylic acid ((S)-1,2,2-trimethyl-propyl)-amide;
2-[5-(Adamantan-1-ylcarbamoyl)-thiophen-2-yl]-5H-pyrrolo[2,3-b]pyrazine-7-carboxylic acid ((S)-1,2,2-trimethyl-propyl)-amide;
2-{5-[1-(4-Fluoro-phenyl)-ethylcarbamoyl]-thiophen-2-yl}-5H-pyrrolo[2,3-b]pyrazine-7-carboxylic acid ((S)-1,2,2-trimethyl-propyl)-amide;
2-[5-(Methoxy-methyl-carbamoyl)-thiophen-2-yl]-5H-pyrrolo[2,3-b]pyrazine-7-carboxylic acid ((S)-1,2,2-trimethyl-propyl)-amide;
2-(5-Methoxycarbamoyl-thiophen-2-yl)-5H-pyrrolo[2,3-b]pyrazine-7-carboxylic acid ((S)-1,2,2-trimethyl-propyl)-amide;
2-(5-Prop-2-ynylcarbamoyl-thiophen-2-yl)-5H-pyrrolo[2,3-b]pyrazine-7-carboxylic acid ((S)-1,2,2-trimethyl-propyl)-amide;
2-{5-[(R)-2-(3H-Imidazol-4-yl)-1-methyl-ethylcarbamoyl]-thiophen-2-yl}-5H-pyrrolo[2,3-b]pyrazine-7-carboxylic acid ((S)-1,2,2-trimethyl-propyl)-amide;
2-[5-(5,6,7,8-Tetrahydro-naphthalen-2-ylcarbamoyl)-thiophen-2-yl]-5H-pyrrolo[2,3-b]pyrazine-7-carboxylic acid ((S)-1,2,2-trimethyl-propyl)-amide;
2-(5-Phenylcarbamoyl-thiophen-2-yl)-5H-pyrrolo[2,3-b]pyrazine-7-carboxylic acid ((S)-1,2,2-trimethyl-propyl)-amide;
2-[5-((R)-1-p-Tolyl-ethylcarbamoyl)-thiophen-2-yl]-5H-pyrrolo[2,3-b]pyrazine-7-carboxylic acid ((S)-1,2,2-trimethyl-propyl)-amide;
2-[5-(2-Methoxy-benzylcarbamoyl)-thiophen-2-yl]-5H-pyrrolo[2,3-b]pyrazine-7-carboxylic acid ((S)-1,2,2-trimethyl-propyl)-amide;
2-[5-(2,5-Dimethoxy-benzylcarbamoyl)-thiophen-2-yl]-5H-pyrrolo[2,3-b]pyrazine-7-carboxylic acid ((S)-1,2,2-trimethyl-propyl)-amide;
2-{5-[(4-Fluoro-benzyl)-methyl-carbamoyl]-thiophen-2-yl}-5H-pyrrolo[2,3-b]pyrazine-7-carboxylic acid ((S)-1,2,2-trimethyl-propyl)-amide;
2-[5-(3-Methoxy-benzylcarbamoyl)-thiophen-2-yl]-5H-pyrrolo[2,3-b]pyrazine-7-carboxylic acid ((S)-1,2,2-trimethyl-propyl)-amide;
2-[5-(3-Trifluoromethyl-benzylcarbamoyl)-thiophen-2-yl]-5H-pyrrolo[2,3-b]pyrazine-7-carboxylic acid ((S)-1,2,2-trimethyl-propyl)-amide;
2-[5-(2-Chloro-4-iodo-phenylcarbamoyl)-thiophen-2-yl]-5H-pyrrolo[2,3-b]pyrazine-7-carboxylic acid ((S)-1,2,2-trimethyl-propyl)-amide;
2-[5-((R)-1,2,2-Trimethyl-propylcarbamoyl)-thiophen-2-yl]-5H-pyrrolo[2,3-b]pyrazine-7-carboxylic acid ((S)-1,2,2-trimethyl-propyl)-amide;
2-[(5-(2,2-Dimethyl-propylcarbamoyl)-thiophen-2-yl]-5H-pyrrolo[2,3-b]pyrazine-7-carboxylic acid ((S)-1,2,2-trimethyl-propyl)-amide;
2-[5-((R)-2-Methanesulfonyl-1-methyl-ethylcarbamoyl)-thiophen-2-yl]-5H-pyrrolo[2,3-b]pyrazine-7-carboxylic acid ((S)-1,2,2-trimethyl-propyl)-amide;
2-[5-(1,1-Dioxo-hexahydro-1λ⁶-thiopyran-4-ylcarbamoyl)-thiophen-2-yl]-5H-pyrrolo[2,3-b]pyrazine-7-carboxylic acid ((S)-1,2,2-trimethyl-propyl)-amide;
2-[5-(1,1-Dioxo-1λ⁶-thiomorpholine-4-carbonyl)-thiophen-2-yl]-5H-pyrrolo[2,3-b]pyrazine-7-carboxylic acid ((S)-1,2,2-trimethyl-propyl)-amide;
2-[5-(2-Methoxy-1-methyl-ethylcarbamoyl)-thiophen-2-yl]-5H-pyrrolo[2,3-b]pyrazine-7-carboxylic acid ((S)-1,2,2-trimethyl-propyl)-amide;
2-(5-Carbamoyl-thiophen-2-yl)-5H-pyrrolo[2,3-b]pyrazine-7-carboxylic acid ((S)-1,2,2-trimethyl-propyl)-amide;
2-[(5-(3,3,3-Trifluoro-propylcarbamoyl)-thiophen-2-yl]-5H-pyrrolo[2,3-b]pyrazine-7-carboxylic acid ((S)-1,2,2-trimethyl-propyl)-amide;
2-[5-(2-Oxa-6-aza-spiro[3.3]heptane-6-carbonyl)-thiophen-2-yl]-5H-pyrrolo[2,3-b]pyrazine-7-carboxylic acid ((S)-1,2,2-trimethyl-propyl)-amide;
2-[5-(3,3-Bis-hydroxymethyl-azetidine-1-carbonyl)-thiophen-2-yl]-5H-pyrrolo[2,3-b]pyrazine-7-carboxylic acid ((S)-1,2,2-trimethyl-propyl)-amide;
2-[4-Methyl-5-(tetrahydro-pyran-4-ylcarbamoyl)-thiophen-2-yl]-5H-pyrrolo[2,3-b]pyrazine-7-carboxylic acid ((S)-1,2,2-trimethyl-propyl)-amide;
2-[5-(1,1-Dioxo-1λ⁶-thiomorpholine-4-carbonyl)-4-methyl-thiophen-2-yl]-5H-pyrrolo[2,3-b]pyrazine-7-carboxylic acid ((S)-1,2,2-trimethyl-propyl)-amide;
2-[4-Methyl-5-(2-oxa-6-aza-spiro[3.3]heptane-6-carbonyl)-thiophen-2-yl]-5H-pyrrolo[2,3-b]pyrazine-7-carboxylic acid ((S)-1,2,2-trimethyl-propyl)-amide;
2-[5-(3,3-Bis-hydroxymethyl-azetidine-1-carbonyl)-4-methyl-thiophen-2-yl]-5H-pyrrolo[2,3-b]pyrazine-7-carboxylic acid ((S)-1,2,2-trimethyl-propyl)-amide;
2-[5-(Tetrahydro-pyran-4-ylcarbamoyl)-thiophen-2-yl]-5H-pyrrolo[2,3-b]pyrazine-7-carboxylic acid ((S)-2-cyano-1,2,2-trimethyl-ethyl)-amide;
2-[5-(Piperidine-1-carbonyl)-thiophen-2-yl]-5H-pyrrolo[2,3-b]pyrazine-7-carboxylic acid ((S)-2-cyano-1,2,2-trimethyl-ethyl)-amide;
2-[5-(Tetrahydro-pyran-4-ylcarbamoyl)-thiophen-2-yl]-5H-pyrrolo[2,3-b]pyrazine-7-carboxylic acid ((S)-1,2,2-trimethyl-propyl)-amide;
2-(5-Benzylcarbamoyl-thiophen-2-yl)-5H-pyrrolo[2,3-b]pyrazine-7-carboxylic acid ((S)-1,2,2-trimethyl-propyl)-amide; and
2-[5-(3-Cyano-benzylcarbamoyl)-thiophen-2-yl]-5H-pyrrolo[2,3-b]pyrazine-7-carboxylic acid ((S)-1,2,2-trimethyl-propyl)-amide
or a pharmaceutically acceptable salt thereof.
